(19) United States
(12) Patent Application Publication
(10) Pub. No.: US 2024/0208939 A1
(43) Pub. Date: Jun. 27, 2024

(54) TREATMENT OF AUTOIMMUNE AND INFLAMMATORY DISORDERS WITH INHIBITORS OF BET FAMILY BDII BROMODOMAIN

(71) Applicants: POSEIDON INNOVATION 1, INC., New York, NY (US); THE REGENTS OF THE UNIVERSITY OF CALIFORNIA, Oakland, CA (US)

(72) Inventors: Andrew William Stamford, Chatham, NJ (US); Anthony M. Barsoti, New York, NY (US); Mehmet Kahraman, La Jolla, CA (US); Robert L. Davis, Oceanside, CA (US); Andrew Shiau, San Diego, CA (US)

(21) Appl. No.: 18/532,671

(22) Filed: Dec. 7, 2023

Related U.S. Application Data
(60) Provisional application No. 63/430,799, filed on Dec. 7, 2022.

(51) Int. Cl. C07D 401/14; A61K 31/496; A61K 45/06; A61P 17/06; A61P 29/00; C07D 403/10

(52) U.S. Cl. CPC C07D 401/14; A61K 31/496; A61K 45/06; A61P 17/06; A61P 29/00; C07D 403/10

(57) ABSTRACT

Disclosed are methods of treating an autoimmune or inflammatory disease or disorder.

### RELATED APPLICA TONS

This application claims the benefit of priority to U.S. Provisional Application No. 63/430,799, filed Dec. 7, 2022.

### BACKGROUND

Bromodomains (BDs) are protein modules that recognize acetylated lysine in histones and other proteins and these domains have been identified in 46 different human proteins (Ghimire et al. 2015). BRD2, BRD3, BRD4 and BRDT comprise the bromodomain and extra-terminal (BET) family of proteins. These proteins harbor two tandem bromodomains (BD1 and BD2) and represent major regulators of gene transcription (Sun et al. 2015; Cochran et al. 2019). Development of inhibitors that specifically target the bromodomains of BET proteins has generated interested in their therapeutic potential. Some inhibitors of bromodomains of BET proteins have been shown to possess significant anti-inflammatory properties and can modulate the expression of key inflammatory genes in both adaptive and innate immune cells, including T cells and macrophages.

### SUMMARY OF THE INVENTION

In one aspect, the present disclosure provides a method of treating an autoimmune or inflammatory disease or disorder in a subject in need thereof, comprising administering to the subject a therapeutically effective amount of a compound, wherein the compound has a structure represented by formula (I) or a pharmaceutically acceptable salt thereof:

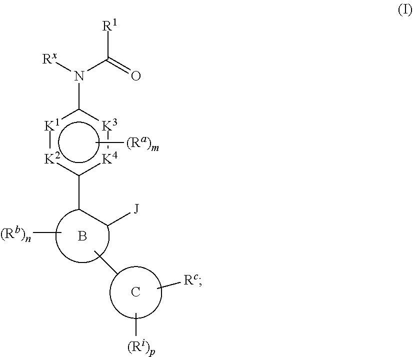

or a pharmaceutically acceptable salt thereof;
wherein:
each of K¹-K⁴ is independently CH or N;
   wherein at least one of K¹-K⁴ is CH;
Ring B represents substituted or unsubstituted phenylene or 6-membered heteroarylene;
Ring C represents substituted or unsubstituted arylene or heteroarylene;
R¹ represents alkyl, alkenyl, haloalkyl, —O(alkyl), —S(alkyl), —NH(alkyl), or —N(alkyl)₂;
R^{x} represents H, alkyl, or —C(O)alkyl;
or R¹ and R^{x}, taken together with the intervening atoms, form an optionally substituted heterocycloalkyl ring, heterocycloalkenyl ring, or heteroaryl ring;
each occurrence of R^{a} is independently selected from the group consisting of halo, —NH₂, —NH(alkyl), —NH(cycloalkyl), —N(alkyl)₂, hydroxyl, alkyl, alkoxy, cycloalkyl, cycloalkoxy, haloalkoxy, heterocycloalkoxy, cyano, aryloxy, heteroaryloxy, and haloalkyl;
J represents —OH, —O(alkyl), —OC(O)(alkyl), —OC(O)O(alkyl), —OC(O)NH(alkyl), —OC(O)N(alkyl)₂, —OCH₂OC(O)O(alkyl), or —NH₂;
each occurrence of R^{b} is independently selected from the group consisting of halo, oxo, alkyl, alkoxyl, haloalkyl, cyano, cycloalkyl, aryl, aryloxy, —OH, —NH(alkyl), —C(O)H, —CO₂(alkyl) and —CO₂H;
R^{c} represents optionally substituted heterocycloalkyl, cycloalkyl, alkyl, aryl, heteroaryl, (heterocycloalkyl)alkyl, heterocycloalkenyl, alkoxyl, alkynyl, aryloxy, haloalkyl, haloalkoxy, cycloalkoxyl, or heterocycloalkoxyl, or represents halo, —S(alkyl), —NH₂, —CO₂H, —CO₂(alkyl), or —NHCO(alkyl);
each occurrence of Rⁱ is independently halo, oxo, —S(alkyl), —NH₂, —NH(alkyl), —N(alkyl)₂, —OH, or cyano, or is selected from the group consisting of optionally substituted alkyl, haloalkyl, haloalkoxyl, alkoxyl, heterocycloalkyl, heterocyclyl, and cycloalkoxyl;
   or R^{c} and an occurrence of Rⁱ, taken together with the intervening atoms, form an optionally substituted aryl, heteroaryl, cycloalkyl, or heterocycloalkyl ring;
   or two adjacent occurrences of Rⁱ, taken together with the intervening atoms, form an optionally substituted aryl, heteroaryl, cycloalkyl, or heterocycloalkyl ring; and
   m, n, and p are each independently 0, 1, or 2.

In another aspect, the present disclosure provides a method of treating an autoimmune or inflammatory disease or disorder in a subject in need thereof, comprising administering to the subject a therapeutically effective amount of a compound, wherein the compound has a structure represented by formula (II) or a pharmaceutically acceptable salt thereof:

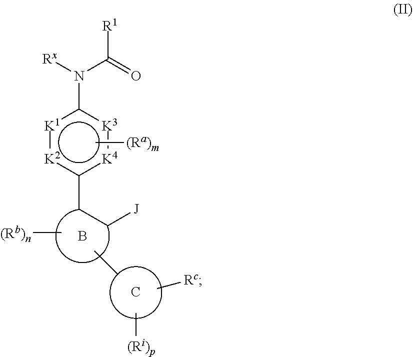

or a pharmaceutically acceptable salt thereof;
wherein:
each of K¹-K⁴ is independently CH or N;
   wherein at least one of K¹-K⁴ is CH;
Ring B represents substituted or unsubstituted phenylene or 6-membered heteroarylene;
Ring C represents substituted or unsubstituted arylene or heteroarylene;
R¹ represents alkyl, alkenyl, haloalkyl, —O(alkyl), —S(alkyl), —NH(alkyl), or —N(alkyl)₂;
R^{x} represents H, alkyl, or —C(O)alkyl;
   or R¹ and R^{x}, taken together with the intervening atoms, form an optionally substituted heterocycloalkyl ring, heterocycloalkenyl ring, or heteroaryl ring;
each occurrence of R^{a} is independently selected from the group consisting of halo, —NH₂, —NH(alkyl), —NH(cycloalkyl), —N(alkyl)₂, hydroxyl, alkyl, alkoxy, cycloalkyl, cycloalkoxy, haloalkoxy, heterocycloalkoxy, cyano, aryloxy, heteroaryloxy, and haloalkyl;
J represents —OH, —O(alkyl), —OC(O)(alkyl), —OC(O)O(alkyl), —OC(O)NH(alkyl), —OC(O)N(alkyl)₂, —OCH₂OC(O)O(alkyl), or —NH₂;
R^{j} is alkyl, alkenyl, alkynyl, aryl, heteroaryl, arylalkyl, heteroarylalkyl, cycloalkyl, heterocycloalkyl, (cycloalkyl)alkyl, or (heterocycloalkyl)alkyl;
each occurrence of R^{b} is independently selected from the group consisting of halo, oxo, alkyl, alkoxyl, haloalkyl, cyano, cycloalkyl, aryl, aryloxy, —OH, —NH(alkyl), —C(O)H, —CO₂(alkyl) and —CO₂H;
R^{c} represents H, F, or Cl;
each occurrence of Rⁱ is independently halo, oxo, —S(alkyl), —NH₂, —NH(alkyl), —N(alkyl)₂, —OH, or cyano, or is selected from the group consisting of optionally substituted alkyl, haloalkyl, haloalkoxyl, alkoxyl, heterocycloalkyl, heterocyclyl, and cycloalkoxyl;
   or two adjacent occurrences of Rⁱ, taken together with the intervening atoms, form an optionally substituted aryl, heteroaryl, cycloalkyl, or heterocycloalkyl ring; and
   m, n, and p are each independently 0, 1, or 2.

In another aspect, the present disclosure provides a method of treating an autoimmune or inflammatory disease or disorder in a subject in need thereof, comprising administering to the subject a therapeutically effective amount of a compound, wherein the compound has a structure represented by formula (III) or a pharmaceutically acceptable salt thereof:

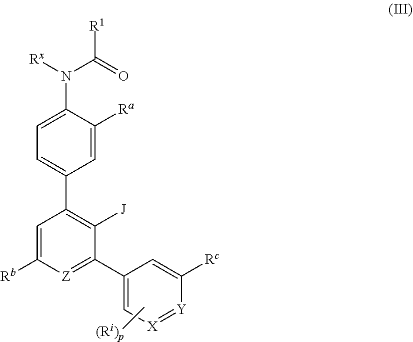

further wherein,
J is —OH, —O(alkyl), —OC(O)(alkyl), —OC(O)O(alkyl), —OC(O)NH(alkyl), —OC(O)N(alkyl)₂, —OCH₂OC(O)O(alkyl), or —NH₂;
X and Y are each independently selected from CH and N provided that at least one of X and Y is CH, or X is C(O) and Y is N(alkyl);
Z is N or CH;
R¹ is alkyl, alkenyl, haloalkyl, —O(alkyl), —S(alkyl), —NH(alkyl), or —N(alkyl)₂;
R^{x} represents H, alkyl, or —C(O)alkyl;
   or R¹ and R^{x}, taken together with the intervening atoms, form an optionally substituted heterocycloalkyl ring, heterocycloalkenyl ring, or heteroaryl ring;
each R^{a} is independently selected from the group consisting of halo, —NH₂, —NH(alkyl), —NH(cycloalkyl), —N(alkyl)₂, alkyl, alkoxy, cycloalkoxy, haloalkoxy, heterocycloalkoxy, cyano, aryloxy, heteroaryloxy, and haloalkyl;
each R^{b} is independently selected from the group consisting of halo, alkyl, alkoxyl, cyano, cycloalkyl, aryl, aryloxy, —CO₂(alkyl) and —CO₂H;
R^{c} is heterocycloalkyl, cycloalkyl, alkyl, aryl, heteroaryl, heterocyclyl, alkoxyl, alkynyl, aryloxy, haloalkyl, haloalkoxy, cycloalkoxyl, heterocycloalkoxyl, halo, —S(alkyl), —NH₂, —CO₂H, —CO₂(alkyl), or —NHCO(alkyl);
each Rⁱ is independently halo, oxo, —S(alkyl), cyano, alkyl, haloalkyl, haloalkoxyl, alkoxyl, heterocycloalkyl, heterocyclyl, or cycloalkoxyl;
   or R^{c} and an occurrence of Rⁱ, taken together with the intervening atoms, form an aryl, heteroaryl, cycloalkyl, or heterocycloalkyl ring;
   or two adjacent occurrences of Rⁱ, taken together with the intervening atoms, form an aryl, heteroaryl, cycloalkyl, or heterocycloalkyl ring; and
p is 0, 1, or 2.

### BRIEF DESCRIPTION OF THE DRAWINGS

FIG. 1A: Psoriasis was induced by daily imiquimod topical application in mice. Daily dosing of I-1 (po) significantly reduced the total psoriasis (PASI) score compared to vehicle treatment in the IMQ-mouse model of psoriasis.

FIG. 1B: Daily dosing of I-1 (po) significantly reduced the total PASI score compared to vehicle treatment, causing significant decreases in erythema.

FIG. 1C: Daily dosing of I-1 (po) significantly reduced the total PASI score compared to vehicle treatment, causing significant decreases in scaling.

FIG. 1D: Daily dosing of I-1 (po) significantly reduced the total PASI score compared to vehicle treatment, causing significant decreases in skin thickening.

FIG. 2: I-1 significantly reduced serum IL-17 levels compared to vehicle treatment alone in the the IMQ-mouse model of psoriasis. Statistical analysis was carried out by One-way ANOVA. Each group was compared with G5, and p. values les sthan 0.05 were considered significant. *p<0.05,**p<0.01.

FIG. 3A: I-1 significantly reduces mRNA of pro-inflammatory cytokines in the skin of mice treated with IMQ. Compared with vehicle treatment alone, I-1 significantly reduced mRNA levels of IL-17A from the back skin of mice treated with IMQ (the site of imiquimod topical application). Statistical analysis was carried out by One-way ANOVA. Each group was compared with G5, and p. values les sthan 0.05 were considered significant. *p<0.05, **p<0.01. G1: Vehicle+Vaseline; G5: Vehicle for I-1 (IMQ); G6: I-1 (10 mg/kg) (IMQ); G7: Dexamethasone (60 mg) (IMQ).

FIG. 3B: Compared with vehicle treatment alone, I-1 significantly reduced mRNA levels of IL-22 from the back skin of mice treated with IMQ (the site of imiquimod topical application). Statistical analysis was carried out by One-way ANOVA. Each group was compared with G5, and p. values les sthan 0.05 were considered significant. *p<0.05, **p<0.01. G1: Vehicle+Vaseline; G5: Vehicle for I-1 (IMQ); G6: I-1 (10 mg/kg) (IMQ); G7: Dexamethasone (60 mg) (IMQ).

FIG. 3C: Compared with vehicle treatment alone, I-1 significantly reduced mRNA levels of IL-23 from the back skin of mice treated with IMQ (the site of imiquimod topical application). Statistical analysis was carried out by One-way ANOVA. Each group was compared with G5, and p. values les sthan 0.05 were considered significant. *p<0.05, **p<0.01. G1: Vehicle+Vaseline; G5: Vehicle for I-1 (IMQ); G6: I-1 (10 mg/kg) (IMQ); G7: Dexamethasone (60 mg) (IMQ).

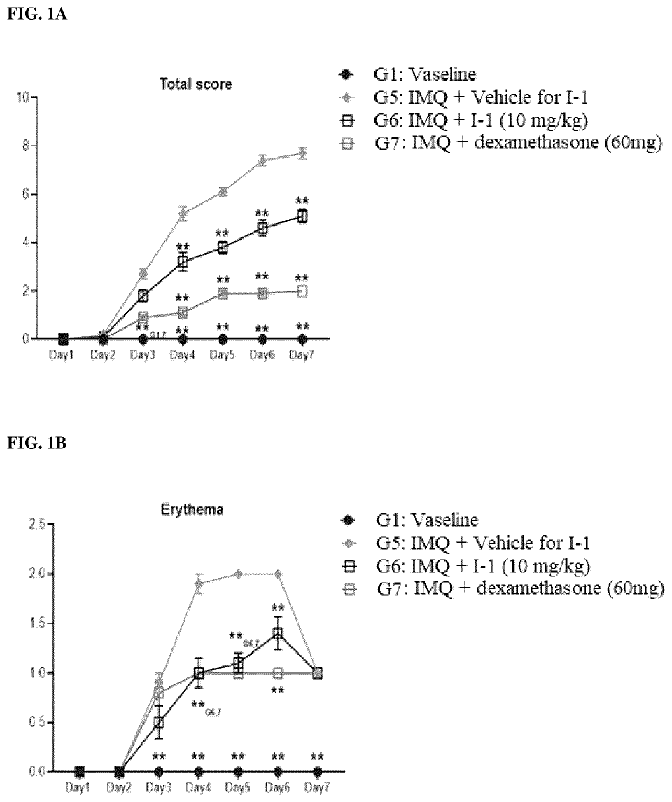

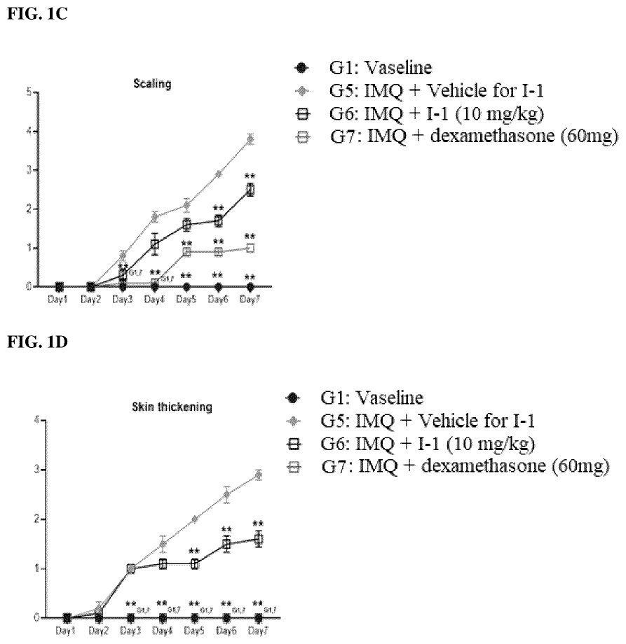

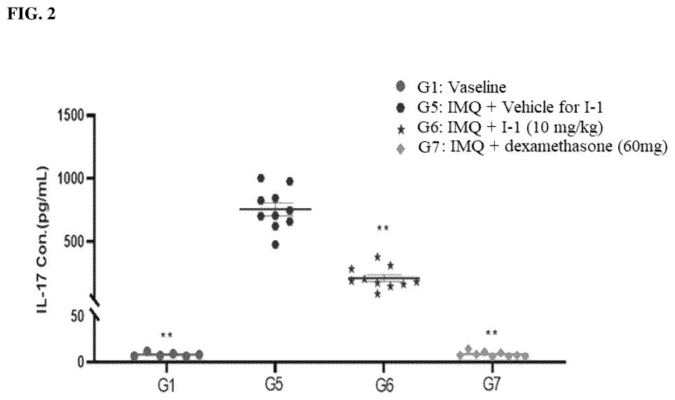

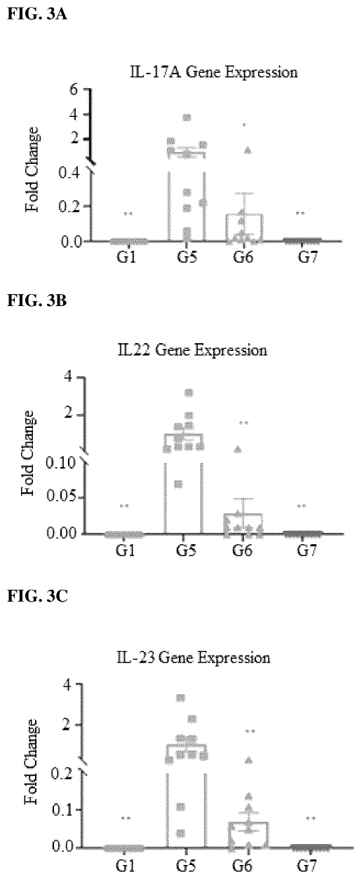

### DETAILED DESCRIPTION OF THE INVENTION

Disclosed are methods of treating an autoimmune or inflammatory disease or disorder in with a monotherapy (e.g., a BET inhibitor) or with the conjoint administration of one or more additional therapies.

In one aspect, the present disclosure provides a method of treating an autoimmune or inflammatory disease or disorder in a subject in need thereof, comprising administering to the subject a therapeutically effective amount of a compound, wherein the compound has a structure represented by formula (I) or a pharmaceutically acceptable salt thereof:

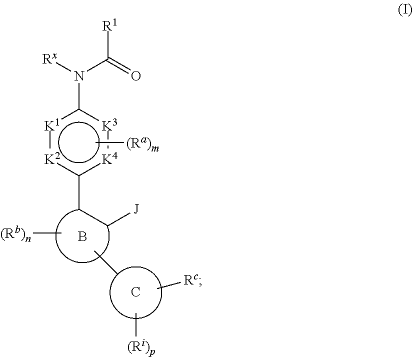

or a pharmaceutically acceptable salt thereof;
wherein:
each of K¹-K⁴ is independently CH or N;
   wherein at least one of K¹-K⁴ is CH;
Ring B represents substituted or unsubstituted phenylene or 6-membered heteroarylene;
Ring C represents substituted or unsubstituted arylene or heteroarylene;
R¹ represents alkyl, alkenyl, haloalkyl, —O(alkyl), —S(alkyl), —NH(alkyl), or —N(alkyl)₂;
R^{x} represents H, alkyl, or —C(O)alkyl;
   or R¹ and R^{x}, taken together with the intervening atoms, form an optionally substituted heterocycloalkyl ring, heterocycloalkenyl ring, or heteroaryl ring;
each occurrence of R^{a} is independently selected from the group consisting of halo, —NH₂, —NH(alkyl), —NH(cycloalkyl), —N(alkyl)₂, hydroxyl, alkyl, alkoxy, cycloalkyl, cycloalkoxy, haloalkoxy, heterocycloalkoxy, cyano, aryloxy, heteroaryloxy, and haloalkyl;
J represents —OH, —O(alkyl), —OC(O)(alkyl), —OC(O)O(alkyl), —OC(O)NH(alkyl), —OC(O)N(alkyl)₂, —OCH₂OC(O)O(alkyl), or —NH₂;
R^{j} is alkyl, alkenyl, alkynyl, aryl, heteroaryl, arylalkyl, heteroarylalkyl, cycloalkyl, heterocycloalkyl, (cycloalkyl)alkyl, or (heterocycloalkyl)alkyl;
each occurrence of R^{b} is independently selected from the group consisting of halo, oxo, alkyl, alkoxyl, haloalkyl, cyano, cycloalkyl, aryl, aryloxy, —OH, —NH(alkyl), —C(O)H, —CO₂(alkyl) and —CO₂H;
R^{c} represents optionally substituted heterocycloalkyl, cycloalkyl, alkyl, aryl, heteroaryl, (heterocycloalkyl)alkyl, heterocycloalkenyl, alkoxyl, alkynyl, aryloxy, haloalkyl, haloalkoxyl, cycloalkoxyl, or heterocycloalkoxyl, or represents halo, S(alkyl), —NH₂, —CO₂H, —CO₂(alkyl), or —NHCO(alkyl);
each occurrence of Rⁱ is independently halo, oxo, —S(alkyl), —NH₂, —NH(alkyl), —N(alkyl)₂, —OH, or cyano, or is selected from the group consisting of optionally substituted alkyl, haloalkyl, haloalkoxyl, alkoxyl, heterocycloalkyl, heterocyclyl, and cycloalkoxyl;
   or R^{c} and an occurrence of Rⁱ, taken together with the intervening atoms, form an optionally substituted aryl, heteroaryl, cycloalkyl, or heterocycloalkyl ring;
   or two adjacent occurrences of Rⁱ, taken together with the intervening atoms, form an optionally substituted aryl, heteroaryl, cycloalkyl, or heterocycloalkyl ring; and
   m, n, and p are each independently 0, 1, or 2.

In certain embodiments of the compound of formula (I):
each of K¹-K⁴ is independently CH or N;
   wherein at least one of K¹-K⁴ is CH;
Ring B represents substituted or unsubstituted phenylene or 6-membered heteroarylene;
Ring C represents substituted or unsubstituted arylene or heteroarylene;
R¹ represents alkyl, alkenyl, haloalkyl, —O(alkyl), —S(alkyl), —NH(alkyl), or —N(alkyl)₂;
R^{x} represents H, alkyl, or —C(O)alkyl;
   or R¹ and R^{x}, taken together with the intervening atoms, form an optionally substituted heterocycloalkyl ring, heterocycloalkenyl ring, or heteroaryl ring;
each occurrence of R^{a} is independently selected from the group consisting of halo, —NH₂, —NH(alkyl), —NH(cycloalkyl), —N(alkyl)₂, alkyl, alkoxy, cycloalkoxy, haloalkoxy, heterocycloalkoxy, cyano, aryloxy, heteroaryloxy, and haloalkyl;
J represents —OH, —O(alkyl), —OC(O)(alkyl), —OC(O)O(alkyl), —OC(O)NH(alkyl), —OC(O)N(alkyl)₂, —OCH₂OC(O)O(alkyl), or —NH₂;
each occurrence of R^{b} is independently selected from the group consisting of halo, alkyl, alkoxyl, cyano, cycloalkyl, aryl, aryloxy, —CO₂(alkyl) and —CO₂H;
R^{c} represents optionally substituted heterocycloalkyl, cycloalkyl, alkyl, aryl, heteroaryl, alkoxyl, alkynyl, aryloxy, haloalkyl, haloalkoxy, cycloalkoxyl, or heterocycloalkoxyl, or represents halo, —S(alkyl), —NH₂, —CO₂H, —CO₂(alkyl), or —NHCO(alkyl);
each occurrence of Rⁱ is independently halo, oxo, —S(alkyl), or cyano, or is selected from the group consisting of optionally substituted alkyl, haloalkyl, haloalkoxyl, alkoxyl, heterocycloalkyl, and cycloalkoxyl;
   or R^{c} and an occurrence of Rⁱ, taken together with the intervening atoms, form an optionally substituted aryl, heteroaryl, cycloalkyl, or heterocycloalkyl ring;
   or two adjacent occurrences of Rⁱ, taken together with the intervening atoms, form an optionally substituted aryl, heteroaryl, cycloalkyl, or heterocycloalkyl ring; and
m, n, and p are each independently 0, 1, or 2.

In certain embodiments, each of K¹-K⁴ is CH. In such embodiments, any one or more of the hydrogen atoms of the CH groups of K¹-K⁴ is optionally replaced by an occurrence of R^{a}.

For example, in a method of the present invention, the compound may have the structure of formula (Ia):

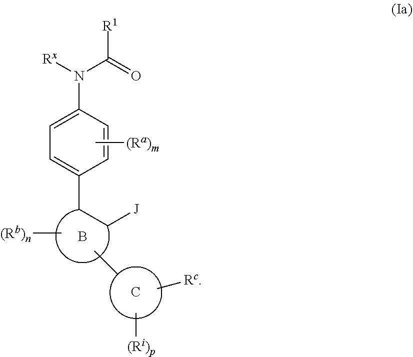

In certain embodiments, m is 0.

In certain embodiments, m is 1. For example, in a method of the present invention, the compound may have the structure of formula (Iai):

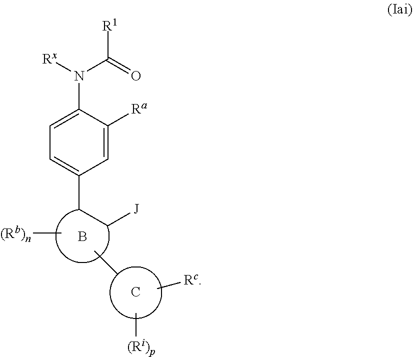

In certain embodiments, m is 2. For example, in a method of the present invention, the compound may have the structure of formula (Iaii):

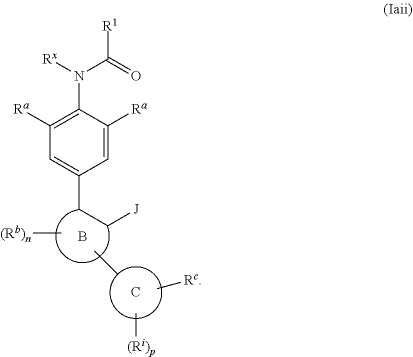

In certain embodiments, one of K¹-K⁴ is N, and the remaining of K¹-K⁴ is CH. In such embodiments, any one or more of the hydrogen atoms of the CH groups of K¹-K⁴ is optionally replaced by an occurrence of R^{a}.

For example, in certain such embodiments, K¹ is N. In alternative such embodiments, K² is N.

In certain embodiments, two of K¹-K⁴ is N, and the remaining of K¹-K⁴ is CH. In such embodiments, any one or more of the hydrogen atoms of the CH groups of K¹-K⁴ is optionally replaced by an occurrence of R^{a}.

For example, in certain such embodiments, K¹ and K³ are N. In alternative such embodiments, K¹ and K⁴ are N. Alternatively still, K¹ and K² may be N.

In certain embodiments, Ring B represents substituted or unsubstituted phenylene (i.e., a 6-membered carbocyclic aromatic ring). For example, the compound of the invention may have the structure of formula (Ib):

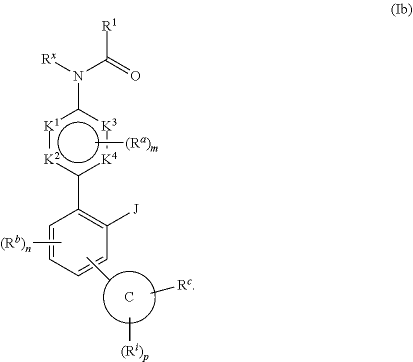

In certain embodiments, Ring C occupies a position ortho to group J. For example, in a method of the present invention, the compound may have the structure of formula (Ibi):

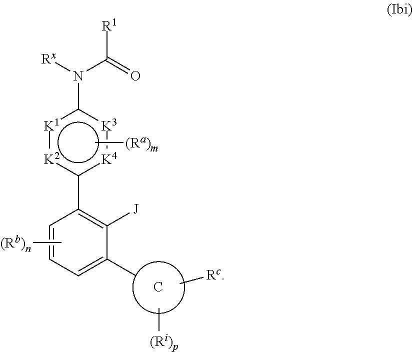

In certain such embodiments, n is 1. For example, in a method of the present invention, the compound may have the structure of formula (Ibii):

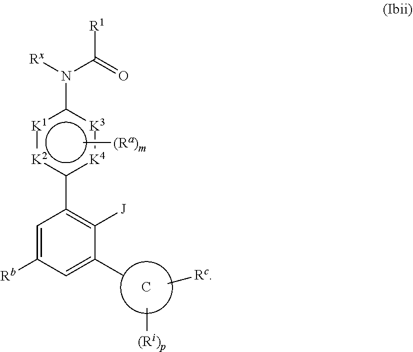

In other embodiments, n is 0. For example, in a method of the present invention, the compound may have the structure of formula (Ibiii):

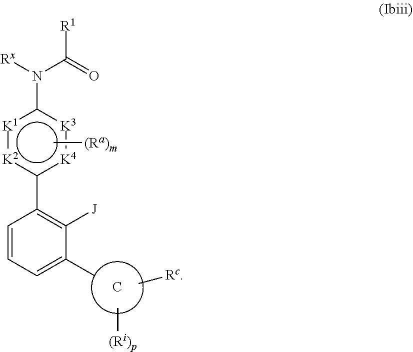

In certain embodiments, Ring B represents substituted or unsubstituted 6-membered heteroarylene.

In certain such embodiments, in a method of the present invention the compound has the structure of formula (Ibh):

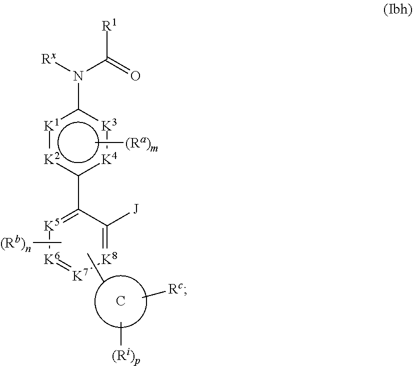

wherein each of K⁵-K⁸ is independently selected from CH and N; and at least one of K⁵-K⁸ is N.

For example, in some embodiments, K⁶ is N.

In certain embodiments, one of K⁵-K⁸ is N. In alternative embodiments, two of K⁵-K⁸ are N.

In certain embodiments, Ring B represents substituted or unsubstituted pyridine.

In certain such embodiments, in a method of the present invention the compound has the structure of formula (Ibhi):

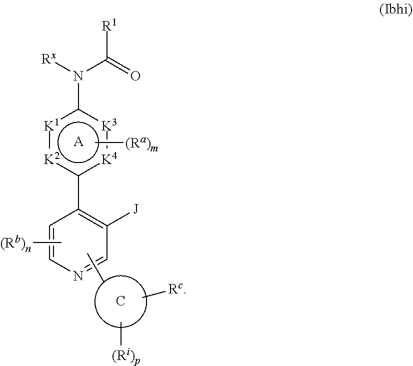

In certain embodiments, Ring C occupies a position ortho to group J. In certain such embodiments, in a method of the present invention the compound has the structure of formula (Ibhii):

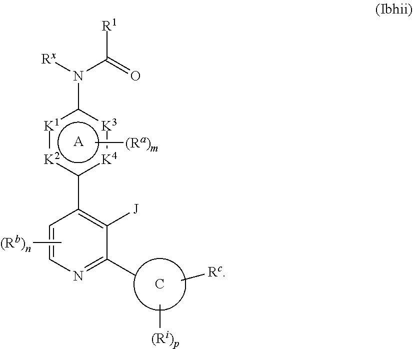

In certain such embodiments, n is 1. In certain such embodiments, in a method of the present invention the compound has the structure of formula (Ibhiii):

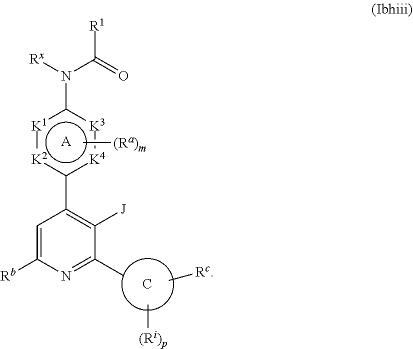

In other embodiments, n is 0. In certain such embodiments, in a method of the present invention the compound has the structure of formula (Ibhiv):

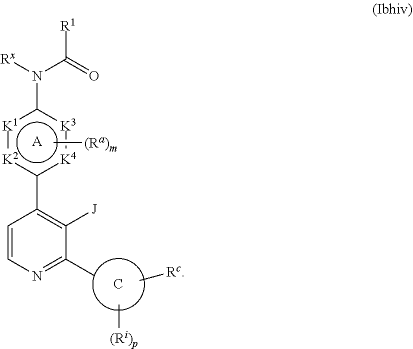

In some embodiments, Ring C represents substituted or unsubstituted heteroarylene, for example, a substituted or unsubstituted 5-membered heteroarylene. For example, Ring C can be a substituted or unsubstituted 1,2-oxazole, 1,2-thiazole, 1,2-diazole, 1,3-oxazole, 1,3-thiazole, 1,3-diazole, or 1,3,4-triazole.

Alternatively, in some embodiments, Ring C is a substituted or unsubstituted bicyclic heteroarylene group.

Alternatively, in some embodiments, Ring C represents substituted or unsubstituted 6-membered arylene (i.e., phenylene) or 6-membered heteroarylene.

In certain such embodiments, the R^{c} substituent on Ring C is in the meta position relative to Ring B.

Thus, in certain embodiments, the compound has the structure of formula (Icm):

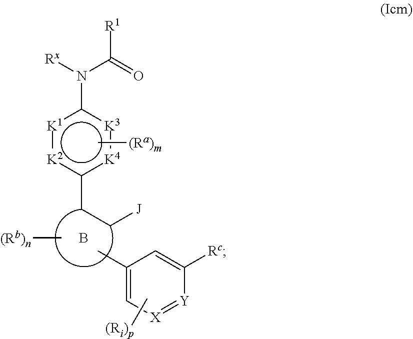

wherein X and Y are each independently selected from CH and N; and
at least one of X and Y is CH.

In certain such embodiments, Ring C represents a substituted or unsubstituted phenylene (i.e., wherein both of X and Y are CH). Accordingly, in some embodiments, in a method of the present invention the compound has the structure of formula (Icmi):

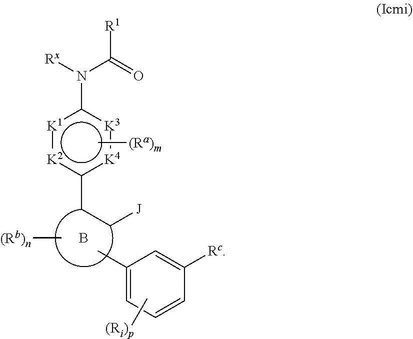

In other such embodiments, Ring C represents a substituted or unsubstituted 6-membered heteroarylene (e.g., wherein one of X and Y is N). Accordingly, in some embodiments, in a method of the present invention the compound has the structure of formula (Icmii):

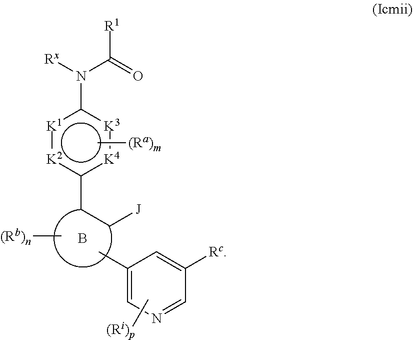

In other embodiments, in a method of the present invention the compound has the structure of formula (Icmiii):

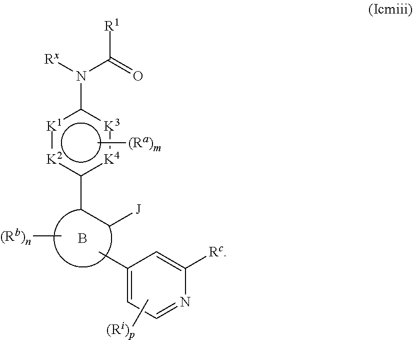

In other embodiments, the R^{c} substituent on Ring C is in the para position relative to Ring B.

In other embodiments, the R^{c} substituent on Ring C is in the ortho position relative to Ring B.

Thus, in certain embodiments, in a method of the present invention the compound has the structure of formula (Ico):

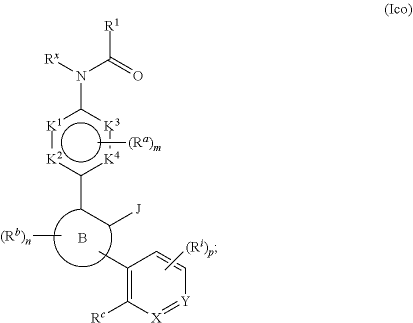

wherein X and Y are each independently selected from CH and N; and
at least one of X and Y is CH.

In certain such embodiments, Ring C represents a substituted or unsubstituted phenylene (i.e., wherein both of X and Y are CH). Accordingly, in some embodiments, in a method of the present invention the compound has the structure of formula (Icoi):

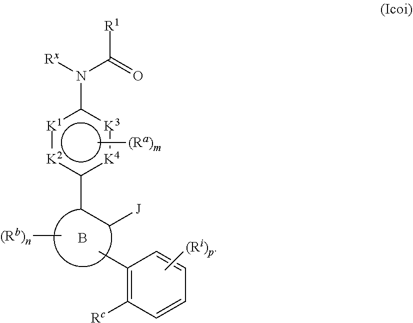

In other such embodiments, Ring C represents a substituted or unsubstituted 6-membered heteroarylene (e.g., wherein one of X and Y is N). Accordingly, in some embodiments, in a method of the present invention the compound has the structure of formula (Icoii):

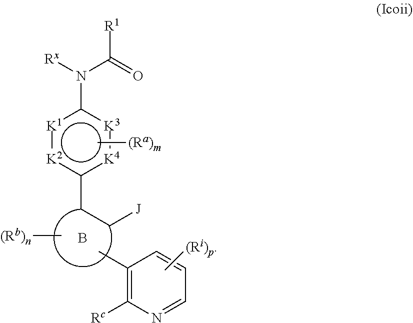

In other embodiments, in a method of the present invention the compound has the structure of formula (Icoiii):

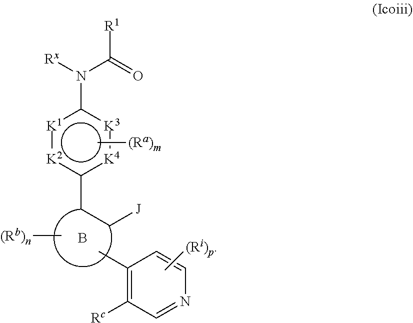

In certain embodiments, in a method of the present invention the compound has the structure of formula (Ie):

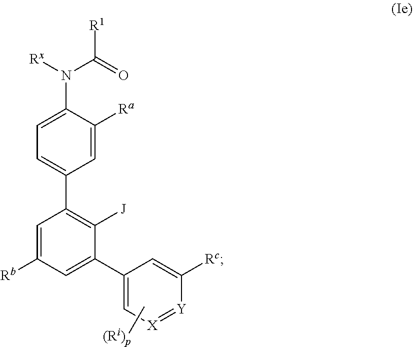

wherein X and Y are each independently selected from CH and N; and
at least one of X and Y is CH.

In certain such embodiments, in a method of the present invention the compound has the structure of formula (Iei):

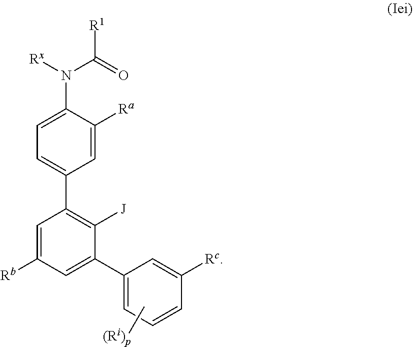

Alternatively, in some embodiments, in a method of the present invention the compound has the structure of formula (Ieii):

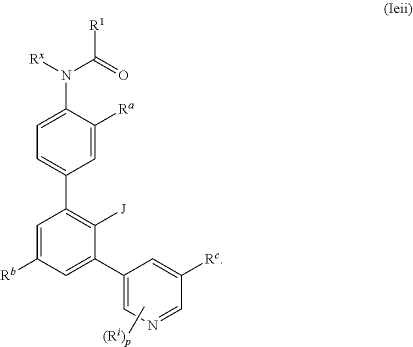

In yet further embodiments, in a method of the present invention the compound has the structure of formula (Ieiii):

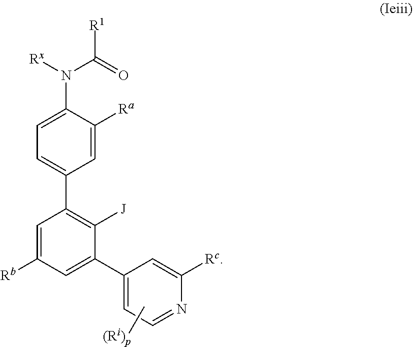

In certain alternative embodiments wherein the B ring is pyridine, in a method of the present invention the compound has the structure of formula (If):

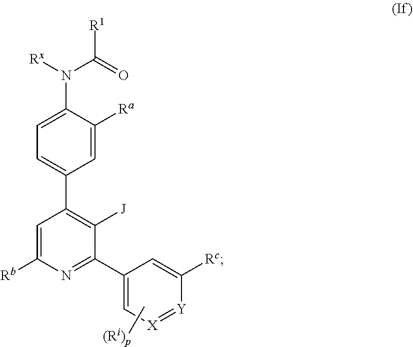

wherein X and Y are each independently selected from CH and N; and
at least one of X and Y is CH.

In certain such embodiments, in a method of the present invention the compound has the structure of formula (Ifi):

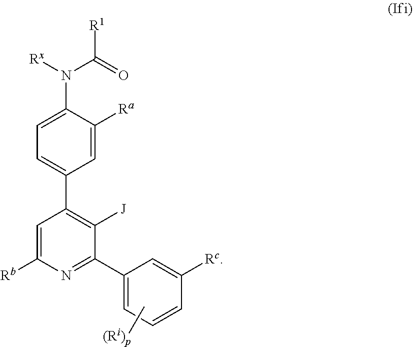

Alternatively, in a method of the present invention the compound has the structure of formula (Ifii):

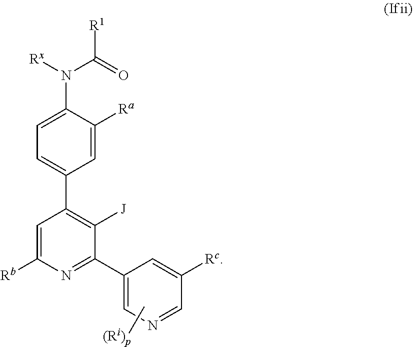

In other alternative embodiments, in a method of the present invention the compound has the structure of formula (Ifiii):

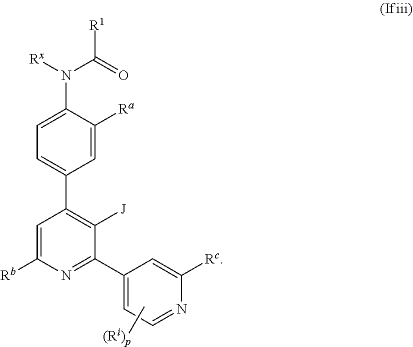

In certain alternative embodiments wherein the B ring is pyridine, in a method of the present invention the compound has the structure of formula (Ifu):

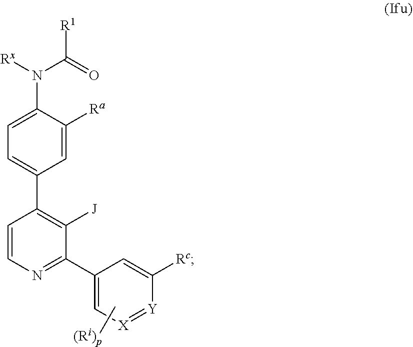

wherein X and Y are each independently selected from CH and N; and
at least one of X and Y is CH.

In certain embodiments, Ring C represents a substituted or unsubstituted 2-pyridone.

For example, in certain embodiments, in a method of the present invention the compound has the structure of formula (Igi):

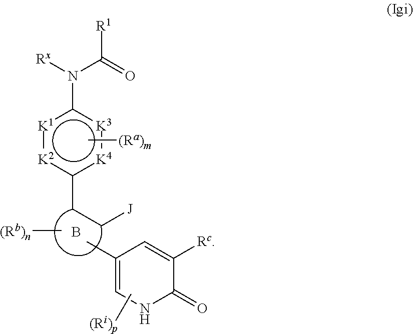

In certain such embodiments, the nitrogen of the pyridone is substituted with Rⁱ. For example, in a method of the present invention the compound has the structure of formula (Igia):

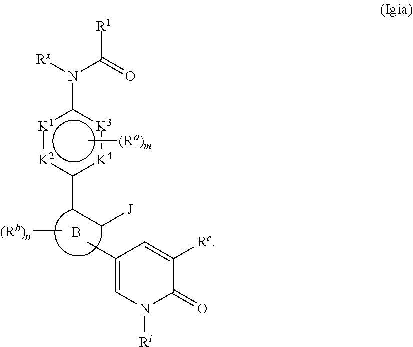

In certain such embodiments, Rings A and B are phenylene rings, and in a method of the present invention the compound has the structure of formula (Igib):

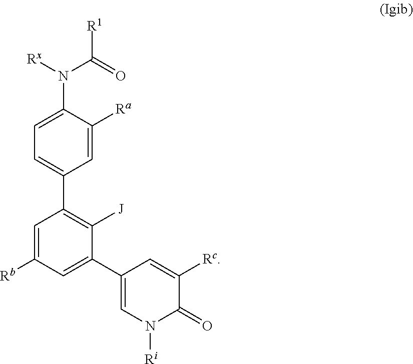

In alternative embodiments, in a method of the present invention the compound has the structure of formula (Igii):

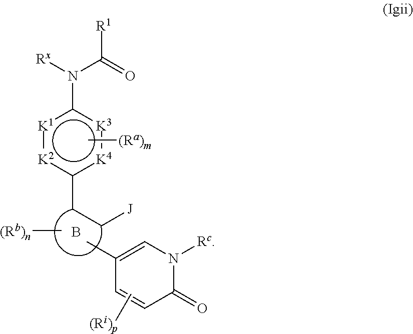

In certain such embodiments, Rings A and B are phenylene rings, and in a method of the present invention the compound has the structure of formula (Igiia):

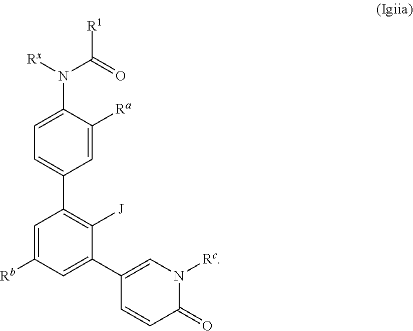

In alternative embodiments, in a method of the present invention the compound has the structure of formula (Igiii):

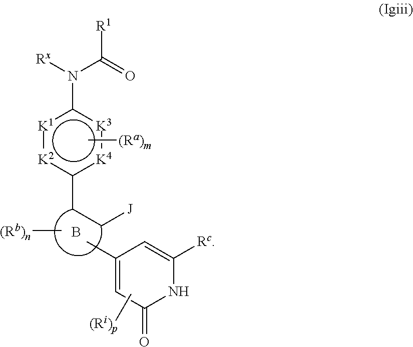

In certain such embodiments, the nitrogen of the pyridone is substituted with Rⁱ. For example, the compound of the invention may have the structure of formula (Igiiia):

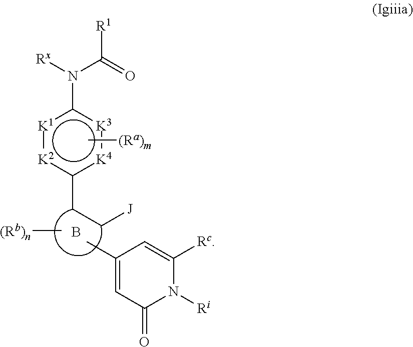

In certain such embodiments, Rings A and B are phenylene rings, and in a method of the present invention the compound has the structure of formula (Igiiib):

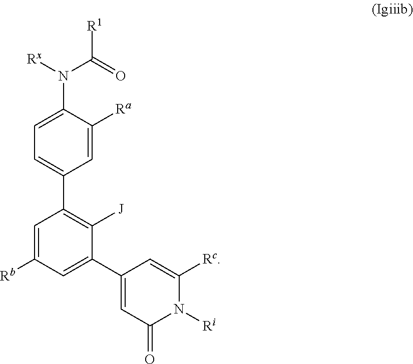

In certain embodiments, R¹ represents alkyl.

In certain embodiments, R¹ represents (C₁-C₆)alkyl, wherein at least one hydrogen atom (¹H) is replaced by a deuterium (²H or D).

In further embodiments, R¹ and R^{x}, taken together with the intervening atoms, form an optionally substituted heterocycloalkyl ring, heterocycloalkenyl ring, or heteroaryl ring.

In certain embodiments, R¹ and R^{x}, taken together with the intervening atoms, form a heterocycloalkyl ring, heterocycloalkenyl ring, or heteroaryl ring, wherein the ring is substituted by alkyl. In some embodiments, at least one hydrogen atom (¹H) of the alkyl substituent is replaced by a deuterium (²H or D).

In certain such embodiments,

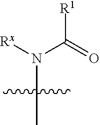

is selected from the group consisting of

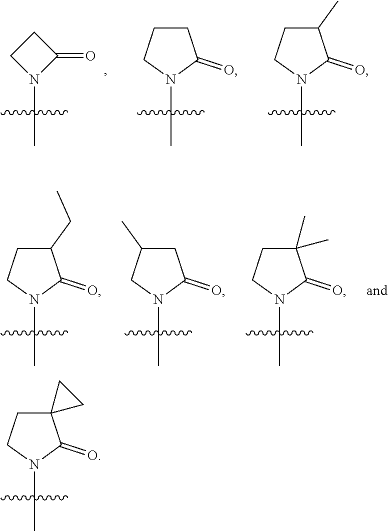

In further such embodiments,

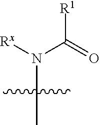

is selected from the group consisting of

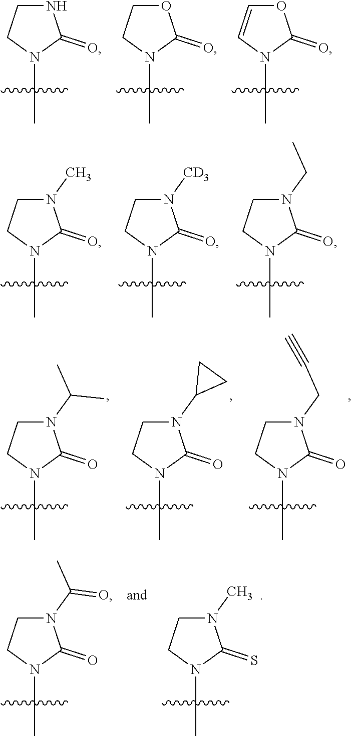

In still further embodiments,

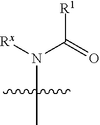

is selected from the group consisting of

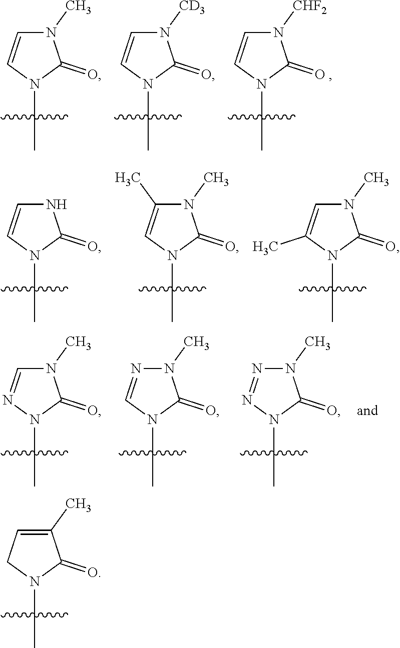

In yet further embodiments,

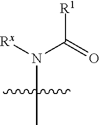

is selected from the group consisting of

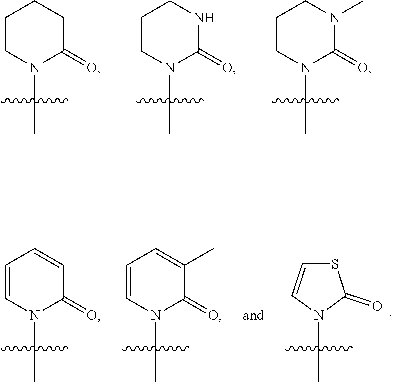

In some embodiments, m is 1.

In certain such embodiments, R^{a} is halo, alkyl, alkoxy, or cycloalkoxy. For example, R^{a} may be halo, e.g., fluoro or chloro.

In other embodiments, m is 2.

In certain such embodiments, R^{a} is independently halo, alkyl, alkoxy, or cycloalkoxy. In some embodiments, at least one occurrence of R^{a} is halo; e.g, at least one occurrence of R^{a} is fluoro or chloro.

In certain embodiments, J represents —OH or —NH₂. For example, J may be —OH.

In other embodiments, J represents an —O— bound to a prodrug moiety. For example, J may be —OC(O)(alkyl), —OC(O)O(alkyl), —OC(O)NH(alkyl), —OC(O)N(alkyl)₂, or —OCH₂OC(O)O(alkyl).

In certain embodiments, n is 0.

Alternatively, n may be 1. In certain such embodiments, R^{b} is halo or methyl. For example, R^{b} may be halo, e.g. fluoro.

In certain embodiments, p is 0. Alternatively, p may be 1. In certain such embodiments, Rⁱ is alkyl or alkoxyl.

In certain embodiments, R^{c} represents optionally substituted heterocycloalkyl. For example, in some embodiments, R^{c} may represent optionally substituted piperazinyl, piperidinyl, morpholinyl, pyrrolidinyl, azepanyl, 3,8-diazabicyclo[3.2.1]octanyl, or 2,6-diazaspiro[3.3]heptanyl.

In further embodiments, R^{c} represents piperazinyl, piperidinyl, or pyrrolidinyl, each optionally substituted by one or more substituents selected from the group consisting of amino, alkylamino, aminoalkyl, alkyl, alkoxyalkyl, halo, oxo, hydroxyl, heterocycloalkyl, (heterocycloalkyl)alkyl, cycloalkyl, (cycloalkyl)alkyl, amido, and alkoxyl.

For example, R^{c} may represent piperazinyl substituted by alkyl.

Exemplary R^{c} groups include, but are not limited to, the following:

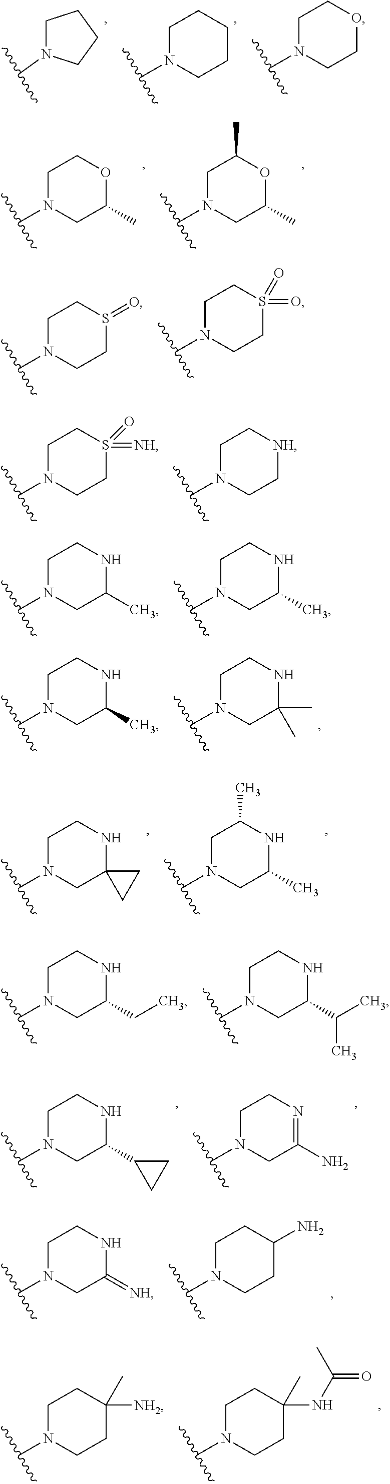

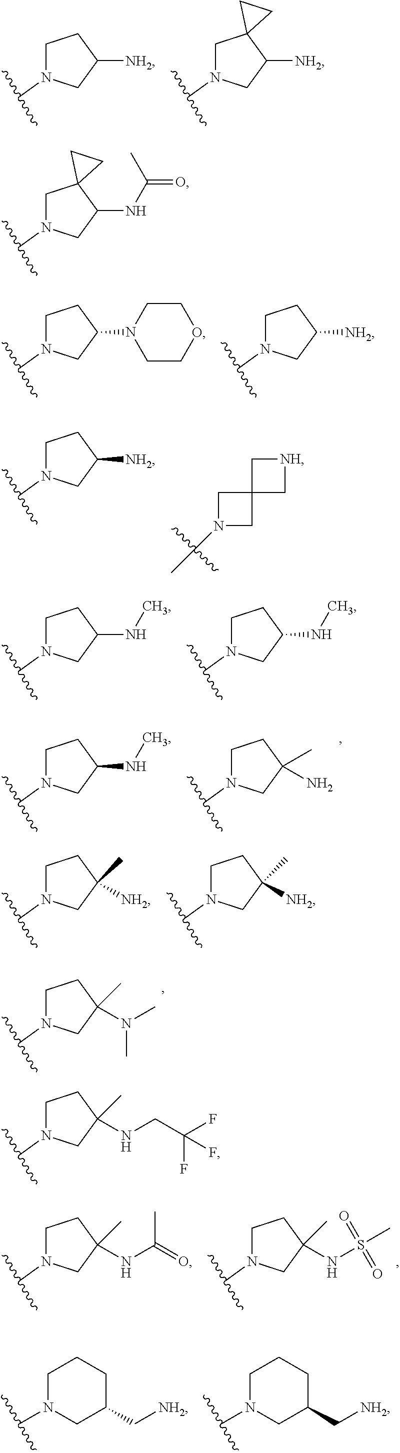

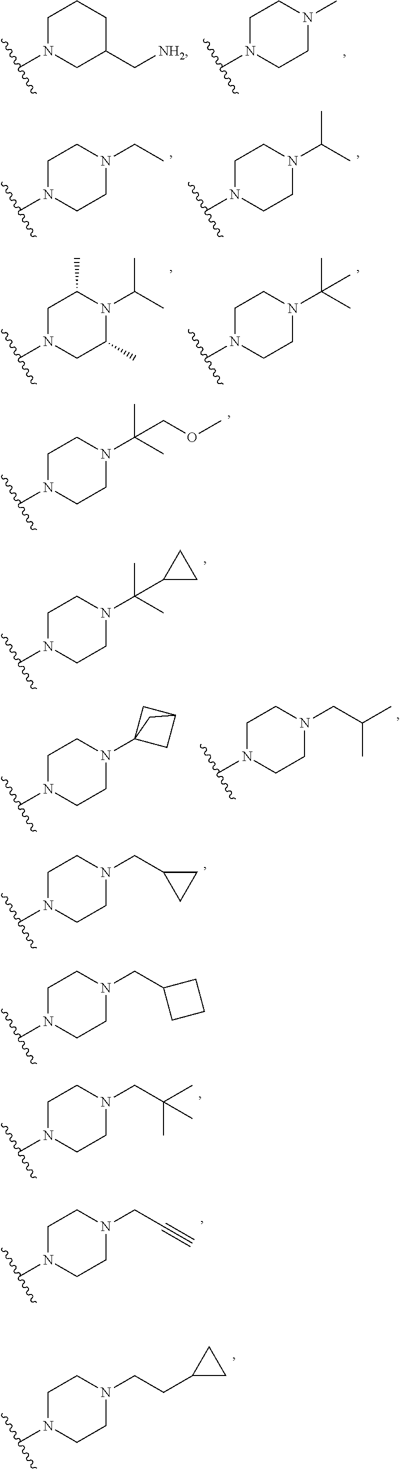

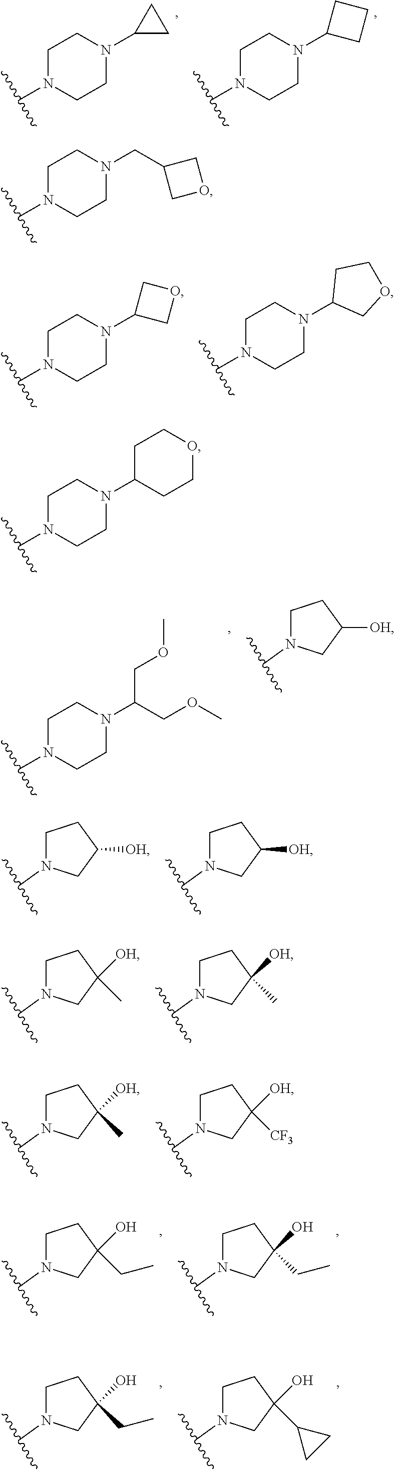

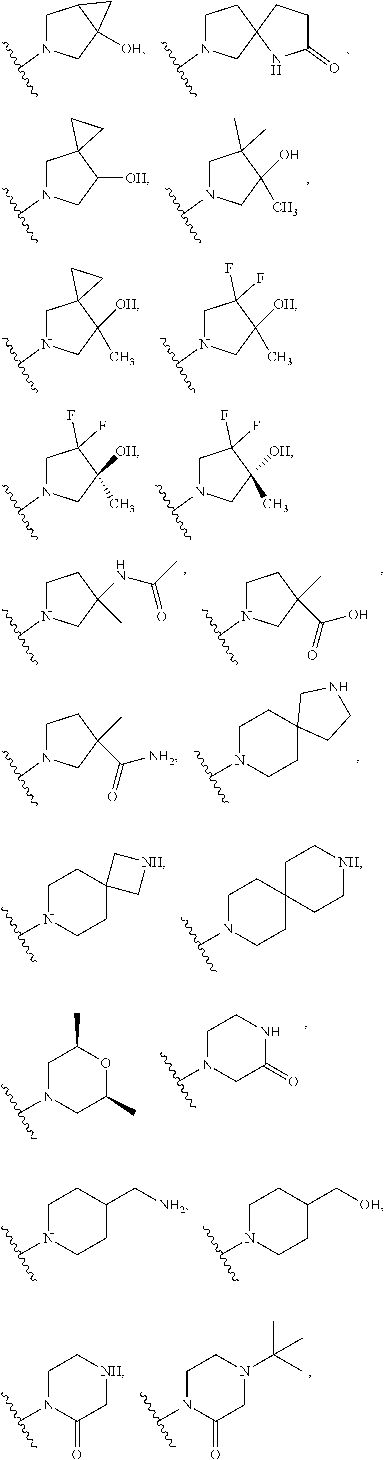

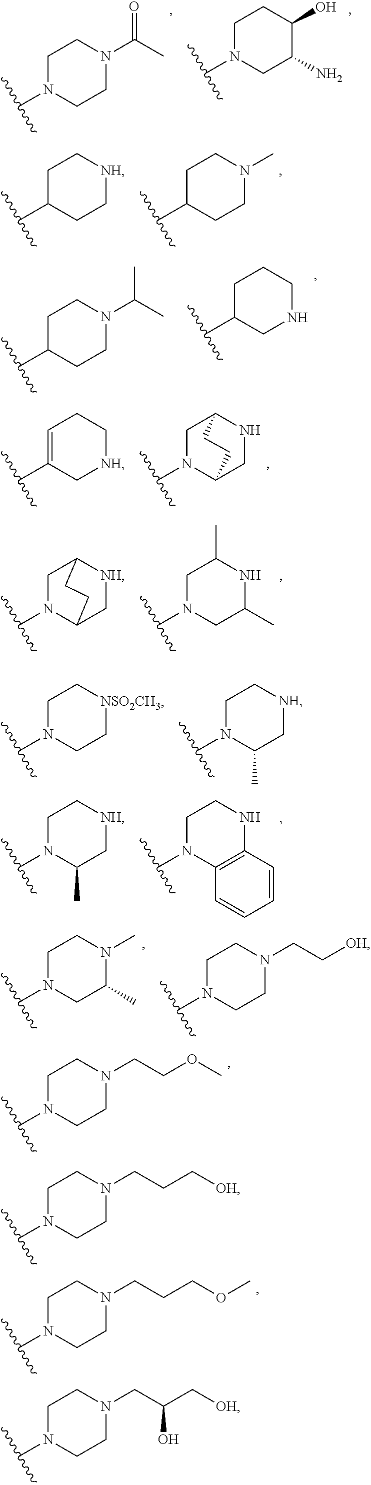

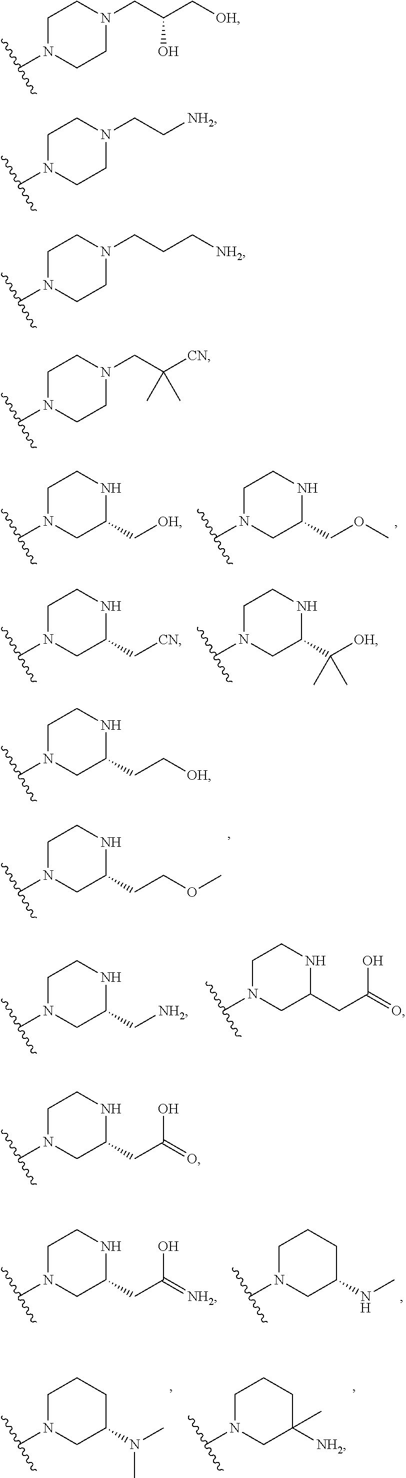

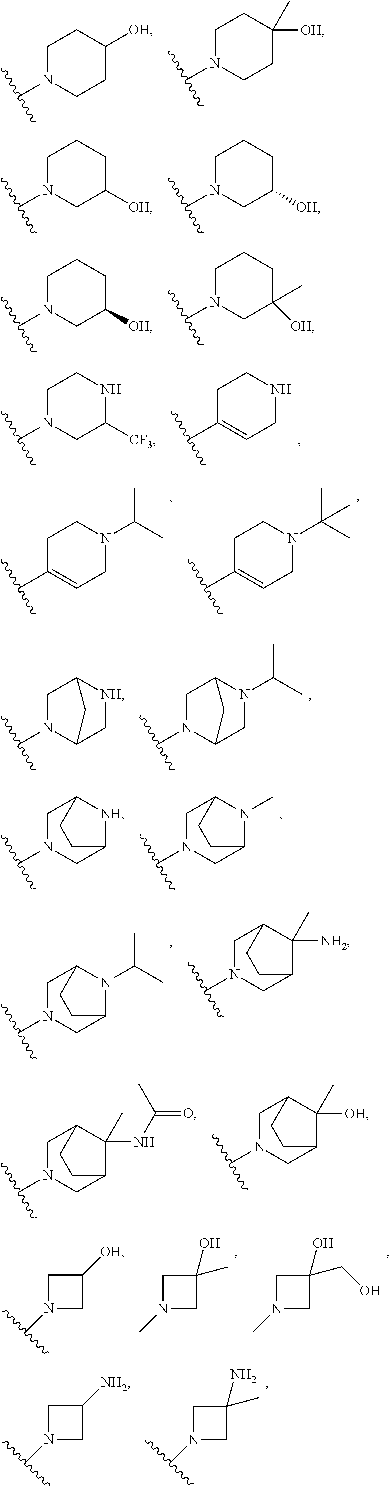

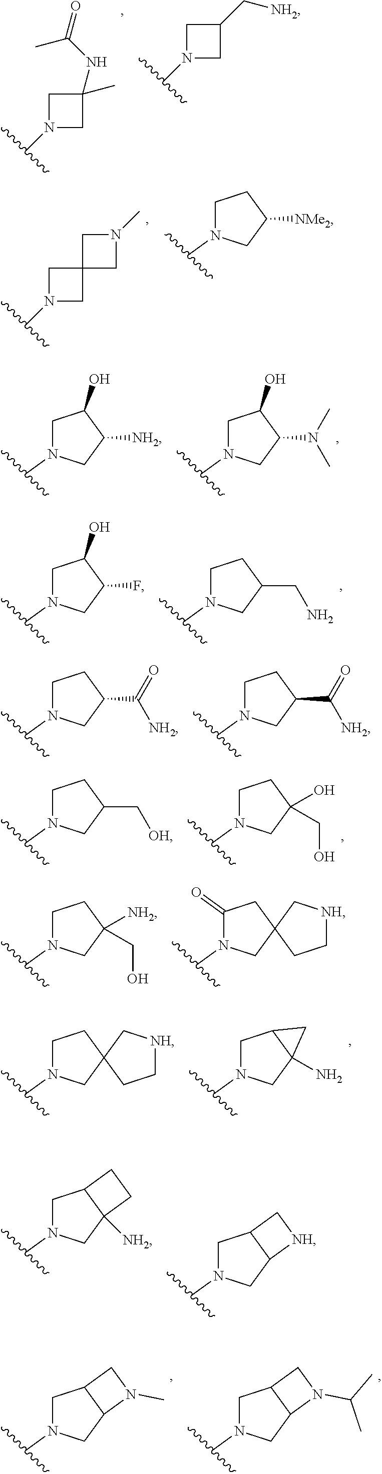

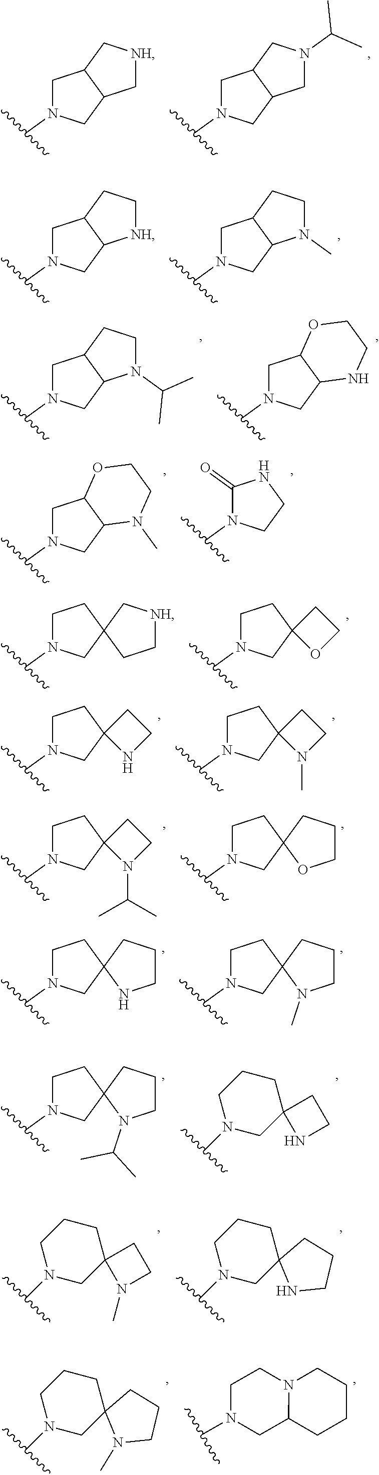

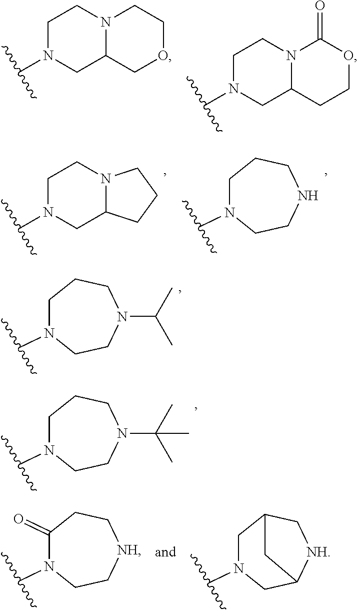

Exemplary compounds useful for a method of the present invention include:

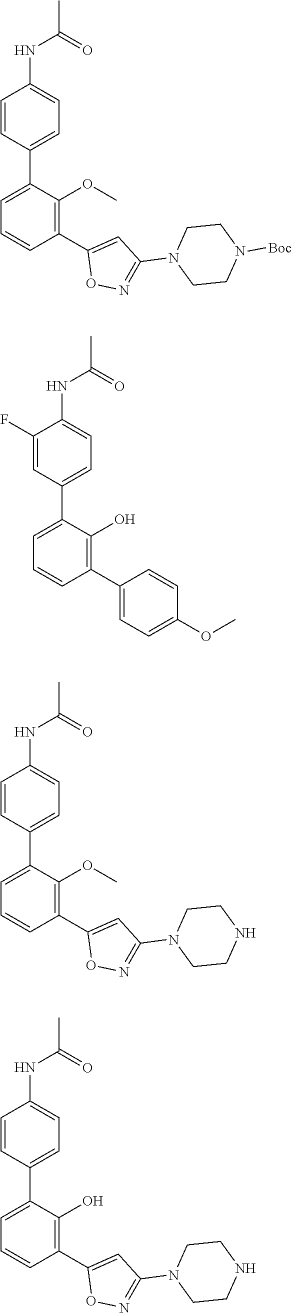 [SMILES: COc1c(-c2ccc(NC(C)=O)cc2)cccc1-c1cc(N2CCN(C(=O)OC(C)(C)C)CC2)no1 ; COc1ccc(-c2cccc(-c3ccc(NC(C)=O)c(F)c3)c2O)cc1 ; COc1c(-c2ccc(NC(C)=O)cc2)cccc1-c1cc(N2CCNCC2)no1 ; CC(=O)Nc1ccc(-c2cccc(-c3cc(N4CCNCC4)no3)c2O)cc1]

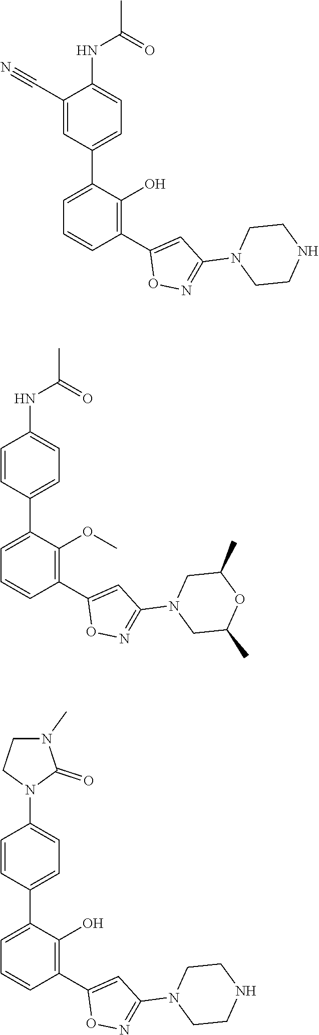 [SMILES: CC(=O)Nc1ccc(-c2cccc(-c3cc(N4CCNCC4)no3)c2O)cc1C#N ; COc1c(-c2ccc(NC(C)=O)cc2)cccc1-c1cc(N2C[C@@H](C)O[C@@H](C)C2)no1 ; CN1CCN(c2ccc(-c3cccc(-c4cc(N5CCNCC5)no4)c3O)cc2)C1=O]

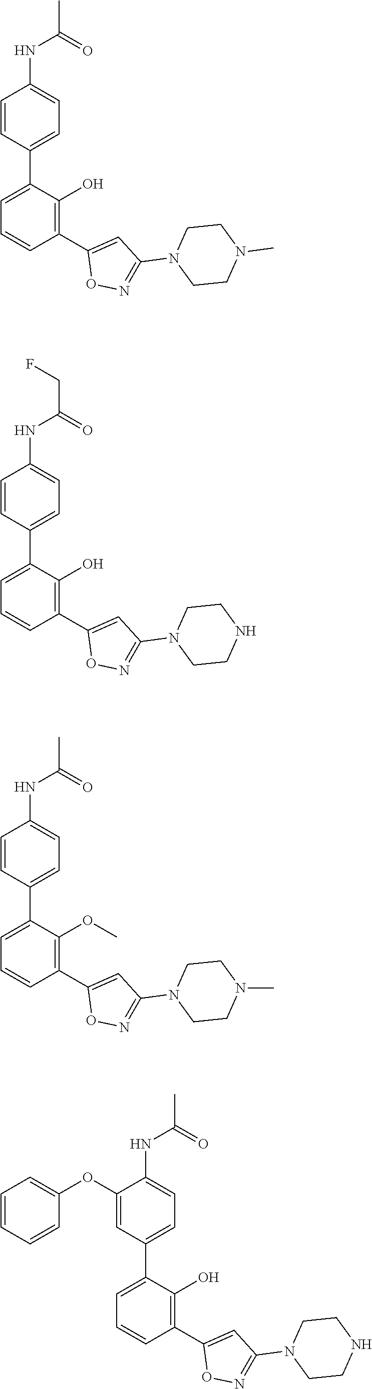 [SMILES: CC(=O)Nc1ccc(-c2cccc(-c3cc(N4CCN(C)CC4)no3)c2O)cc1 ; O=C(CF)Nc1ccc(-c2cccc(-c3cc(N4CCNCC4)no3)c2O)cc1 ; COc1c(-c2ccc(NC(C)=O)cc2)cccc1-c1cc(N2CCN(C)CC2)no1 ; CC(=O)Nc1ccc(-c2cccc(-c3cc(N4CCNCC4)no3)c2O)cc1Oc1ccccc1]

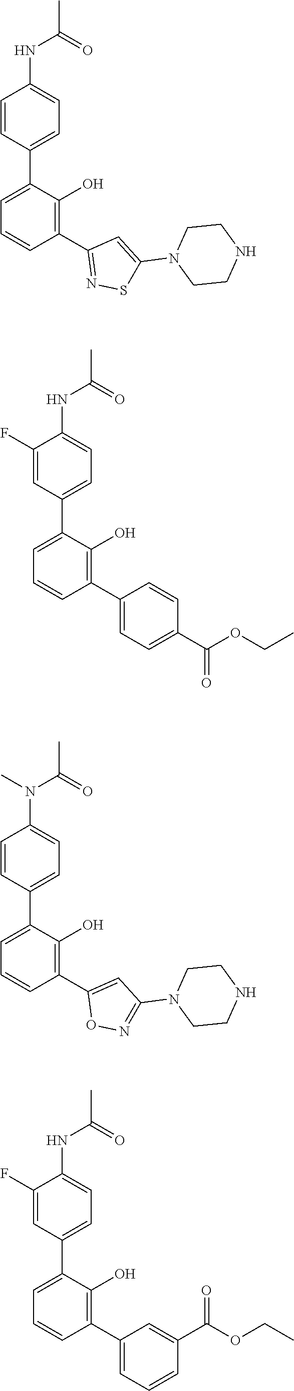 [SMILES: CC(=O)Nc1ccc(-c2cccc(-c3cc(N4CCNCC4)sn3)c2O)cc1 ; CCOC(=O)c1ccc(-c2cccc(-c3ccc(NC(C)=O)c(F)c3)c2O)cc1 ; CC(=O)N(C)c1ccc(-c2cccc(-c3cc(N4CCNCC4)no3)c2O)cc1 ; CCOC(=O)c1cccc(-c2cccc(-c3ccc(NC(C)=O)c(F)c3)c2O)c1]

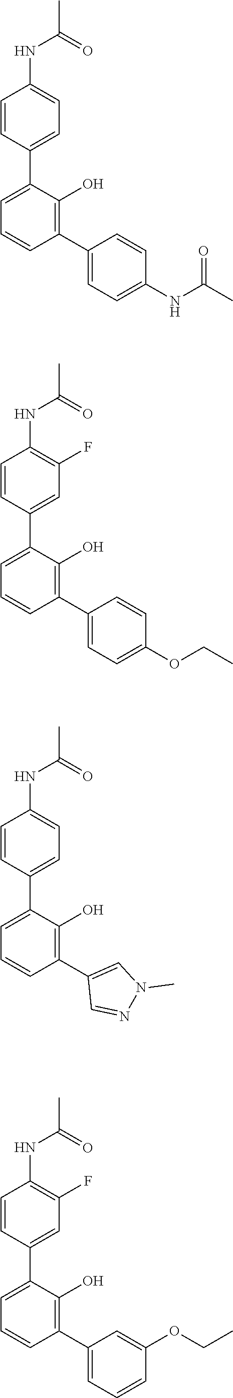 [SMILES: CC(=O)Nc1ccc(-c2cccc(-c3ccc(NC(C)=O)cc3)c2O)cc1 ; CCOc1ccc(-c2cccc(-c3ccc(NC(C)=O)c(F)c3)c2O)cc1 ; CC(=O)Nc1ccc(-c2cccc(-c3cnn(C)c3)c2O)cc1 ; CCOc1cccc(-c2cccc(-c3ccc(NC(C)=O)c(F)c3)c2O)c1]

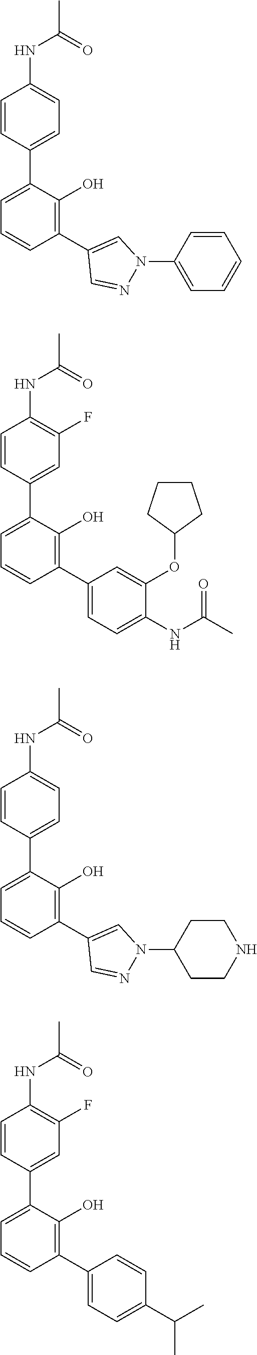 [SMILES: CC(=O)Nc1ccc(-c2cccc(-c3cnn(-c4ccccc4)c3)c2O)cc1 ; CC(=O)Nc1ccc(-c2cccc(-c3ccc(NC(C)=O)c(OC4CCCC4)c3)c2O)cc1F ; CC(=O)Nc1ccc(-c2cccc(-c3cnn(C4CCNCC4)c3)c2O)cc1 ; CC(=O)Nc1ccc(-c2cccc(-c3ccc(C(C)C)cc3)c2O)cc1F]

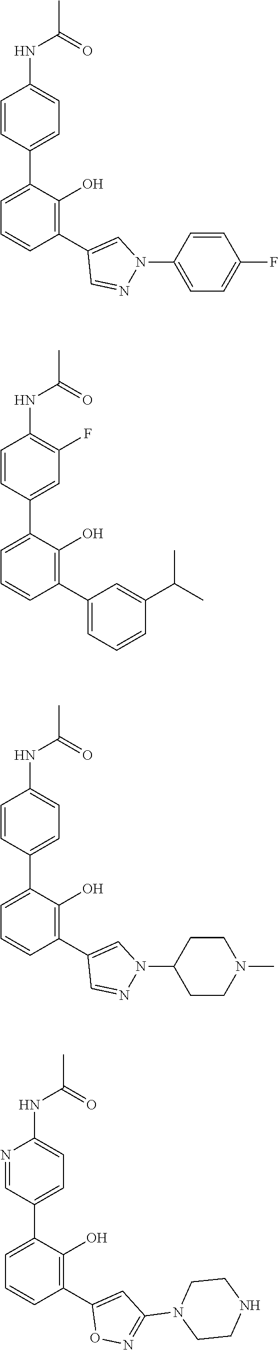 [SMILES: CC(=O)Nc1ccc(-c2cccc(-c3cnn(-c4ccc(F)cc4)c3)c2O)cc1 ; CC(=O)Nc1ccc(-c2cccc(-c3cccc(C(C)C)c3)c2O)cc1F ; CC(=O)Nc1ccc(-c2cccc(-c3cnn(C4CCN(C)CC4)c3)c2O)cc1 ; CC(=O)Nc1ccc(-c2cccc(-c3cc(N4CCNCC4)no3)c2O)cn1]

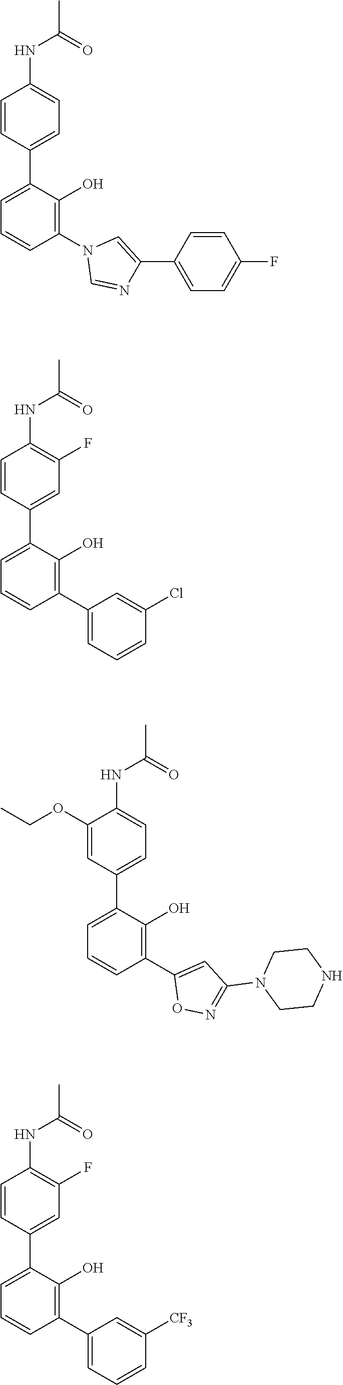 [SMILES: CC(=O)Nc1ccc(-c2cccc(-n3cnc(-c4ccc(F)cc4)c3)c2O)cc1 ; CC(=O)Nc1ccc(-c2cccc(-c3cccc(Cl)c3)c2O)cc1F ; CCOc1cc(-c2cccc(-c3cc(N4CCNCC4)no3)c2O)ccc1NC(C)=O ; CC(=O)Nc1ccc(-c2cccc(-c3cccc(C(F)(F)F)c3)c2O)cc1F]

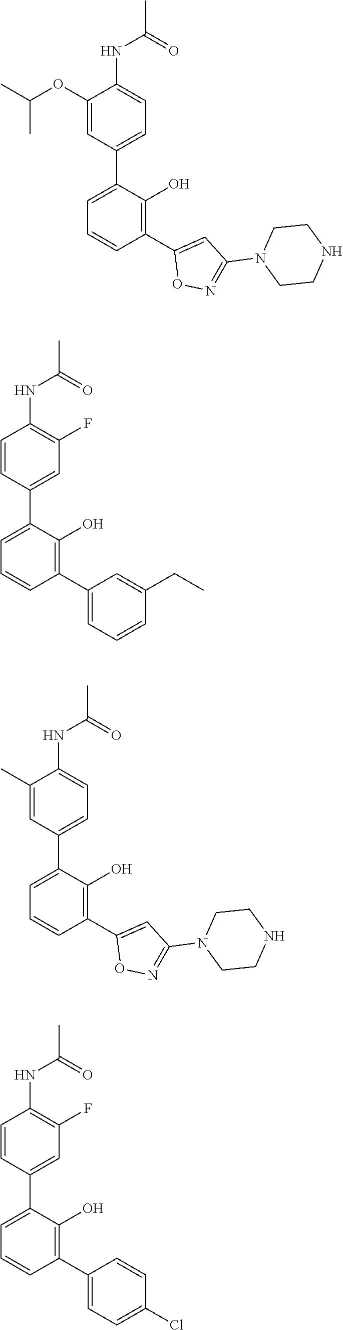

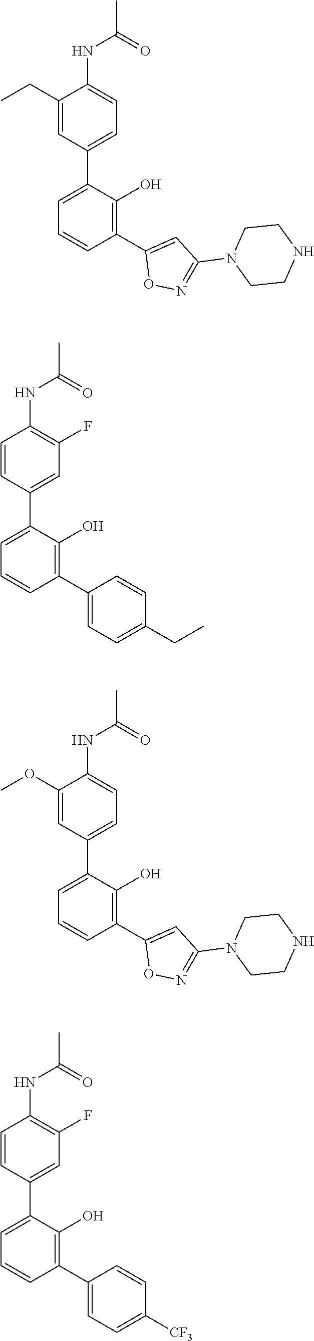

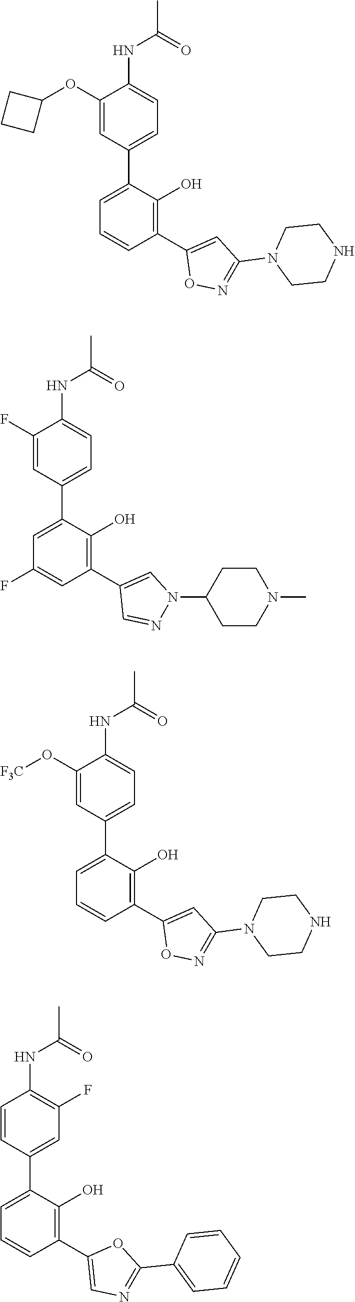 [SMILES: CC(=O)Nc1ccc(-c2cccc(-c3cc(N4CCNCC4)no3)c2O)cc1OC1CCC1 ; CC(=O)Nc1ccc(-c2cc(F)cc(-c3cnn(C4CCN(C)CC4)c3)c2O)cc1F ; CC(=O)Nc1ccc(-c2cccc(-c3cc(N4CCNCC4)no3)c2O)cc1OC(F)(F)F ; CC(=O)Nc1ccc(-c2cccc(-c3cnc(-c4ccccc4)o3)c2O)cc1F]

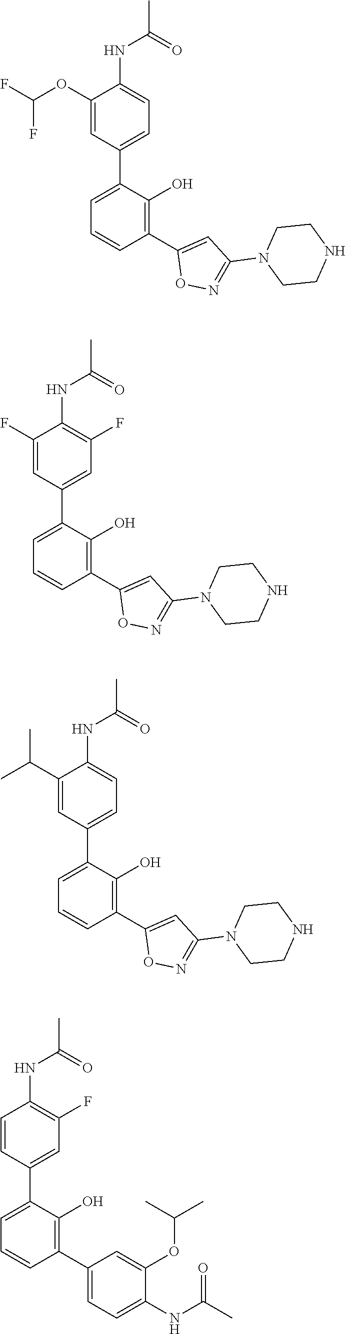 [SMILES: CC(=O)Nc1ccc(-c2cccc(-c3cc(N4CCNCC4)no3)c2O)cc1OC(F)F ; CC(=O)Nc1c(F)cc(-c2cccc(-c3cc(N4CCNCC4)no3)c2O)cc1F ; CC(=O)Nc1ccc(-c2cccc(-c3cc(N4CCNCC4)no3)c2O)cc1C(C)C ; CC(=O)Nc1ccc(-c2cccc(-c3ccc(NC(C)=O)c(OC(C)C)c3)c2O)cc1F]

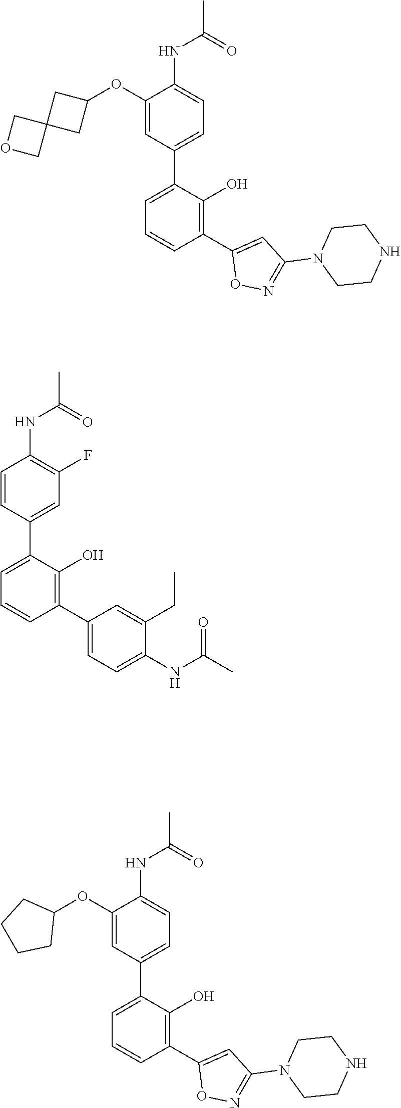 [SMILES: CC(=O)Nc1ccc(-c2cccc(-c3cc(N4CCNCC4)no3)c2O)cc1OC1CC2(COC2)C1 ; CCc1cc(-c2cccc(-c3ccc(NC(C)=O)c(F)c3)c2O)ccc1NC(C)=O ; CC(=O)Nc1ccc(-c2cccc(-c3cc(N4CCNCC4)no3)c2O)cc1OC1CCCC1]

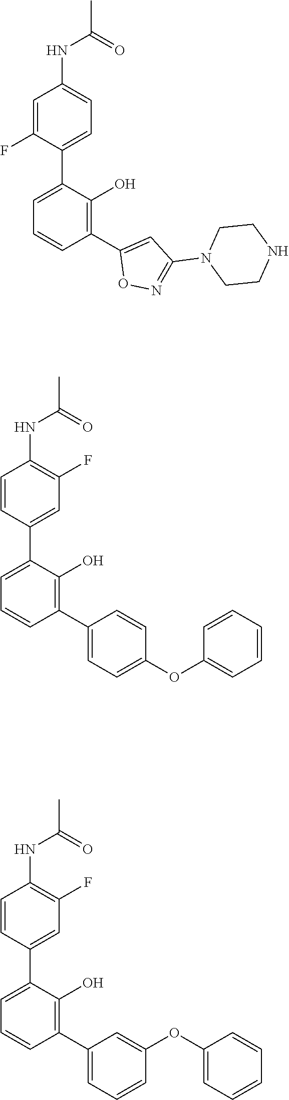 [SMILES: CC(=O)Nc1ccc(-c2cccc(-c3cc(N4CCNCC4)no3)c2O)c(F)c1 ; CC(=O)Nc1ccc(-c2cccc(-c3ccc(Oc4ccccc4)cc3)c2O)cc1F ; CC(=O)Nc1ccc(-c2cccc(-c3cccc(Oc4ccccc4)c3)c2O)cc1F]

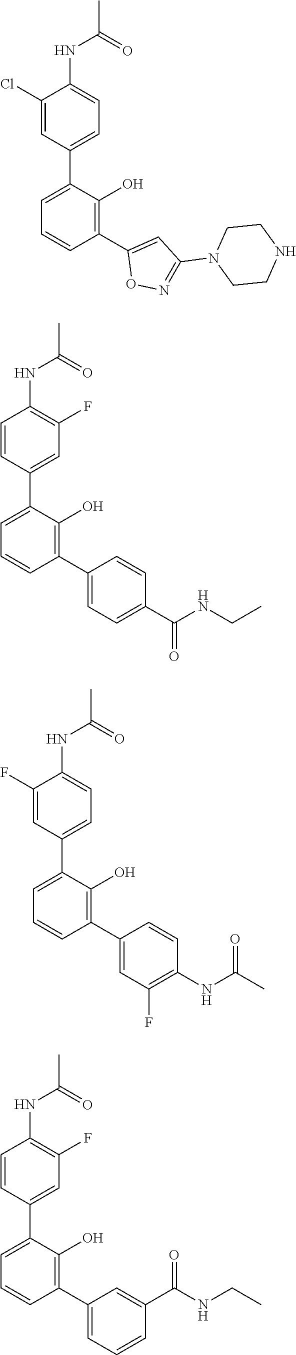 [SMILES: CC(=O)Nc1ccc(-c2cccc(-c3cc(N4CCNCC4)no3)c2O)cc1Cl ; CCNC(=O)c1ccc(-c2cccc(-c3ccc(NC(C)=O)c(F)c3)c2O)cc1 ; CC(=O)Nc1ccc(-c2cccc(-c3ccc(NC(C)=O)c(F)c3)c2O)cc1F ; CCNC(=O)c1cccc(-c2cccc(-c3ccc(NC(C)=O)c(F)c3)c2O)c1]

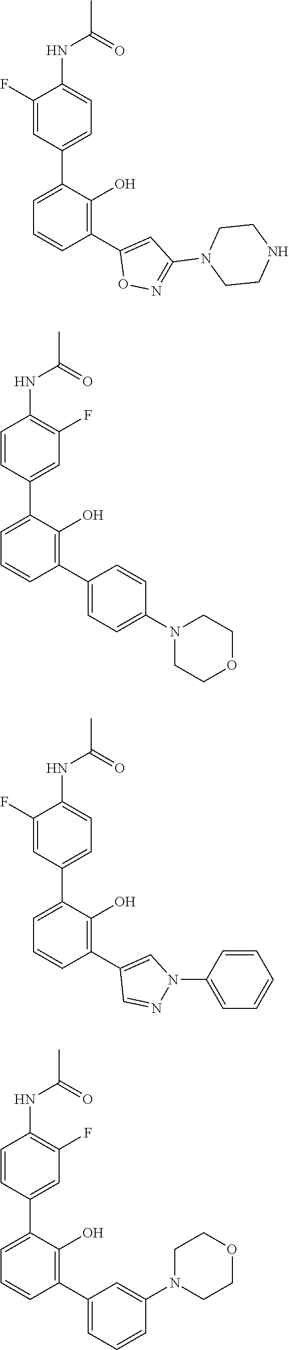 [SMILES: CC(=O)Nc1ccc(-c2cccc(-c3cc(N4CCNCC4)no3)c2O)cc1F ; CC(=O)Nc1ccc(-c2cccc(-c3ccc(N4CCOCC4)cc3)c2O)cc1F ; CC(=O)Nc1ccc(-c2cccc(-c3cnn(-c4ccccc4)c3)c2O)cc1F ; CC(=O)Nc1ccc(-c2cccc(-c3cccc(N4CCOCC4)c3)c2O)cc1F]

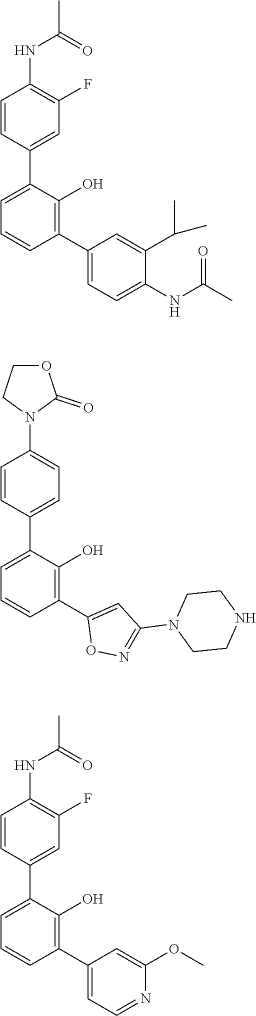 [SMILES: CC(=O)Nc1ccc(-c2cccc(-c3ccc(NC(C)=O)c(C(C)C)c3)c2O)cc1F ; O=C1OCCN1c1ccc(-c2cccc(-c3cc(N4CCNCC4)no3)c2O)cc1 ; COc1cc(-c2cccc(-c3ccc(NC(C)=O)c(F)c3)c2O)ccn1]

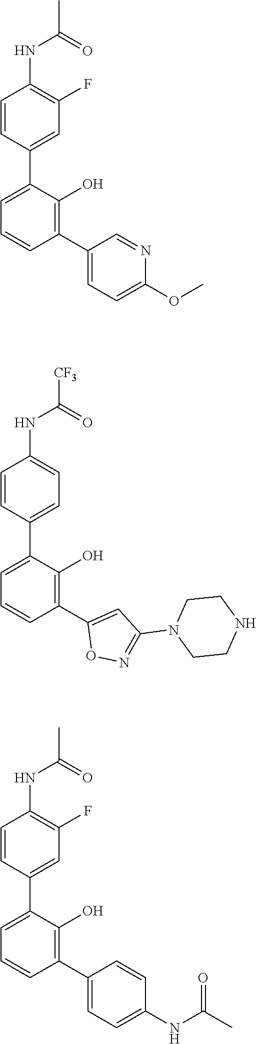 [SMILES: COc1ccc(-c2cccc(-c3ccc(NC(C)=O)c(F)c3)c2O)cn1 ; O=C(Nc1ccc(-c2cccc(-c3cc(N4CCNCC4)no3)c2O)cc1)C(F)(F)F ; CC(=O)Nc1ccc(-c2cccc(-c3ccc(NC(C)=O)c(F)c3)c2O)cc1]

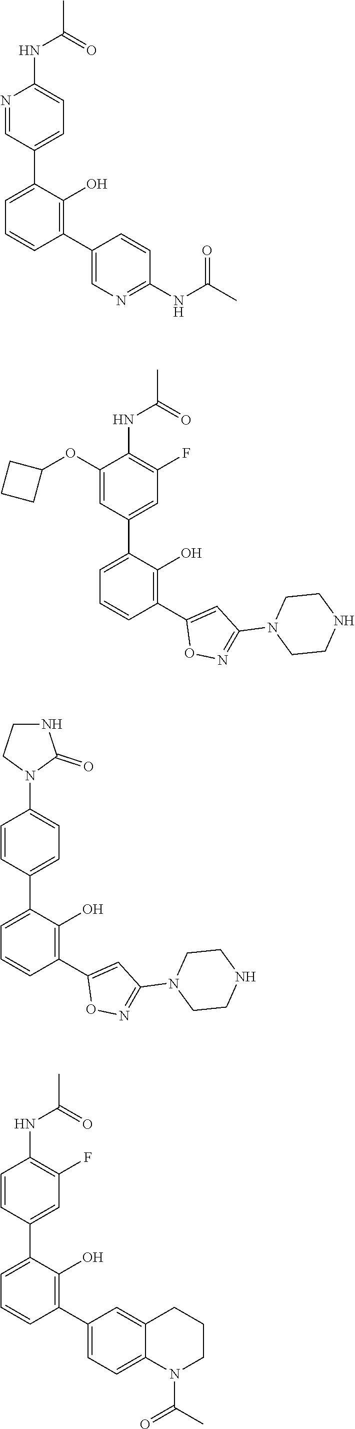 [SMILES: CC(=O)Nc1ccc(-c2cccc(-c3ccc(NC(C)=O)nc3)c2O)cn1 ; CC(=O)Nc1c(F)cc(-c2cccc(-c3cc(N4CCNCC4)no3)c2O)cc1OC1CCC1 ; O=C1NCCN1c1ccc(-c2cccc(-c3cc(N4CCNCC4)no3)c2O)cc1 ; CC(=O)Nc1ccc(-c2cccc(-c3ccc4c(c3)CCCN4C(C)=O)c2O)cc1F]

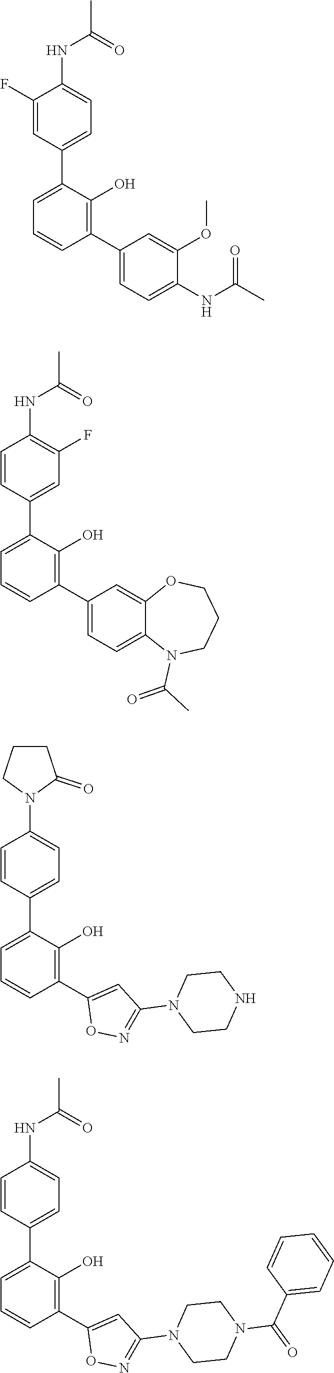 [SMILES: COc1cc(-c2cccc(-c3ccc(NC(C)=O)c(F)c3)c2O)ccc1NC(C)=O ; CC(=O)Nc1ccc(-c2cccc(-c3ccc4c(c3)OCCCN4C(C)=O)c2O)cc1F ; O=C1CCCN1c1ccc(-c2cccc(-c3cc(N4CCNCC4)no3)c2O)cc1 ; CC(=O)Nc1ccc(-c2cccc(-c3cc(N4CCN(C(=O)c5ccccc5)CC4)no3)c2O)cc1]

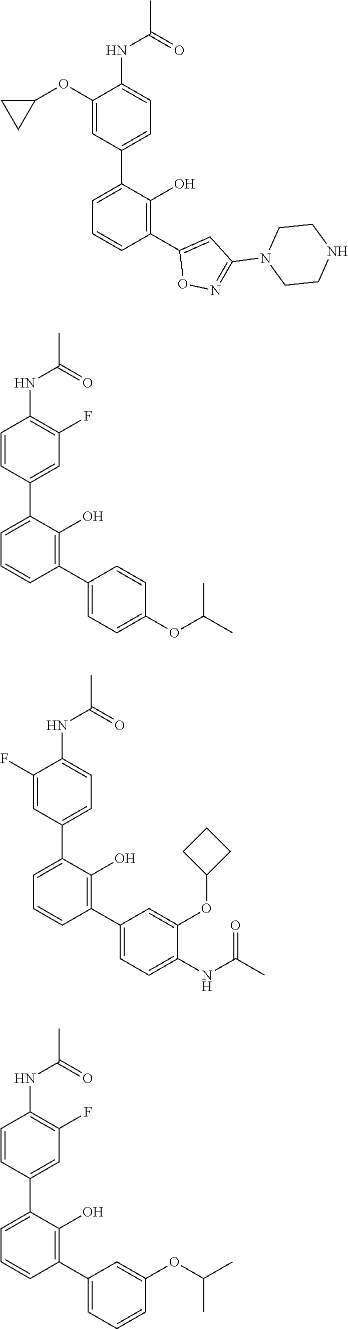 [SMILES: CC(=O)Nc1ccc(-c2cccc(-c3cc(N4CCNCC4)no3)c2O)cc1OC1CC1 ; CC(=O)Nc1ccc(-c2cccc(-c3ccc(OC(C)C)cc3)c2O)cc1F ; CC(=O)Nc1ccc(-c2cccc(-c3ccc(NC(C)=O)c(OC4CCC4)c3)c2O)cc1F ; CC(=O)Nc1ccc(-c2cccc(-c3cccc(OC(C)C)c3)c2O)cc1F]

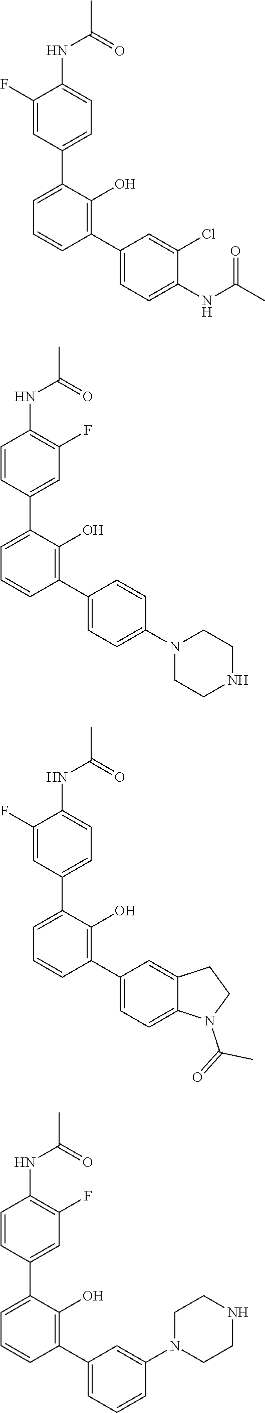 [SMILES: CC(=O)Nc1ccc(-c2cccc(-c3ccc(NC(C)=O)c(Cl)c3)c2O)cc1F ; CC(=O)Nc1ccc(-c2cccc(-c3ccc(N4CCNCC4)cc3)c2O)cc1F ; CC(=O)Nc1ccc(-c2cccc(-c3ccc4c(c3)CCN4C(C)=O)c2O)cc1F ; CC(=O)Nc1ccc(-c2cccc(-c3cccc(N4CCNCC4)c3)c2O)cc1F]

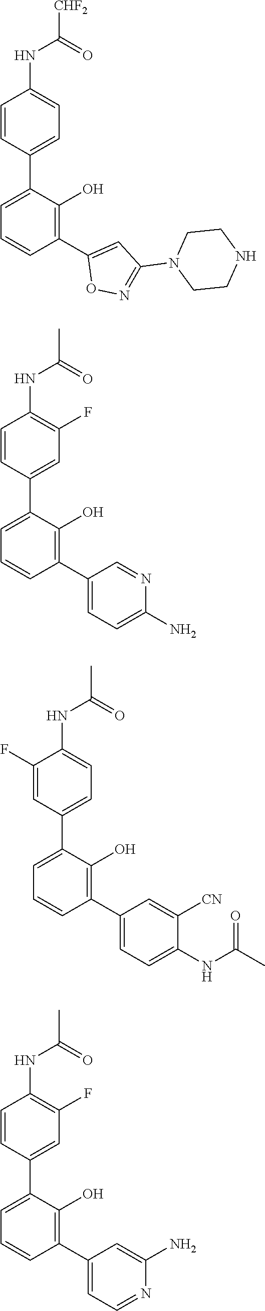

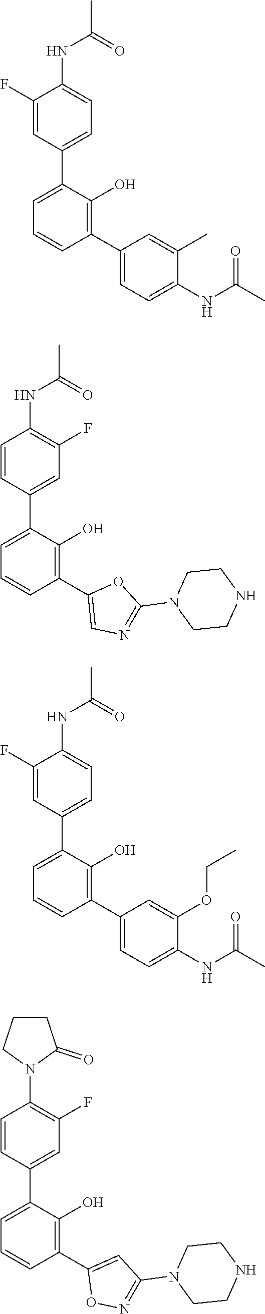

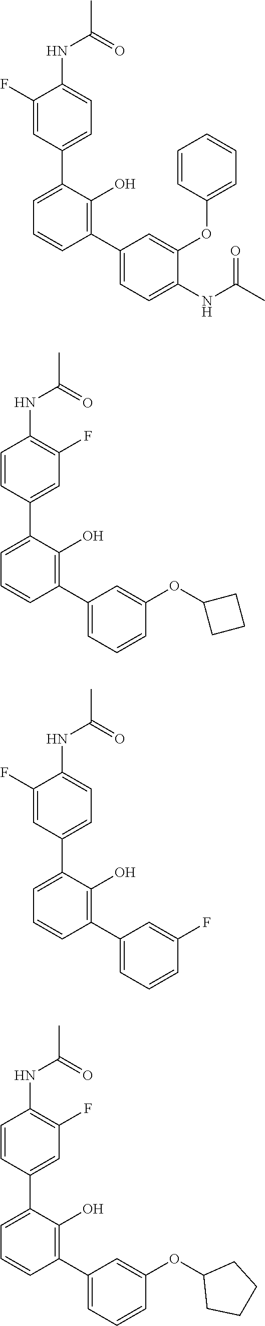

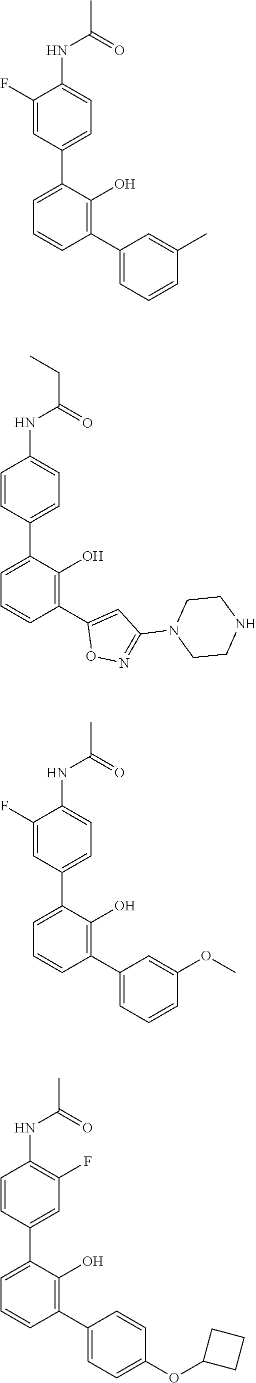 [SMILES: CC(=O)Nc1ccc(-c2cccc(-c3cccc(C)c3)c2O)cc1F ; CCC(=O)Nc1ccc(-c2cccc(-c3cc(N4CCNCC4)no3)c2O)cc1 ; COc1cccc(-c2cccc(-c3ccc(NC(C)=O)c(F)c3)c2O)c1 ; CC(=O)Nc1ccc(-c2cccc(-c3ccc(OC4CCC4)cc3)c2O)cc1F]

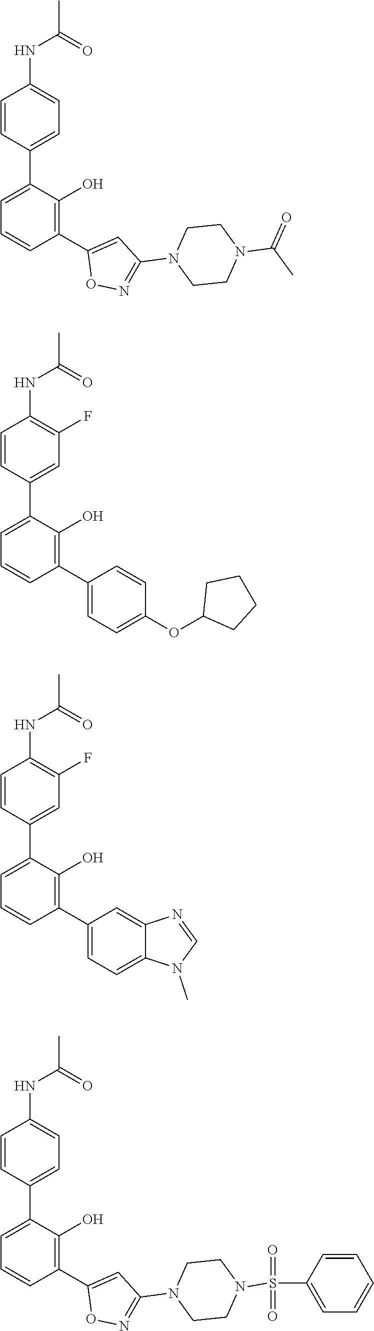 [SMILES: CC(=O)Nc1ccc(-c2cccc(-c3cc(N4CCN(C(C)=O)CC4)no3)c2O)cc1 ; CC(=O)Nc1ccc(-c2cccc(-c3ccc(OC4CCCC4)cc3)c2O)cc1F ; CC(=O)Nc1ccc(-c2cccc(-c3ccc4c(c3)ncn4C)c2O)cc1F ; CC(=O)Nc1ccc(-c2cccc(-c3cc(N4CCN(S(=O)(=O)c5ccccc5)CC4)no3)c2O)cc1]

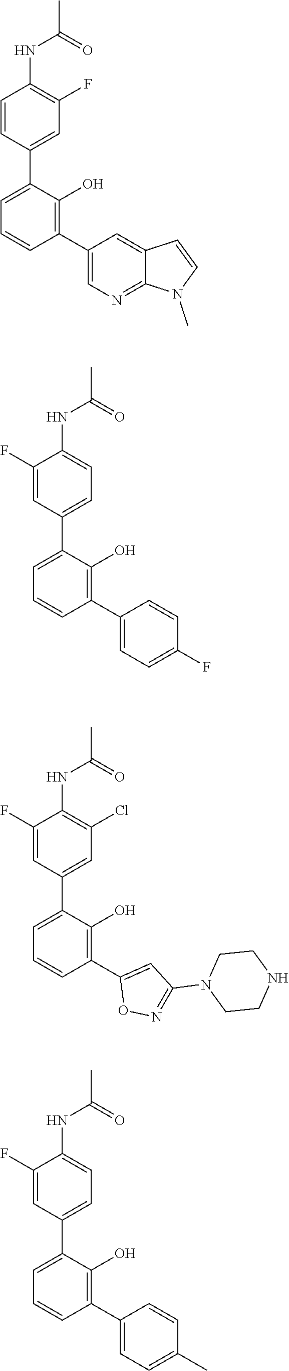 [SMILES: CC(=O)Nc1ccc(-c2cccc(-c3cnc4c(ccn4C)c3)c2O)cc1F ; CC(=O)Nc1ccc(-c2cccc(-c3ccc(F)cc3)c2O)cc1F ; CC(=O)Nc1c(F)cc(-c2cccc(-c3cc(N4CCNCC4)no3)c2O)cc1Cl ; CC(=O)Nc1ccc(-c2cccc(-c3ccc(C)cc3)c2O)cc1F]

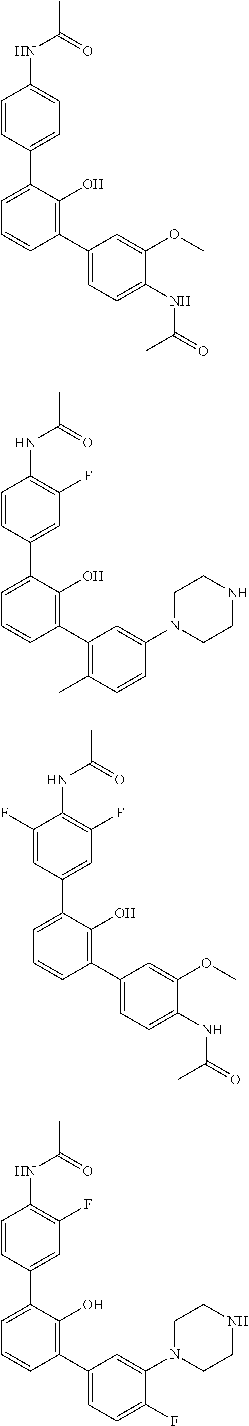

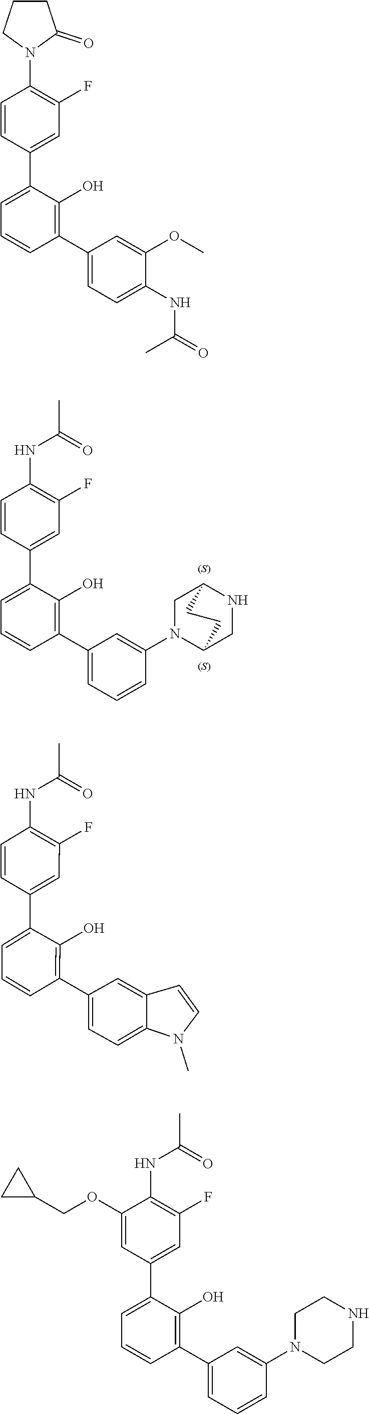

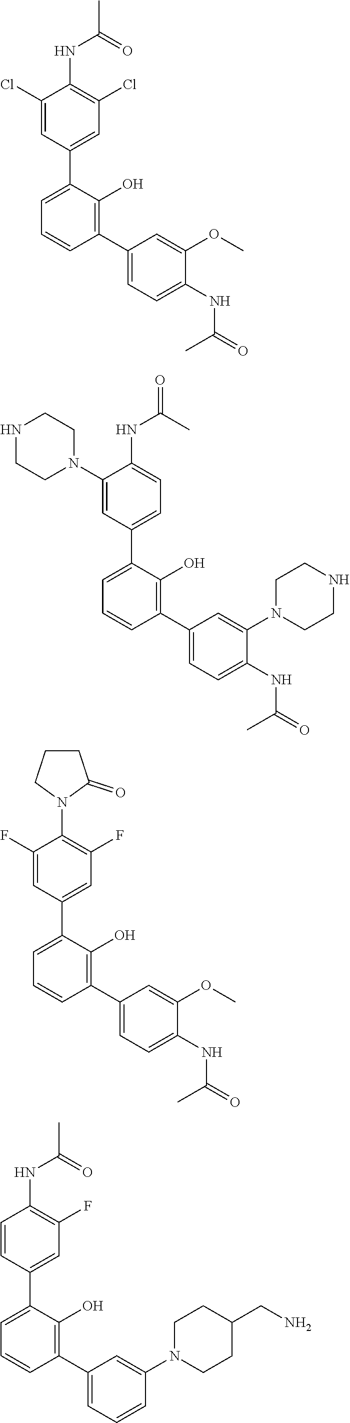

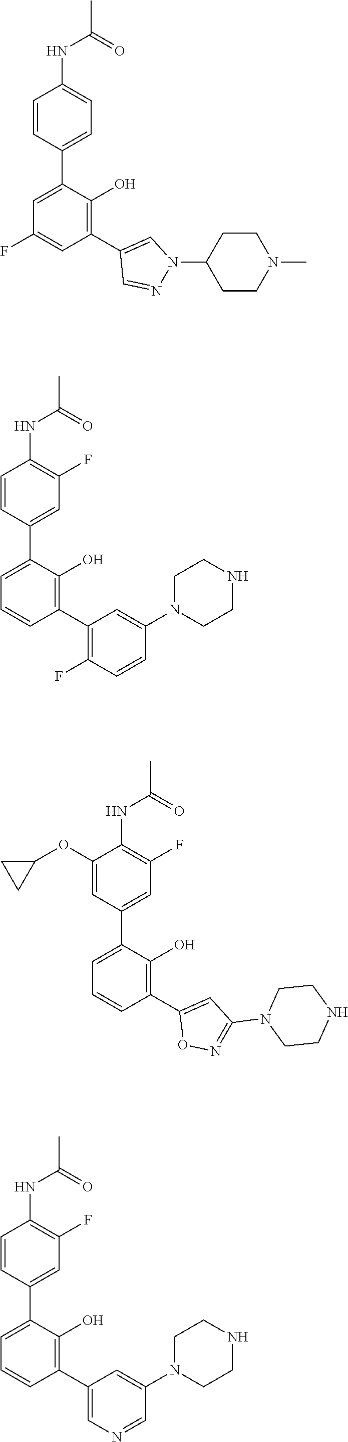 [SMILES: CC(=O)Nc1ccc(-c2cc(F)cc(-c3cnn(C4CCN(C)CC4)c3)c2O)cc1 ; CC(=O)Nc1ccc(-c2cccc(-c3cc(N4CCNCC4)ccc3F)c2O)cc1F ; CC(=O)Nc1c(F)cc(-c2cccc(-c3cc(N4CCNCC4)no3)c2O)cc1OC1CC1 ; CC(=O)Nc1ccc(-c2cccc(-c3cncc(N4CCNCC4)c3)c2O)cc1F]

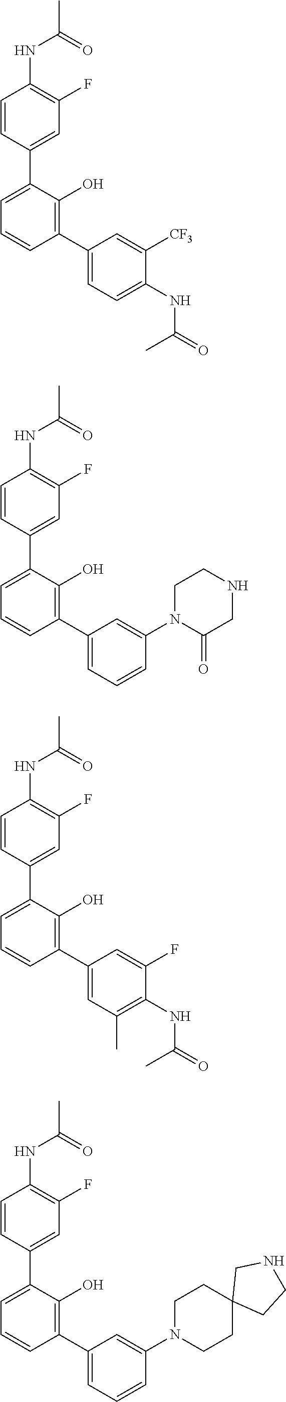 [SMILES: CC(=O)Nc1ccc(-c2cccc(-c3ccc(NC(C)=O)c(C(F)(F)F)c3)c2O)cc1F ; CC(=O)Nc1ccc(-c2cccc(-c3cccc(N4CCNCC4=O)c3)c2O)cc1F ; CC(=O)Nc1ccc(-c2cccc(-c3cc(C)c(NC(C)=O)c(F)c3)c2O)cc1F ; CC(=O)Nc1ccc(-c2cccc(-c3cccc(N4CCC5(CCNC5)CC4)c3)c2O)cc1F]

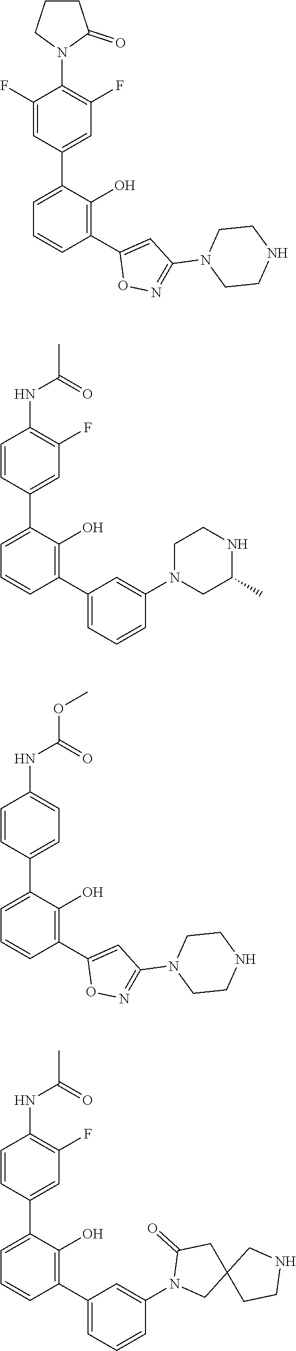 [SMILES: O=C1CCCN1c1c(F)cc(-c2cccc(-c3cc(N4CCNCC4)no3)c2O)cc1F ; CC(=O)Nc1ccc(-c2cccc(-c3cccc(N4CCN[C@H](C)C4)c3)c2O)cc1F ; COC(=O)Nc1ccc(-c2cccc(-c3cc(N4CCNCC4)no3)c2O)cc1 ; CC(=O)Nc1ccc(-c2cccc(-c3cccc(N4CC5(CCNC5)CC4=O)c3)c2O)cc1F]

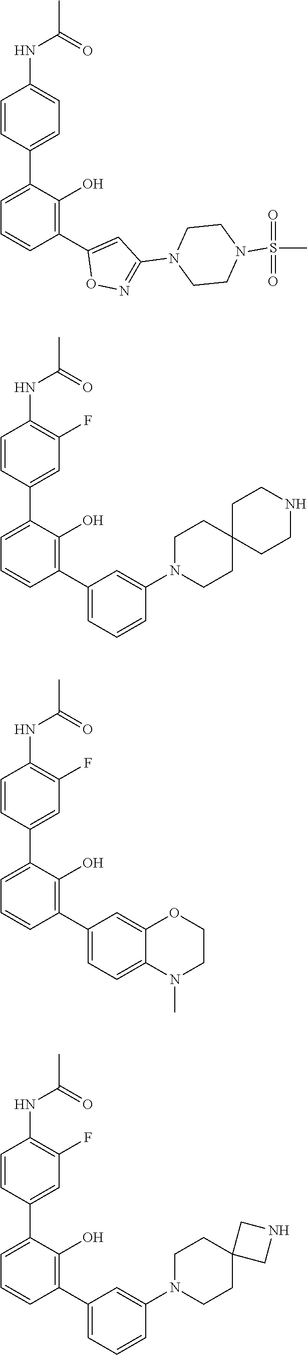 [SMILES: CC(=O)Nc1ccc(-c2cccc(-c3cc(N4CCN(S(C)(=O)=O)CC4)no3)c2O)cc1 ; CC(=O)Nc1ccc(-c2cccc(-c3cccc(N4CCC5(CCNCC5)CC4)c3)c2O)cc1F ; CC(=O)Nc1ccc(-c2cccc(-c3ccc4c(c3)OCCN4C)c2O)cc1F ; CC(=O)Nc1ccc(-c2cccc(-c3cccc(N4CCC5(CC4)CNC5)c3)c2O)cc1F]

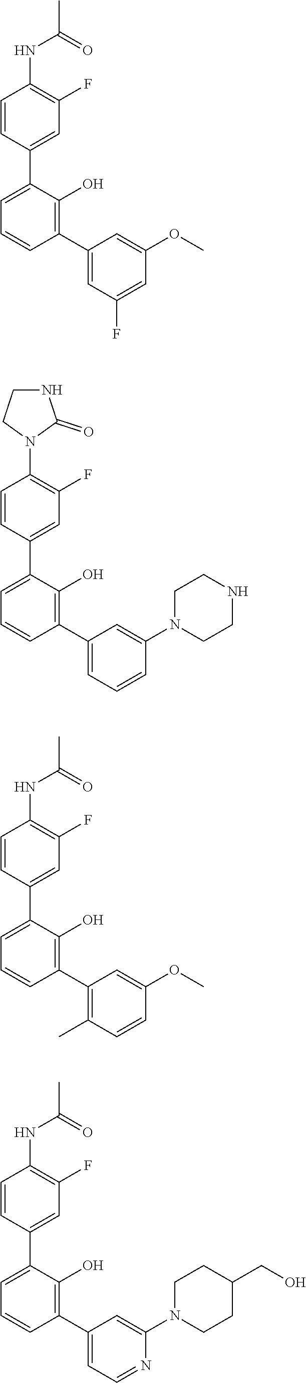

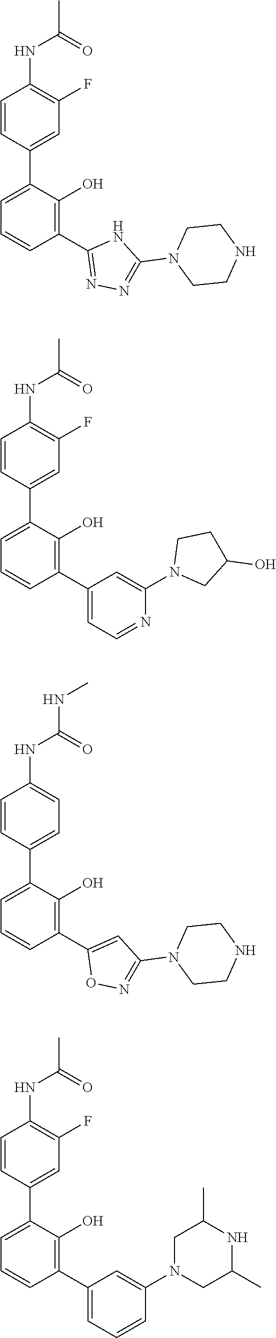 [SMILES: CC(=O)Nc1ccc(-c2cccc(-c3nnc(N4CCNCC4)[nH]3)c2O)cc1F ; CC(=O)Nc1ccc(-c2cccc(-c3ccnc(N4CCC(O)C4)c3)c2O)cc1F ; CNC(=O)Nc1ccc(-c2cccc(-c3cc(N4CCNCC4)no3)c2O)cc1 ; CC(=O)Nc1ccc(-c2cccc(-c3cccc(N4CC(C)NC(C)C4)c3)c2O)cc1F]

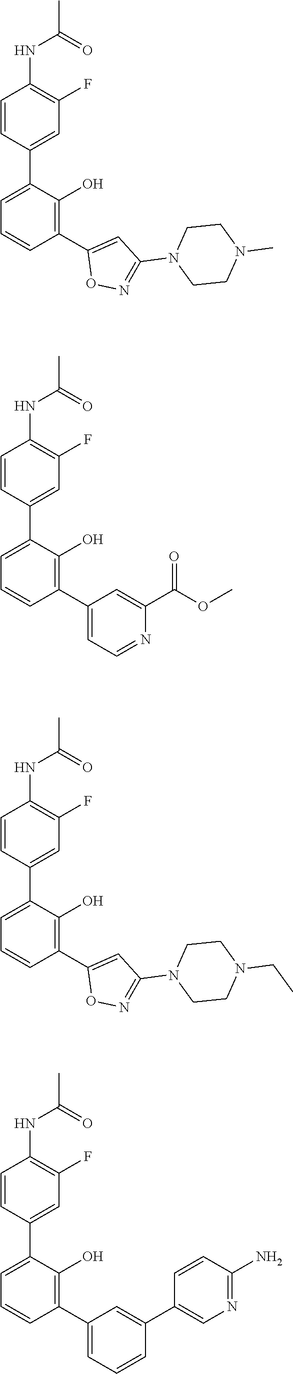 [SMILES: CC(=O)Nc1ccc(-c2cccc(-c3cc(N4CCN(C)CC4)no3)c2O)cc1F ; COC(=O)c1cc(-c2cccc(-c3ccc(NC(C)=O)c(F)c3)c2O)ccn1 ; CCN1CCN(c2cc(-c3cccc(-c4ccc(NC(C)=O)c(F)c4)c3O)on2)CC1 ; CC(=O)Nc1ccc(-c2cccc(-c3cccc(-c4ccc(N)nc4)c3)c2O)cc1F]

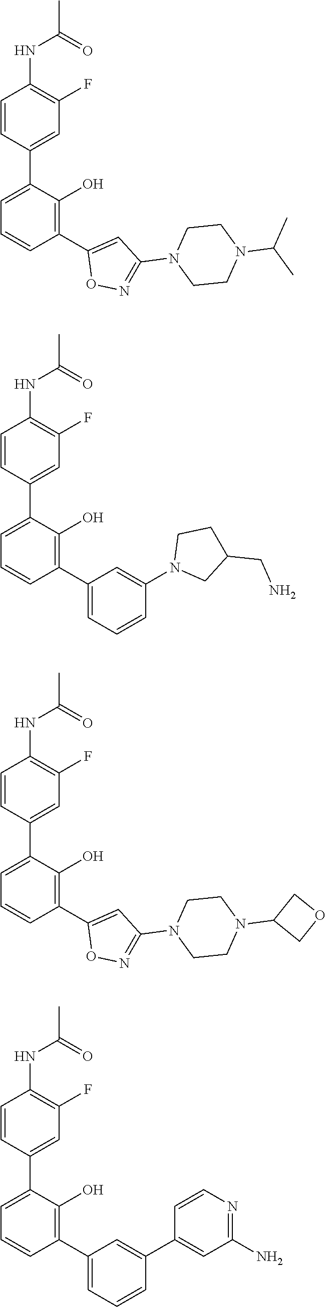 [SMILES: CC(=O)Nc1ccc(-c2cccc(-c3cc(N4CCN(C(C)C)CC4)no3)c2O)cc1F ; CC(=O)Nc1ccc(-c2cccc(-c3cccc(N4CCC(CN)C4)c3)c2O)cc1F ; CC(=O)Nc1ccc(-c2cccc(-c3cc(N4CCN(C5COC5)CC4)no3)c2O)cc1F ; CC(=O)Nc1ccc(-c2cccc(-c3cccc(-c4ccnc(N)c4)c3)c2O)cc1F]

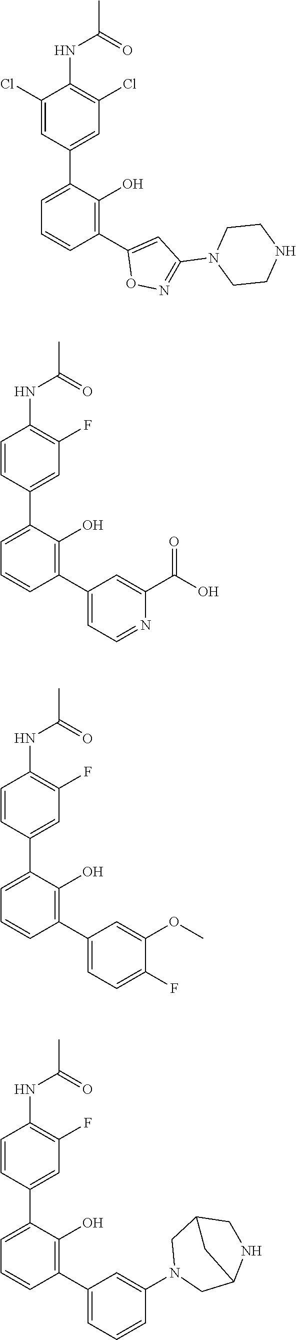 [SMILES: CC(=O)Nc1c(Cl)cc(-c2cccc(-c3cc(N4CCNCC4)no3)c2O)cc1Cl ; CC(=O)Nc1ccc(-c2cccc(-c3ccnc(C(=O)O)c3)c2O)cc1F ; COc1cc(-c2cccc(-c3ccc(NC(C)=O)c(F)c3)c2O)ccc1F ; CC(=O)Nc1ccc(-c2cccc(-c3cccc(N4CC5CNC(C5)C4)c3)c2O)cc1F]

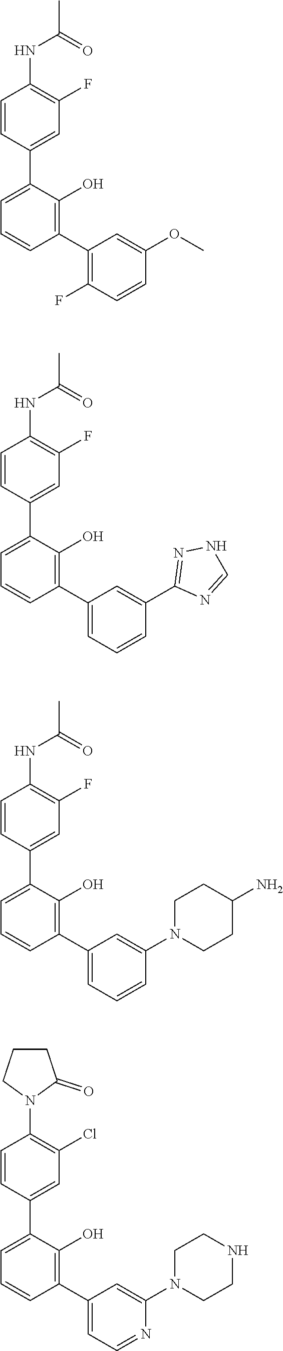 [SMILES: COc1ccc(F)c(-c2cccc(-c3ccc(NC(C)=O)c(F)c3)c2O)c1 ; CC(=O)Nc1ccc(-c2cccc(-c3cccc(-c4nc[nH]n4)c3)c2O)cc1F ; CC(=O)Nc1ccc(-c2cccc(-c3cccc(N4CCC(N)CC4)c3)c2O)cc1F ; O=C1CCCN1c1ccc(-c2cccc(-c3ccnc(N4CCNCC4)c3)c2O)cc1Cl]

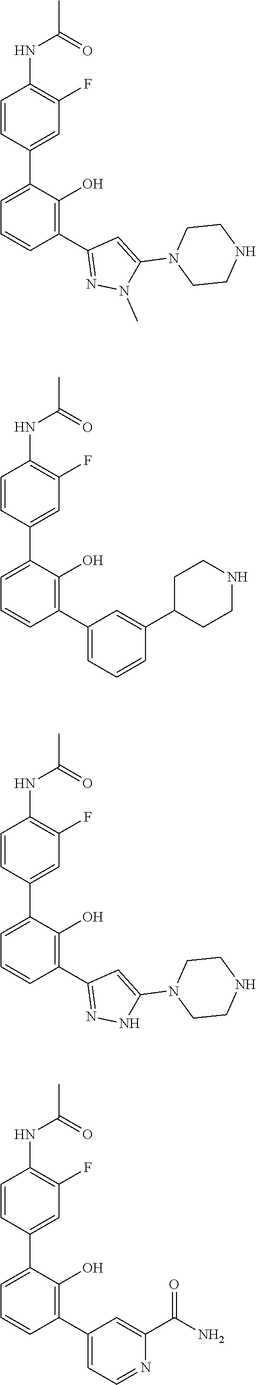 [SMILES: CC(=O)Nc1ccc(-c2cccc(-c3cc(N4CCNCC4)n(C)n3)c2O)cc1F ; CC(=O)Nc1ccc(-c2cccc(-c3cccc(C4CCNCC4)c3)c2O)cc1F ; CC(=O)Nc1ccc(-c2cccc(-c3cc(N4CCNCC4)[nH]n3)c2O)cc1F ; CC(=O)Nc1ccc(-c2cccc(-c3ccnc(C(N)=O)c3)c2O)cc1F]

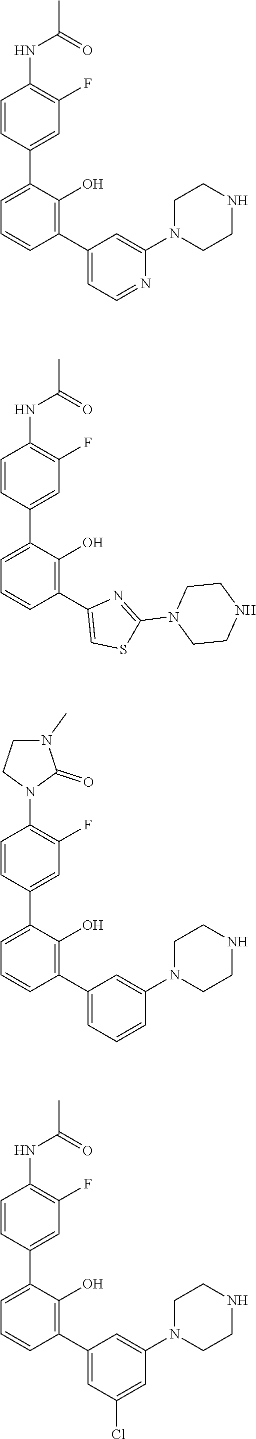 [SMILES: CC(=O)Nc1ccc(-c2cccc(-c3ccnc(N4CCNCC4)c3)c2O)cc1F ; CC(=O)Nc1ccc(-c2cccc(-c3csc(N4CCNCC4)n3)c2O)cc1F ; CN1CCN(c2ccc(-c3cccc(-c4cccc(N5CCNCC5)c4)c3O)cc2F)C1=O ; CC(=O)Nc1ccc(-c2cccc(-c3cc(Cl)cc(N4CCNCC4)c3)c2O)cc1F]

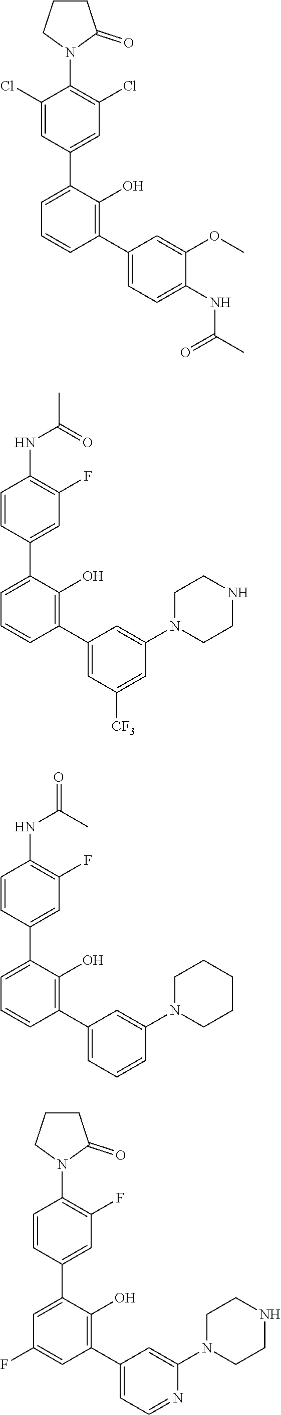 [SMILES: COc1cc(-c2cccc(-c3cc(Cl)c(N4CCCC4=O)c(Cl)c3)c2O)ccc1NC(C)=O ; CC(=O)Nc1ccc(-c2cccc(-c3cc(N4CCNCC4)cc(C(F)(F)F)c3)c2O)cc1F ; CC(=O)Nc1ccc(-c2cccc(-c3cccc(N4CCCCC4)c3)c2O)cc1F ; O=C1CCCN1c1ccc(-c2cc(F)cc(-c3ccnc(N4CCNCC4)c3)c2O)cc1F]

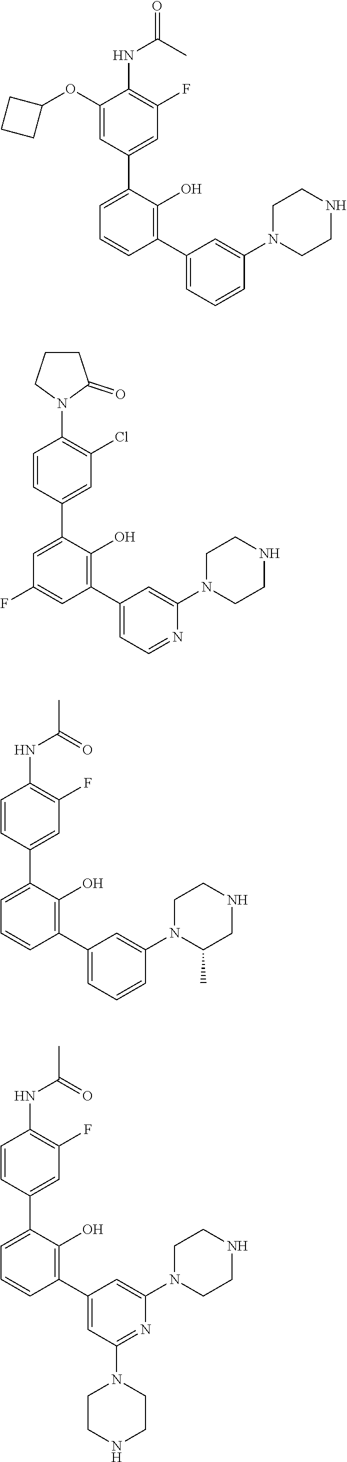 [SMILES: CC(=O)Nc1c(F)cc(-c2cccc(-c3cccc(N4CCNCC4)c3)c2O)cc1OC1CCC1 ; O=C1CCCN1c1ccc(-c2cc(F)cc(-c3ccnc(N4CCNCC4)c3)c2O)cc1Cl ; CC(=O)Nc1ccc(-c2cccc(-c3cccc(N4CCNC[C@@H]4C)c3)c2O)cc1F ; CC(=O)Nc1ccc(-c2cccc(-c3cc(N4CCNCC4)nc(N4CCNCC4)c3)c2O)cc1F]

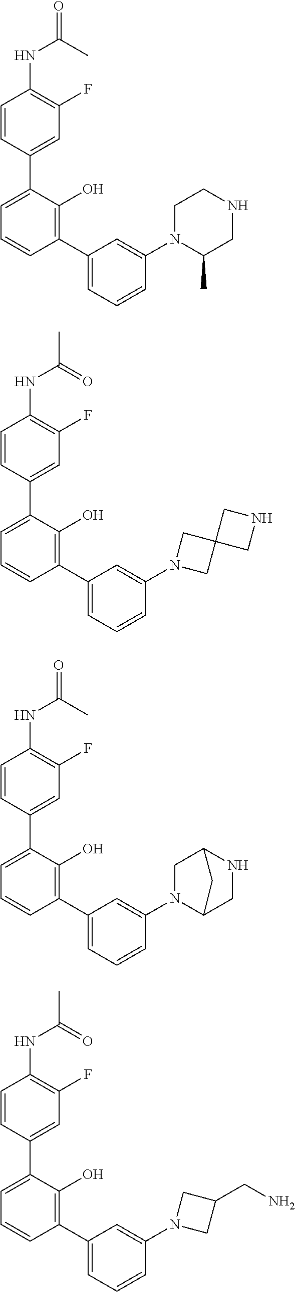 [SMILES: CC(=O)Nc1ccc(-c2cccc(-c3cccc(N4CCNC[C@H]4C)c3)c2O)cc1F ; CC(=O)Nc1ccc(-c2cccc(-c3cccc(N4CC5(CNC5)C4)c3)c2O)cc1F ; CC(=O)Nc1ccc(-c2cccc(-c3cccc(N4CC5CC4CN5)c3)c2O)cc1F ; CC(=O)Nc1ccc(-c2cccc(-c3cccc(N4CC(CN)C4)c3)c2O)cc1F]

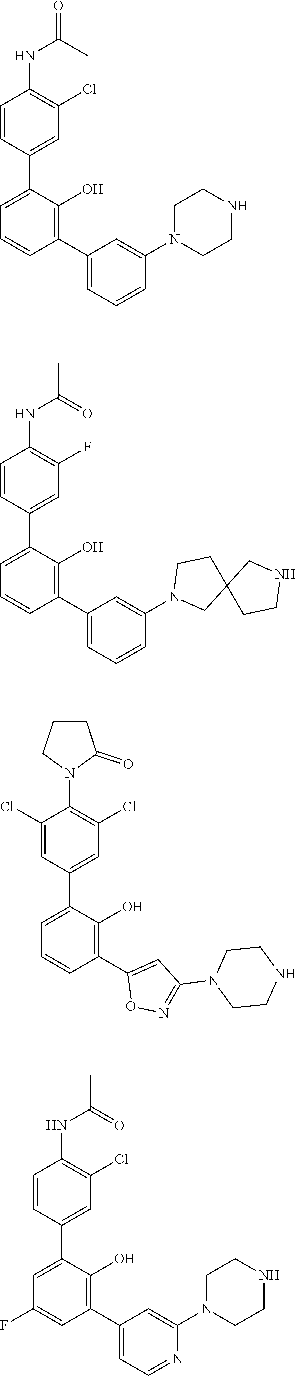 [SMILES: CC(=O)Nc1ccc(-c2cccc(-c3cccc(N4CCNCC4)c3)c2O)cc1Cl ; CC(=O)Nc1ccc(-c2cccc(-c3cccc(N4CCC5(CCNC5)C4)c3)c2O)cc1F ; O=C1CCCN1c1c(Cl)cc(-c2cccc(-c3cc(N4CCNCC4)no3)c2O)cc1Cl ; CC(=O)Nc1ccc(-c2cc(F)cc(-c3ccnc(N4CCNCC4)c3)c2O)cc1Cl]

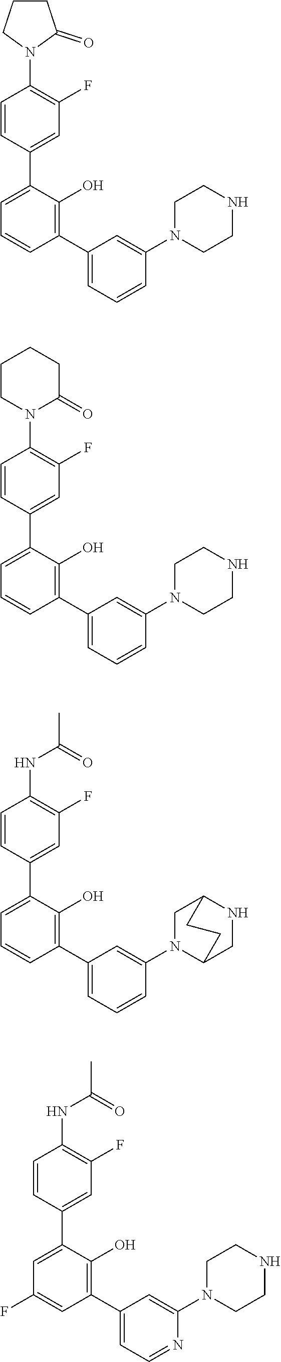 [SMILES: O=C1CCCN1c1ccc(-c2cccc(-c3cccc(N4CCNCC4)c3)c2O)cc1F ; O=C1CCCCN1c1ccc(-c2cccc(-c3cccc(N4CCNCC4)c3)c2O)cc1F ; CC(=O)Nc1ccc(-c2cccc(-c3cccc(N4CC5CCC4CN5)c3)c2O)cc1F ; CC(=O)Nc1ccc(-c2cc(F)cc(-c3ccnc(N4CCNCC4)c3)c2O)cc1F]

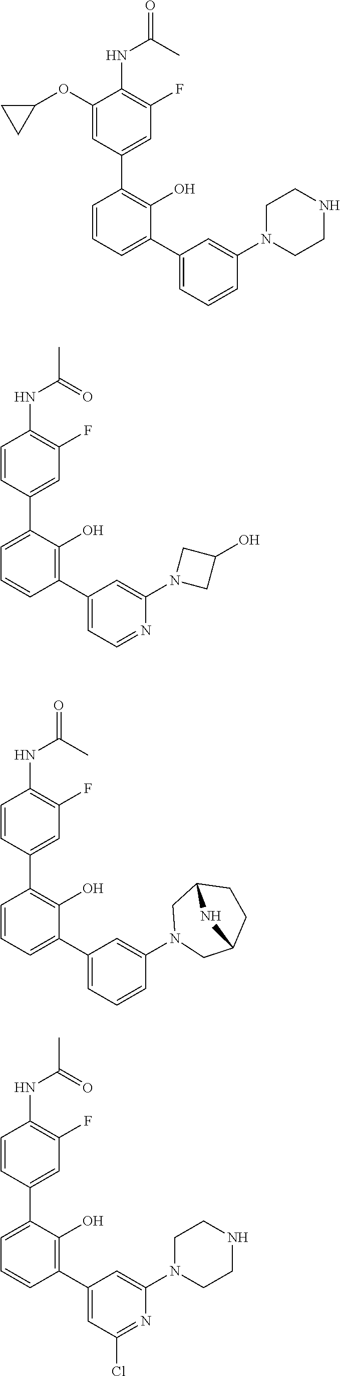 [SMILES: CC(=O)Nc1c(F)cc(-c2cccc(-c3cccc(N4CCNCC4)c3)c2O)cc1OC1CC1 ; CC(=O)Nc1ccc(-c2cccc(-c3ccnc(N4CC(O)C4)c3)c2O)cc1F ; CC(=O)Nc1ccc(-c2cccc(-c3cccc(N4C[C@H]5CC[C@@H](C4)N5)c3)c2O)cc1F ; CC(=O)Nc1ccc(-c2cccc(-c3cc(Cl)nc(N4CCNCC4)c3)c2O)cc1F]

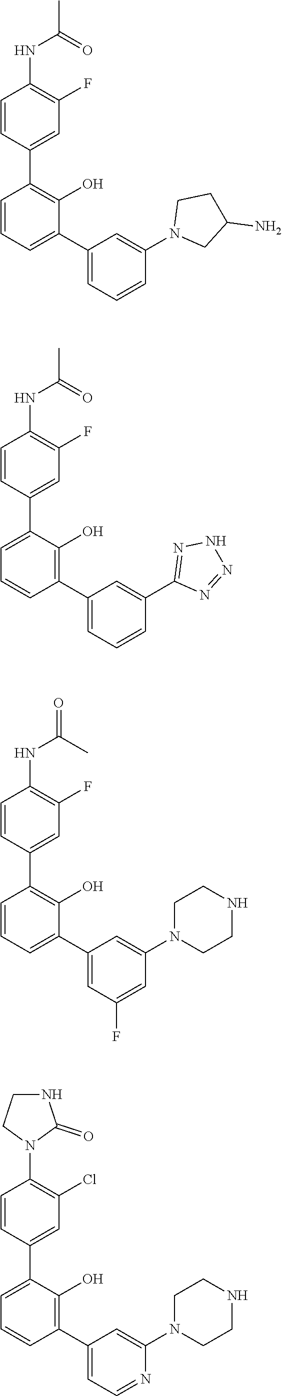 [SMILES: CC(=O)Nc1ccc(-c2cccc(-c3cccc(N4CCC(N)C4)c3)c2O)cc1F ; CC(=O)Nc1ccc(-c2cccc(-c3cccc(-c4nn[nH]n4)c3)c2O)cc1F ; CC(=O)Nc1ccc(-c2cccc(-c3cc(F)cc(N4CCNCC4)c3)c2O)cc1F ; O=C1NCCN1c1ccc(-c2cccc(-c3ccnc(N4CCNCC4)c3)c2O)cc1Cl]

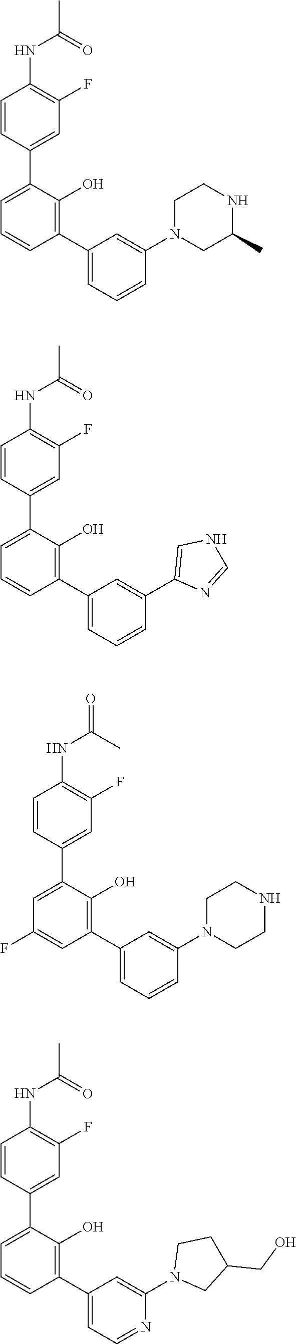 [SMILES: CC(=O)Nc1ccc(-c2cccc(-c3cccc(N4CCN[C@@H](C)C4)c3)c2O)cc1F ; CC(=O)Nc1ccc(-c2cccc(-c3cccc(-c4c[nH]cn4)c3)c2O)cc1F ; CC(=O)Nc1ccc(-c2cc(F)cc(-c3cccc(N4CCNCC4)c3)c2O)cc1F ; CC(=O)Nc1ccc(-c2cccc(-c3ccnc(N4CCC(CO)C4)c3)c2O)cc1F]

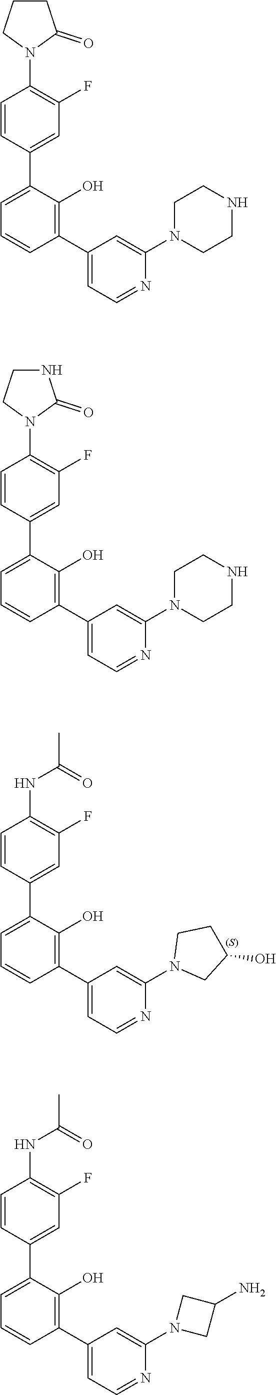

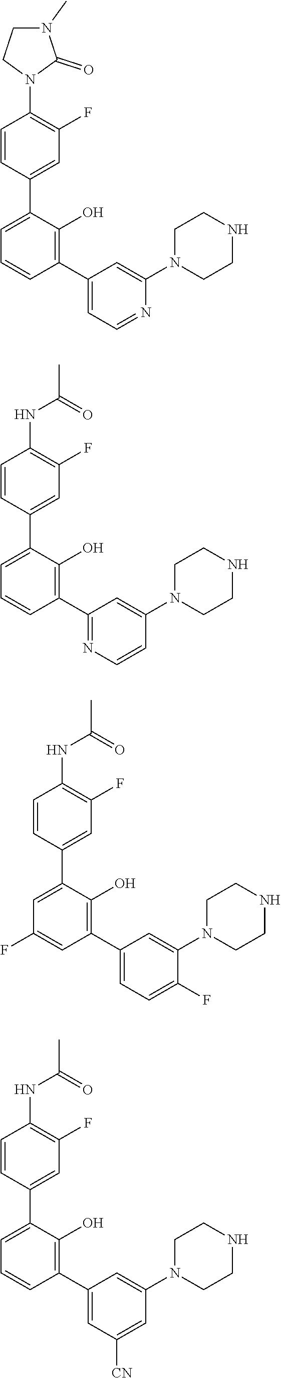

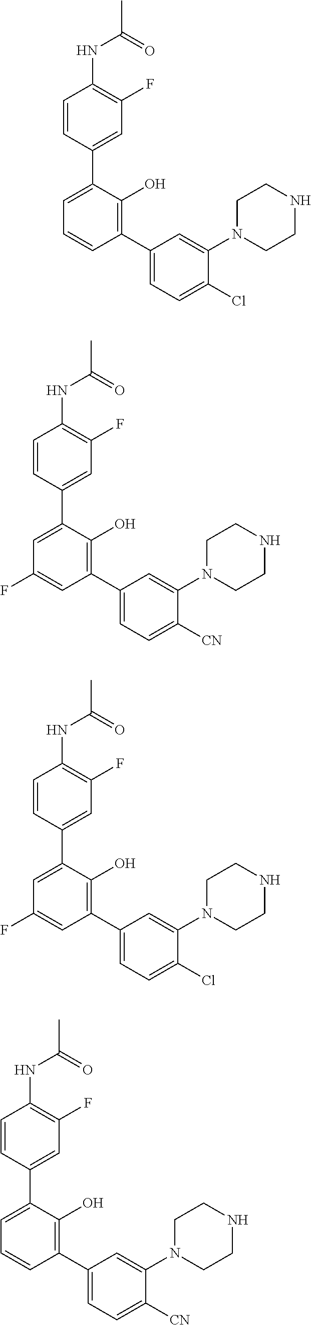 [SMILES: CC(=O)Nc1ccc(-c2cccc(-c3ccc(Cl)c(N4CCNCC4)c3)c2O)cc1F ; CC(=O)Nc1ccc(-c2cc(F)cc(-c3ccc(C#N)c(N4CCNCC4)c3)c2O)cc1F ; CC(=O)Nc1ccc(-c2cc(F)cc(-c3ccc(Cl)c(N4CCNCC4)c3)c2O)cc1F ; CC(=O)Nc1ccc(-c2cccc(-c3ccc(C#N)c(N4CCNCC4)c3)c2O)cc1F]

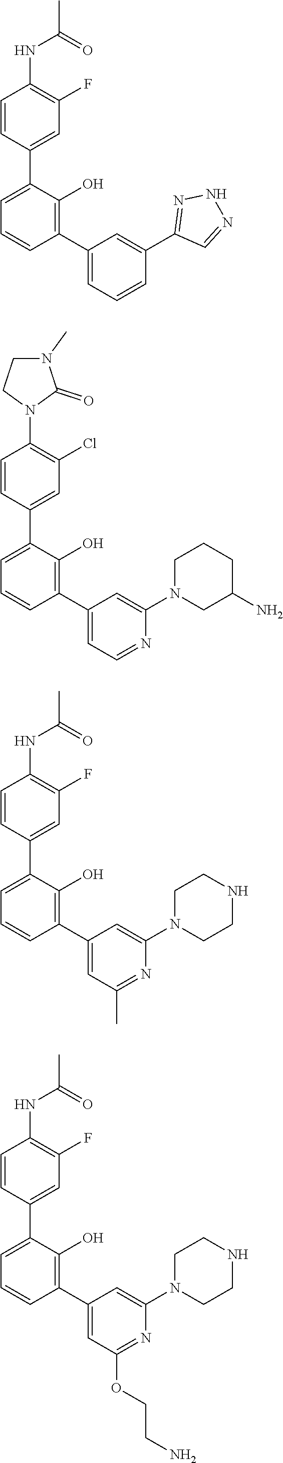 [SMILES: CC(=O)Nc1ccc(-c2cccc(-c3cccc(-c4cn[nH]n4)c3)c2O)cc1F ; CN1CCN(c2ccc(-c3cccc(-c4ccnc(N5CCCC(N)C5)c4)c3O)cc2Cl)C1=O ; CC(=O)Nc1ccc(-c2cccc(-c3cc(C)nc(N4CCNCC4)c3)c2O)cc1F ; CC(=O)Nc1ccc(-c2cccc(-c3cc(OCCN)nc(N4CCNCC4)c3)c2O)cc1F]

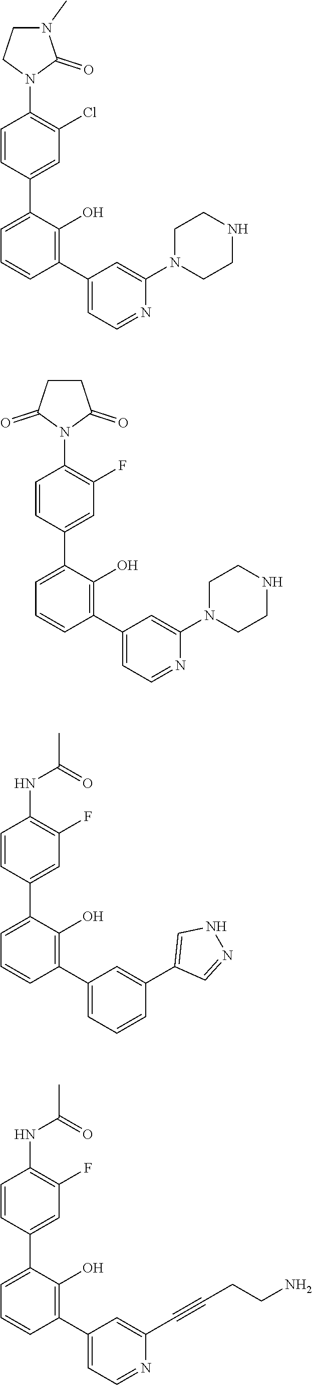 [SMILES: CN1CCN(c2ccc(-c3cccc(-c4ccnc(N5CCNCC5)c4)c3O)cc2Cl)C1=O ; O=C1CCC(=O)N1c1ccc(-c2cccc(-c3ccnc(N4CCNCC4)c3)c2O)cc1F ; CC(=O)Nc1ccc(-c2cccc(-c3cccc(-c4cn[nH]c4)c3)c2O)cc1F ; CC(=O)Nc1ccc(-c2cccc(-c3ccnc(C#CCCN)c3)c2O)cc1F]

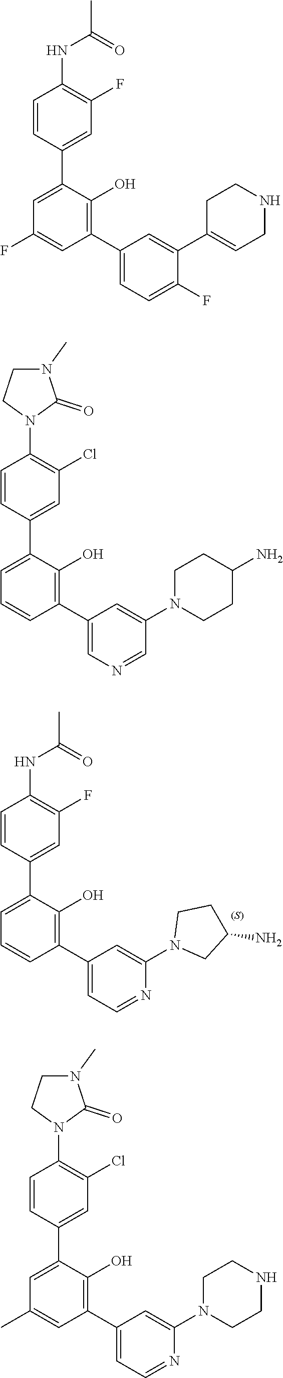

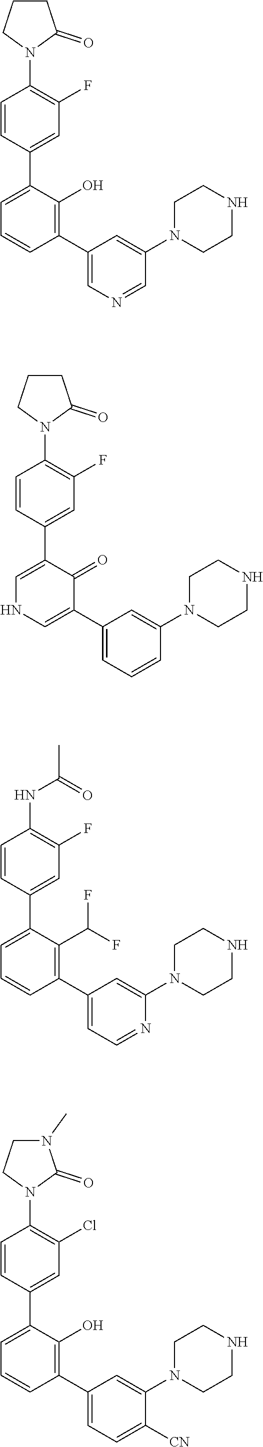 [SMILES: O=C1CCCN1c1ccc(-c2cccc(-c3cncc(N4CCNCC4)c3)c2O)cc1F ; O=C1CCCN1c1ccc(-c2c[nH]cc(-c3cccc(N4CCNCC4)c3)c2=O)cc1F ; CC(=O)Nc1ccc(-c2cccc(-c3ccnc(N4CCNCC4)c3)c2C(F)F)cc1F ; CN1CCN(c2ccc(-c3cccc(-c4ccc(C#N)c(N5CCNCC5)c4)c3O)cc2Cl)C1=O]

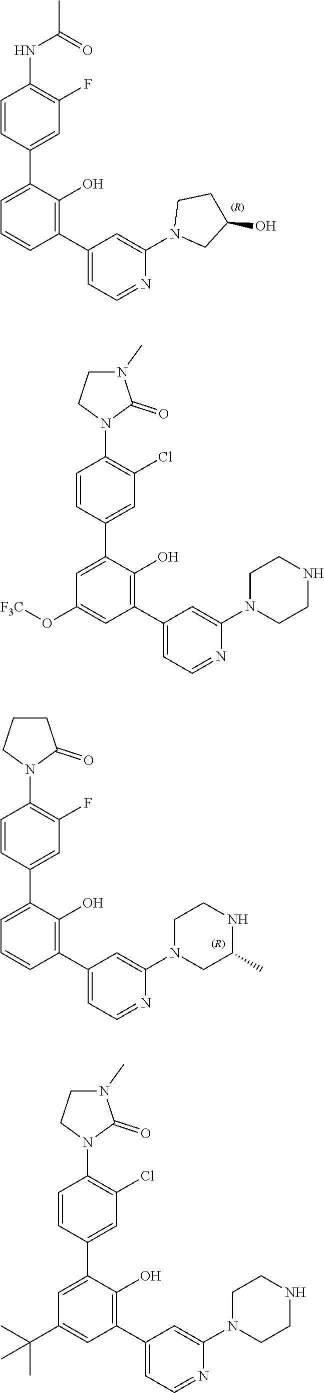

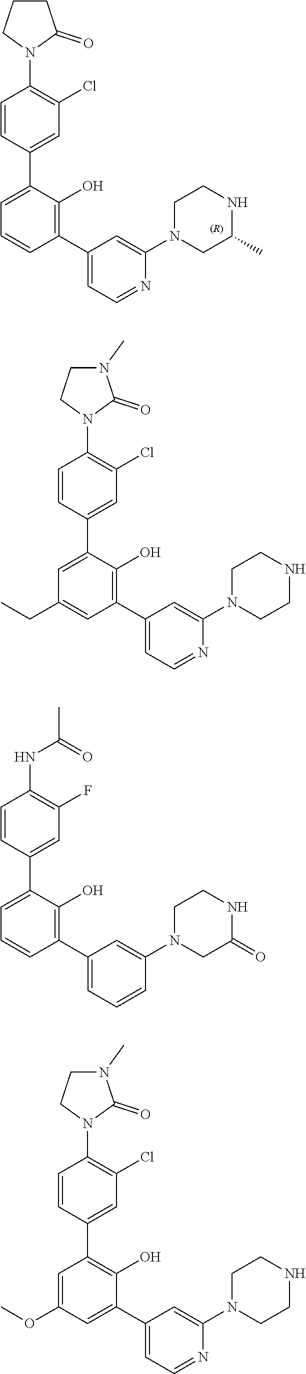

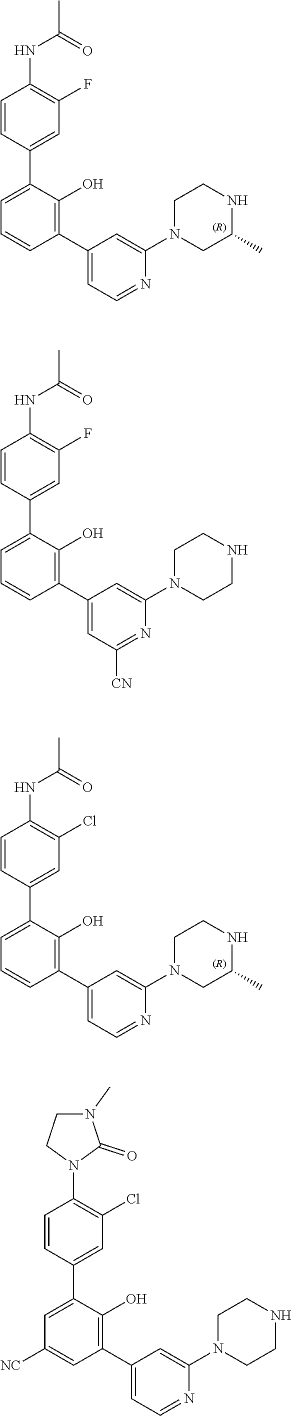

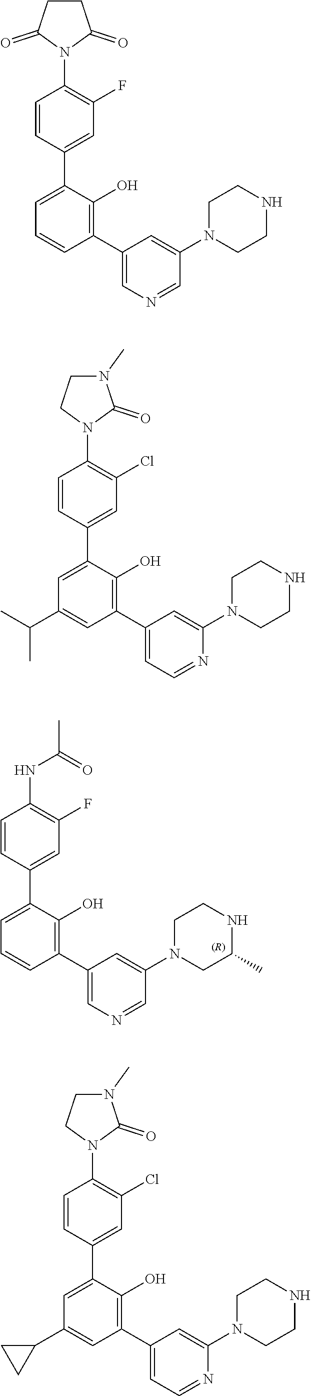

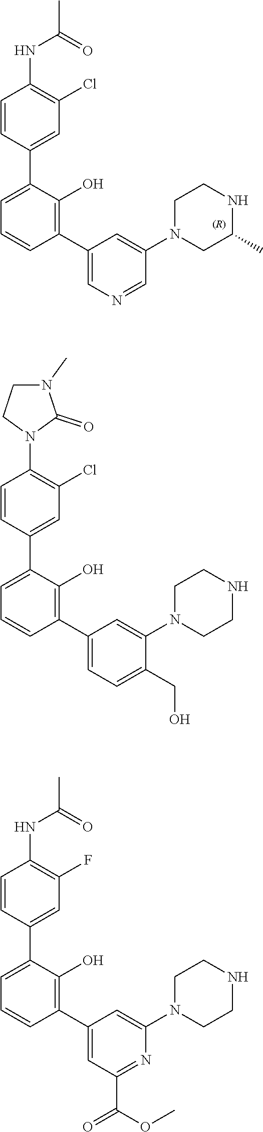

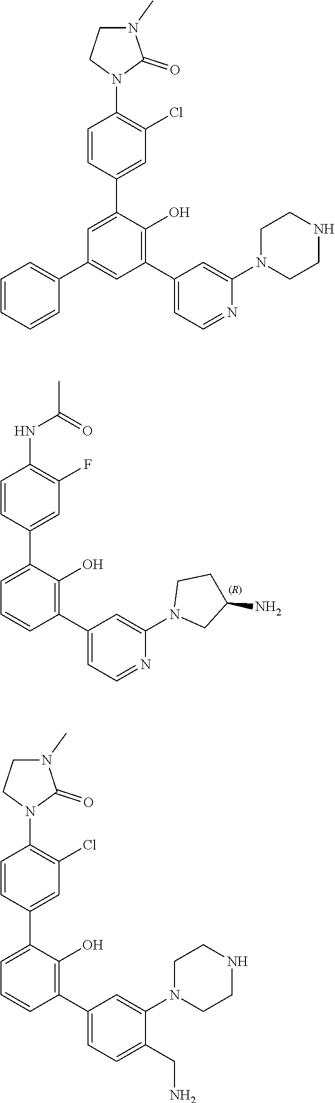

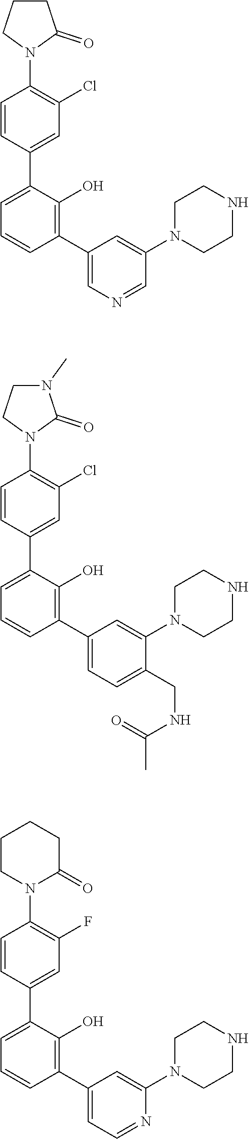 [SMILES: O=C1CCCN1c1ccc(-c2cccc(-c3cncc(N4CCNCC4)c3)c2O)cc1Cl ; CC(=O)NCc1ccc(-c2cccc(-c3ccc(N4CCN(C)C4=O)c(Cl)c3)c2O)cc1N1CCNCC1 ; O=C1CCCCN1c1ccc(-c2cccc(-c3ccnc(N4CCNCC4)c3)c2O)cc1F]

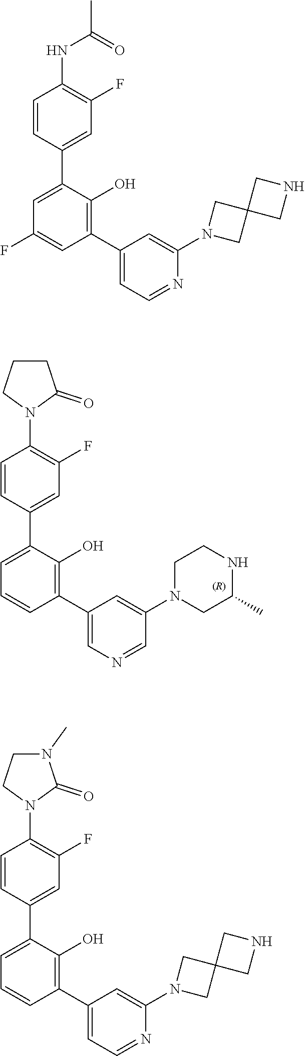

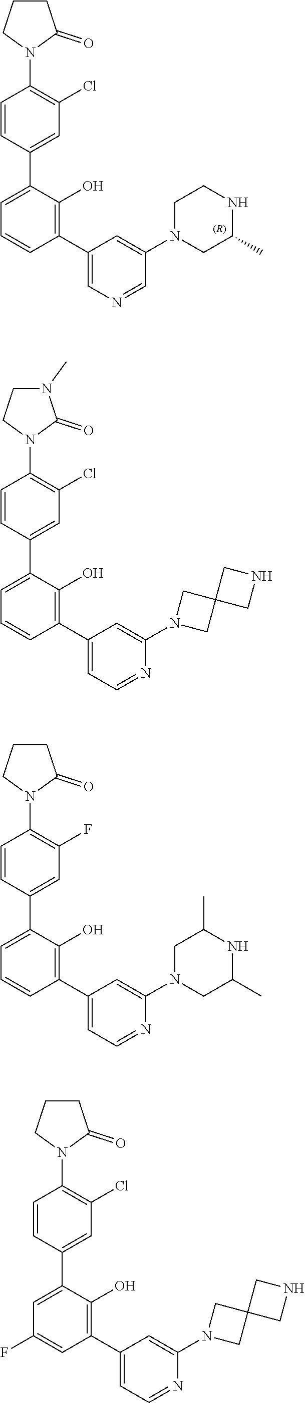

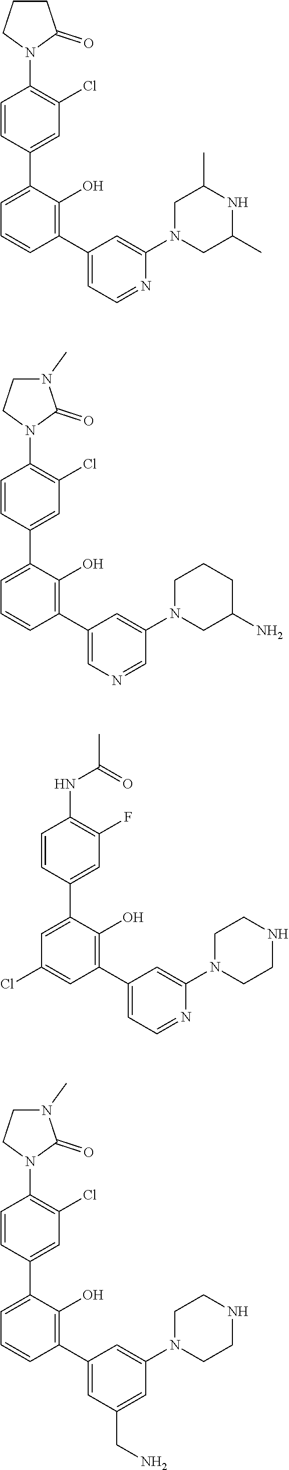 [SMILES: CC1CN(c2cc(-c3cccc(-c4ccc(N5CCCC5=O)c(Cl)c4)c3O)ccn2)CC(C)N1 ; CN1CCN(c2ccc(-c3cccc(-c4cncc(N5CCCC(N)C5)c4)c3O)cc2Cl)C1=O ; CC(=O)Nc1ccc(-c2cc(Cl)cc(-c3ccnc(N4CCNCC4)c3)c2O)cc1F ; CN1CCN(c2ccc(-c3cccc(-c4cc(CN)cc(N5CCNCC5)c4)c3O)cc2Cl)C1=O]

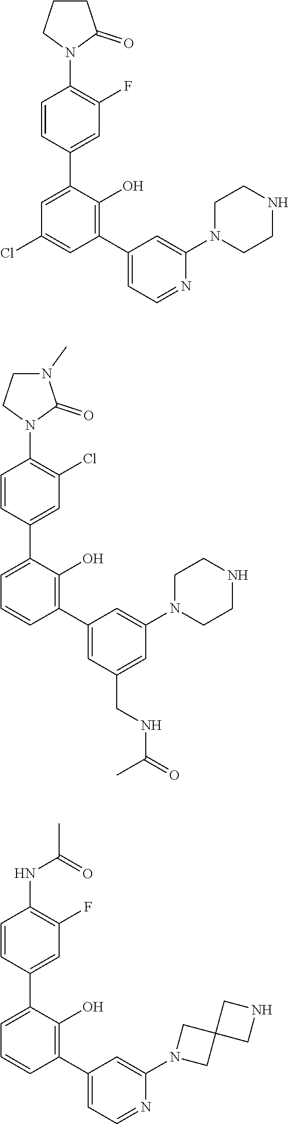 [SMILES: O=C1CCCN1c1ccc(-c2cc(Cl)cc(-c3ccnc(N4CCNCC4)c3)c2O)cc1F ; CC(=O)NCc1cc(-c2cccc(-c3ccc(N4CCN(C)C4=O)c(Cl)c3)c2O)cc(N2CCNCC2)c1 ; CC(=O)Nc1ccc(-c2cccc(-c3ccnc(N4CC5(CNC5)C4)c3)c2O)cc1F]

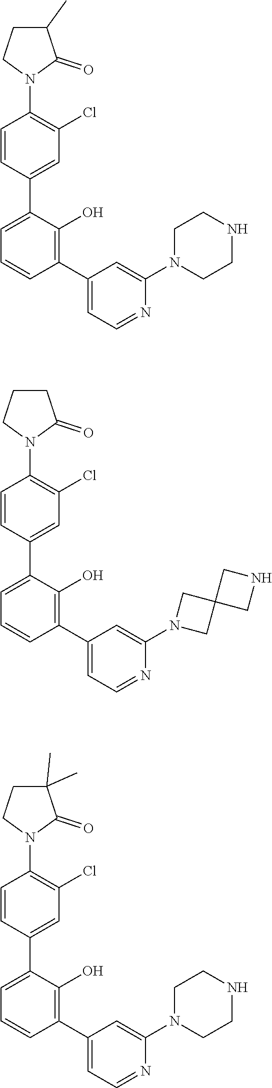 [SMILES: CC1CCN(c2ccc(-c3cccc(-c4ccnc(N5CCNCC5)c4)c3O)cc2Cl)C1=O ; O=C1CCCN1c1ccc(-c2cccc(-c3ccnc(N4CC5(CNC5)C4)c3)c2O)cc1Cl ; CC1(C)CCN(c2ccc(-c3cccc(-c4ccnc(N5CCNCC5)c4)c3O)cc2Cl)C1=O]

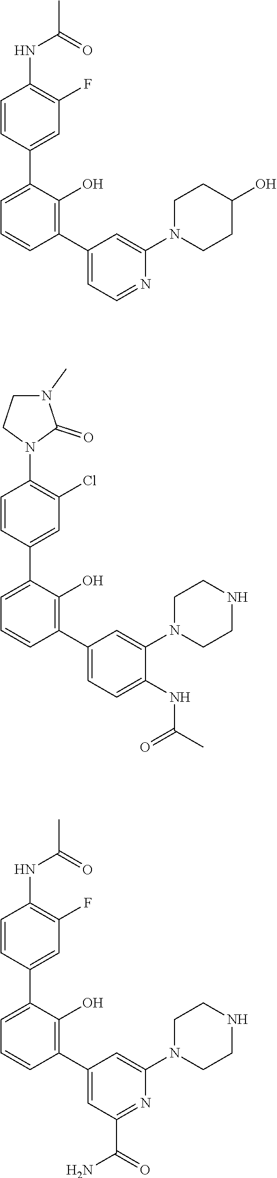 [SMILES: CC(=O)Nc1ccc(-c2cccc(-c3ccnc(N4CCC(O)CC4)c3)c2O)cc1F ; CC(=O)Nc1ccc(-c2cccc(-c3ccc(N4CCN(C)C4=O)c(Cl)c3)c2O)cc1N1CCNCC1 ; CC(=O)Nc1ccc(-c2cccc(-c3cc(C(N)=O)nc(N4CCNCC4)c3)c2O)cc1F]

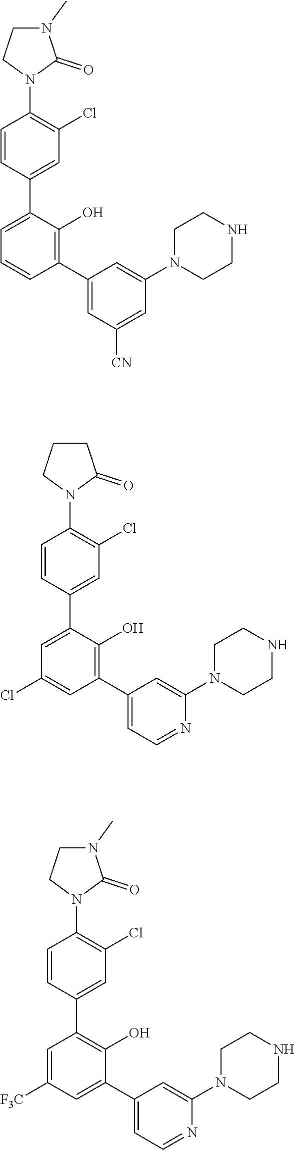 [SMILES: CN1CCN(c2ccc(-c3cccc(-c4cc(C#N)cc(N5CCNCC5)c4)c3O)cc2Cl)C1=O ; O=C1CCCN1c1ccc(-c2cc(Cl)cc(-c3ccnc(N4CCNCC4)c3)c2O)cc1Cl ; CN1CCN(c2ccc(-c3cc(C(F)(F)F)cc(-c4ccnc(N5CCNCC5)c4)c3O)cc2Cl)C1=O]

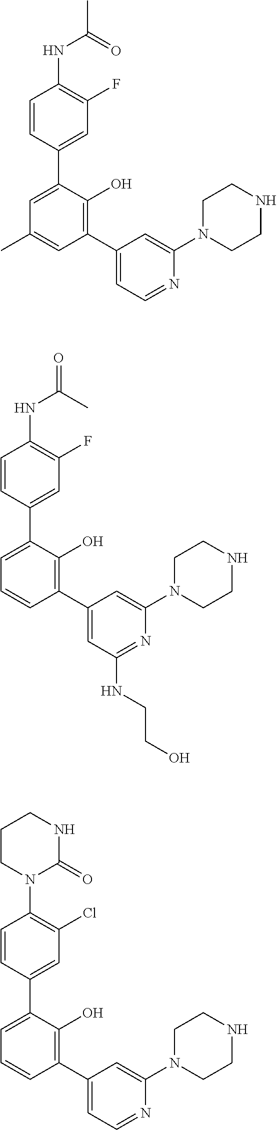 [SMILES: CC(=O)Nc1ccc(-c2cc(C)cc(-c3ccnc(N4CCNCC4)c3)c2O)cc1F ; CC(=O)Nc1ccc(-c2cccc(-c3cc(NCCO)nc(N4CCNCC4)c3)c2O)cc1F ; O=C1NCCCN1c1ccc(-c2cccc(-c3ccnc(N4CCNCC4)c3)c2O)cc1Cl]

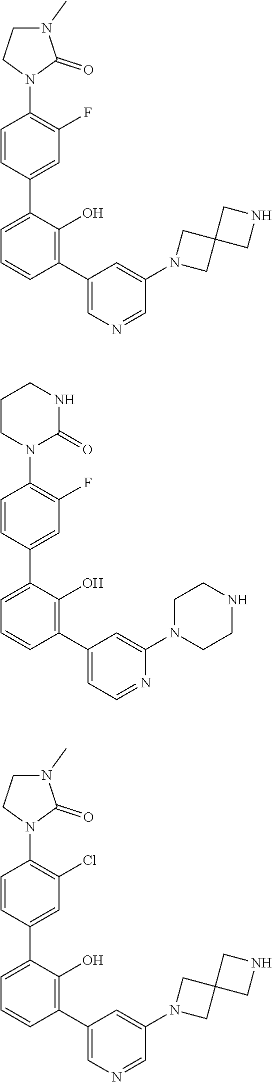 [SMILES: CN1CCN(c2ccc(-c3cccc(-c4cncc(N5CC6(CNC6)C5)c4)c3O)cc2F)C1=O ; O=C1NCCCN1c1ccc(-c2cccc(-c3ccnc(N4CCNCC4)c3)c2O)cc1F ; CN1CCN(c2ccc(-c3cccc(-c4cncc(N5CC6(CNC6)C5)c4)c3O)cc2Cl)C1=O]

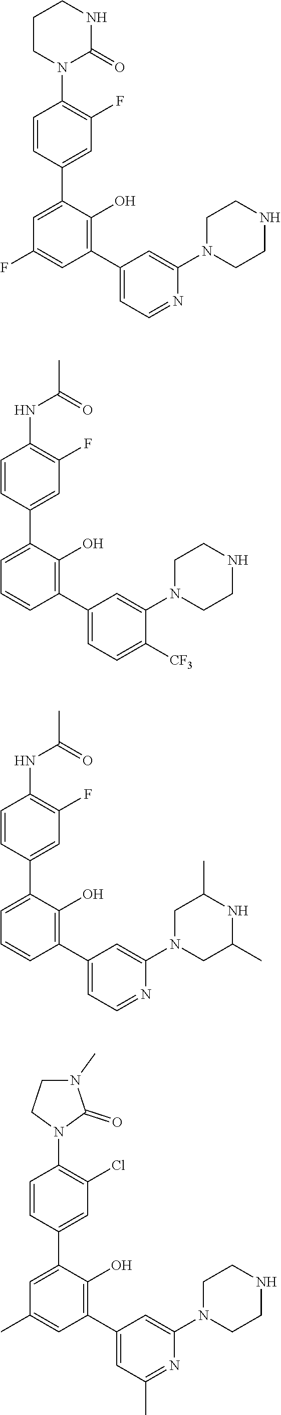

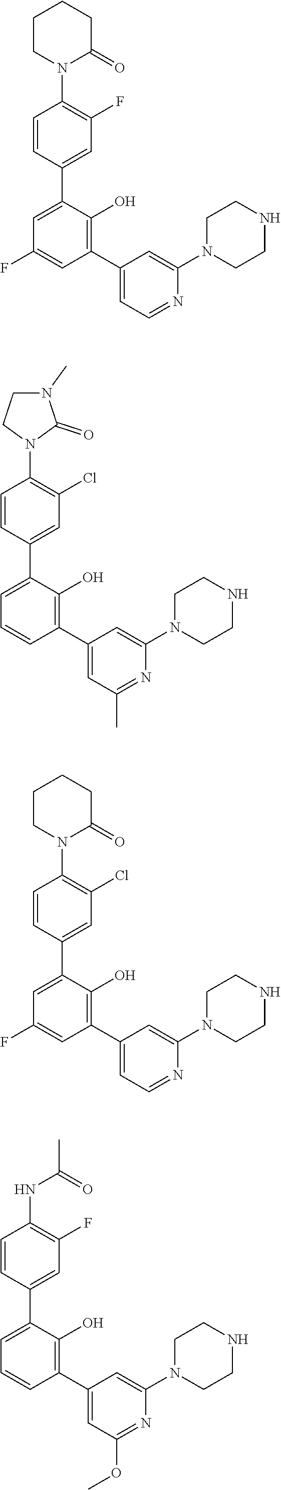 [SMILES: O=C1CCCCN1c1ccc(-c2cc(F)cc(-c3ccnc(N4CCNCC4)c3)c2O)cc1F ; Cc1cc(-c2cccc(-c3ccc(N4CCN(C)C4=O)c(Cl)c3)c2O)cc(N2CCNCC2)n1 ; O=C1CCCCN1c1ccc(-c2cc(F)cc(-c3ccnc(N4CCNCC4)c3)c2O)cc1Cl ; COc1cc(-c2cccc(-c3ccc(NC(C)=O)c(F)c3)c2O)cc(N2CCNCC2)n1]

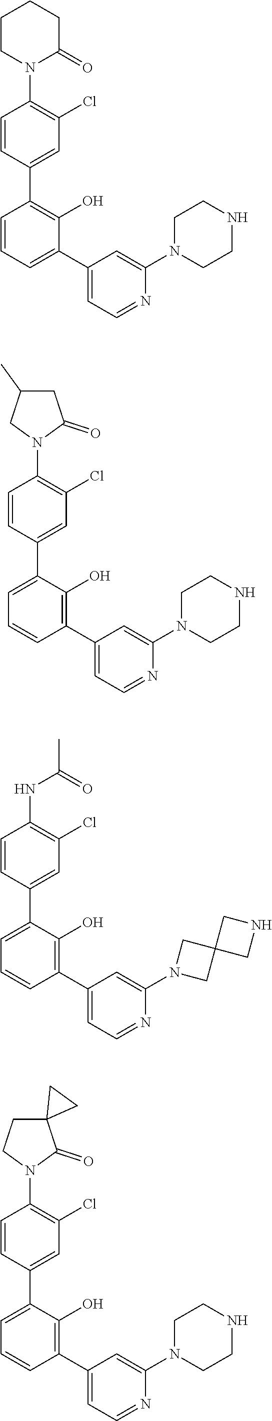 [SMILES: O=C1CCCCN1c1ccc(-c2cccc(-c3ccnc(N4CCNCC4)c3)c2O)cc1Cl ; CC1CC(=O)N(c2ccc(-c3cccc(-c4ccnc(N5CCNCC5)c4)c3O)cc2Cl)C1 ; CC(=O)Nc1ccc(-c2cccc(-c3ccnc(N4CC5(CNC5)C4)c3)c2O)cc1Cl ; O=C1N(c2ccc(-c3cccc(-c4ccnc(N5CCNCC5)c4)c3O)cc2Cl)CCC12CC2]

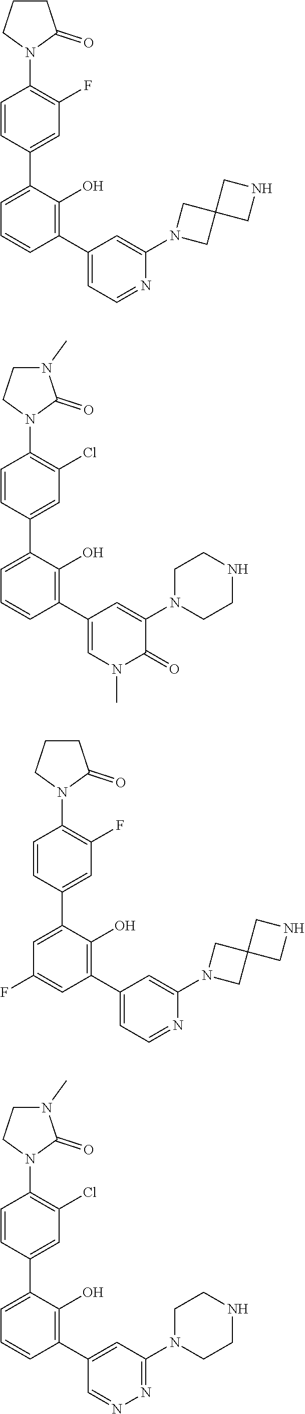 [SMILES: O=C1CCCN1c1ccc(-c2cccc(-c3ccnc(N4CC5(CNC5)C4)c3)c2O)cc1F ; CN1CCN(c2ccc(-c3cccc(-c4cc(N5CCNCC5)c(=O)n(C)c4)c3O)cc2Cl)C1=O ; O=C1CCCN1c1ccc(-c2cc(F)cc(-c3ccnc(N4CC5(CNC5)C4)c3)c2O)cc1F ; CN1CCN(c2ccc(-c3cccc(-c4cnnc(N5CCNCC5)c4)c3O)cc2Cl)C1=O]

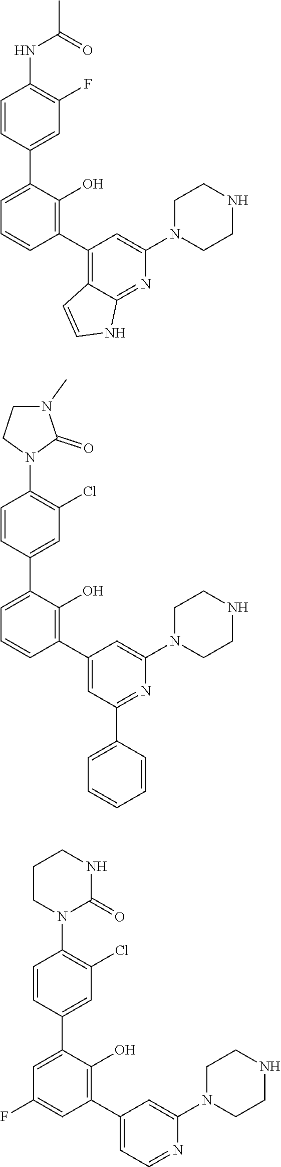

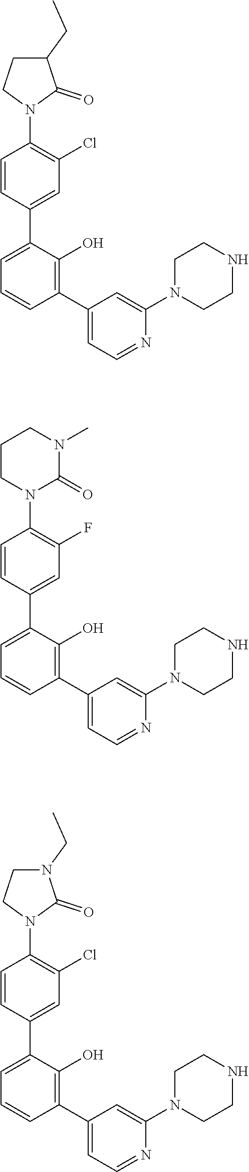 [SMILES: CCC1CCN(c2ccc(-c3cccc(-c4ccnc(N5CCNCC5)c4)c3O)cc2Cl)C1=O ; CN1CCCN(c2ccc(-c3cccc(-c4ccnc(N5CCNCC5)c4)c3O)cc2F)C1=O ; CCN1CCN(c2ccc(-c3cccc(-c4ccnc(N5CCNCC5)c4)c3O)cc2Cl)C1=O]

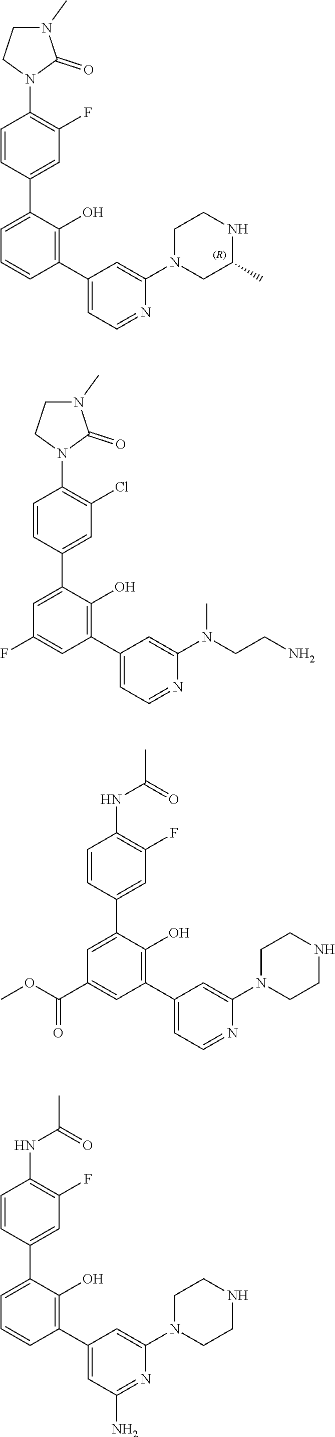

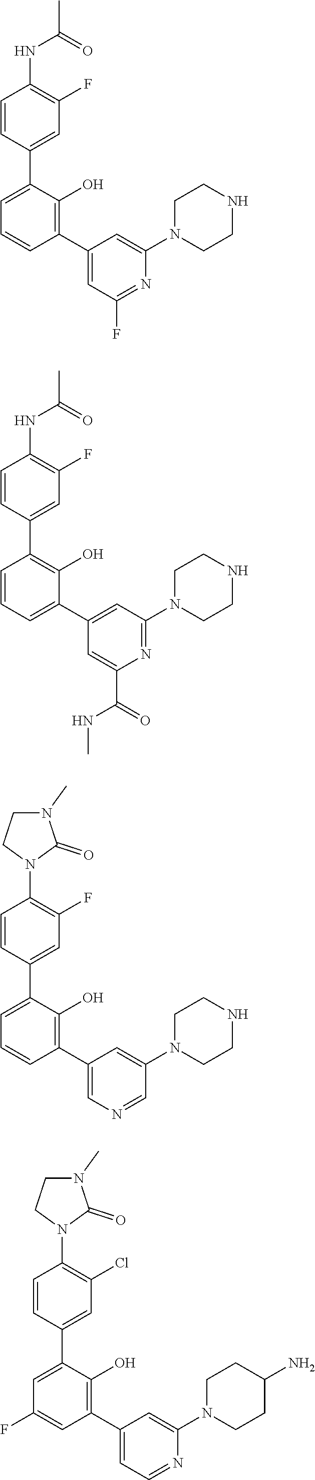

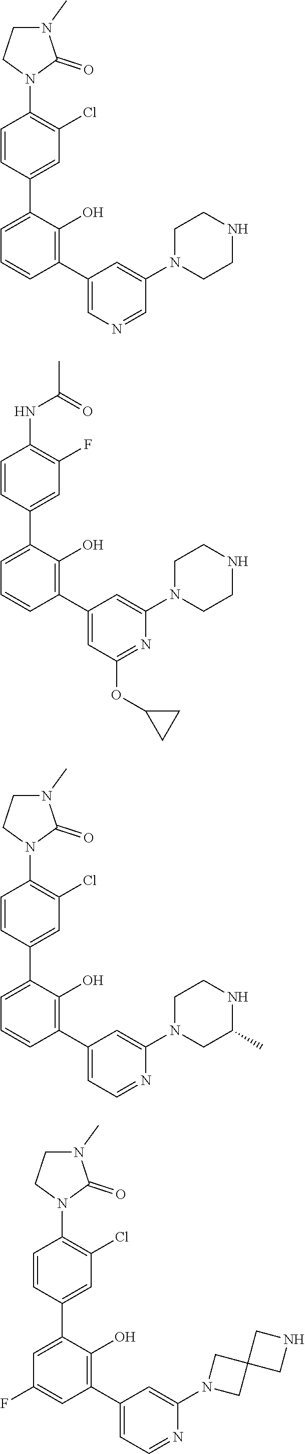

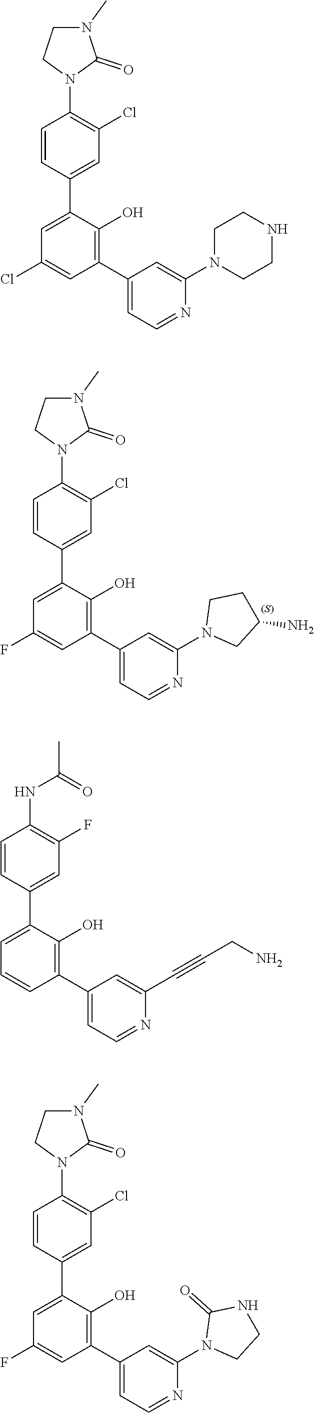

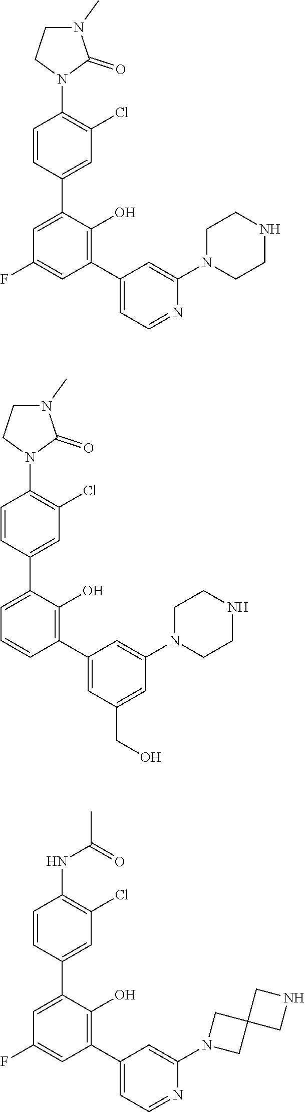 [SMILES: CN1CCN(c2ccc(-c3cc(F)cc(-c4ccnc(N5CCNCC5)c4)c3O)cc2Cl)C1=O ; CN1CCN(c2ccc(-c3cccc(-c4cc(CO)cc(N5CCNCC5)c4)c3O)cc2Cl)C1=O ; CC(=O)Nc1ccc(-c2cc(F)cc(-c3ccnc(N4CC5(CNC5)C4)c3)c2O)cc1Cl]

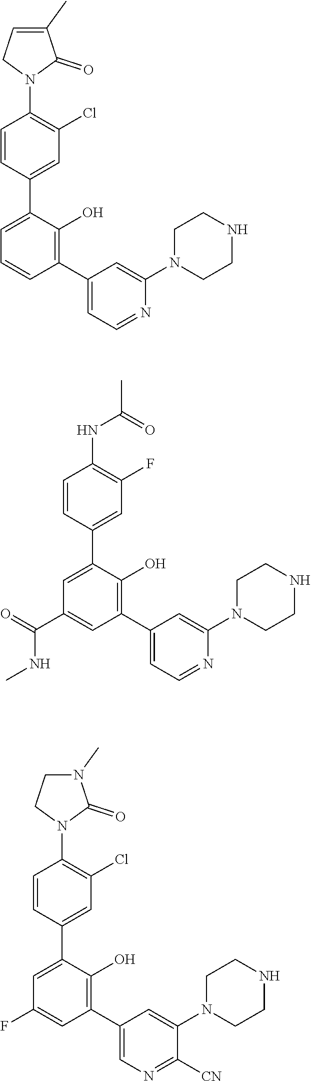 [SMILES: CC1=CCN(c2ccc(-c3cccc(-c4ccnc(N5CCNCC5)c4)c3O)cc2Cl)C1=O ; CNC(=O)c1cc(-c2ccnc(N3CCNCC3)c2)c(O)c(-c2ccc(NC(C)=O)c(F)c2)c1 ; CN1CCN(c2ccc(-c3cc(F)cc(-c4cnc(C#N)c(N5CCNCC5)c4)c3O)cc2Cl)C1=O]

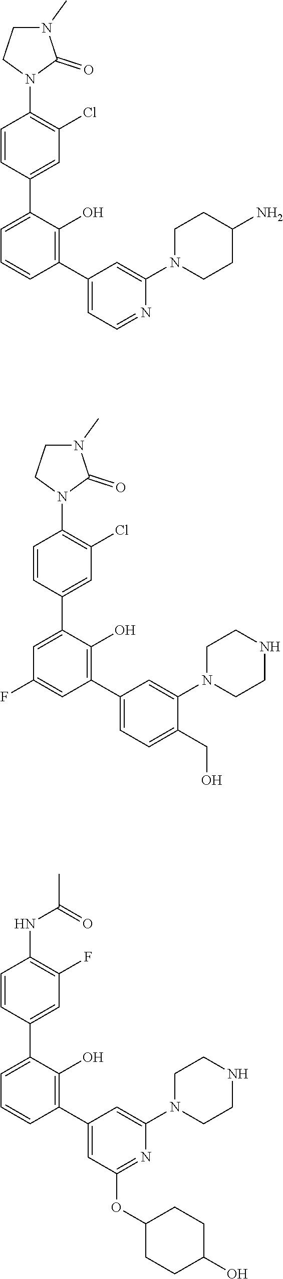 [SMILES: CN1CCN(c2ccc(-c3cccc(-c4ccnc(N5CCC(N)CC5)c4)c3O)cc2Cl)C1=O ; CN1CCN(c2ccc(-c3cc(F)cc(-c4ccc(CO)c(N5CCNCC5)c4)c3O)cc2Cl)C1=O ; CC(=O)Nc1ccc(-c2cccc(-c3cc(OC4CCC(O)CC4)nc(N4CCNCC4)c3)c2O)cc1F]

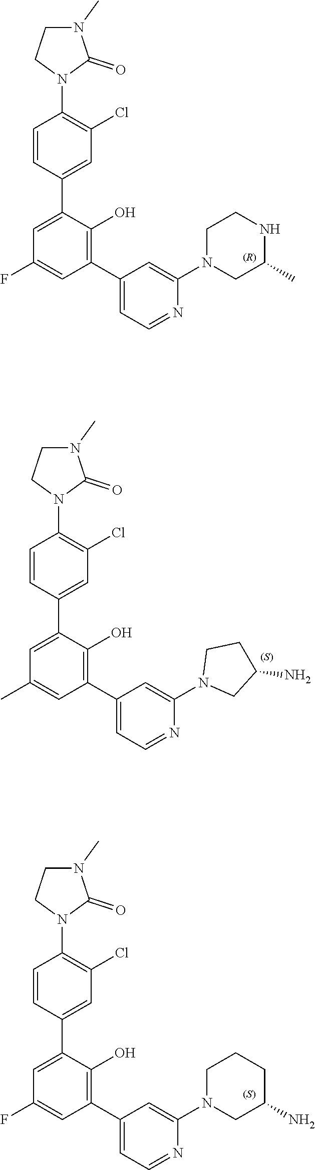

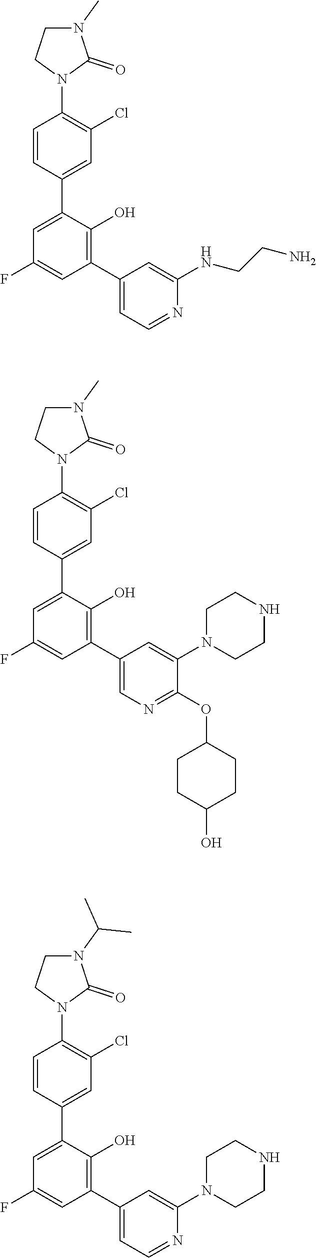 [SMILES: CN1CCN(c2ccc(-c3cc(F)cc(-c4ccnc(NCCN)c4)c3O)cc2Cl)C1=O ; CN1CCN(c2ccc(-c3cc(F)cc(-c4cnc(OC5CCC(O)CC5)c(N5CCNCC5)c4)c3O)cc2Cl)C1=O ; CC(C)N1CCN(c2ccc(-c3cc(F)cc(-c4ccnc(N5CCNCC5)c4)c3O)cc2Cl)C1=O]

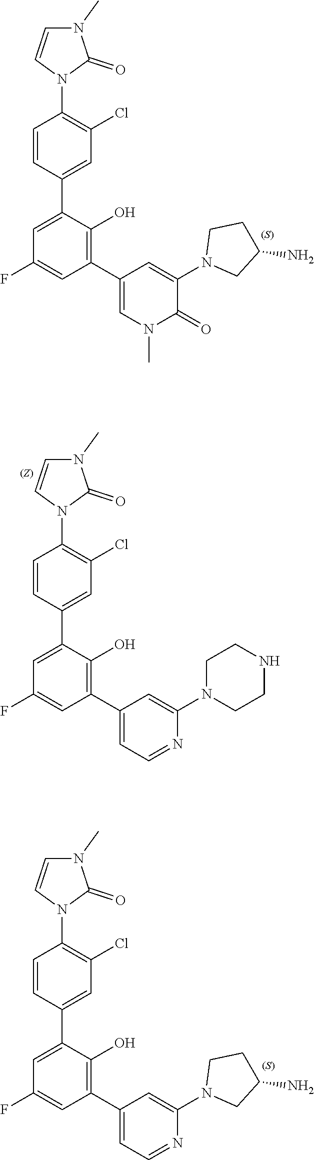

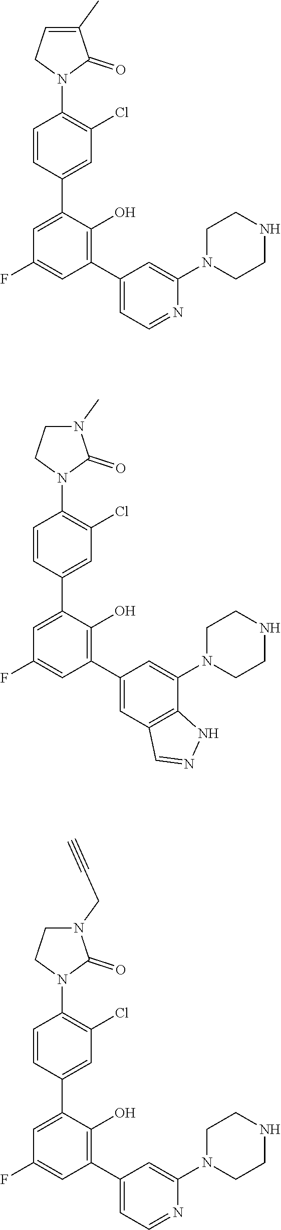 [SMILES: CC1=CCN(c2ccc(-c3cc(F)cc(-c4ccnc(N5CCNCC5)c4)c3O)cc2Cl)C1=O ; CN1CCN(c2ccc(-c3cc(F)cc(-c4cc(N5CCNCC5)c5[nH]ncc5c4)c3O)cc2Cl)C1=O ; C#CCN1CCN(c2ccc(-c3cc(F)cc(-c4ccnc(N5CCNCC5)c4)c3O)cc2Cl)C1=O]

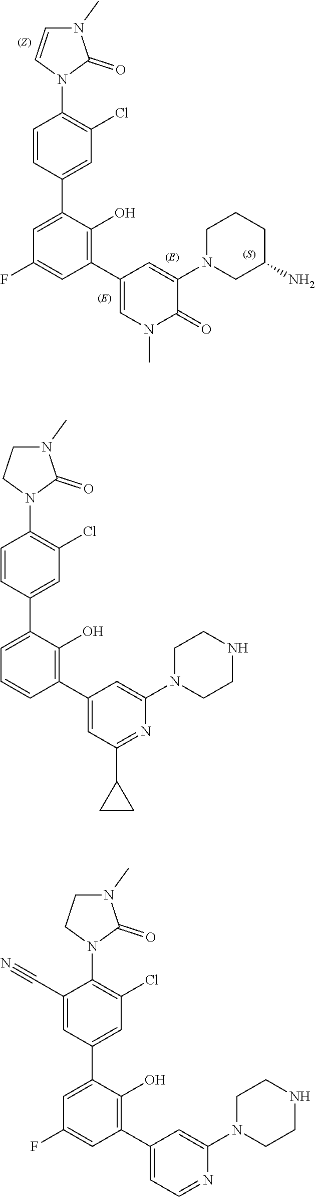

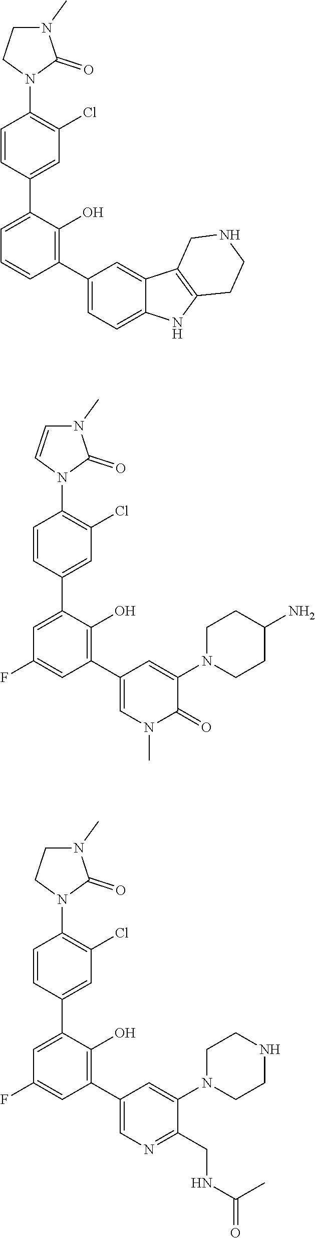 [SMILES: CN1CCN(c2ccc(-c3cccc(-c4ccc5[nH]c6c(c5c4)CNCC6)c3O)cc2Cl)C1=O ; Cn1cc(-c2cc(F)cc(-c3ccc(-n4ccn(C)c4=O)c(Cl)c3)c2O)cc(N2CCC(N)CC2)c1=O ; CC(=O)NCc1ncc(-c2cc(F)cc(-c3ccc(N4CCN(C)C4=O)c(Cl)c3)c2O)cc1N1CCNCC1]

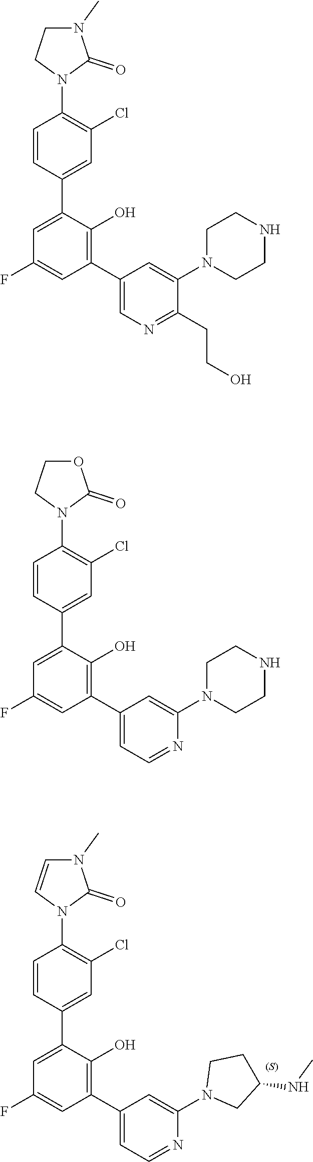

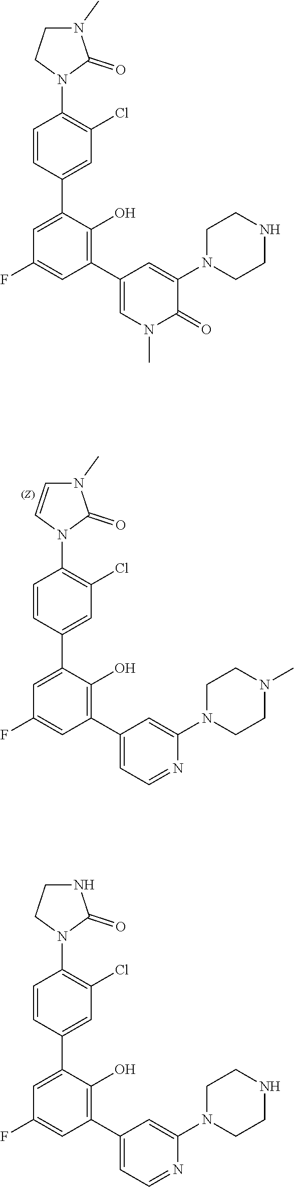 [SMILES: CN1CCN(c2ccc(-c3cc(F)cc(-c4cc(N5CCNCC5)c(=O)n(C)c4)c3O)cc2Cl)C1=O ; CN1CCN(c2cc(-c3cc(F)cc(-c4ccc(-n5ccn(C)c5=O)c(Cl)c4)c3O)ccn2)CC1 ; O=C1NCCN1c1ccc(-c2cc(F)cc(-c3ccnc(N4CCNCC4)c3)c2O)cc1Cl]

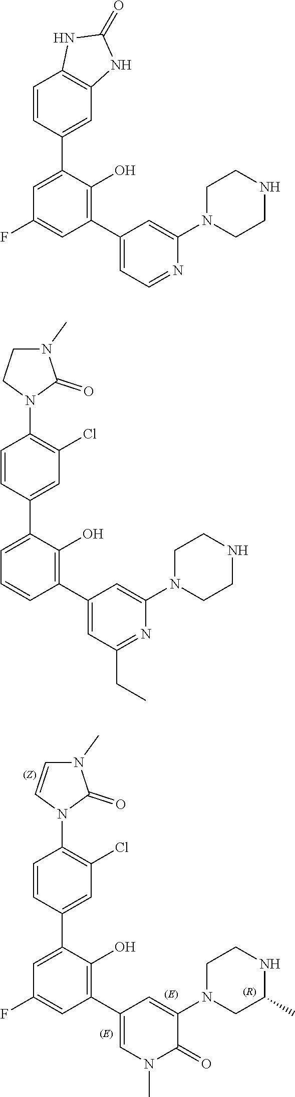

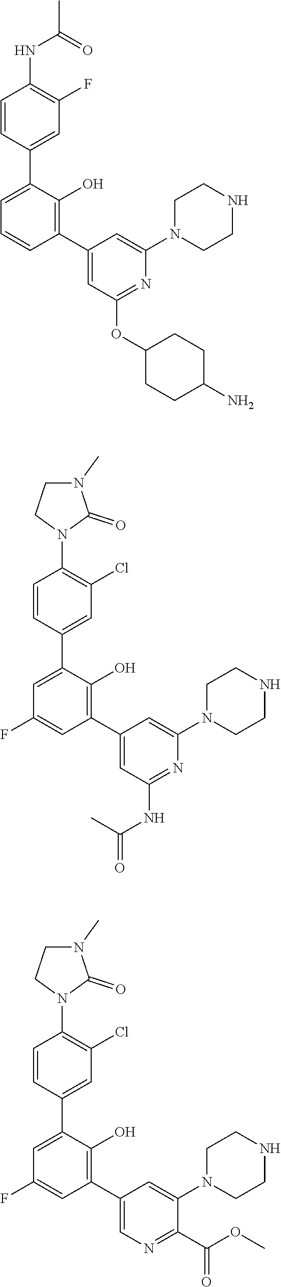 [SMILES: CC(=O)Nc1ccc(-c2cccc(-c3cc(OC4CCC(N)CC4)nc(N4CCNCC4)c3)c2O)cc1F ; CC(=O)Nc1cc(-c2cc(F)cc(-c3ccc(N4CCN(C)C4=O)c(Cl)c3)c2O)cc(N2CCNCC2)n1 ; COC(=O)c1ncc(-c2cc(F)cc(-c3ccc(N4CCN(C)C4=O)c(Cl)c3)c2O)cc1N1CCNCC1]

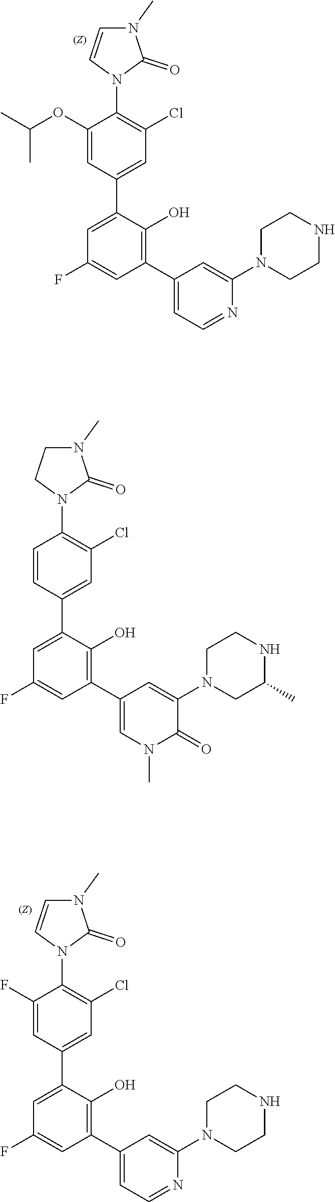 [SMILES: CC(C)Oc1cc(-c2cc(F)cc(-c3ccnc(N4CCNCC4)c3)c2O)cc(Cl)c1-n1ccn(C)c1=O ; C[C@@H]1CN(c2cc(-c3cc(F)cc(-c4ccc(N5CCN(C)C5=O)c(Cl)c4)c3O)cn(C)c2=O)CCN1 ; Cn1ccn(-c2c(F)cc(-c3cc(F)cc(-c4ccnc(N5CCNCC5)c4)c3O)cc2Cl)c1=O]

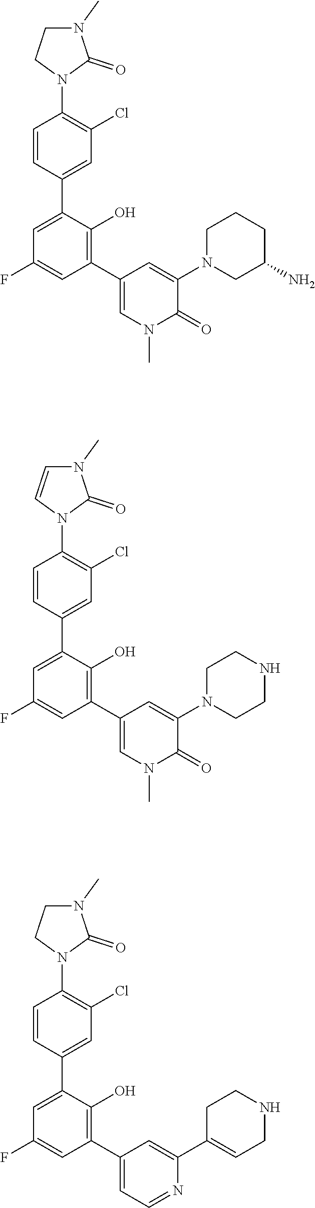 [SMILES: CN1CCN(c2ccc(-c3cc(F)cc(-c4cc(N5CCC[C@H](N)C5)c(=O)n(C)c4)c3O)cc2Cl)C1=O ; Cn1cc(-c2cc(F)cc(-c3ccc(-n4ccn(C)c4=O)c(Cl)c3)c2O)cc(N2CCNCC2)c1=O ; CN1CCN(c2ccc(-c3cc(F)cc(-c4ccnc(C5=CCNCC5)c4)c3O)cc2Cl)C1=O]

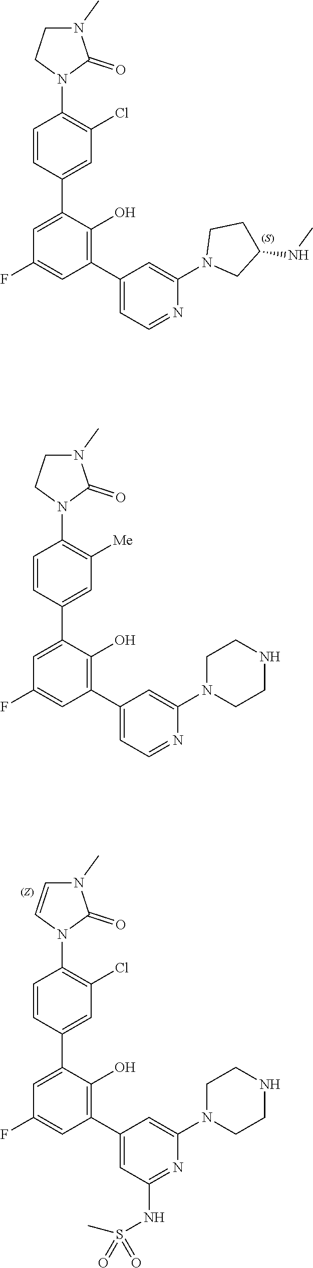

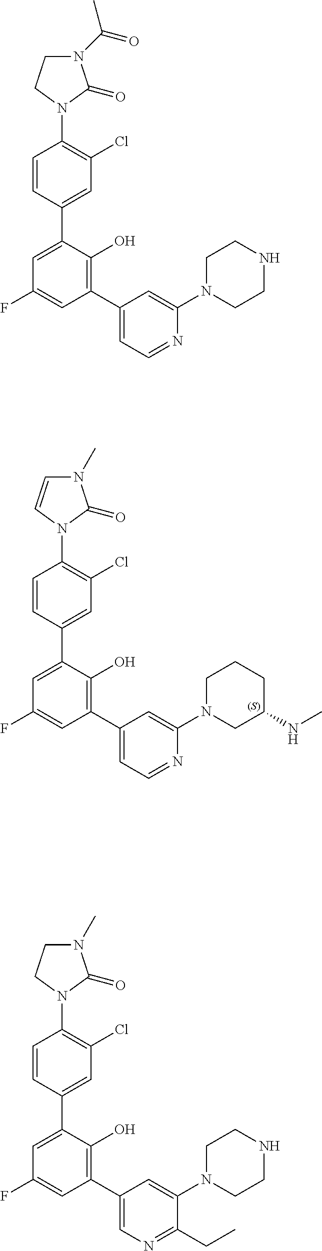

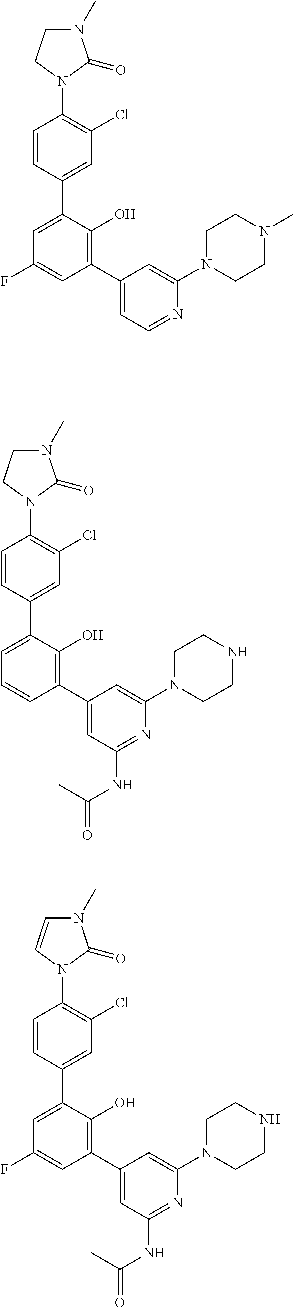 [SMILES: CN1CCN(c2cc(-c3cc(F)cc(-c4ccc(N5CCN(C)C5=O)c(Cl)c4)c3O)ccn2)CC1 ; CC(=O)Nc1cc(-c2cccc(-c3ccc(N4CCN(C)C4=O)c(Cl)c3)c2O)cc(N2CCNCC2)n1 ; CC(=O)Nc1cc(-c2cc(F)cc(-c3ccc(-n4ccn(C)c4=O)c(Cl)c3)c2O)cc(N2CCNCC2)n1]

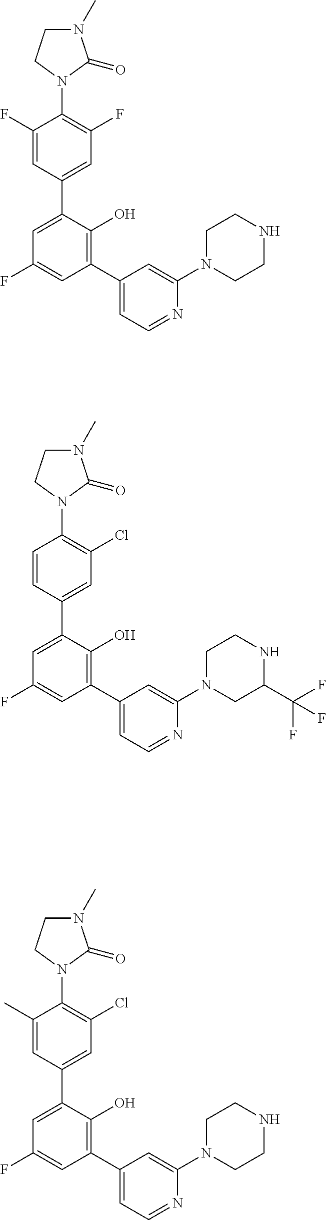 [SMILES: CN1CCN(c2c(F)cc(-c3cc(F)cc(-c4ccnc(N5CCNCC5)c4)c3O)cc2F)C1=O ; CN1CCN(c2ccc(-c3cc(F)cc(-c4ccnc(N5CCNC(C(F)(F)F)C5)c4)c3O)cc2Cl)C1=O ; Cc1cc(-c2cc(F)cc(-c3ccnc(N4CCNCC4)c3)c2O)cc(Cl)c1N1CCN(C)C1=O]

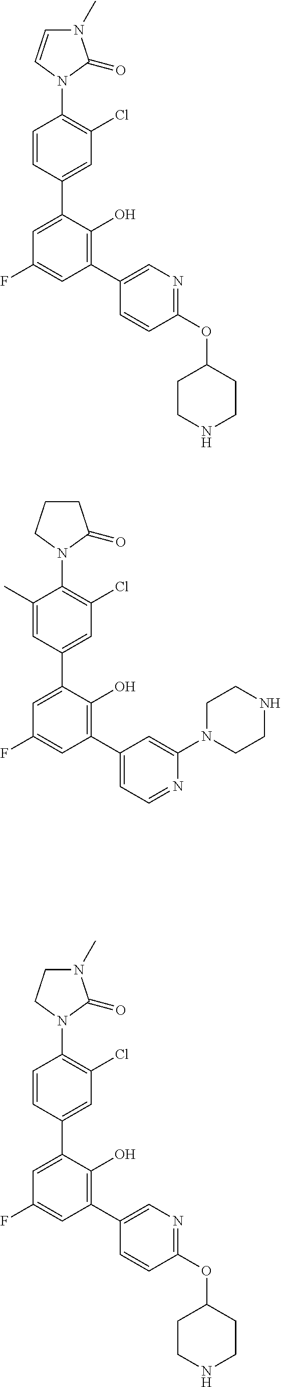 [SMILES: Cn1ccn(-c2ccc(-c3cc(F)cc(-c4ccc(OC5CCNCC5)nc4)c3O)cc2Cl)c1=O ; Cc1cc(-c2cc(F)cc(-c3ccnc(N4CCNCC4)c3)c2O)cc(Cl)c1N1CCCC1=O ; CN1CCN(c2ccc(-c3cc(F)cc(-c4ccc(OC5CCNCC5)nc4)c3O)cc2Cl)C1=O]

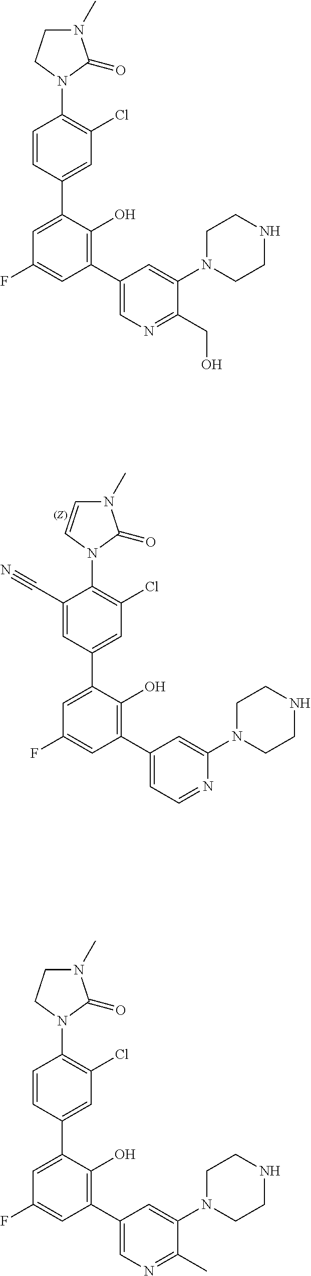 [SMILES: CN1CCN(c2ccc(-c3cc(F)cc(-c4cnc(CO)c(N5CCNCC5)c4)c3O)cc2Cl)C1=O ; Cn1ccn(-c2c(Cl)cc(-c3cc(F)cc(-c4ccnc(N5CCNCC5)c4)c3O)cc2C#N)c1=O ; Cc1ncc(-c2cc(F)cc(-c3ccc(N4CCN(C)C4=O)c(Cl)c3)c2O)cc1N1CCNCC1]

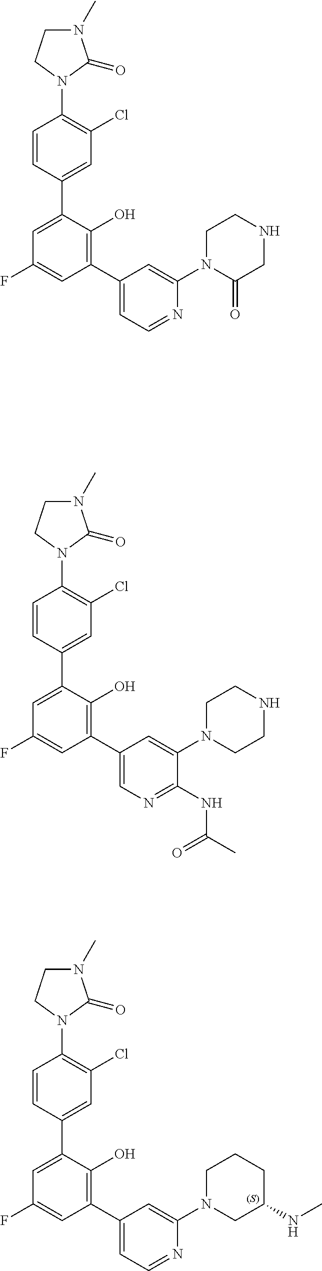

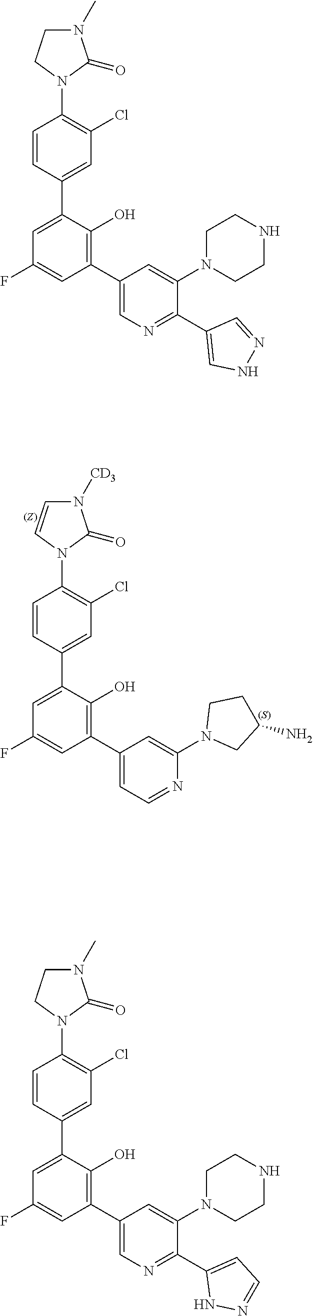

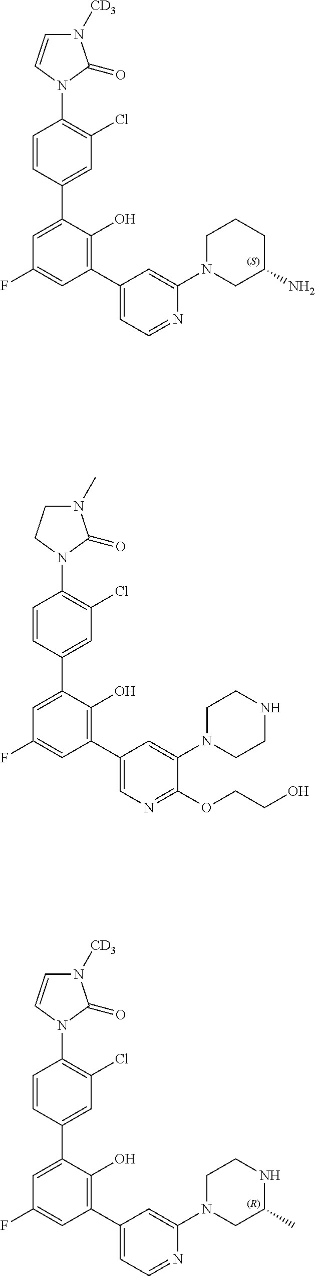

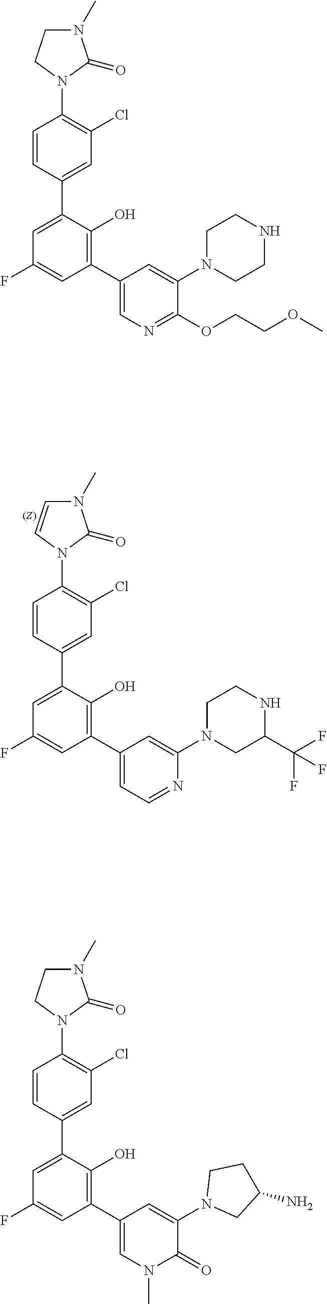 [SMILES: COCCOc1ncc(-c2cc(F)cc(-c3ccc(N4CCN(C)C4=O)c(Cl)c3)c2O)cc1N1CCNCC1 ; Cn1ccn(-c2ccc(-c3cc(F)cc(-c4ccnc(N5CCNC(C(F)(F)F)C5)c4)c3O)cc2Cl)c1=O ; CN1CCN(c2ccc(-c3cc(F)cc(-c4cc(N5CC[C@H](N)C5)c(=O)n(C)c4)c3O)cc2Cl)C1=O]

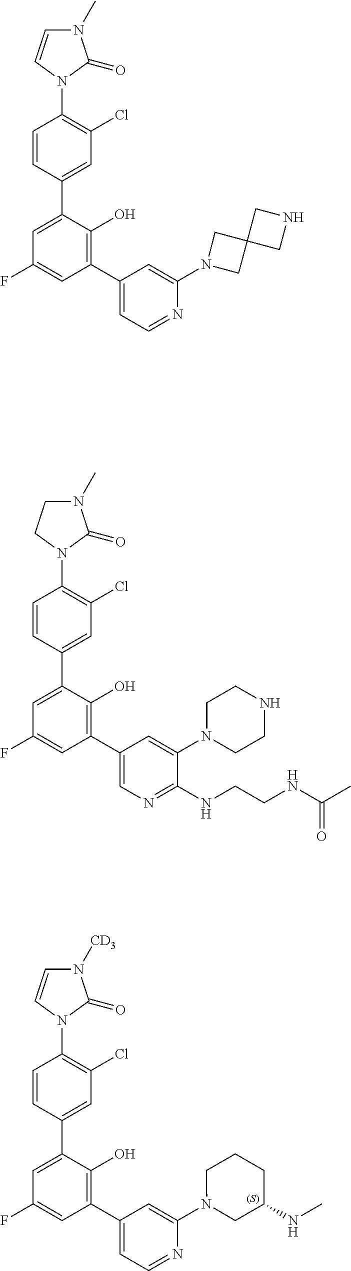

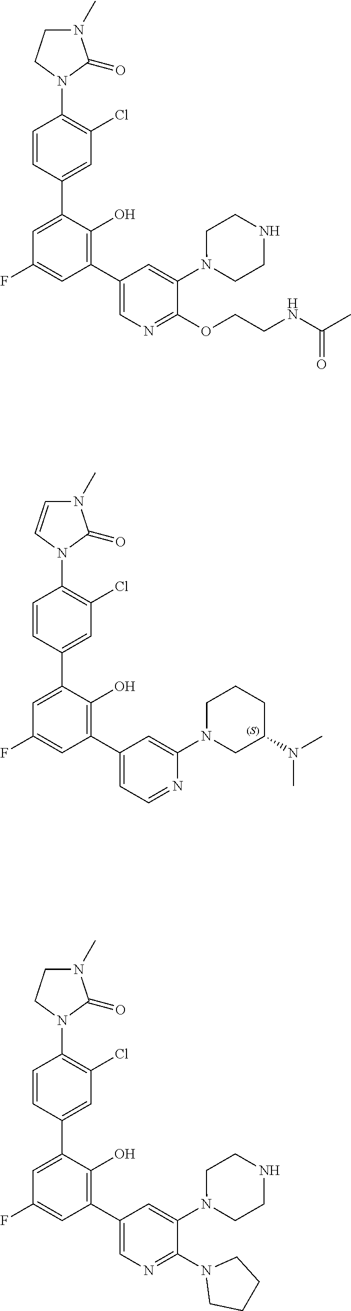

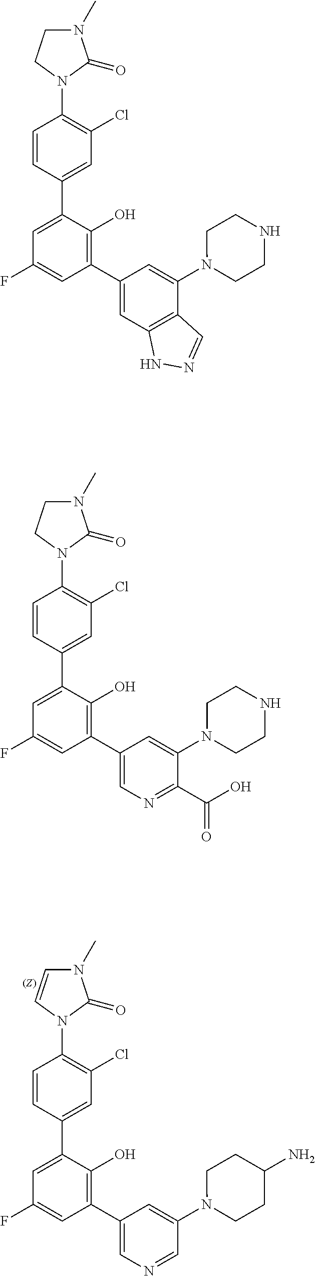 [SMILES: CN1CCN(c2ccc(-c3cc(F)cc(-c4cc(N5CCNCC5)c5cn[nH]c5c4)c3O)cc2Cl)C1=O ; CN1CCN(c2ccc(-c3cc(F)cc(-c4cnc(C(=O)O)c(N5CCNCC5)c4)c3O)cc2Cl)C1=O ; Cn1ccn(-c2ccc(-c3cc(F)cc(-c4cncc(N5CCC(N)CC5)c4)c3O)cc2Cl)c1=O]

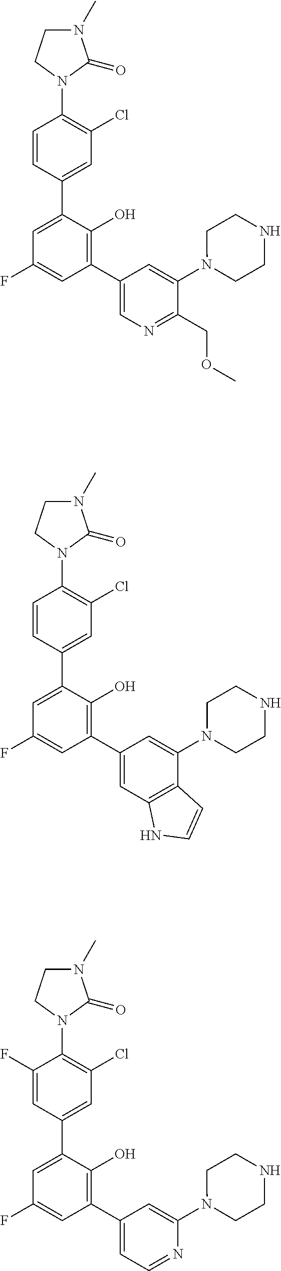 [SMILES: COCc1ncc(-c2cc(F)cc(-c3ccc(N4CCN(C)C4=O)c(Cl)c3)c2O)cc1N1CCNCC1 ; CN1CCN(c2ccc(-c3cc(F)cc(-c4cc(N5CCNCC5)c5cc[nH]c5c4)c3O)cc2Cl)C1=O ; CN1CCN(c2c(F)cc(-c3cc(F)cc(-c4ccnc(N5CCNCC5)c4)c3O)cc2Cl)C1=O]

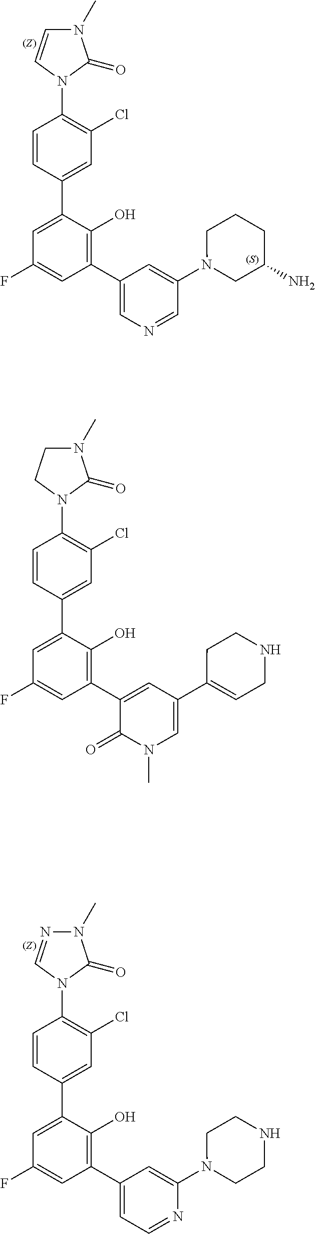

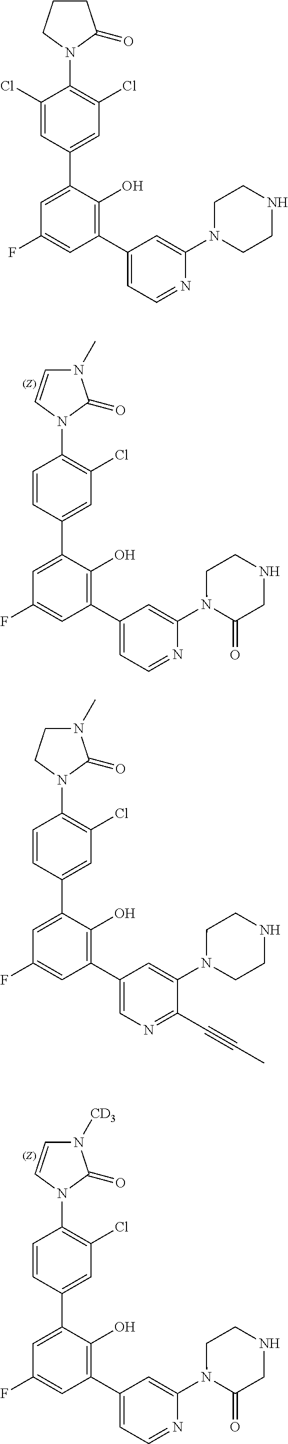 [SMILES: O=C1CCCN1c1c(Cl)cc(-c2cc(F)cc(-c3ccnc(N4CCNCC4)c3)c2O)cc1Cl ; Cn1ccn(-c2ccc(-c3cc(F)cc(-c4ccnc(N5CCNCC5=O)c4)c3O)cc2Cl)c1=O ; CC#Cc1ncc(-c2cc(F)cc(-c3ccc(N4CCN(C)C4=O)c(Cl)c3)c2O)cc1N1CCNCC1 ; [2H]C([2H])([2H])n1ccn(-c2ccc(-c3cc(F)cc(-c4ccnc(N5CCNCC5=O)c4)c3O)cc2Cl)c1=O]

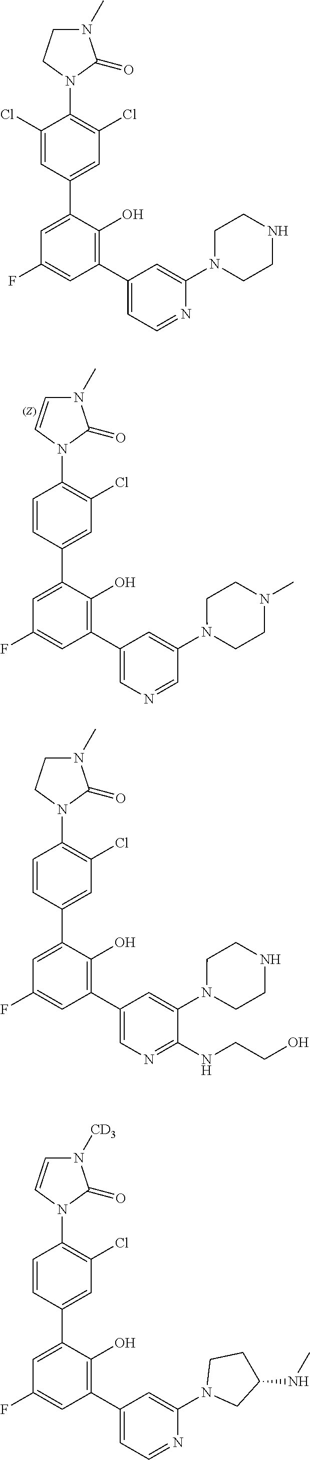 [SMILES: CN1CCN(c2c(Cl)cc(-c3cc(F)cc(-c4ccnc(N5CCNCC5)c4)c3O)cc2Cl)C1=O ; CN1CCN(c2cncc(-c3cc(F)cc(-c4ccc(-n5ccn(C)c5=O)c(Cl)c4)c3O)c2)CC1 ; CN1CCN(c2ccc(-c3cc(F)cc(-c4cnc(NCCO)c(N5CCNCC5)c4)c3O)cc2Cl)C1=O ; [2H]C([2H])([2H])n1ccn(-c2ccc(-c3cc(F)cc(-c4ccnc(N5CC[C@H](NC)C5)c4)c3O)cc2Cl)c1=O]

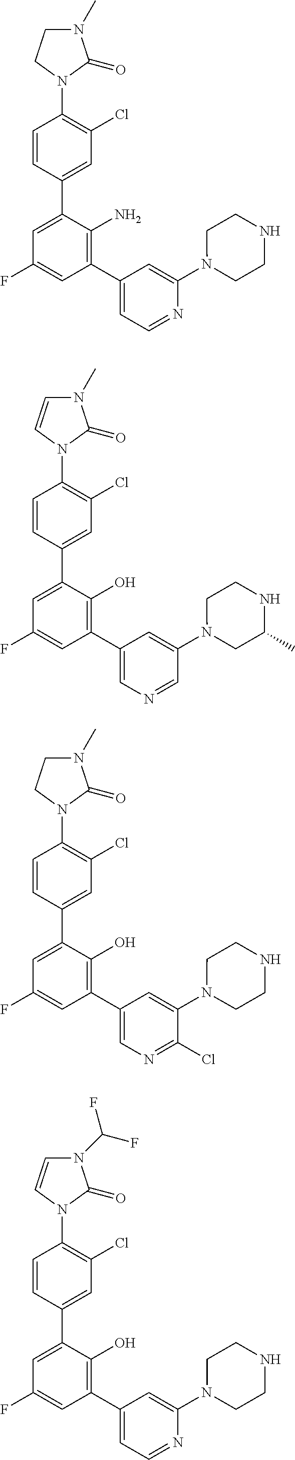 [SMILES: CN1CCN(c2ccc(-c3cc(F)cc(-c4ccnc(N5CCNCC5)c4)c3N)cc2Cl)C1=O ; C[C@@H]1CN(c2cncc(-c3cc(F)cc(-c4ccc(-n5ccn(C)c5=O)c(Cl)c4)c3O)c2)CCN1 ; CN1CCN(c2ccc(-c3cc(F)cc(-c4cnc(Cl)c(N5CCNCC5)c4)c3O)cc2Cl)C1=O ; O=c1n(-c2ccc(-c3cc(F)cc(-c4ccnc(N5CCNCC5)c4)c3O)cc2Cl)ccn1C(F)F]

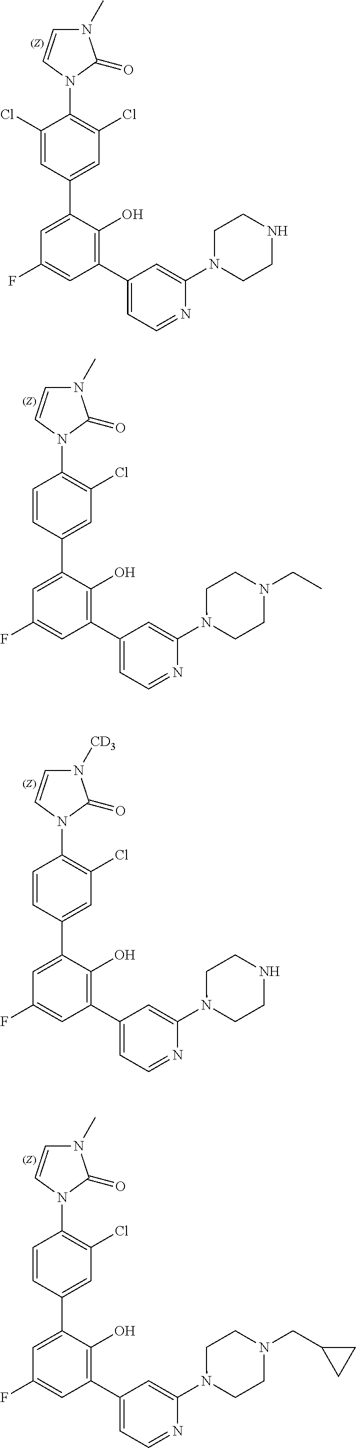 [SMILES: Cn1ccn(-c2c(Cl)cc(-c3cc(F)cc(-c4ccnc(N5CCNCC5)c4)c3O)cc2Cl)c1=O ; CCN1CCN(c2cc(-c3cc(F)cc(-c4ccc(-n5ccn(C)c5=O)c(Cl)c4)c3O)ccn2)CC1 ; [2H]C([2H])([2H])n1ccn(-c2ccc(-c3cc(F)cc(-c4ccnc(N5CCNCC5)c4)c3O)cc2Cl)c1=O ; Cn1ccn(-c2ccc(-c3cc(F)cc(-c4ccnc(N5CCN(CC6CC6)CC5)c4)c3O)cc2Cl)c1=O]

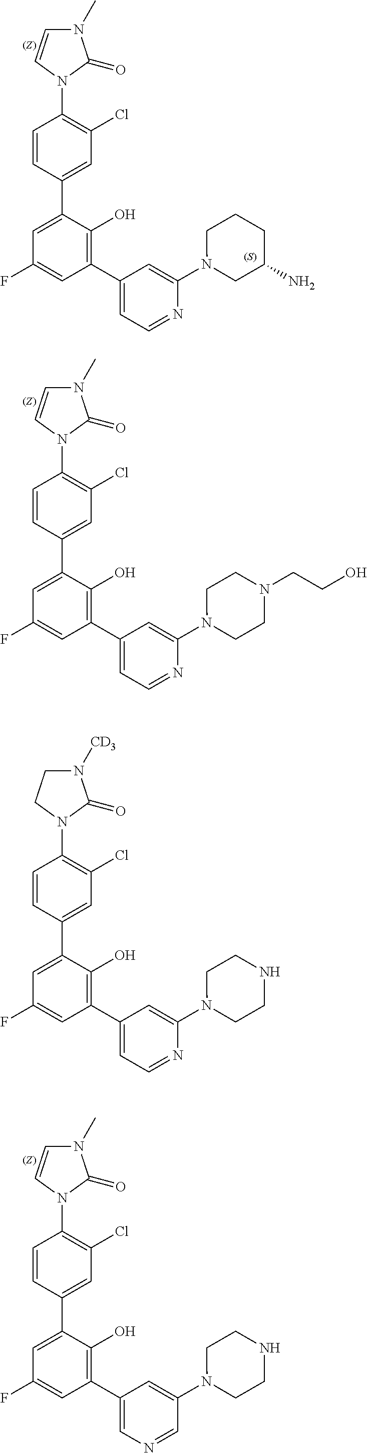

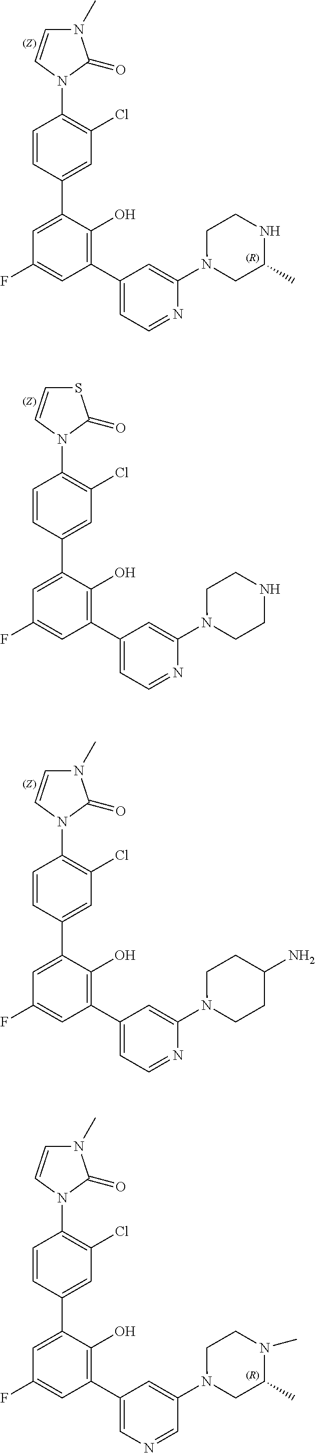

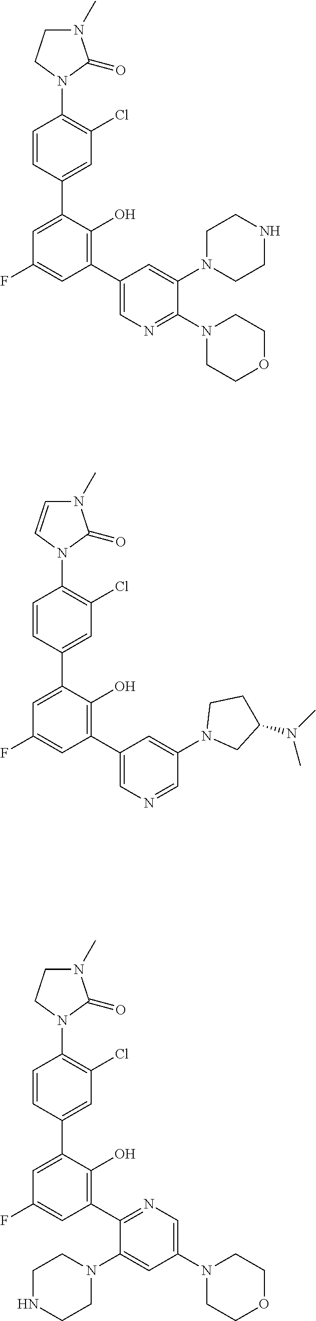 [SMILES: CN1CCN(c2ccc(-c3cc(F)cc(-c4cnc(N5CCOCC5)c(N5CCNCC5)c4)c3O)cc2Cl)C1=O ; CN(C)[C@H]1CCN(c2cncc(-c3cc(F)cc(-c4ccc(-n5ccn(C)c5=O)c(Cl)c4)c3O)c2)C1 ; CN1CCN(c2ccc(-c3cc(F)cc(-c4ncc(N5CCOCC5)cc4N4CCNCC4)c3O)cc2Cl)C1=O]

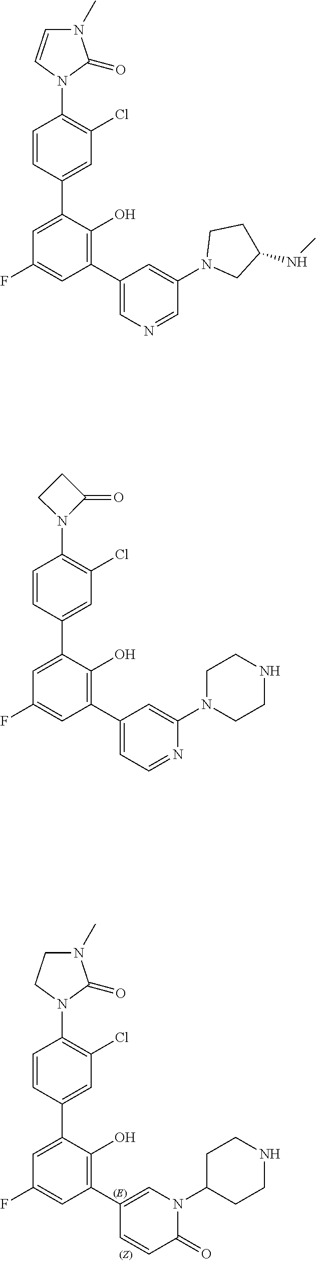 [SMILES: CN[C@H]1CCN(c2cncc(-c3cc(F)cc(-c4ccc(-n5ccn(C)c5=O)c(Cl)c4)c3O)c2)C1 ; O=C1CCN1c1ccc(-c2cc(F)cc(-c3ccnc(N4CCNCC4)c3)c2O)cc1Cl ; CN1CCN(c2ccc(-c3cc(F)cc(-c4ccc(=O)n(C5CCNCC5)c4)c3O)cc2Cl)C1=O]

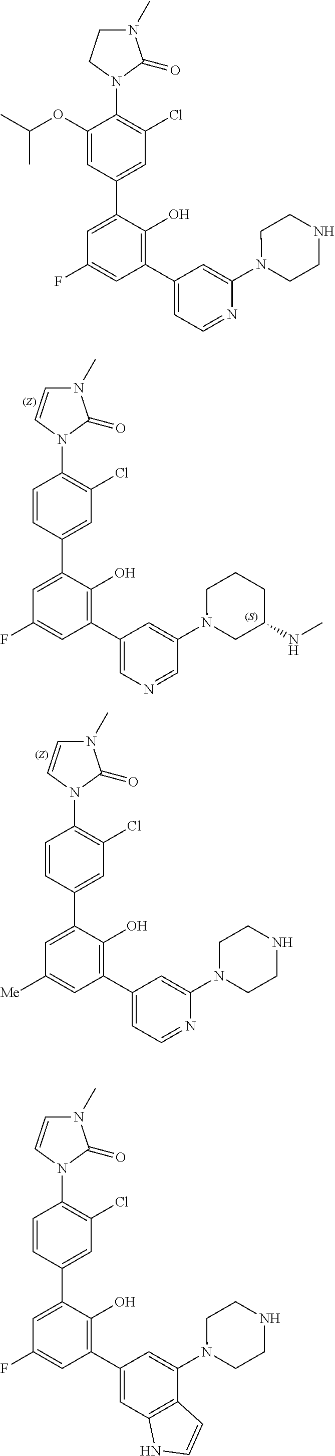

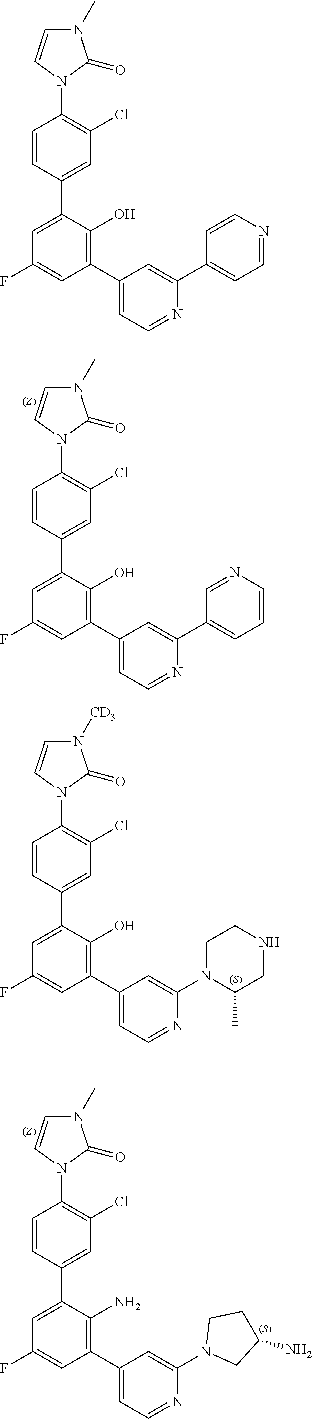

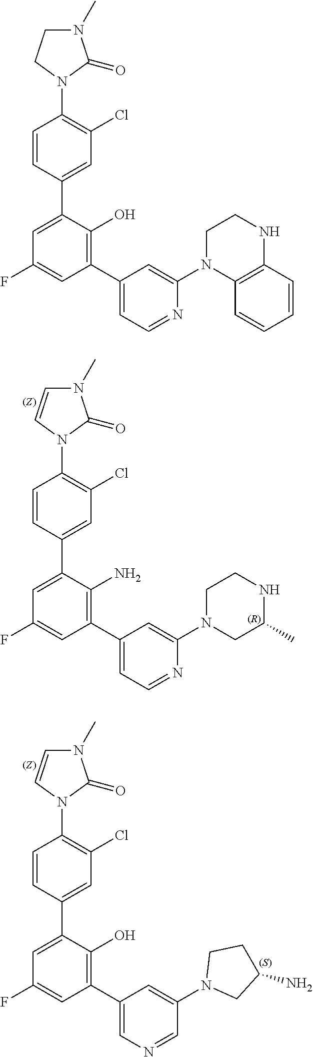

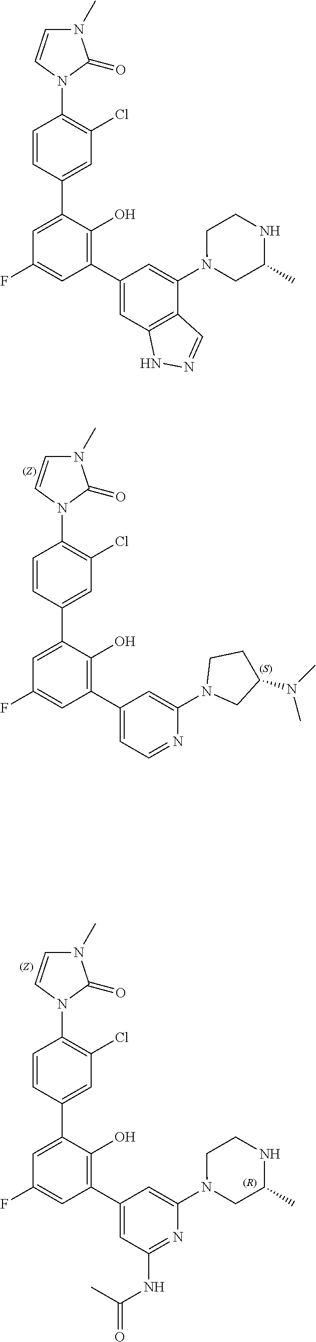

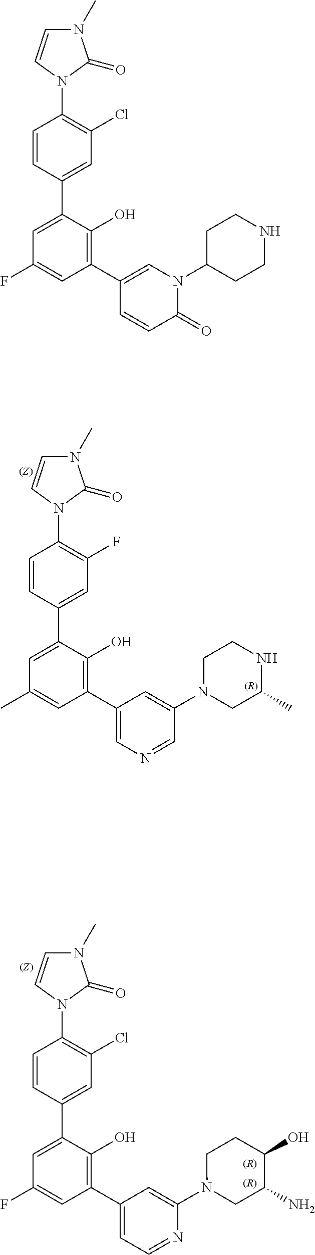

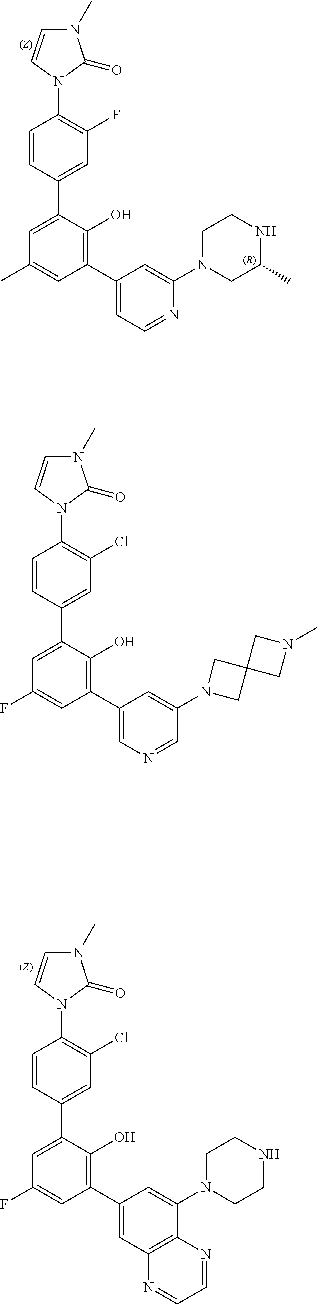

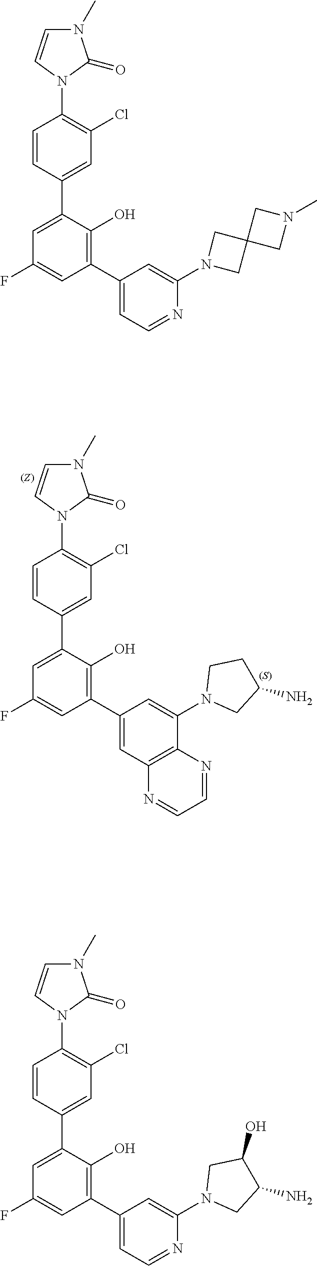

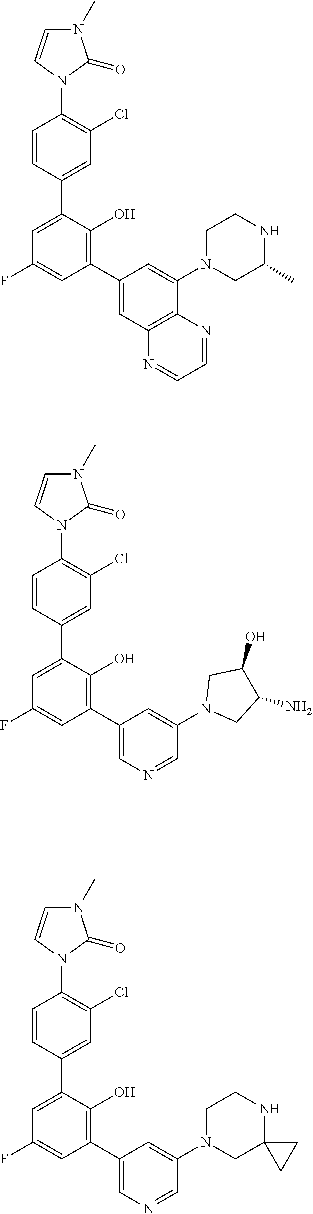 [SMILES: C[C@@H]1CN(c2cc(-c3cc(F)cc(-c4ccc(-n5ccn(C)c5=O)c(Cl)c4)c3O)cc3nccnc23)CCN1 ; Cn1ccn(-c2ccc(-c3cc(F)cc(-c4cncc(N5C[C@@H](N)[C@H](O)C5)c4)c3O)cc2Cl)c1=O ; Cn1ccn(-c2ccc(-c3cc(F)cc(-c4cncc(N5CCNC6(CC6)C5)c4)c3O)cc2Cl)c1=O]

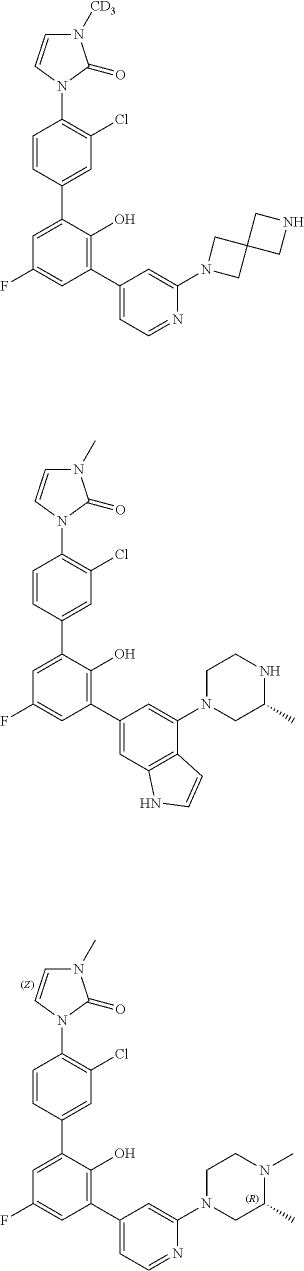

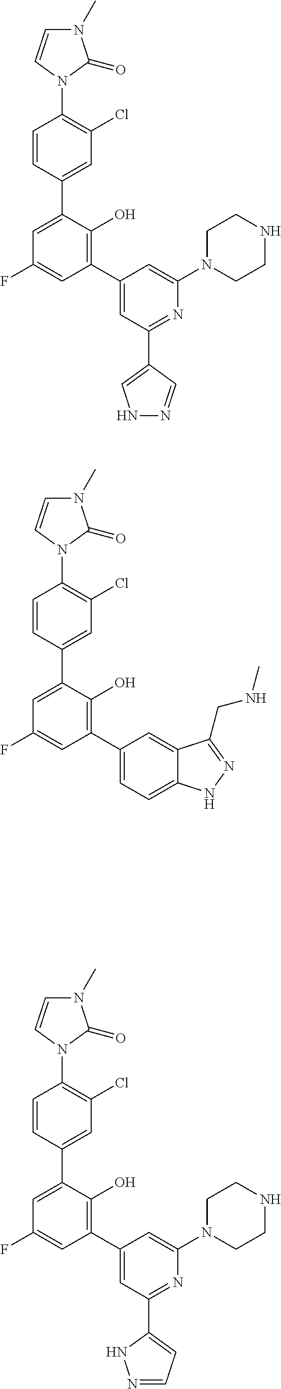 [SMILES: Cn1ccn(-c2ccc(-c3cc(F)cc(-c4cc(-c5cn[nH]c5)nc(N5CCNCC5)c4)c3O)cc2Cl)c1=O ; CNCc1n[nH]c2ccc(-c3cc(F)cc(-c4ccc(-n5ccn(C)c5=O)c(Cl)c4)c3O)cc12 ; Cn1ccn(-c2ccc(-c3cc(F)cc(-c4cc(-c5ccn[nH]5)nc(N5CCNCC5)c4)c3O)cc2Cl)c1=O]

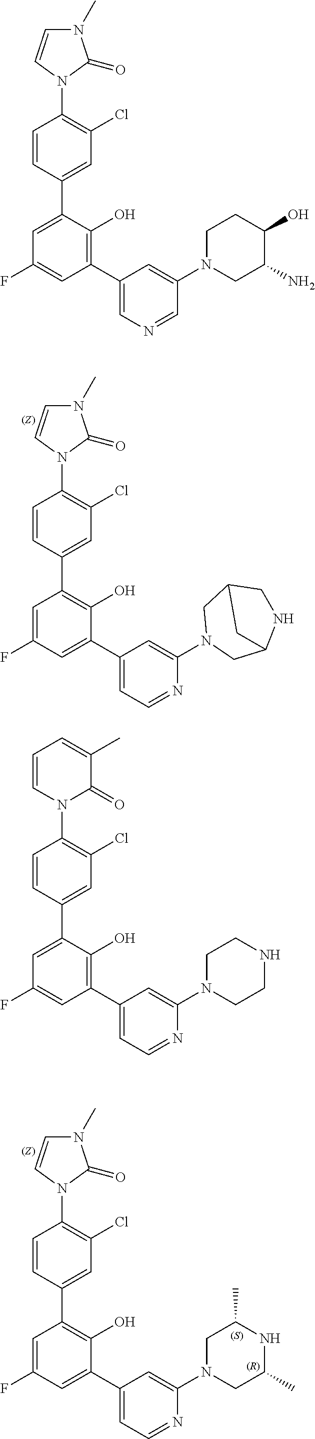

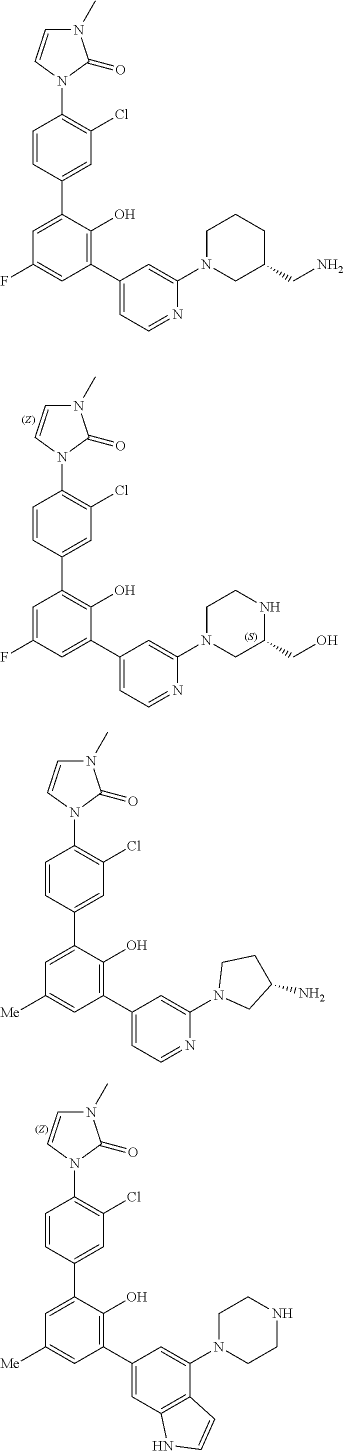

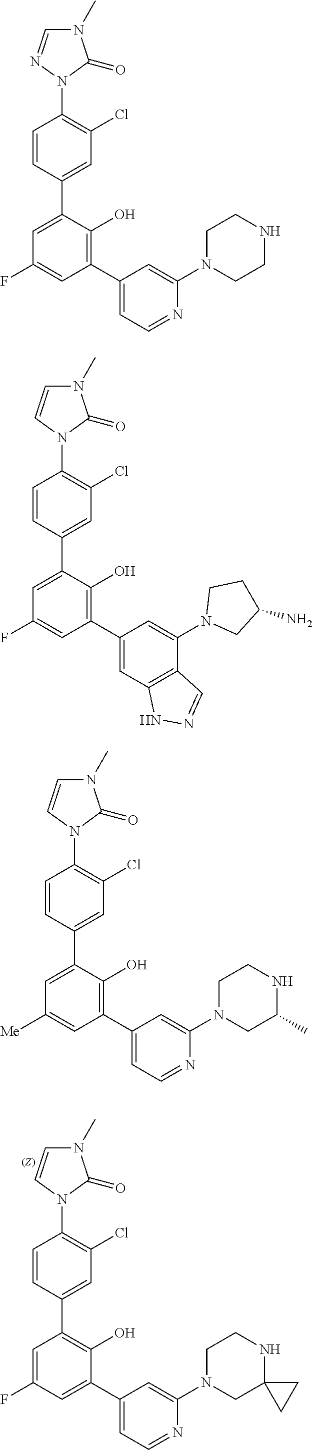 [SMILES: Cn1cnn(-c2ccc(-c3cc(F)cc(-c4ccnc(N5CCNCC5)c4)c3O)cc2Cl)c1=O ; Cn1ccn(-c2ccc(-c3cc(F)cc(-c4cc(N5CC[C@H](N)C5)c5cn[nH]c5c4)c3O)cc2Cl)c1=O ; Cc1cc(-c2ccnc(N3CCN[C@H](C)C3)c2)c(O)c(-c2ccc(-n3ccn(C)c3=O)c(Cl)c2)c1 ; Cn1ccn(-c2ccc(-c3cc(F)cc(-c4ccnc(N5CCNC6(CC6)C5)c4)c3O)cc2Cl)c1=O]

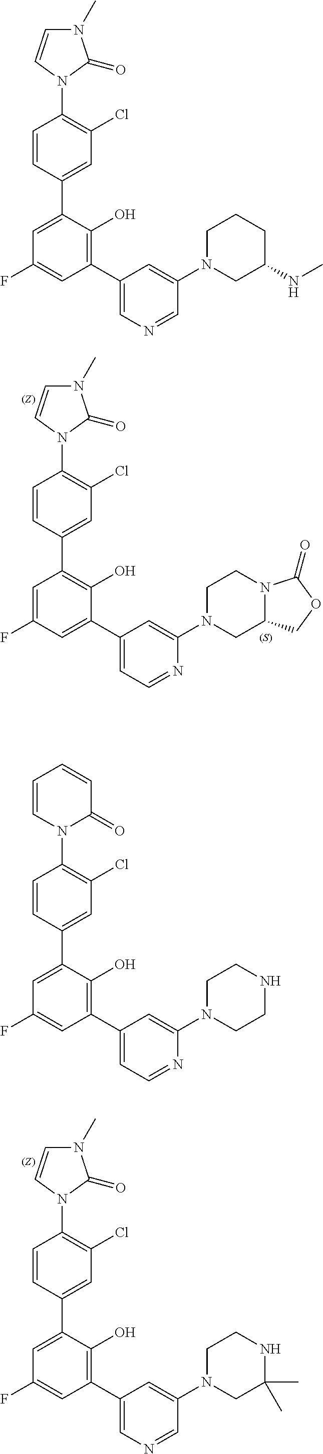

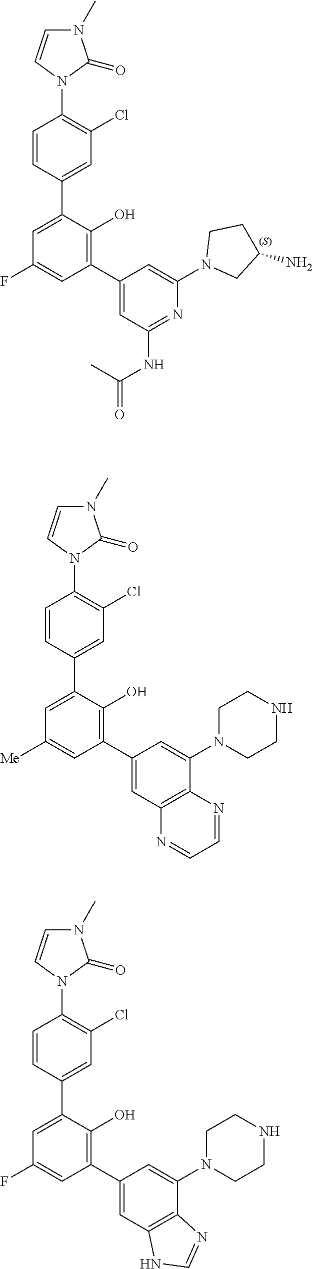

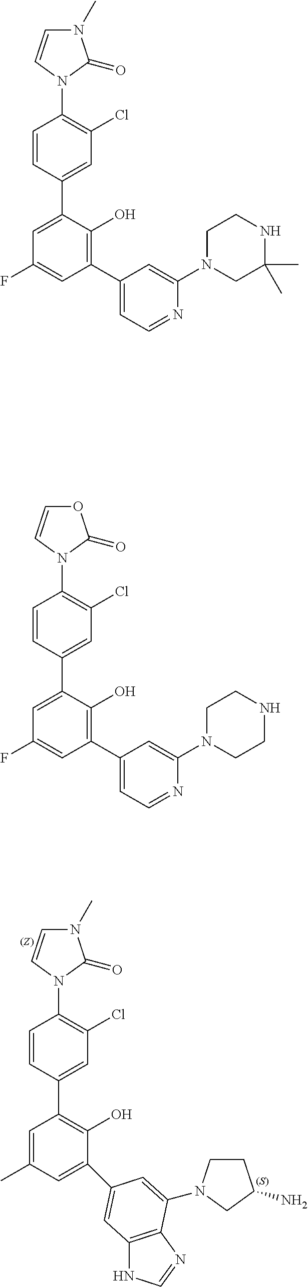

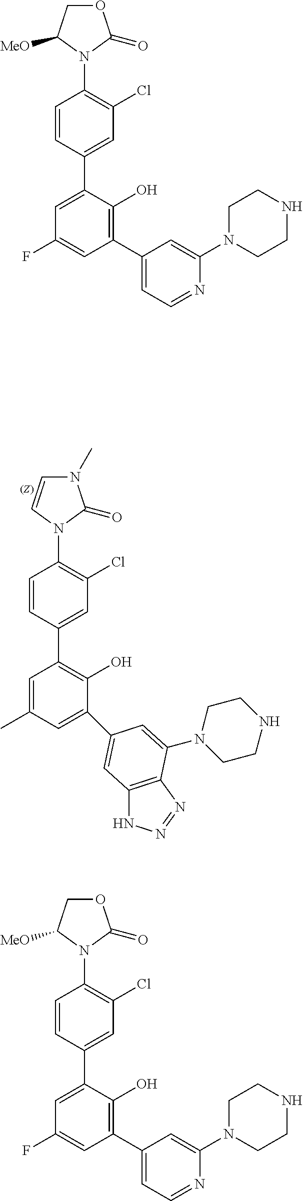 [SMILES: CO[C@H]1COC(=O)N1c1ccc(-c2cc(F)cc(-c3ccnc(N4CCNCC4)c3)c2O)cc1Cl ; Cc1cc(-c2ccc(-n3ccn(C)c3=O)c(Cl)c2)c(O)c(-c2cc(N3CCNCC3)c3nn[nH]c3c2)c1 ; CO[C@@H]1COC(=O)N1c1ccc(-c2cc(F)cc(-c3ccnc(N4CCNCC4)c3)c2O)cc1Cl]

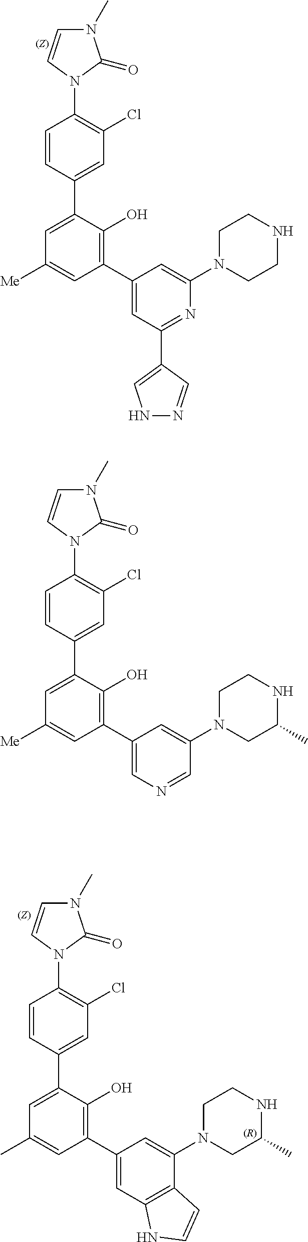

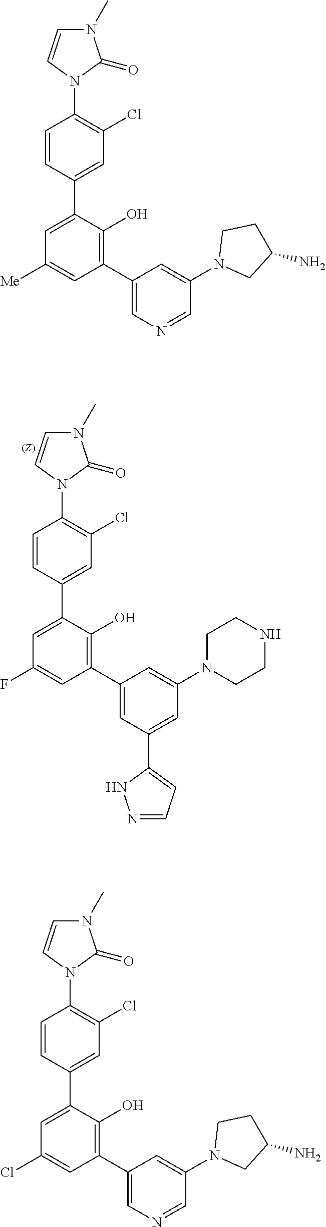 [SMILES: Cc1cc(-c2cncc(N3CC[C@H](N)C3)c2)c(O)c(-c2ccc(-n3ccn(C)c3=O)c(Cl)c2)c1 ; Cn1ccn(-c2ccc(-c3cc(F)cc(-c4cc(-c5ccn[nH]5)cc(N5CCNCC5)c4)c3O)cc2Cl)c1=O ; Cn1ccn(-c2ccc(-c3cc(Cl)cc(-c4cncc(N5CC[C@H](N)C5)c4)c3O)cc2Cl)c1=O]

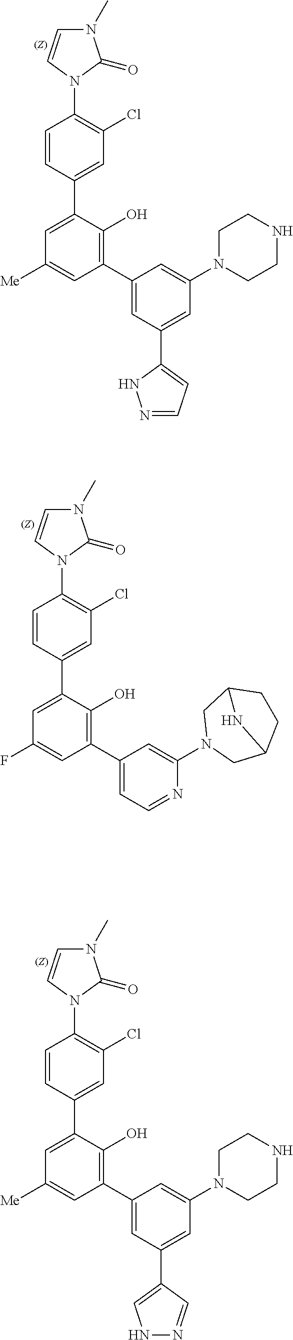 [SMILES: Cc1cc(-c2cc(-c3ccn[nH]3)cc(N3CCNCC3)c2)c(O)c(-c2ccc(-n3ccn(C)c3=O)c(Cl)c2)c1 ; Cn1ccn(-c2ccc(-c3cc(F)cc(-c4ccnc(N5CC6CCC(C5)N6)c4)c3O)cc2Cl)c1=O ; Cc1cc(-c2cc(-c3cn[nH]c3)cc(N3CCNCC3)c2)c(O)c(-c2ccc(-n3ccn(C)c3=O)c(Cl)c2)c1]

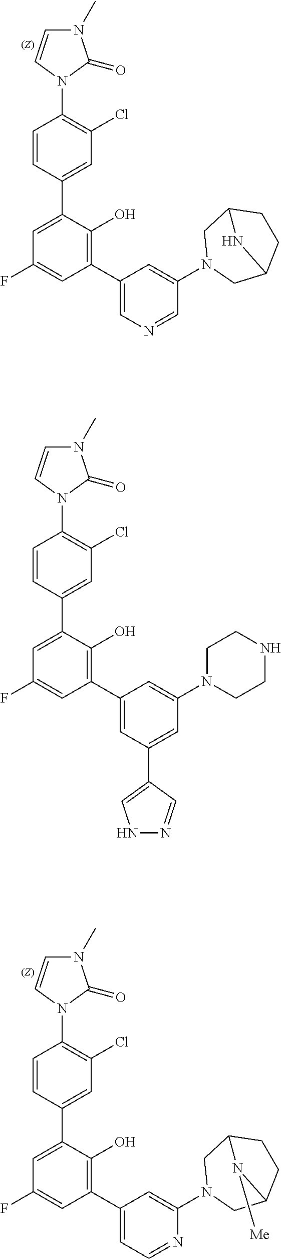 [SMILES: Cn1ccn(-c2ccc(-c3cc(F)cc(-c4cncc(N5CC6CCC(C5)N6)c4)c3O)cc2Cl)c1=O ; Cn1ccn(-c2ccc(-c3cc(F)cc(-c4cc(-c5cn[nH]c5)cc(N5CCNCC5)c4)c3O)cc2Cl)c1=O ; CN1C2CCC1CN(c1cc(-c3cc(F)cc(-c4ccc(-n5ccn(C)c5=O)c(Cl)c4)c3O)ccn1)C2]

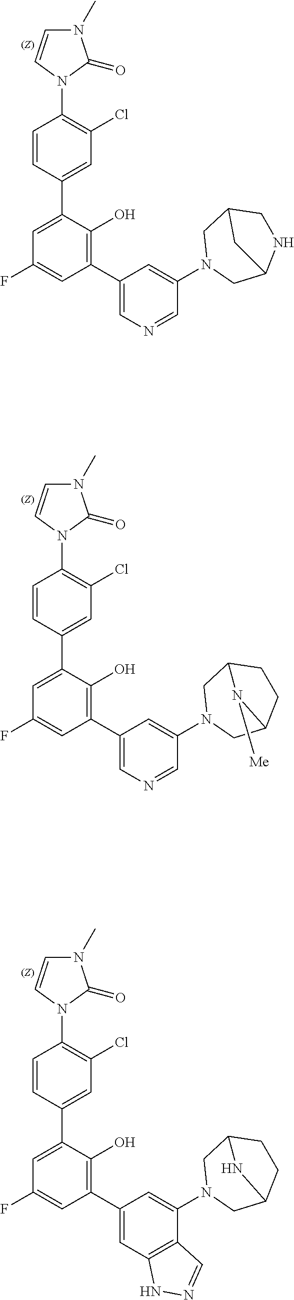 [SMILES: Cn1ccn(-c2ccc(-c3cc(F)cc(-c4cncc(N5CC6CNC(C6)C5)c4)c3O)cc2Cl)c1=O ; CN1C2CCC1CN(c1cncc(-c3cc(F)cc(-c4ccc(-n5ccn(C)c5=O)c(Cl)c4)c3O)c1)C2 ; Cn1ccn(-c2ccc(-c3cc(F)cc(-c4cc(N5CC6CCC(C5)N6)c5cn[nH]c5c4)c3O)cc2Cl)c1=O]

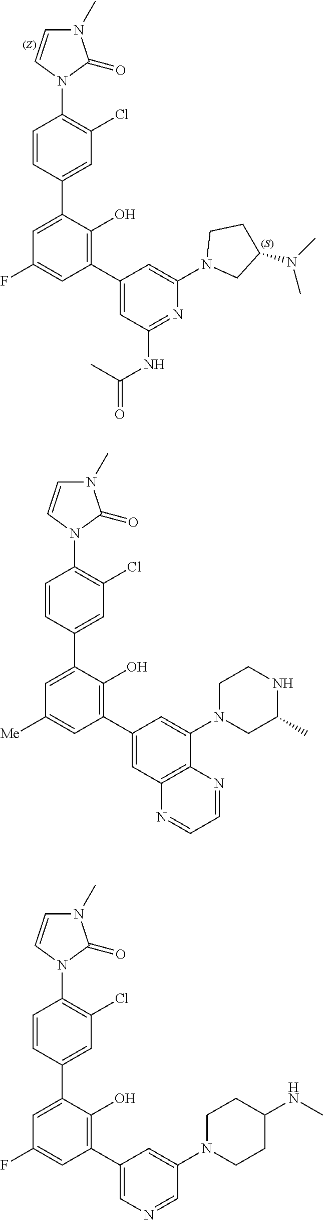

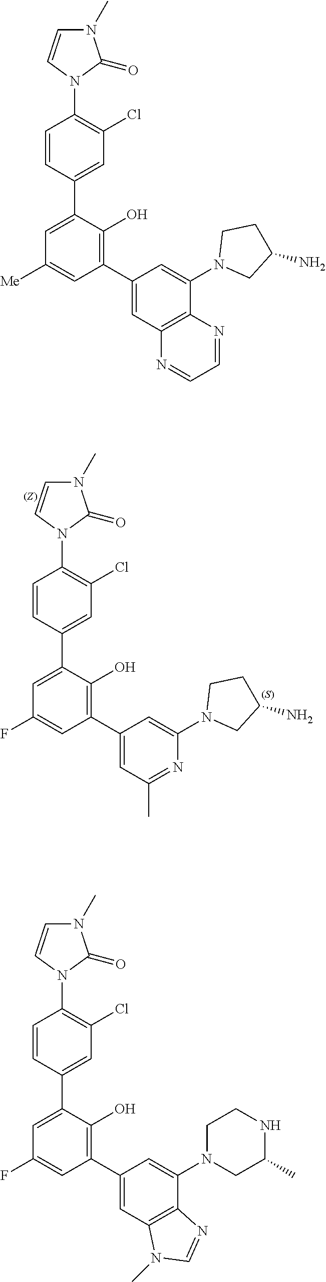

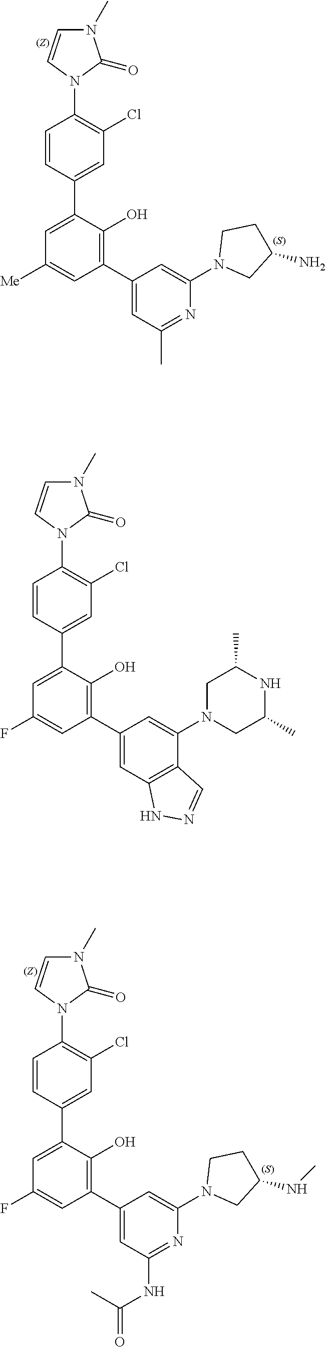

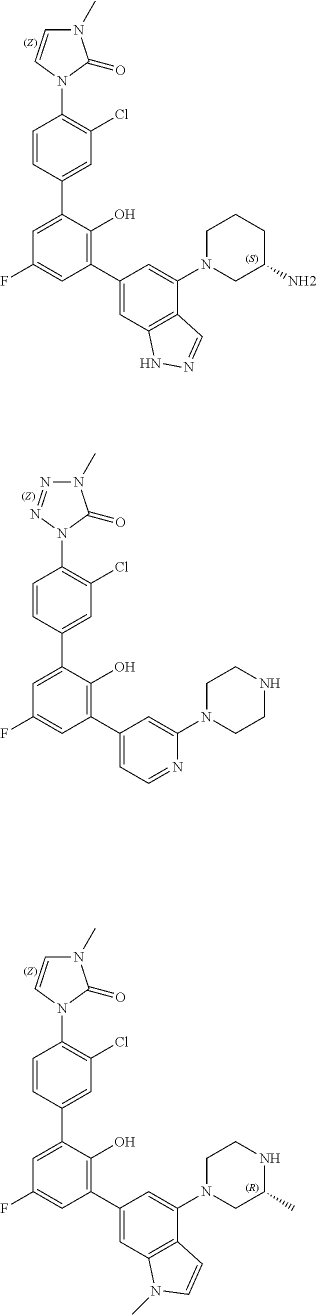

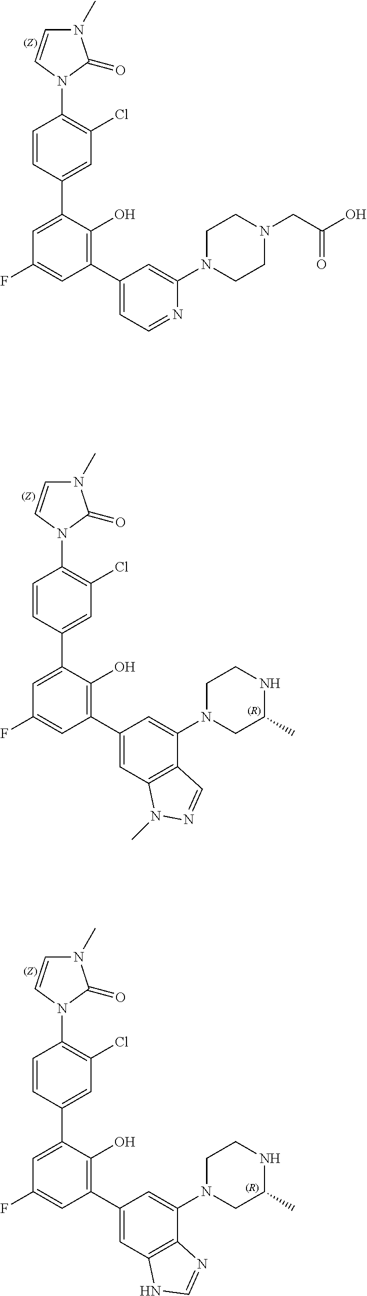

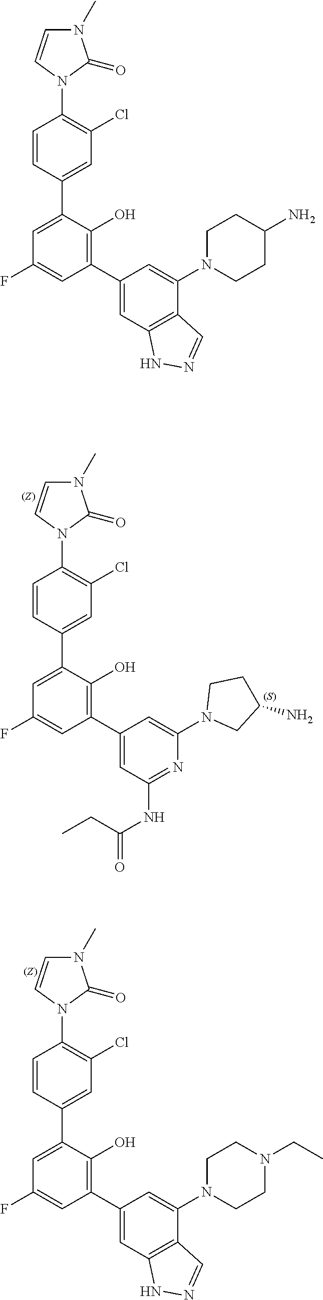

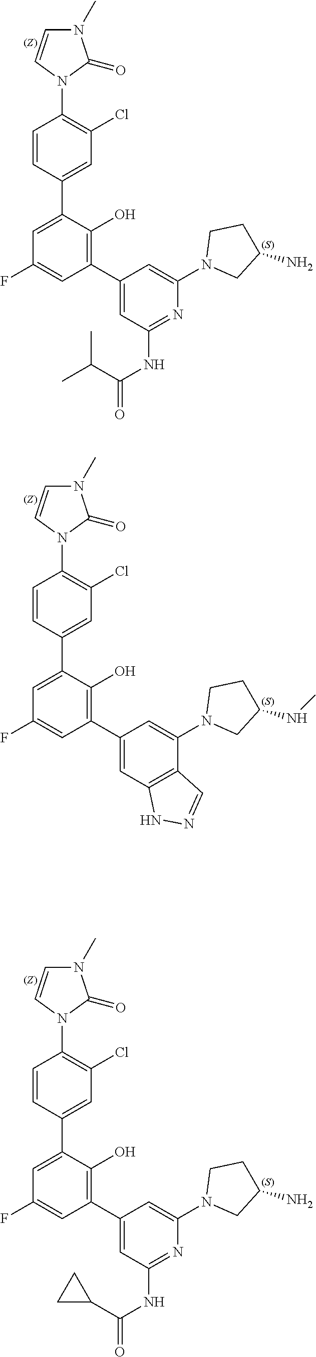

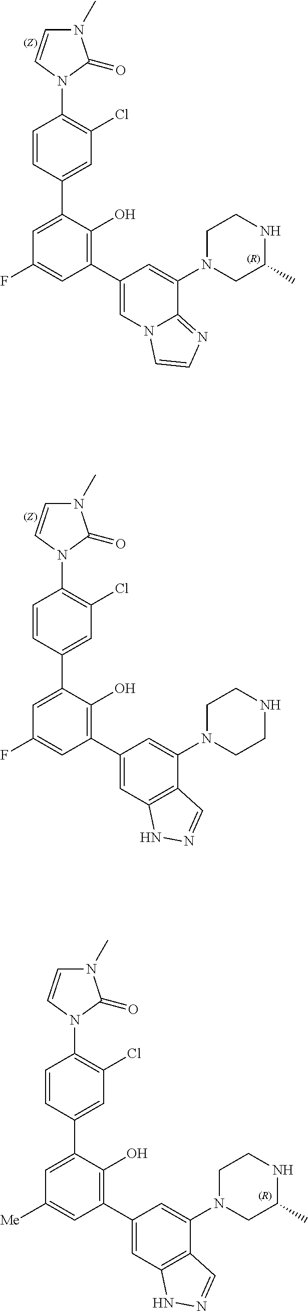

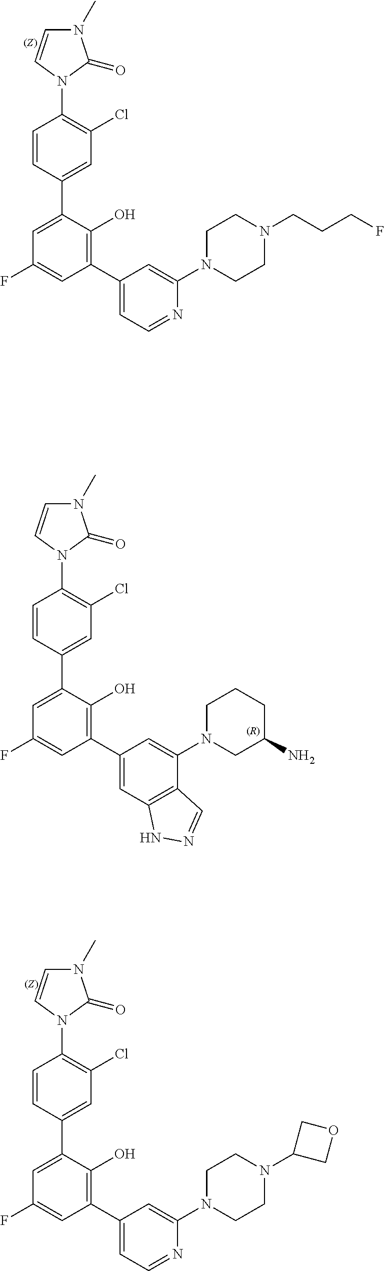

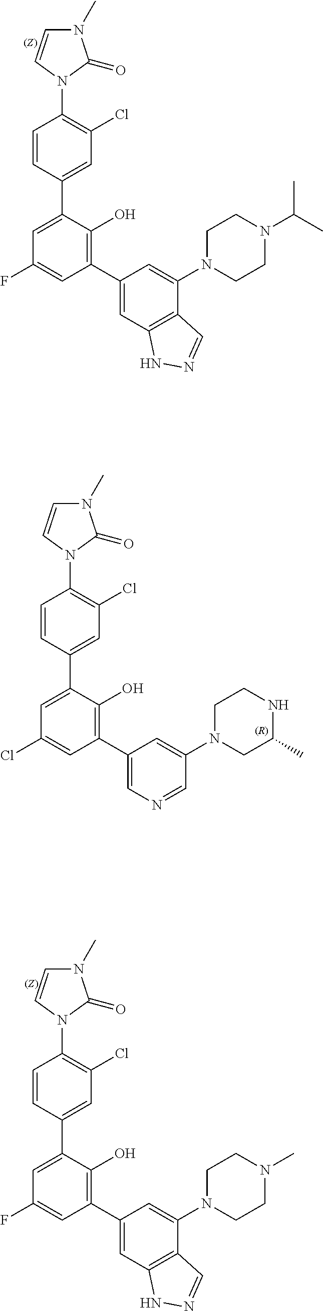

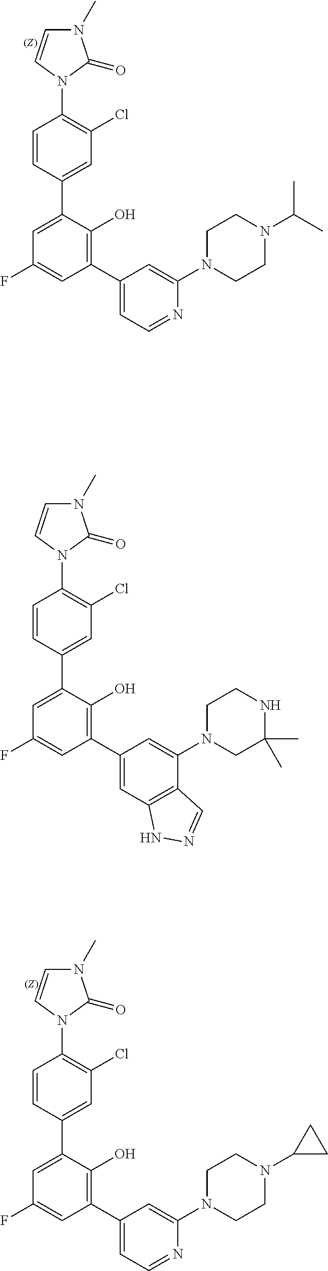 [SMILES: CC(C)N1CCN(c2cc(-c3cc(F)cc(-c4ccc(-n5ccn(C)c5=O)c(Cl)c4)c3O)ccn2)CC1 ; Cn1ccn(-c2ccc(-c3cc(F)cc(-c4cc(N5CCNC(C)(C)C5)c5cn[nH]c5c4)c3O)cc2Cl)c1=O ; Cn1ccn(-c2ccc(-c3cc(F)cc(-c4ccnc(N5CCN(C6CC6)CC5)c4)c3O)cc2Cl)c1=O]

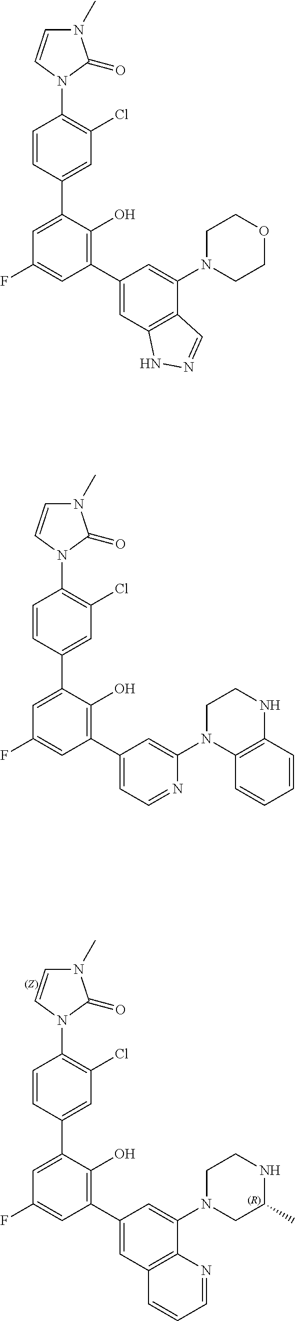

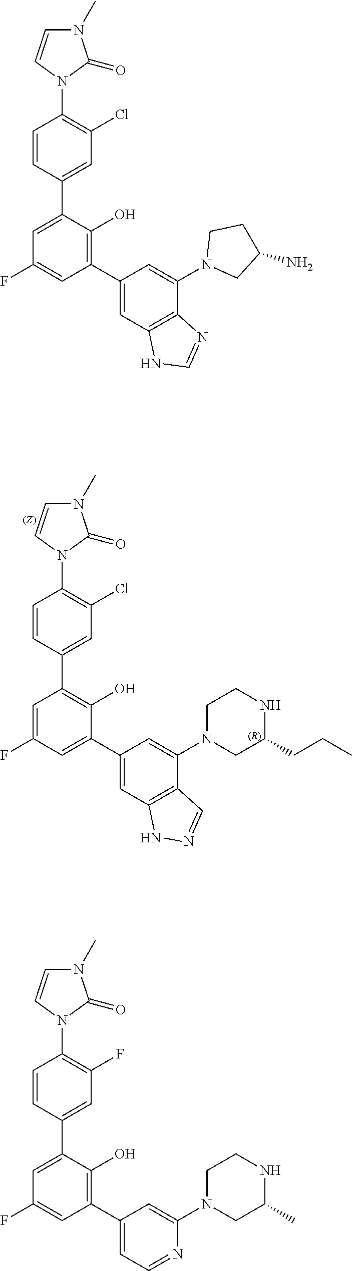

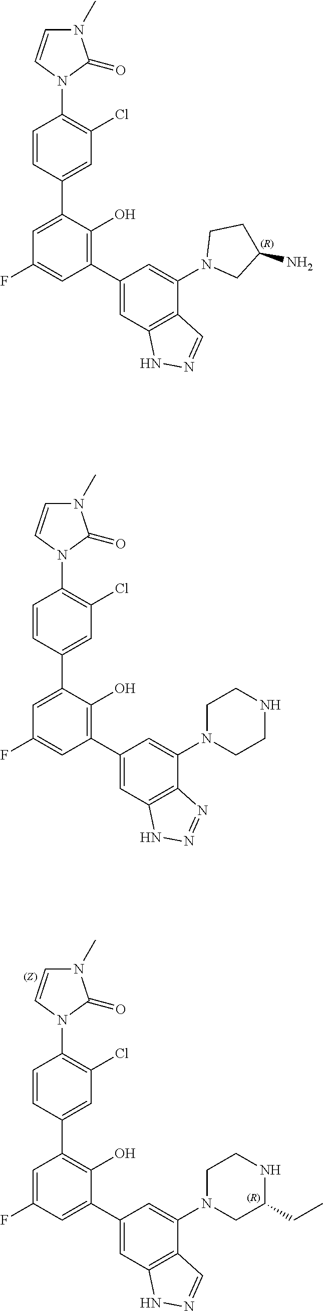

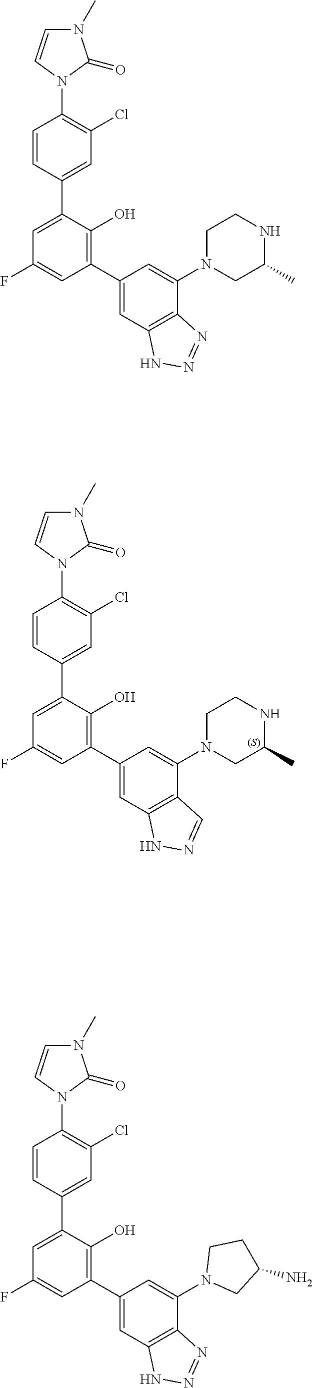

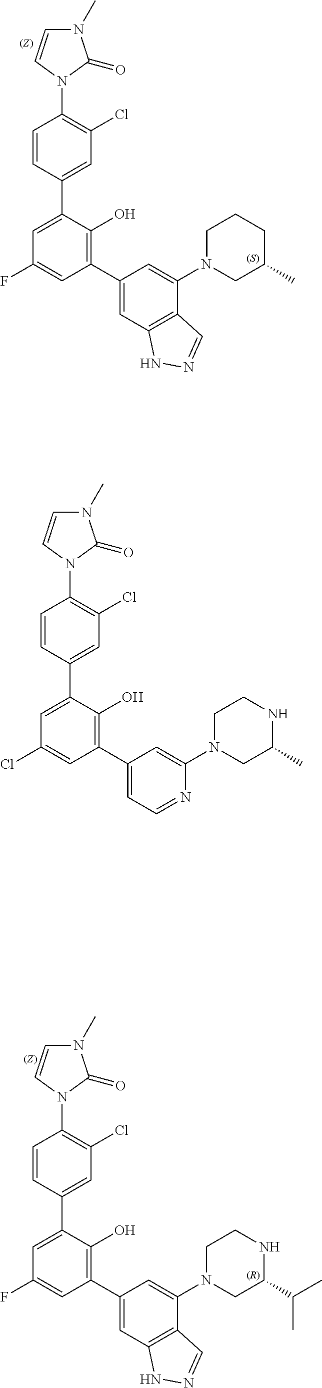

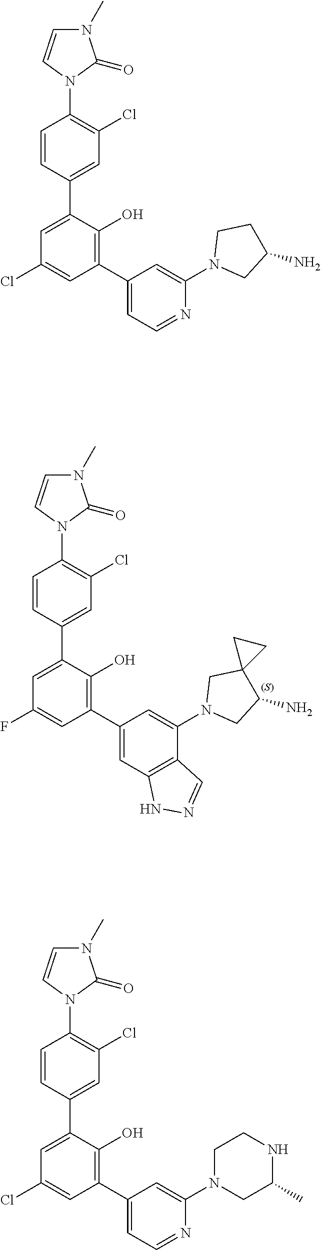

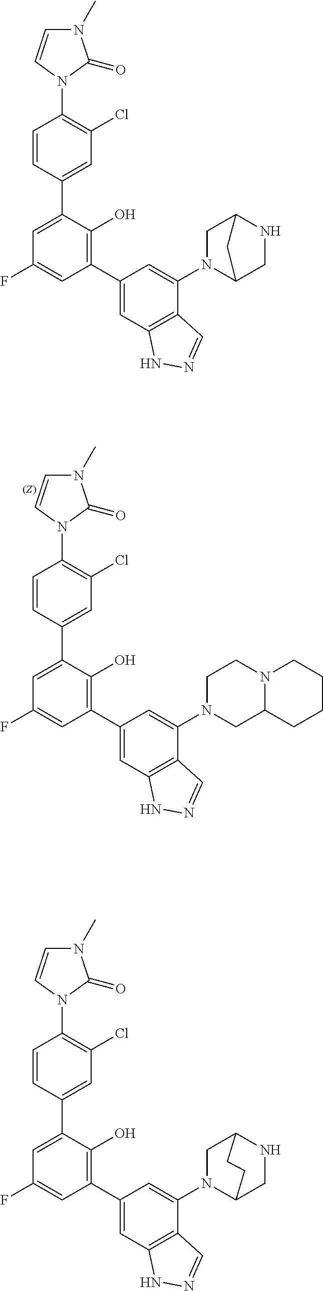 [SMILES: Cn1ccn(-c2ccc(-c3cc(F)cc(-c4cc(N5CC6CC5CN6)c5cn[nH]c5c4)c3O)cc2Cl)c1=O ; Cn1ccn(-c2ccc(-c3cc(F)cc(-c4cc(N5CCN6CCCCC6C5)c5cn[nH]c5c4)c3O)cc2Cl)c1=O ; Cn1ccn(-c2ccc(-c3cc(F)cc(-c4cc(N5CC6CCC5CN6)c5cn[nH]c5c4)c3O)cc2Cl)c1=O]

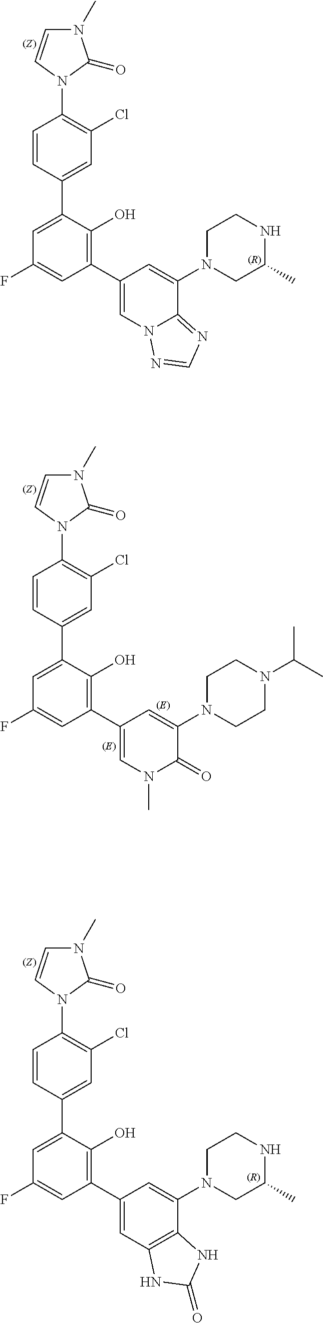

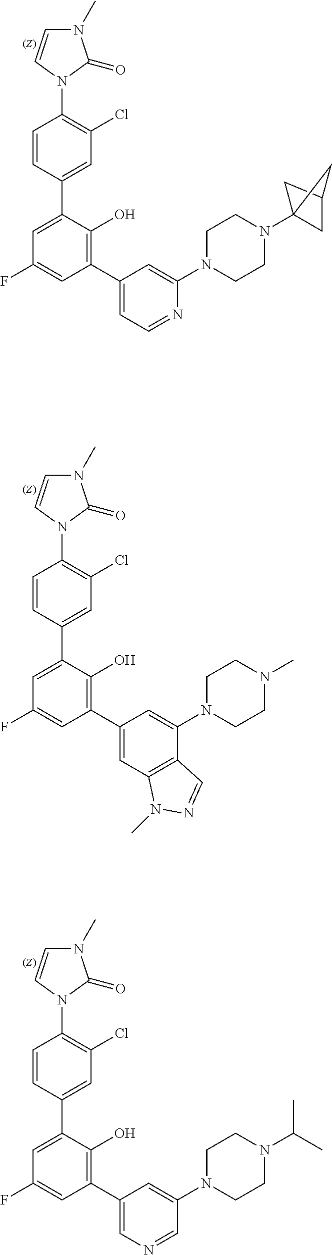 [SMILES: Cn1ccn(-c2ccc(-c3cc(F)cc(-c4ccnc(N5CCN(C67CC(C6)C7)CC5)c4)c3O)cc2Cl)c1=O ; CN1CCN(c2cc(-c3cc(F)cc(-c4ccc(-n5ccn(C)c5=O)c(Cl)c4)c3O)cc3c2cnn3C)CC1 ; CC(C)N1CCN(c2cncc(-c3cc(F)cc(-c4ccc(-n5ccn(C)c5=O)c(Cl)c4)c3O)c2)CC1]

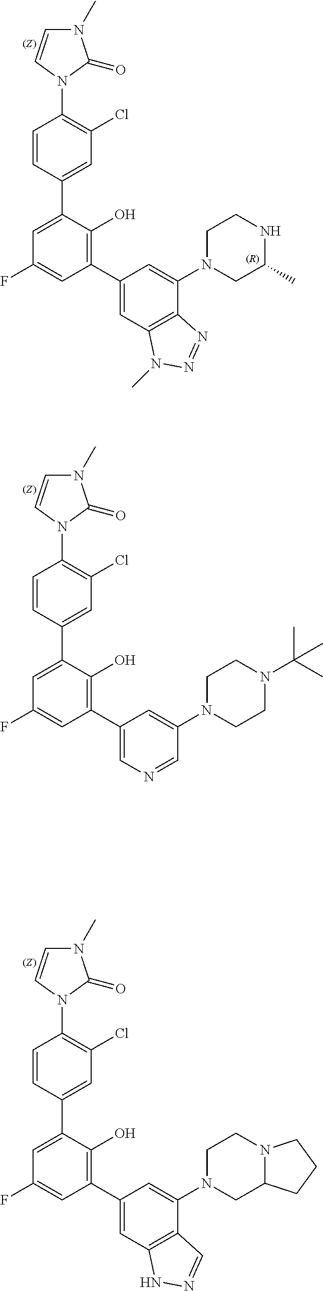

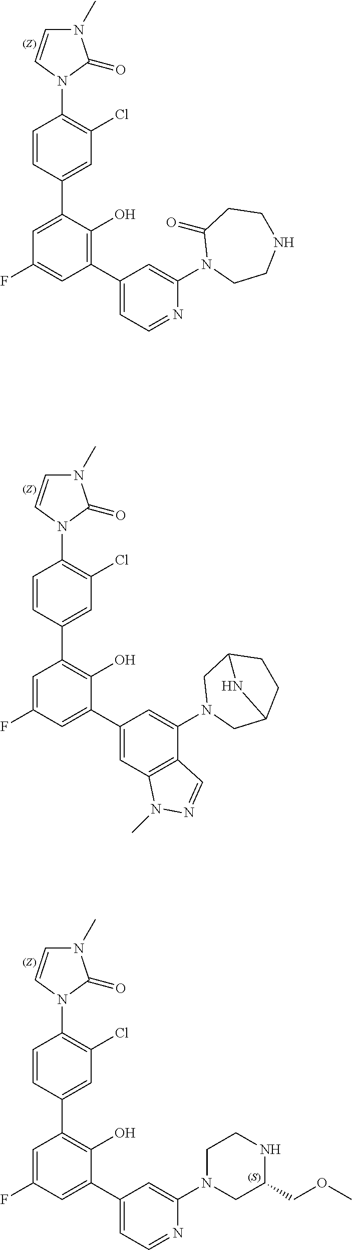

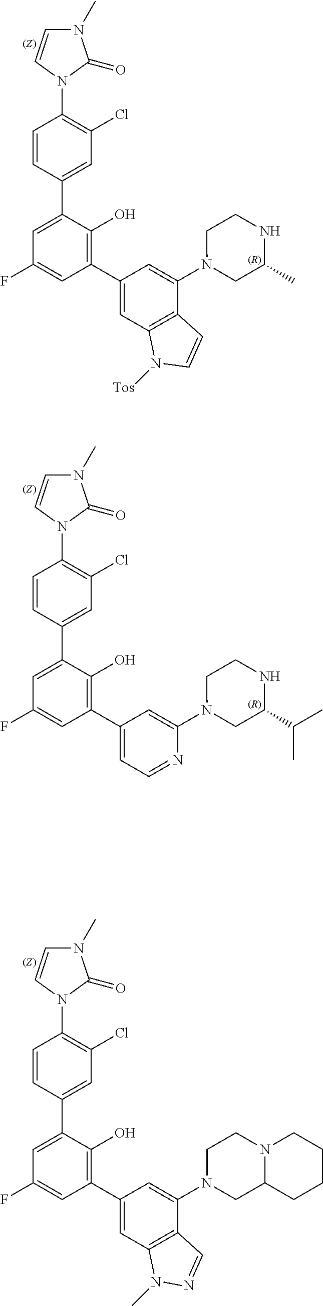

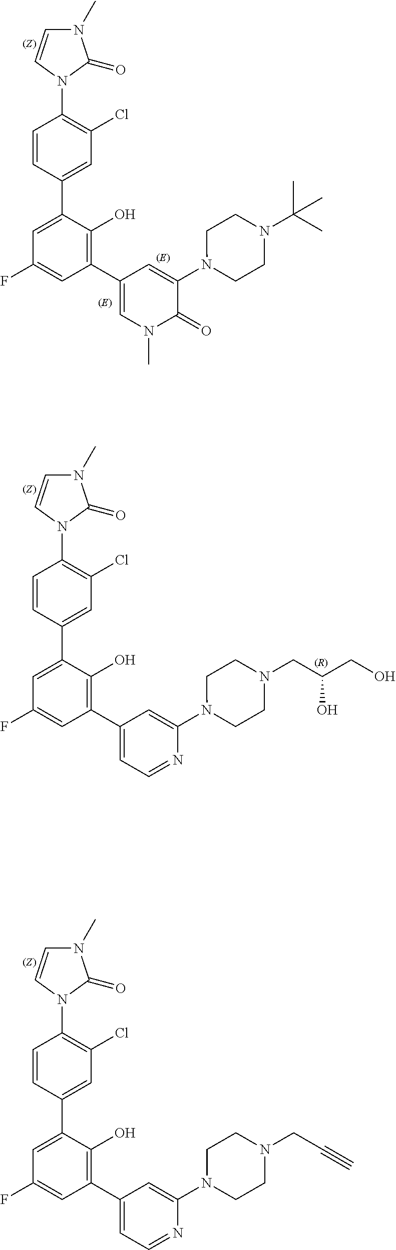

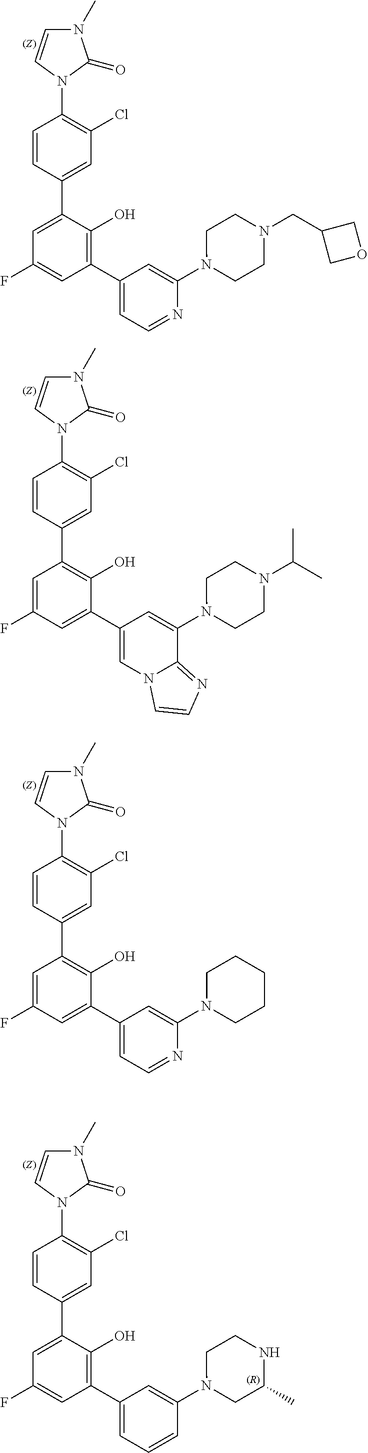

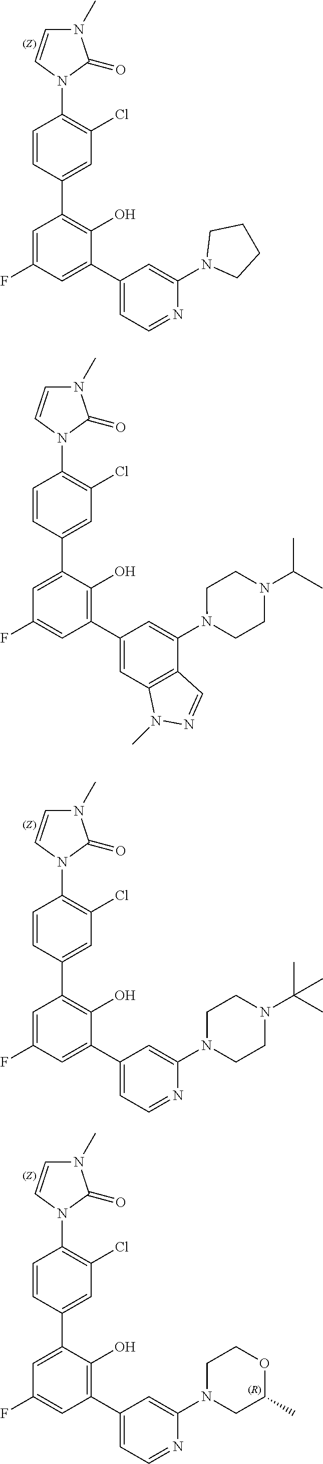

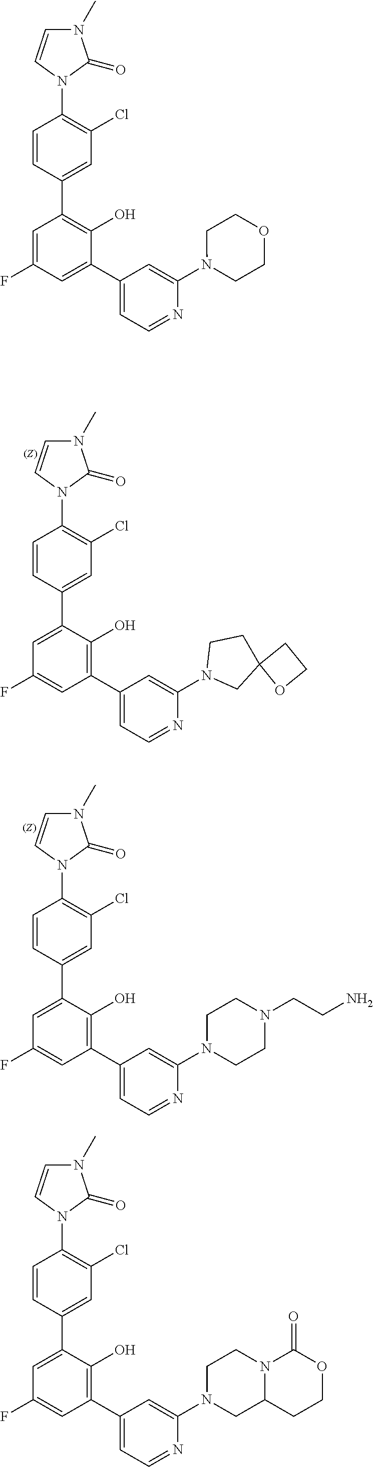

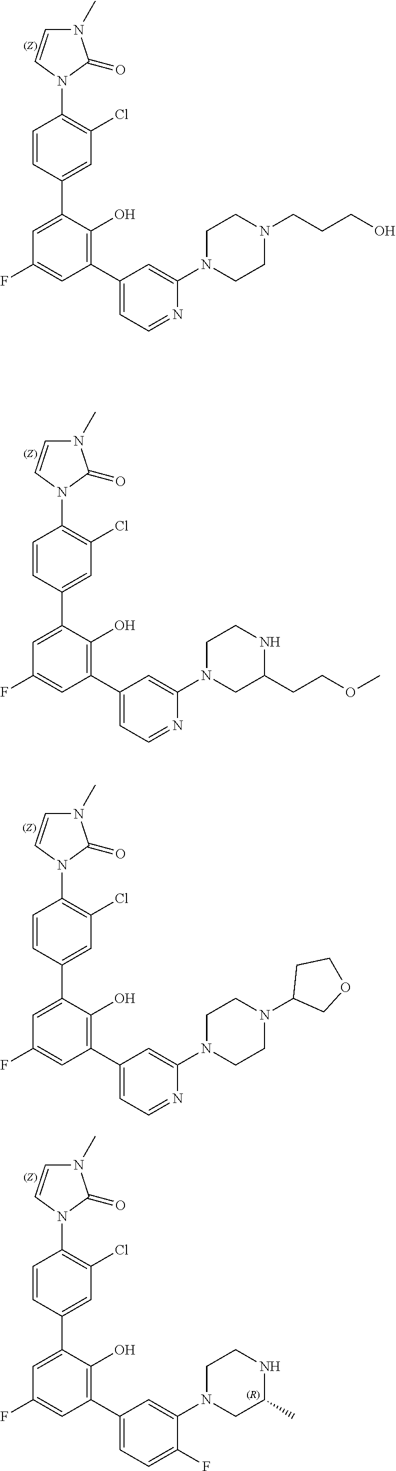

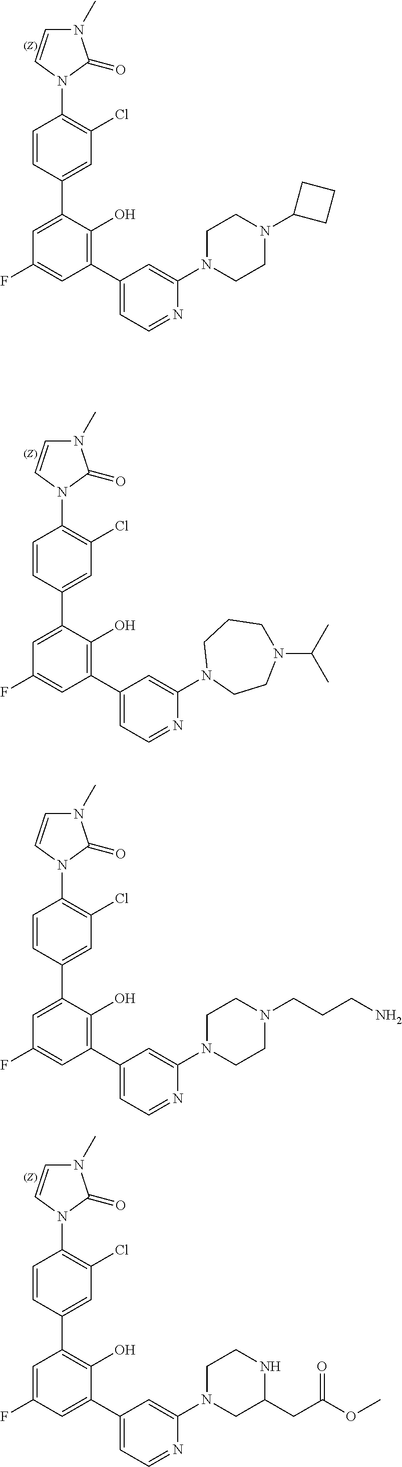

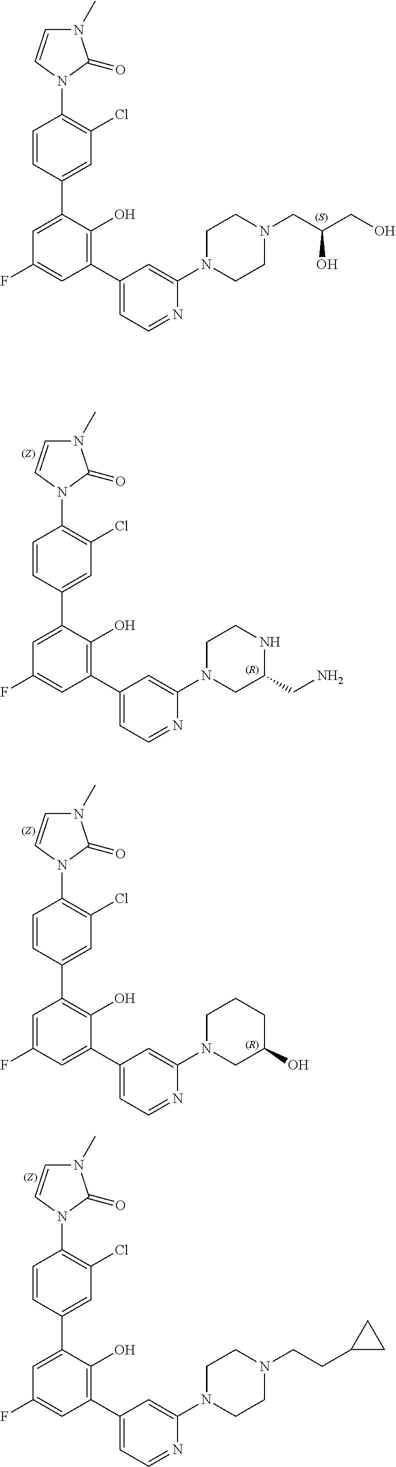

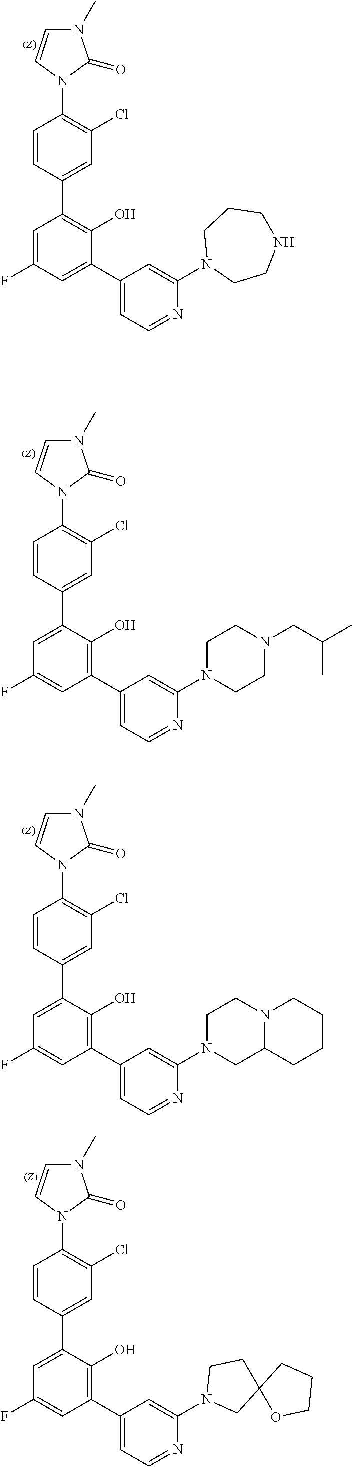

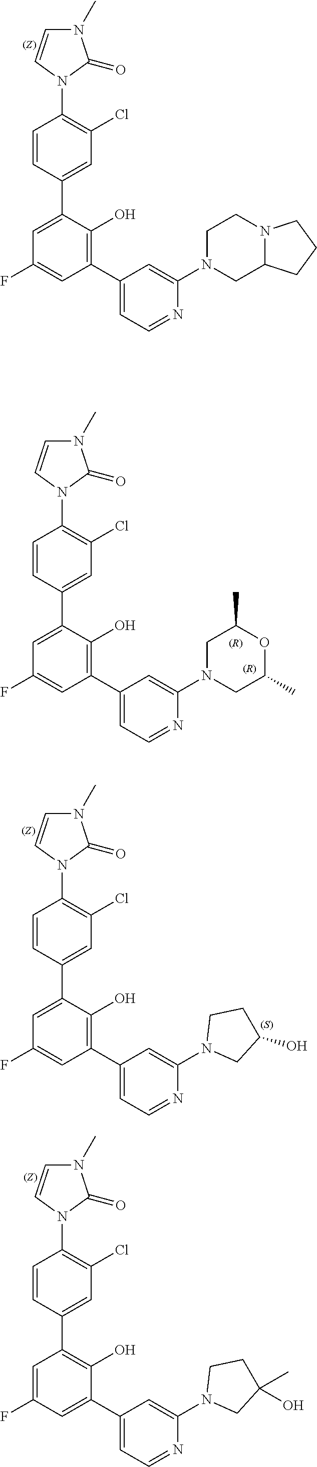

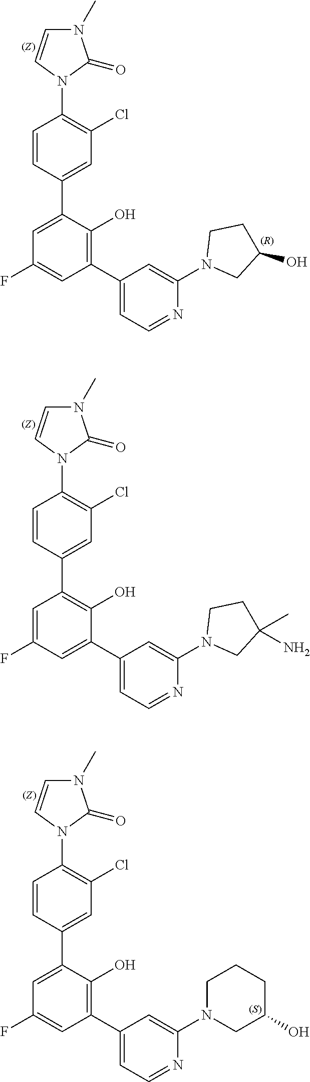

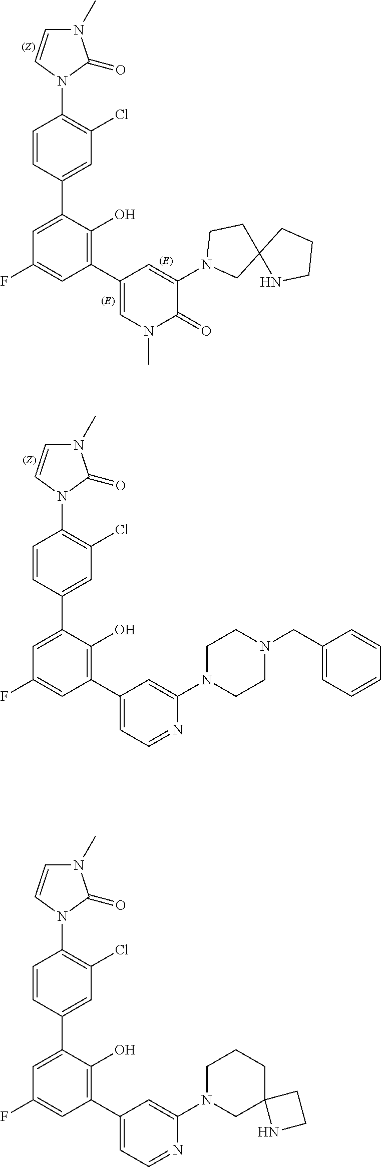 [SMILES: Cn1cc(-c2cc(F)cc(-c3ccc(-n4ccn(C)c4=O)c(Cl)c3)c2O)cc(N2CCC3(CCCN3)C2)c1=O ; Cn1ccn(-c2ccc(-c3cc(F)cc(-c4ccnc(N5CCN(Cc6ccccc6)CC5)c4)c3O)cc2Cl)c1=O ; Cn1ccn(-c2ccc(-c3cc(F)cc(-c4ccnc(N5CCCC6(CCN6)C5)c4)c3O)cc2Cl)c1=O]

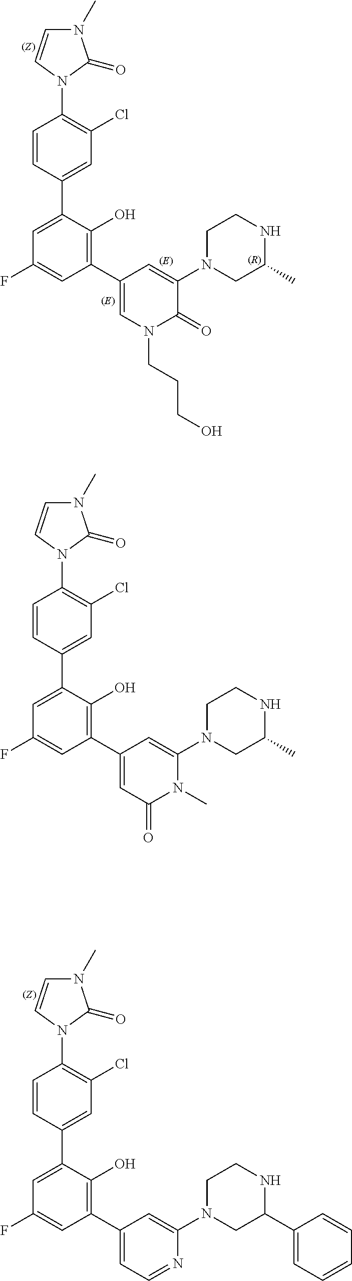

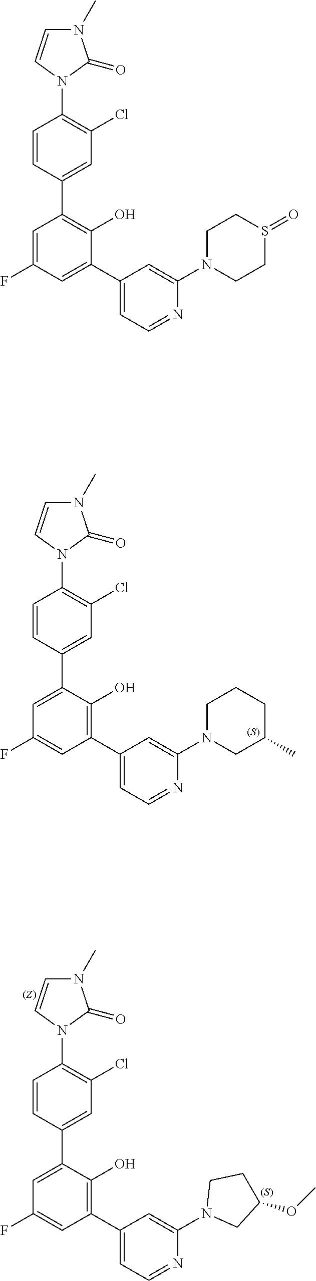

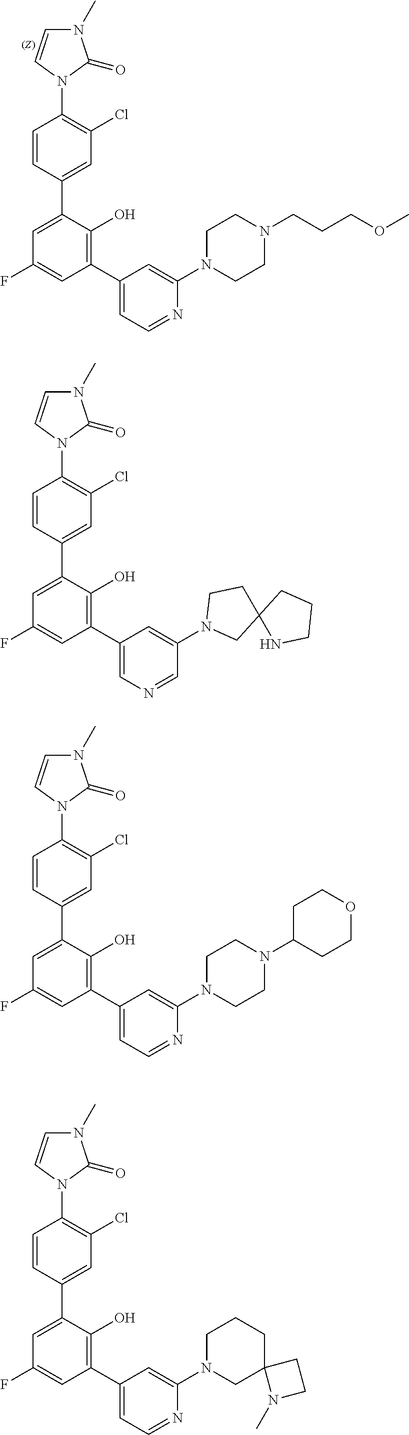 [SMILES: COCCCN1CCN(c2cc(-c3cc(F)cc(-c4ccc(-n5ccn(C)c5=O)c(Cl)c4)c3O)ccn2)CC1 ; Cn1ccn(-c2ccc(-c3cc(F)cc(-c4cncc(N5CCC6(CCCN6)C5)c4)c3O)cc2Cl)c1=O ; Cn1ccn(-c2ccc(-c3cc(F)cc(-c4ccnc(N5CCN(C6CCOCC6)CC5)c4)c3O)cc2Cl)c1=O ; CN1CCC12CCCN(c1cc(-c3cc(F)cc(-c4ccc(-n5ccn(C)c5=O)c(Cl)c4)c3O)ccn1)C2]

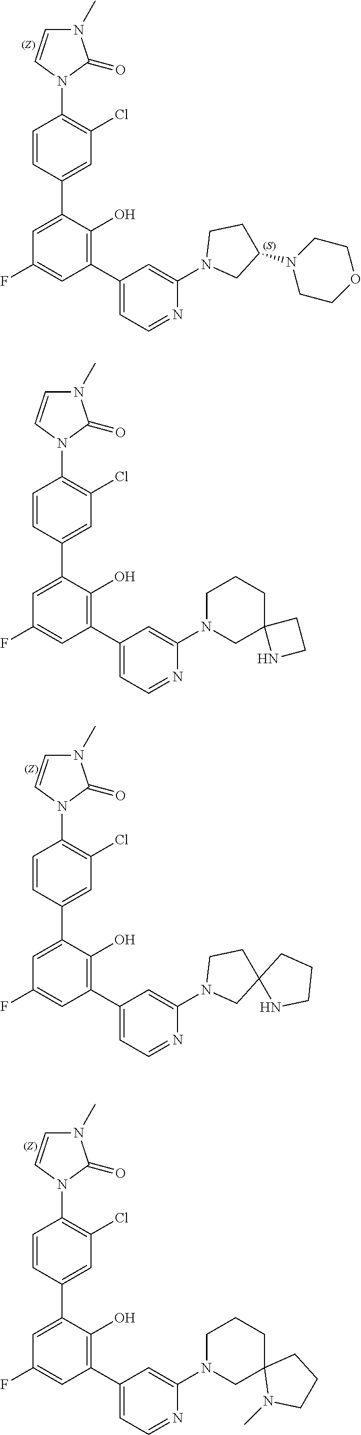

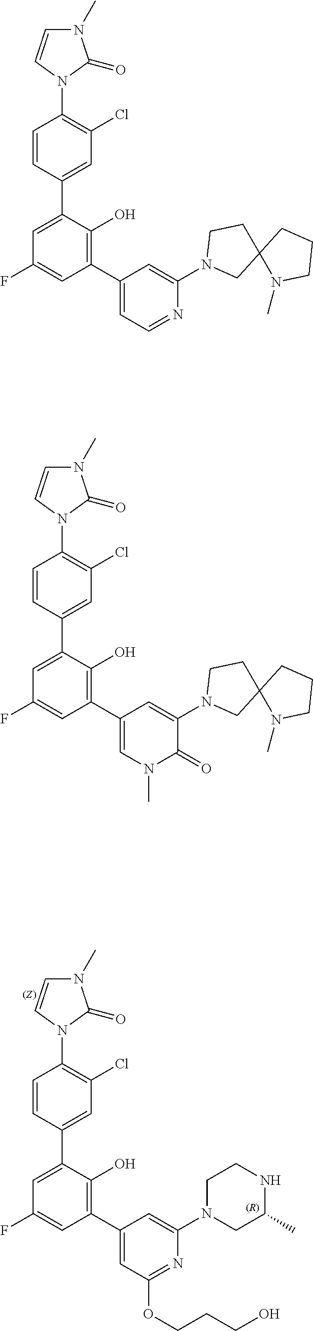

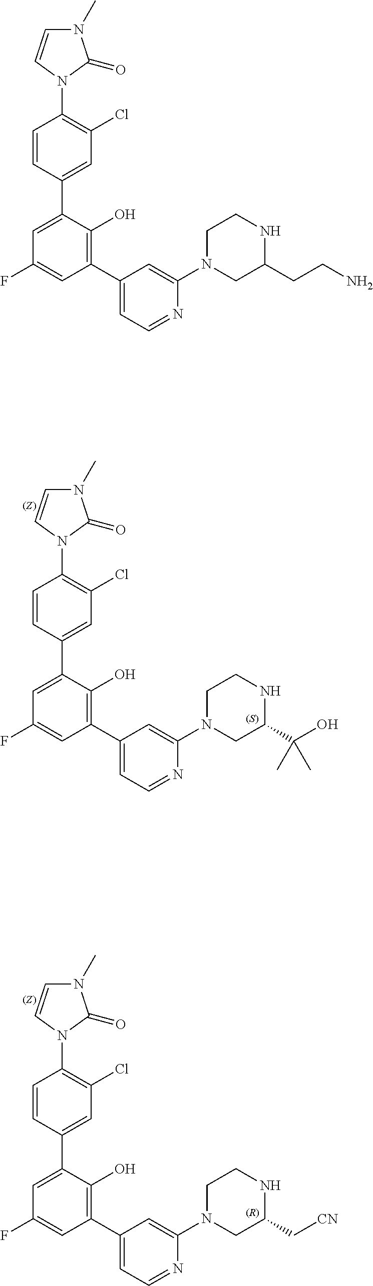

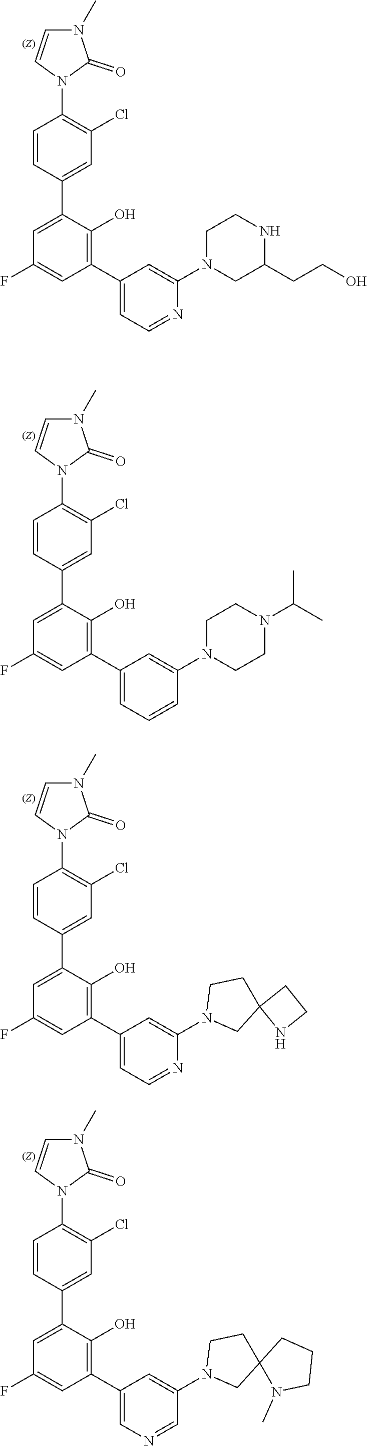 [SMILES: Cn1ccn(-c2ccc(-c3cc(F)cc(-c4ccnc(N5CCNC(CCO)C5)c4)c3O)cc2Cl)c1=O ; CC(C)N1CCN(c2cccc(-c3cc(F)cc(-c4ccc(-n5ccn(C)c5=O)c(Cl)c4)c3O)c2)CC1 ; Cn1ccn(-c2ccc(-c3cc(F)cc(-c4ccnc(N5CCC6(CCN6)C5)c4)c3O)cc2Cl)c1=O ; CN1CCCC12CCN(c1cncc(-c3cc(F)cc(-c4ccc(-n5ccn(C)c5=O)c(Cl)c4)c3O)c1)C2]

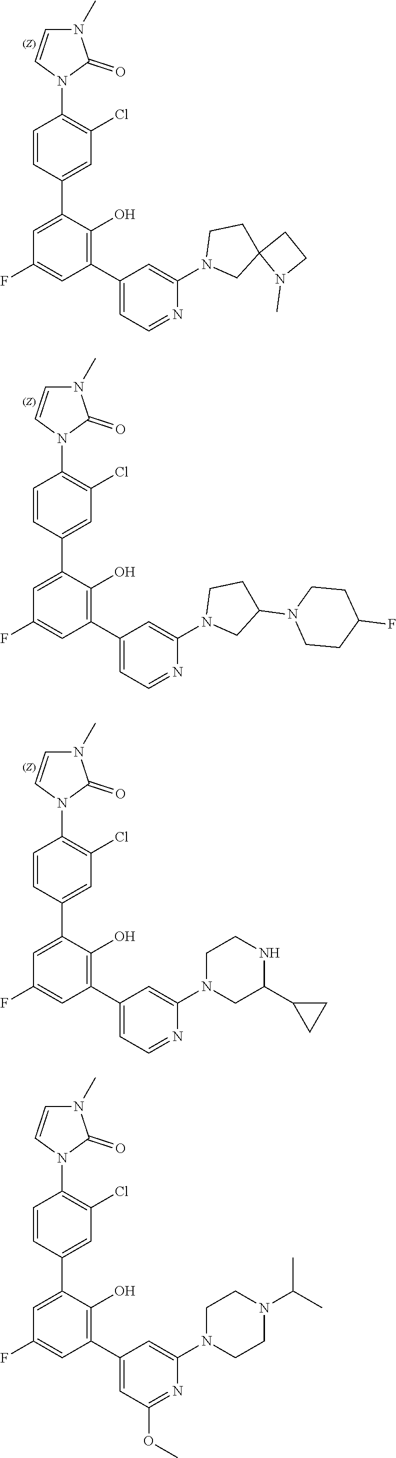 [SMILES: CN1CCC12CCN(c1cc(-c3cc(F)cc(-c4ccc(-n5ccn(C)c5=O)c(Cl)c4)c3O)ccn1)C2 ; Cn1ccn(-c2ccc(-c3cc(F)cc(-c4ccnc(N5CCC(N6CCC(F)CC6)C5)c4)c3O)cc2Cl)c1=O ; Cn1ccn(-c2ccc(-c3cc(F)cc(-c4ccnc(N5CCNC(C6CC6)C5)c4)c3O)cc2Cl)c1=O ; COc1cc(-c2cc(F)cc(-c3ccc(-n4ccn(C)c4=O)c(Cl)c3)c2O)cc(N2CCN(C(C)C)CC2)n1]

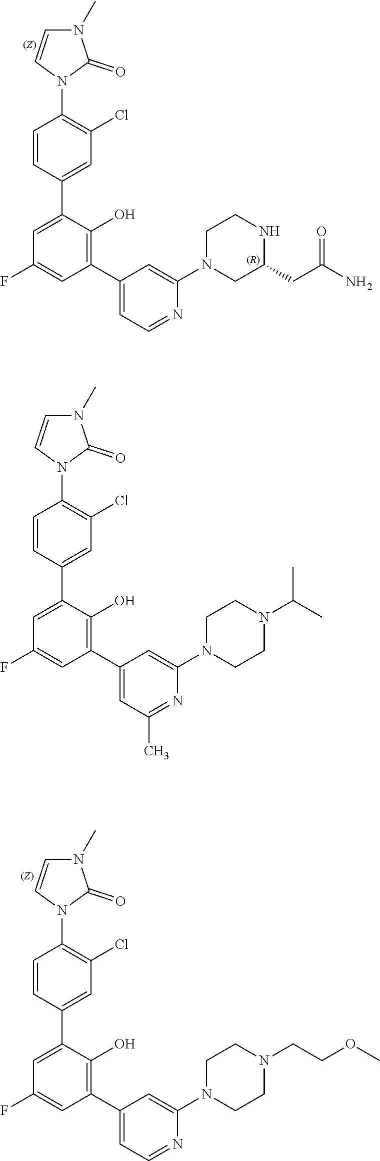

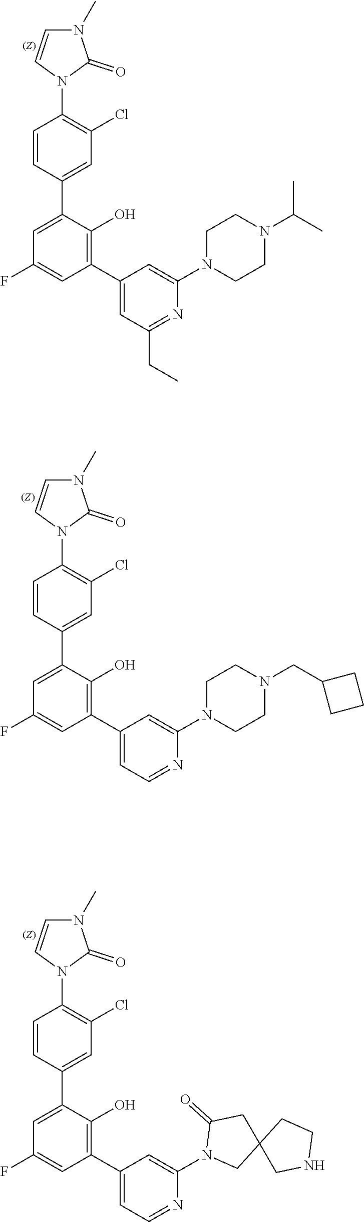 [SMILES: CCc1cc(-c2cc(F)cc(-c3ccc(-n4ccn(C)c4=O)c(Cl)c3)c2O)cc(N2CCN(C(C)C)CC2)n1 ; Cn1ccn(-c2ccc(-c3cc(F)cc(-c4ccnc(N5CCN(CC6CCC6)CC5)c4)c3O)cc2Cl)c1=O ; Cn1ccn(-c2ccc(-c3cc(F)cc(-c4ccnc(N5CC6(CCNC6)CC5=O)c4)c3O)cc2Cl)c1=O]

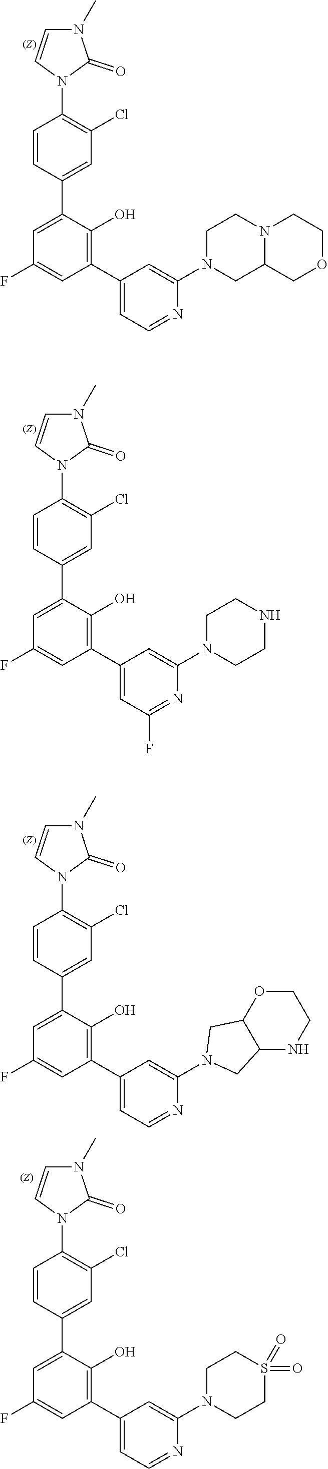

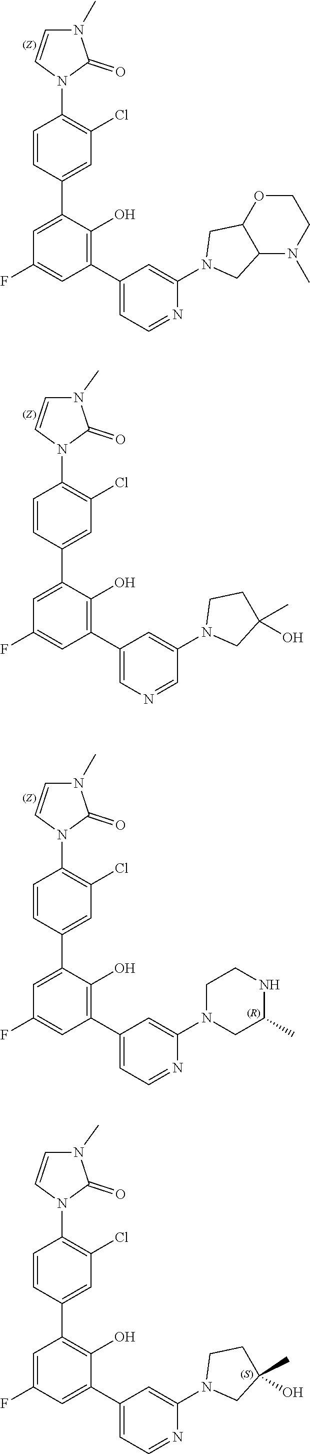

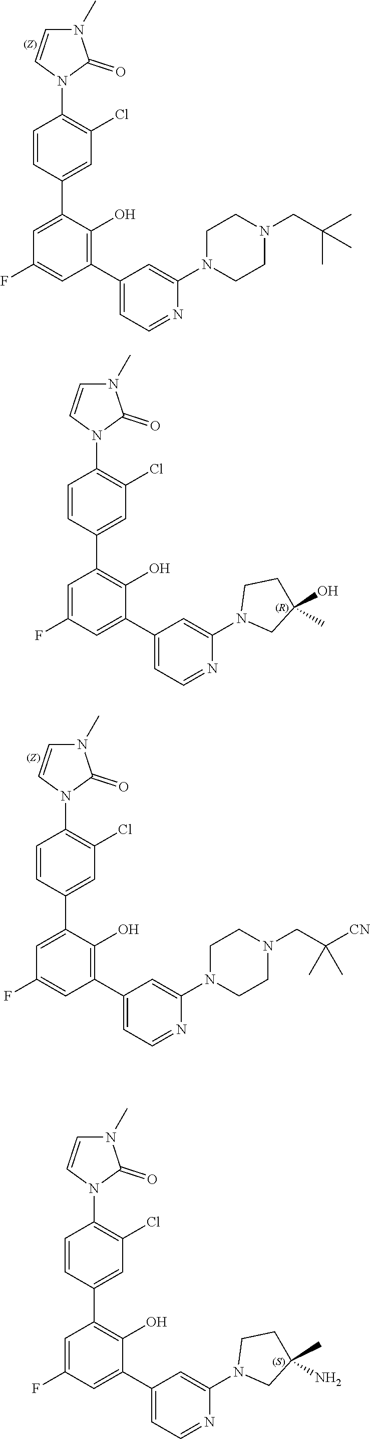

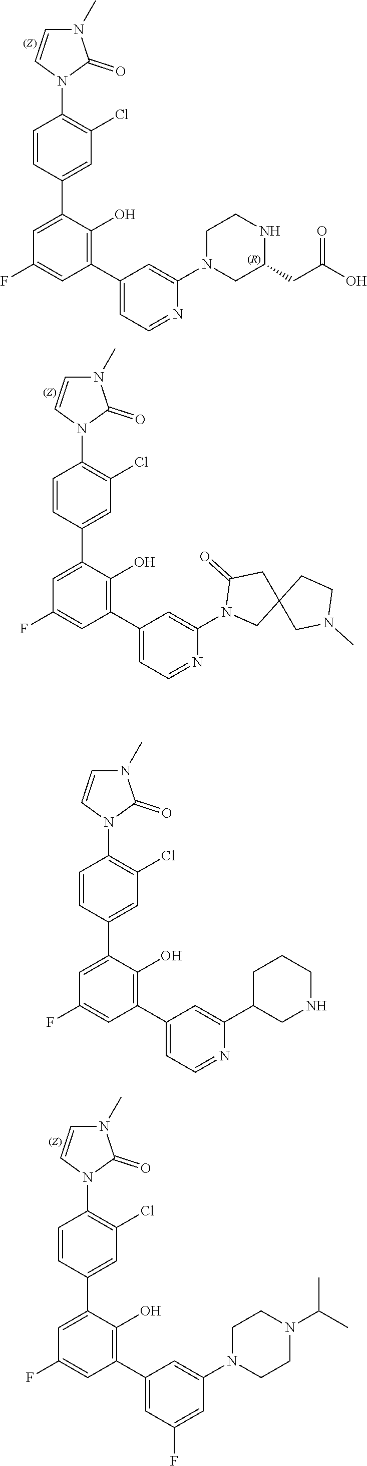

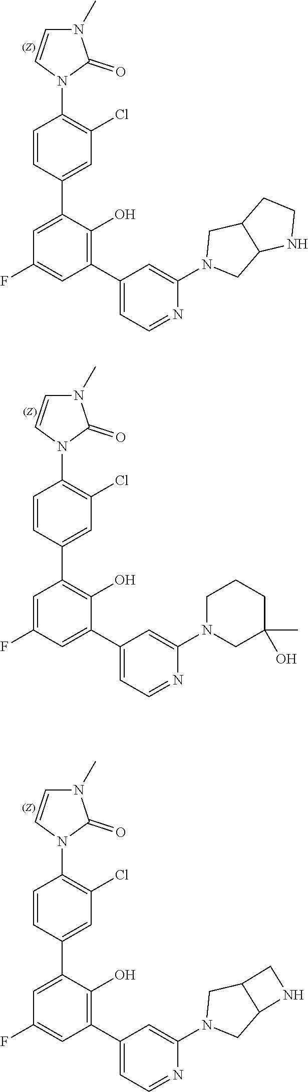 [SMILES: Cn1ccn(-c2ccc(-c3cc(F)cc(-c4ccnc(N5CC6CCNC6C5)c4)c3O)cc2Cl)c1=O ; Cn1ccn(-c2ccc(-c3cc(F)cc(-c4ccnc(N5CCCC(C)(O)C5)c4)c3O)cc2Cl)c1=O ; Cn1ccn(-c2ccc(-c3cc(F)cc(-c4ccnc(N5CC6CNC6C5)c4)c3O)cc2Cl)c1=O]

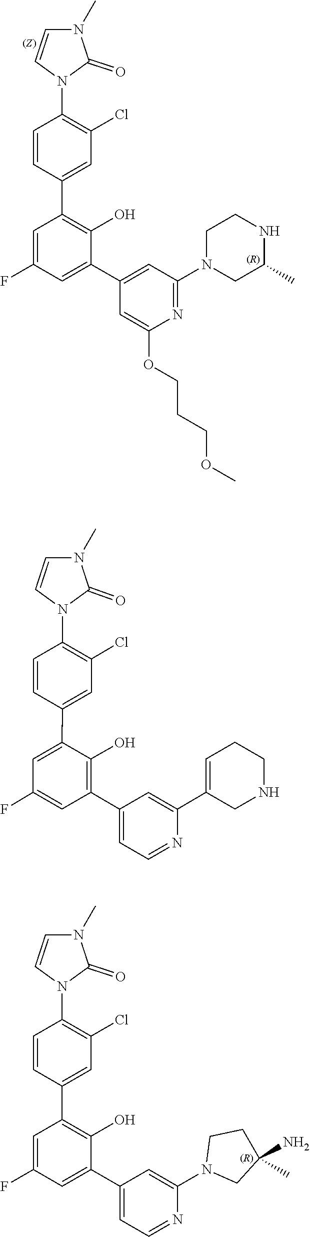

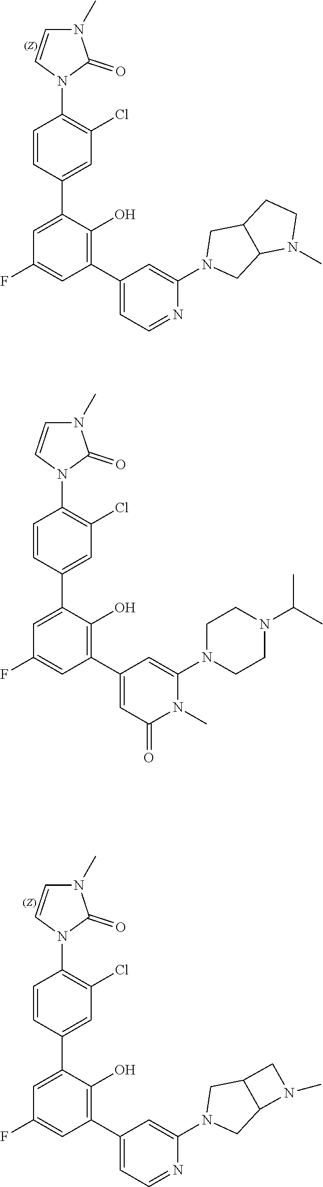 [SMILES: CN1CCC2CN(c3cc(-c4cc(F)cc(-c5ccc(-n6ccn(C)c6=O)c(Cl)c5)c4O)ccn3)CC21 ; CC(C)N1CCN(c2cc(-c3cc(F)cc(-c4ccc(-n5ccn(C)c5=O)c(Cl)c4)c3O)cc(=O)n2C)CC1 ; CN1CC2CN(c3cc(-c4cc(F)cc(-c5ccc(-n6ccn(C)c6=O)c(Cl)c5)c4O)ccn3)CC21]

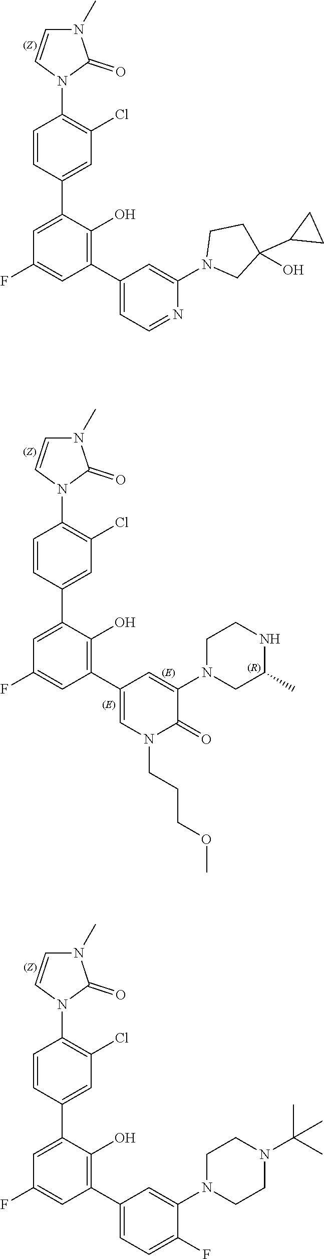

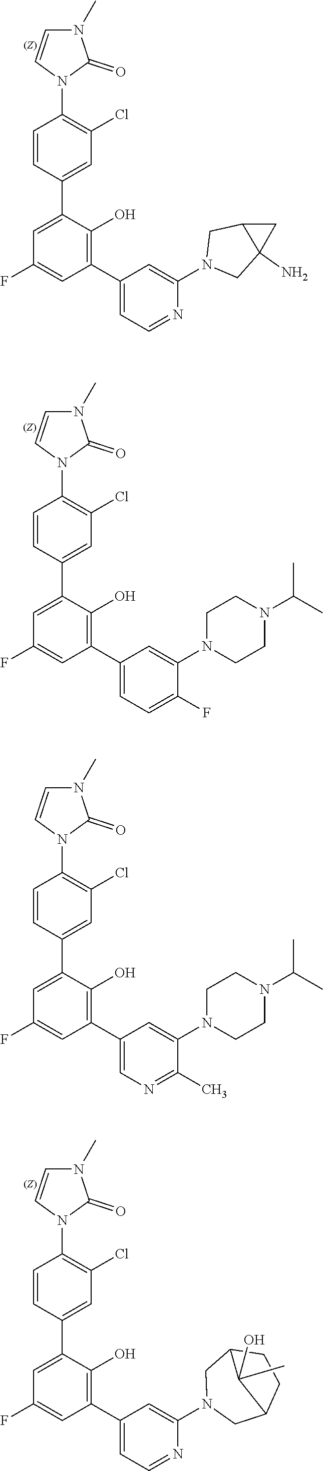 [SMILES: Cn1ccn(-c2ccc(-c3cc(F)cc(-c4ccnc(N5CC6CC6(N)C5)c4)c3O)cc2Cl)c1=O ; CC(C)N1CCN(c2cc(-c3cc(F)cc(-c4ccc(-n5ccn(C)c5=O)c(Cl)c4)c3O)ccc2F)CC1 ; Cc1ncc(-c2cc(F)cc(-c3ccc(-n4ccn(C)c4=O)c(Cl)c3)c2O)cc1N1CCN(C(C)C)CC1 ; Cn1ccn(-c2ccc(-c3cc(F)cc(-c4ccnc(N5CC6CCC(C5)C6(C)O)c4)c3O)cc2Cl)c1=O]

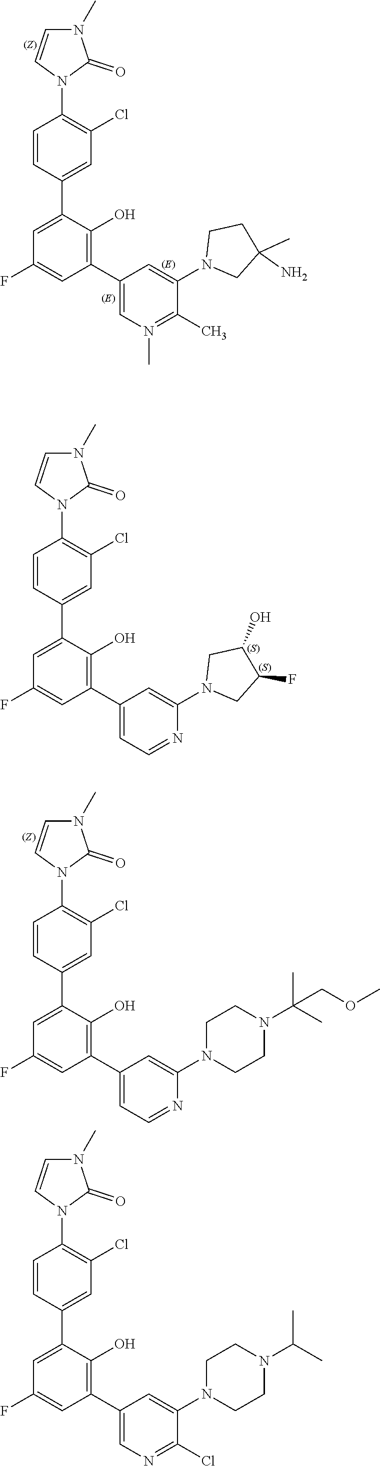

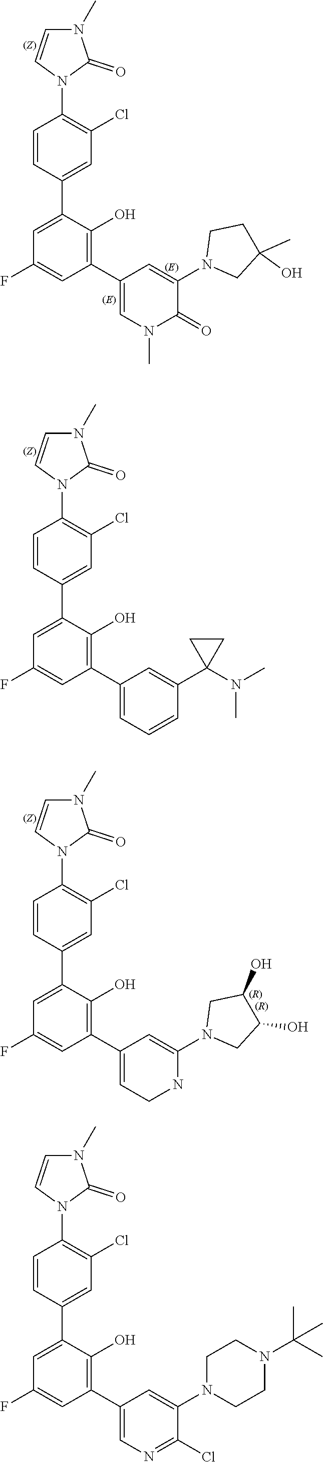

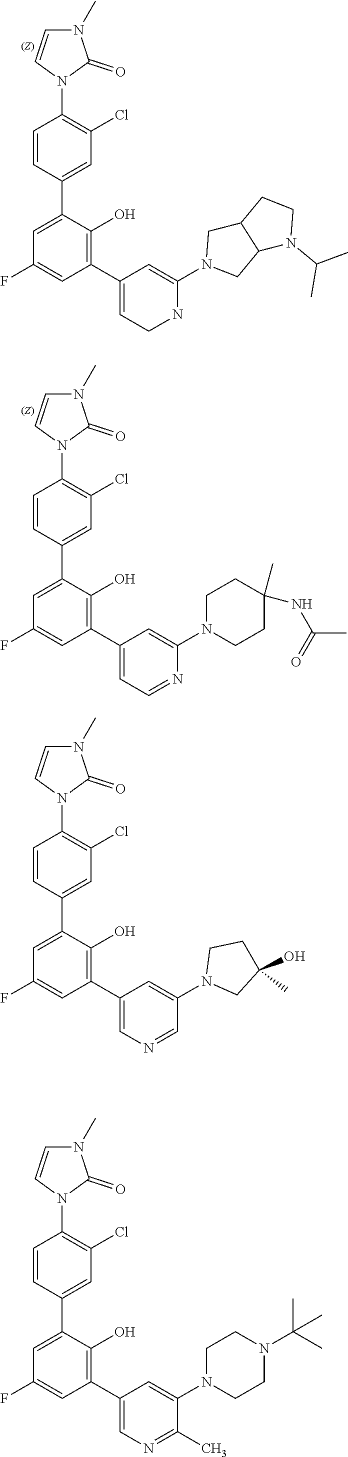

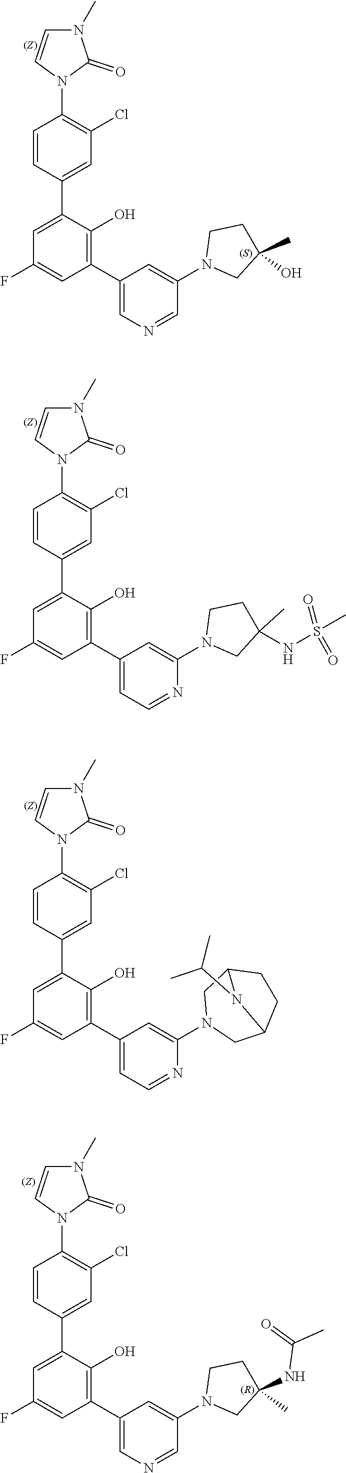

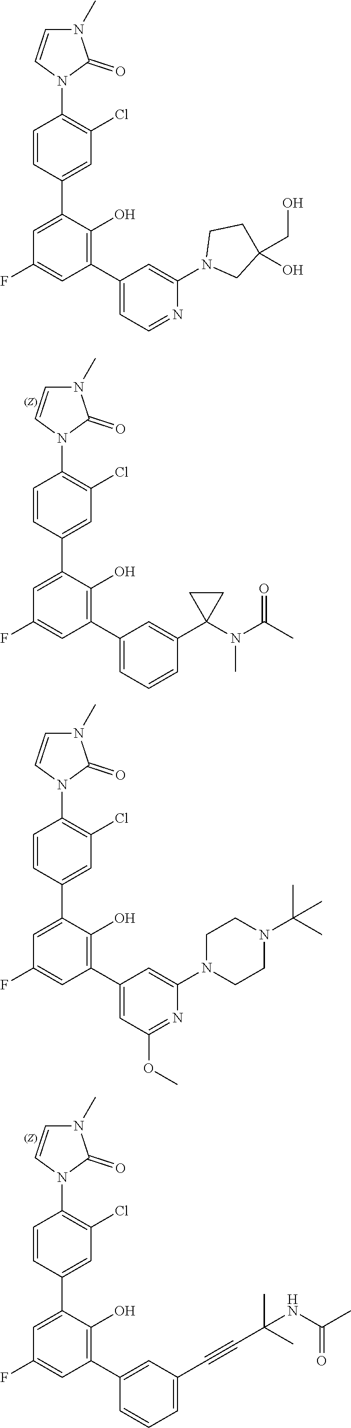

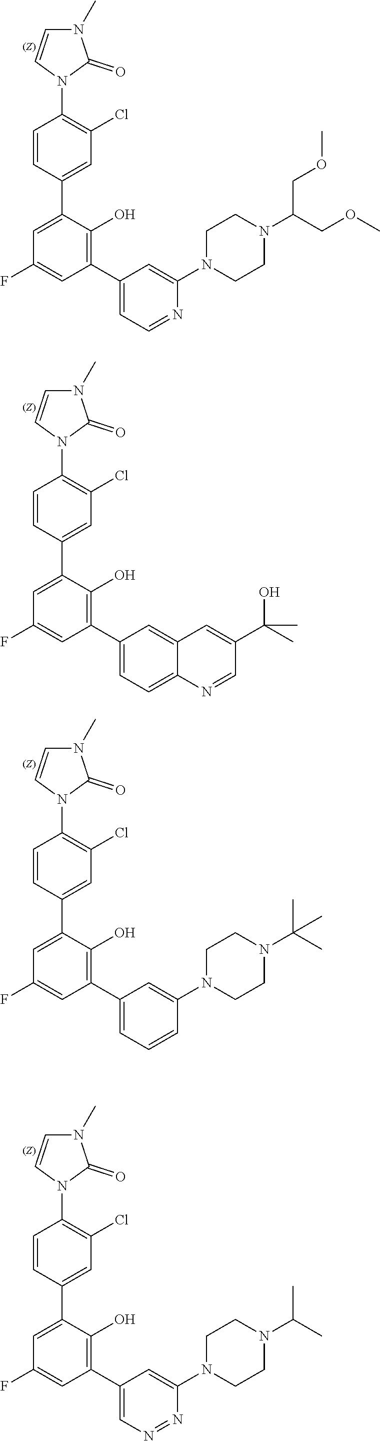 [SMILES: COCC(COC)N1CCN(c2cc(-c3cc(F)cc(-c4ccc(-n5ccn(C)c5=O)c(Cl)c4)c3O)ccn2)CC1 ; Cn1ccn(-c2ccc(-c3cc(F)cc(-c4ccc5ncc(C(C)(C)O)cc5c4)c3O)cc2Cl)c1=O ; Cn1ccn(-c2ccc(-c3cc(F)cc(-c4cccc(N5CCN(C(C)(C)C)CC5)c4)c3O)cc2Cl)c1=O ; CC(C)N1CCN(c2cc(-c3cc(F)cc(-c4ccc(-n5ccn(C)c5=O)c(Cl)c4)c3O)cnn2)CC1]

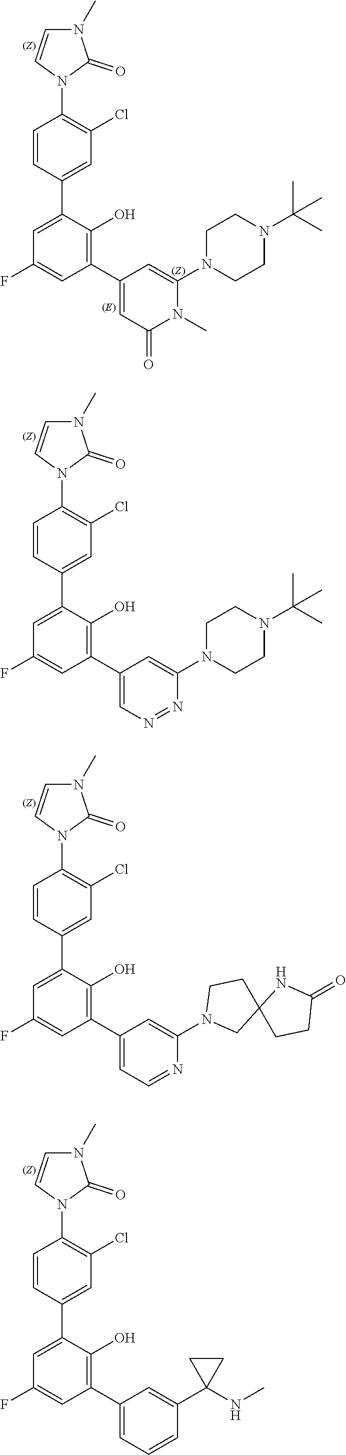 [SMILES: Cn1ccn(-c2ccc(-c3cc(F)cc(-c4cc(N5CCN(C(C)(C)C)CC5)n(C)c(=O)c4)c3O)cc2Cl)c1=O ; Cn1ccn(-c2ccc(-c3cc(F)cc(-c4cnnc(N5CCN(C(C)(C)C)CC5)c4)c3O)cc2Cl)c1=O ; Cn1ccn(-c2ccc(-c3cc(F)cc(-c4ccnc(N5CCC6(CCC(=O)N6)C5)c4)c3O)cc2Cl)c1=O ; CNC1(c2cccc(-c3cc(F)cc(-c4ccc(-n5ccn(C)c5=O)c(Cl)c4)c3O)c2)CC1]

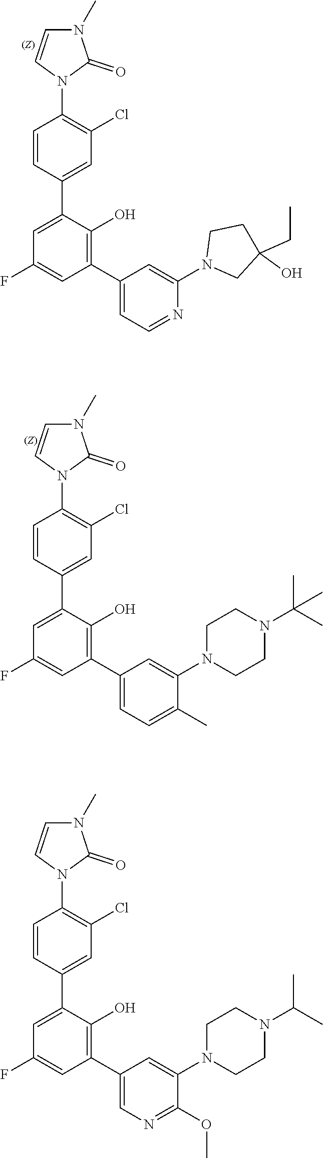 [SMILES: CCC1(O)CCN(c2cc(-c3cc(F)cc(-c4ccc(-n5ccn(C)c5=O)c(Cl)c4)c3O)ccn2)C1 ; Cc1ccc(-c2cc(F)cc(-c3ccc(-n4ccn(C)c4=O)c(Cl)c3)c2O)cc1N1CCN(C(C)(C)C)CC1 ; COc1ncc(-c2cc(F)cc(-c3ccc(-n4ccn(C)c4=O)c(Cl)c3)c2O)cc1N1CCN(C(C)C)CC1]

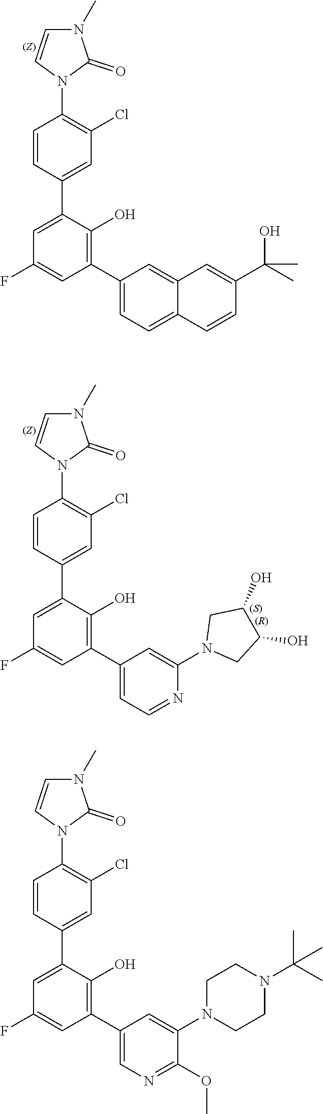

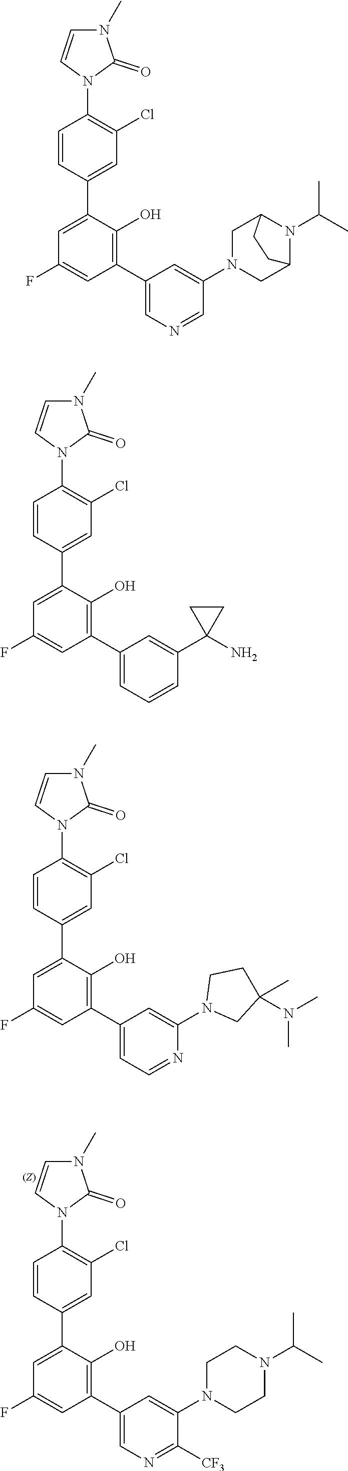 [SMILES: CC(C)N1C2CCC1CN(c1cncc(-c3cc(F)cc(-c4ccc(-n5ccn(C)c5=O)c(Cl)c4)c3O)c1)C2 ; Cn1ccn(-c2ccc(-c3cc(F)cc(-c4cccc(C5(N)CC5)c4)c3O)cc2Cl)c1=O ; CN(C)C1(C)CCN(c2cc(-c3cc(F)cc(-c4ccc(-n5ccn(C)c5=O)c(Cl)c4)c3O)ccn2)C1 ; CC(C)N1CCN(c2cc(-c3cc(F)cc(-c4ccc(-n5ccn(C)c5=O)c(Cl)c4)c3O)cnc2C(F)(F)F)CC1]

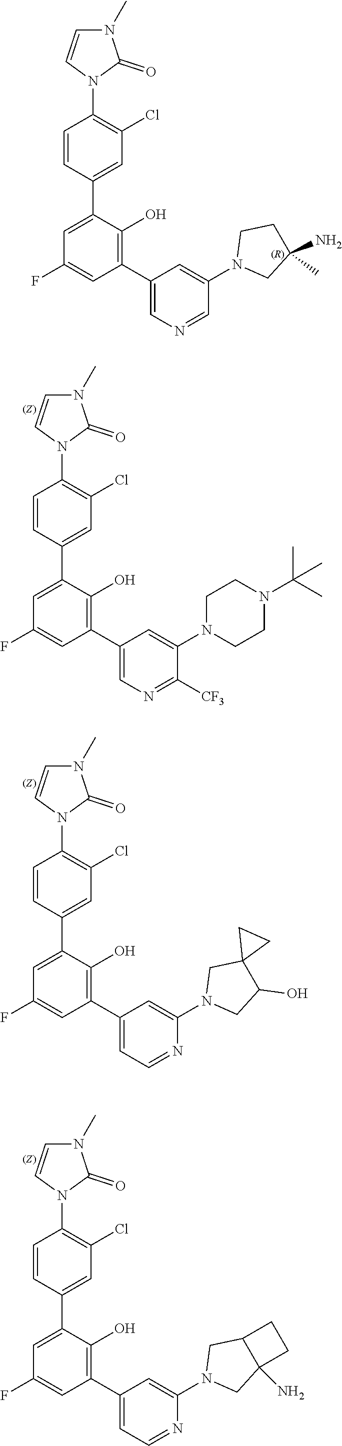

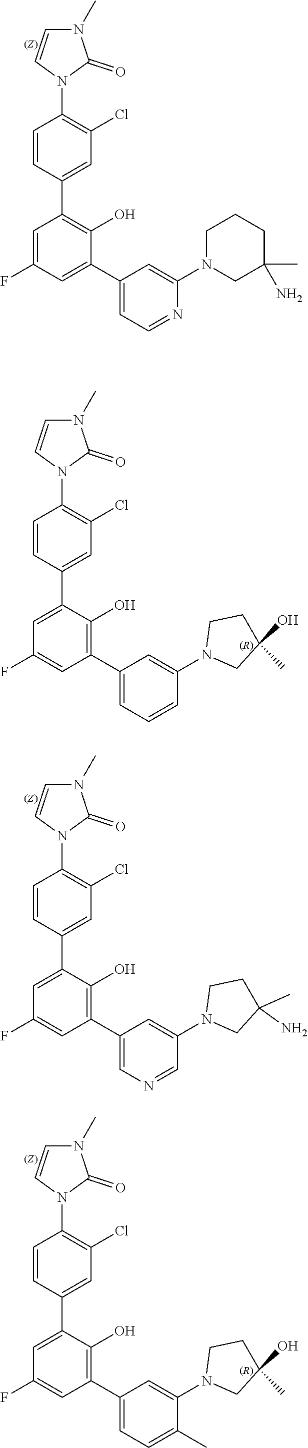

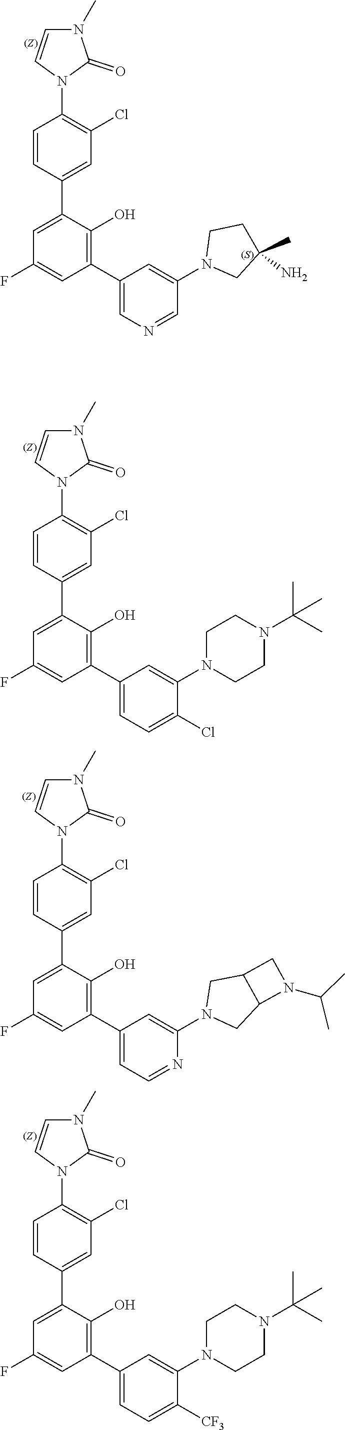

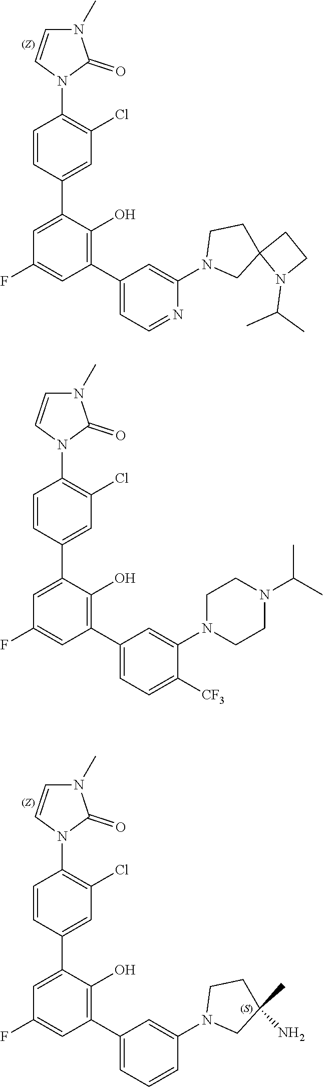

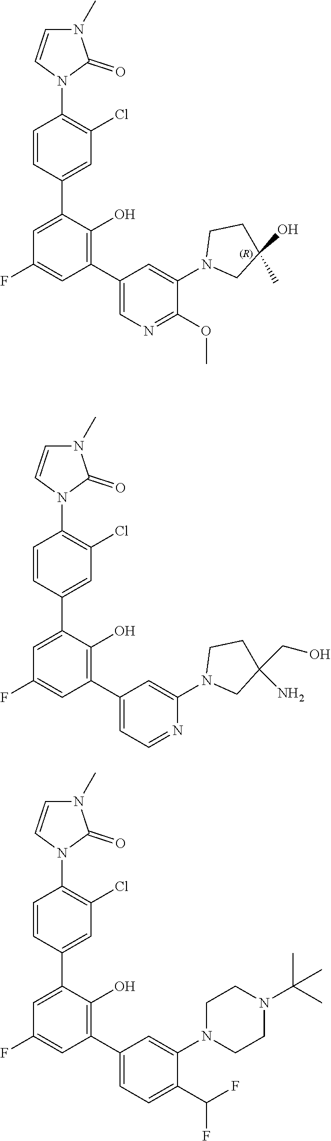

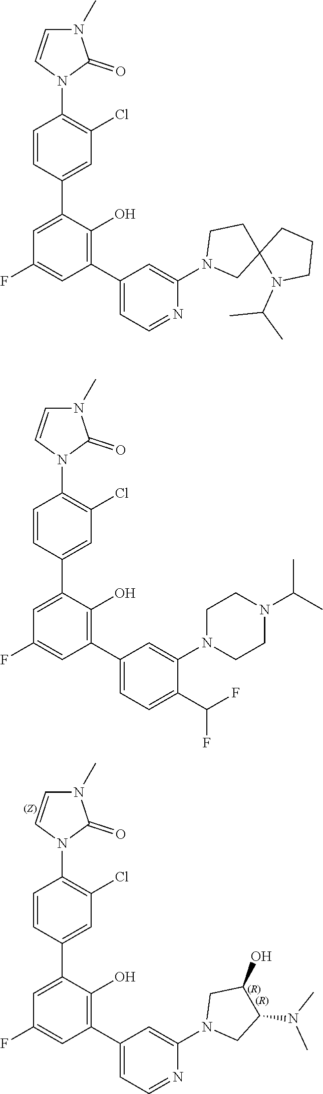

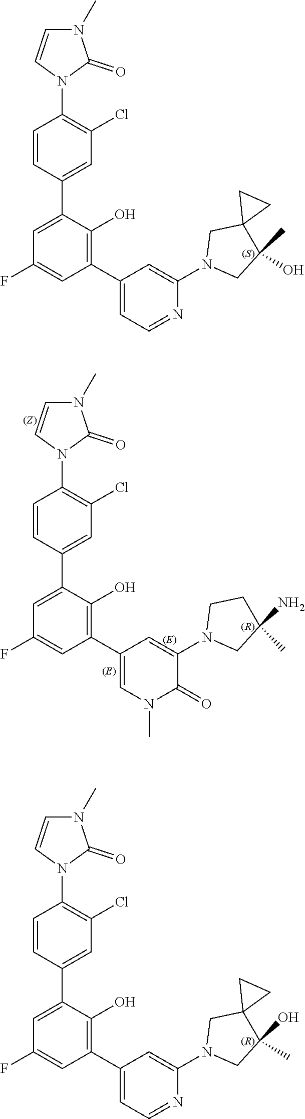

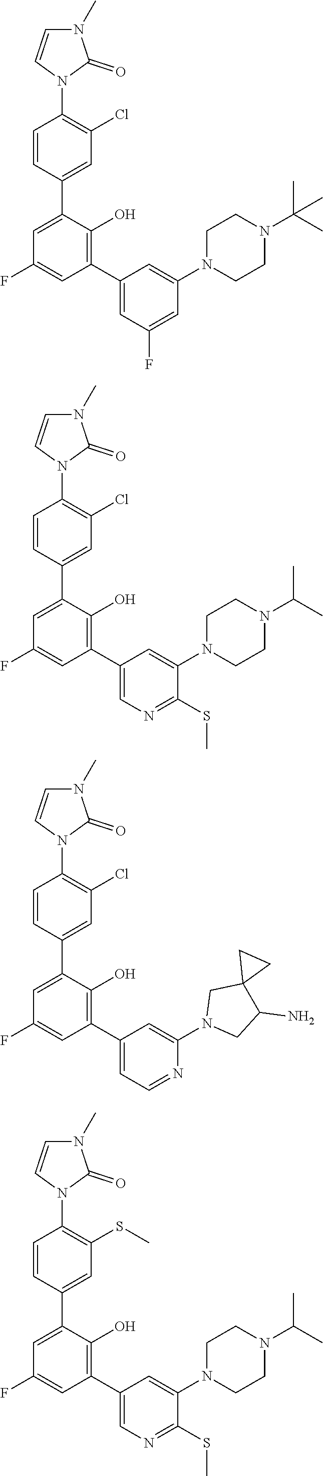

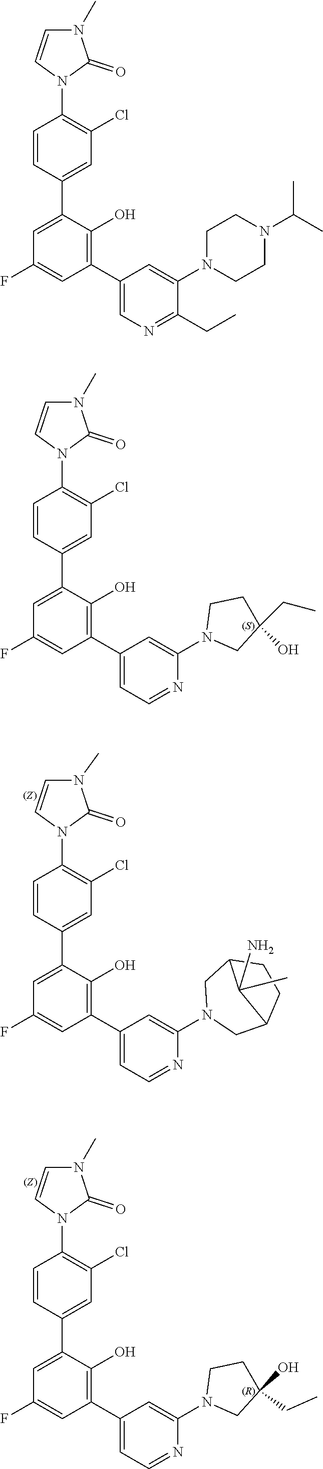

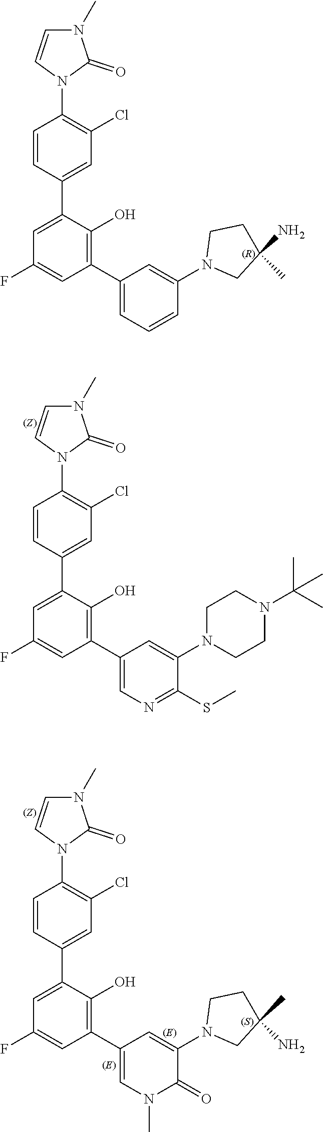

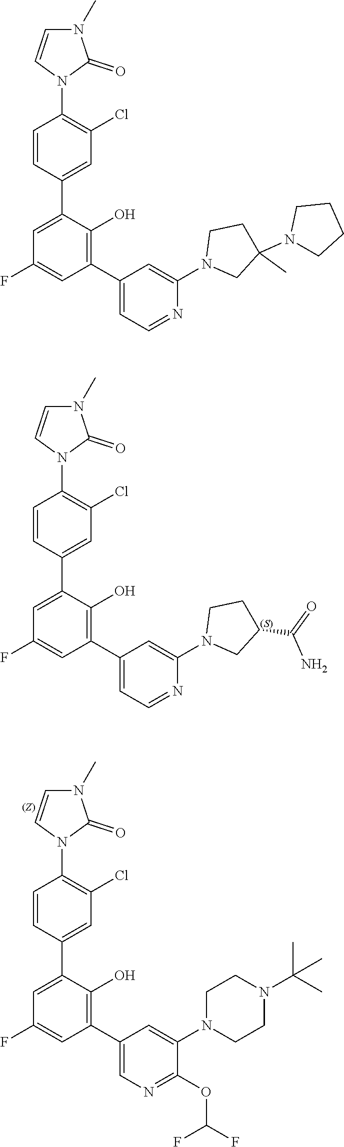

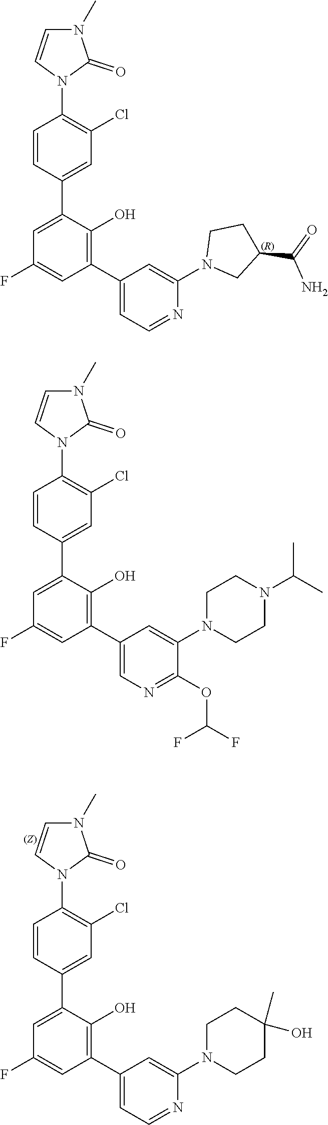

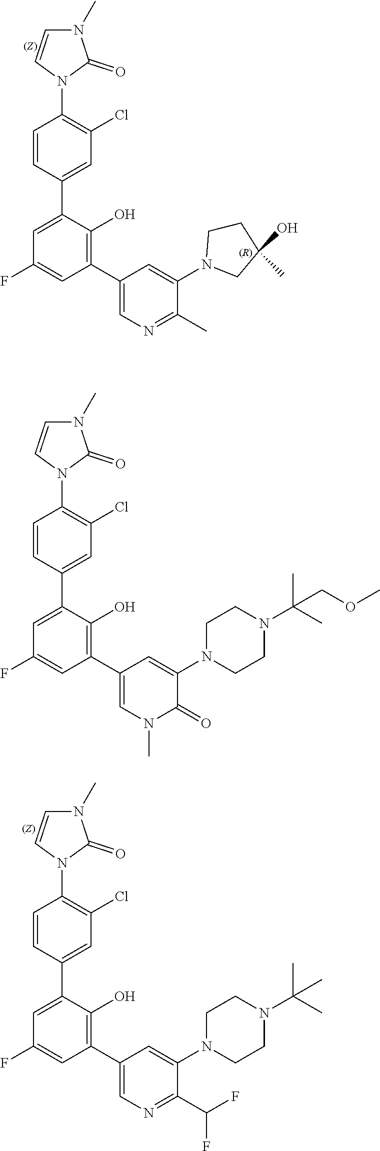

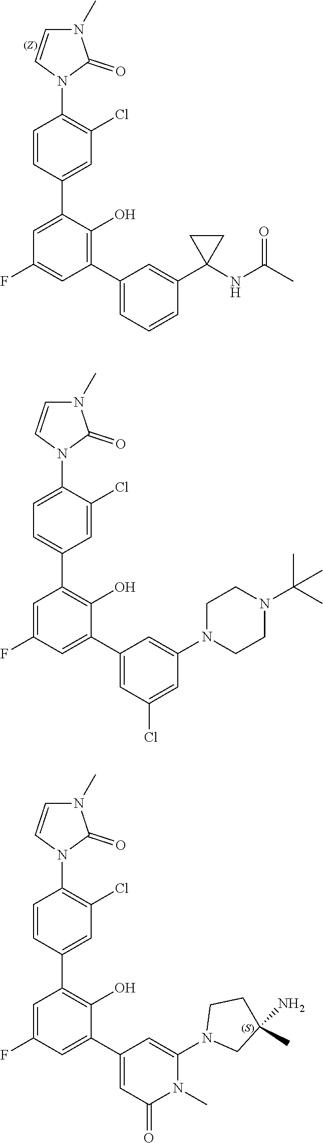

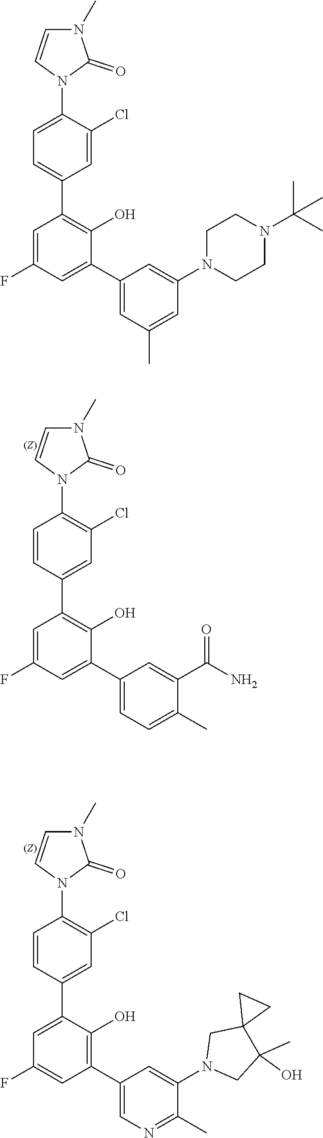

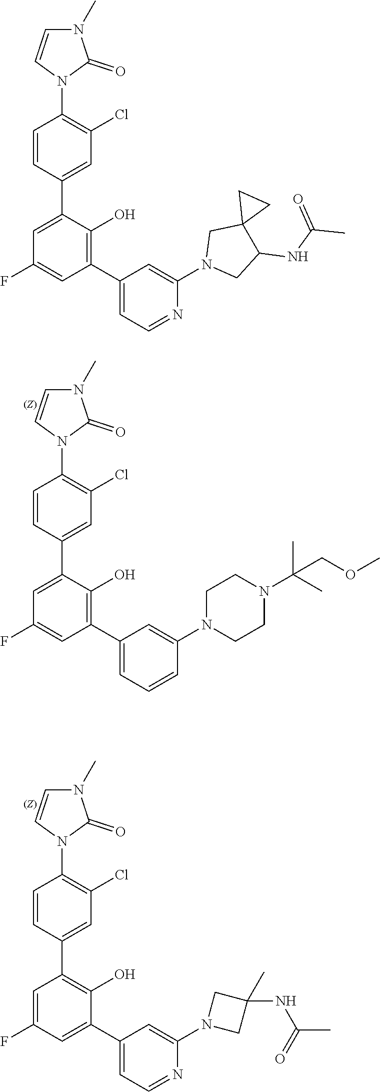

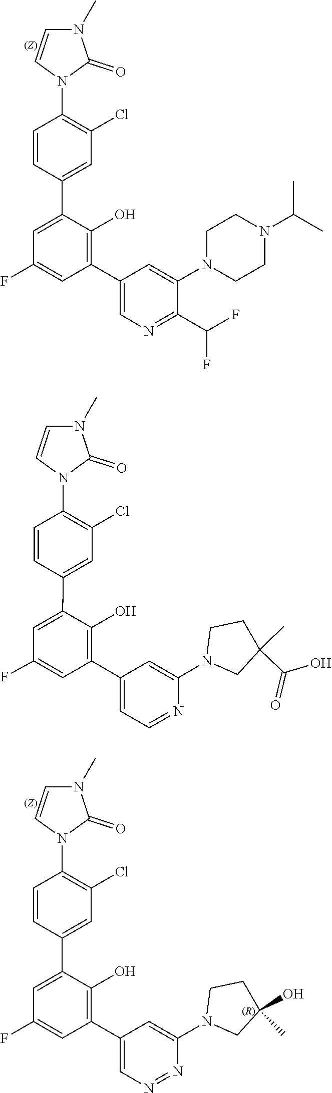

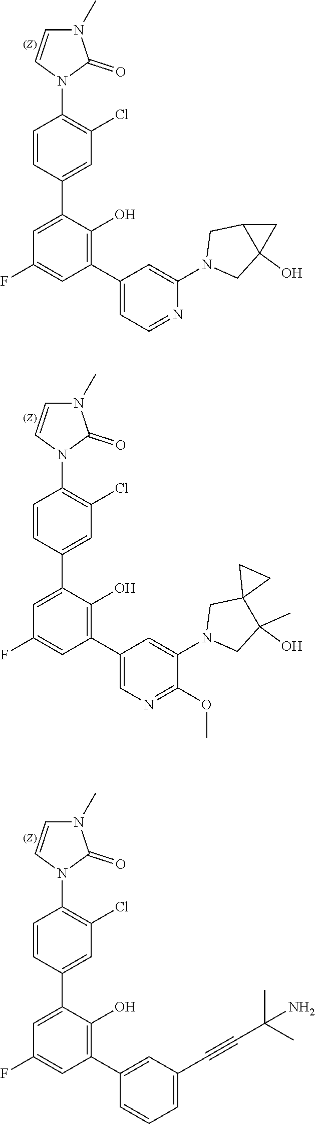

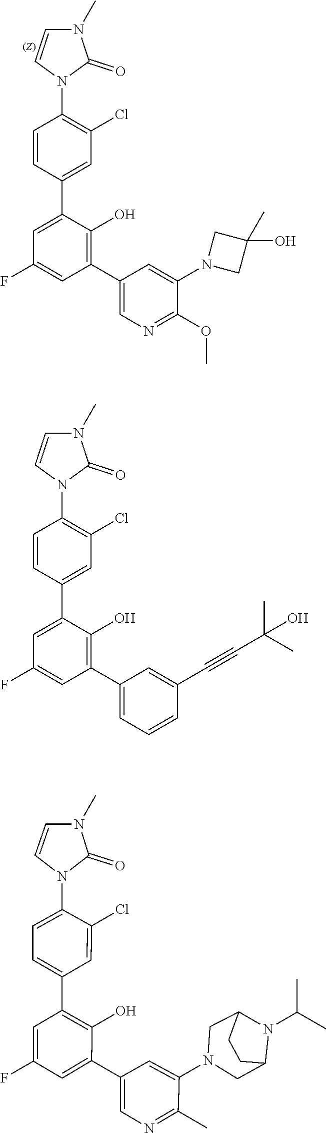

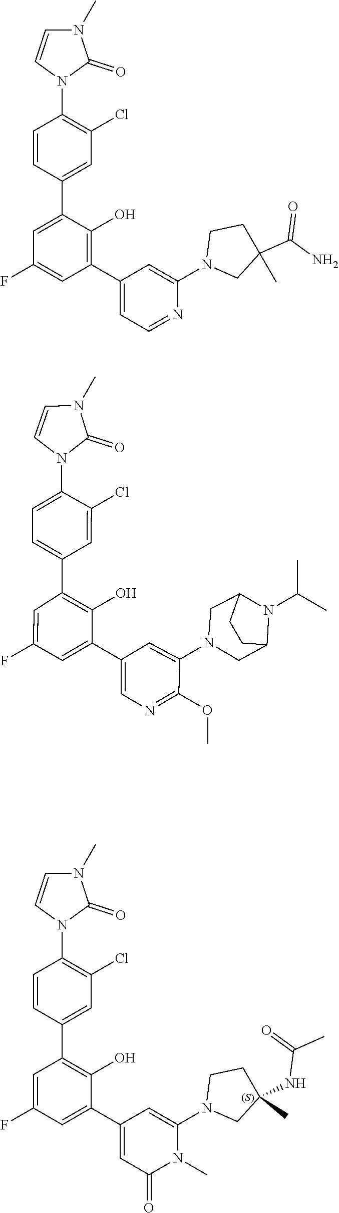

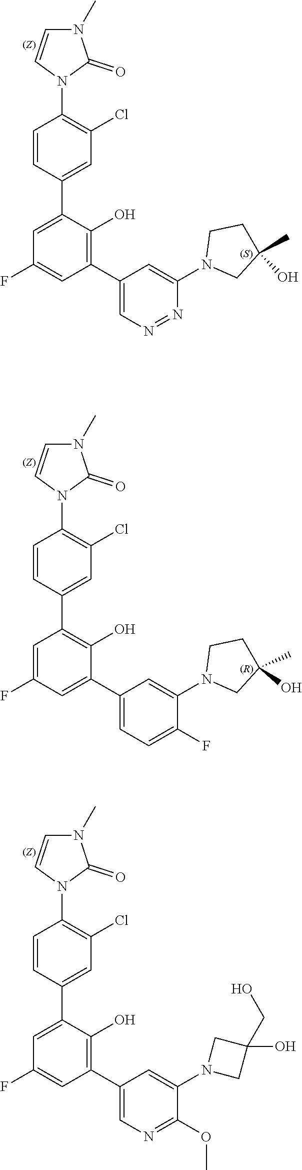

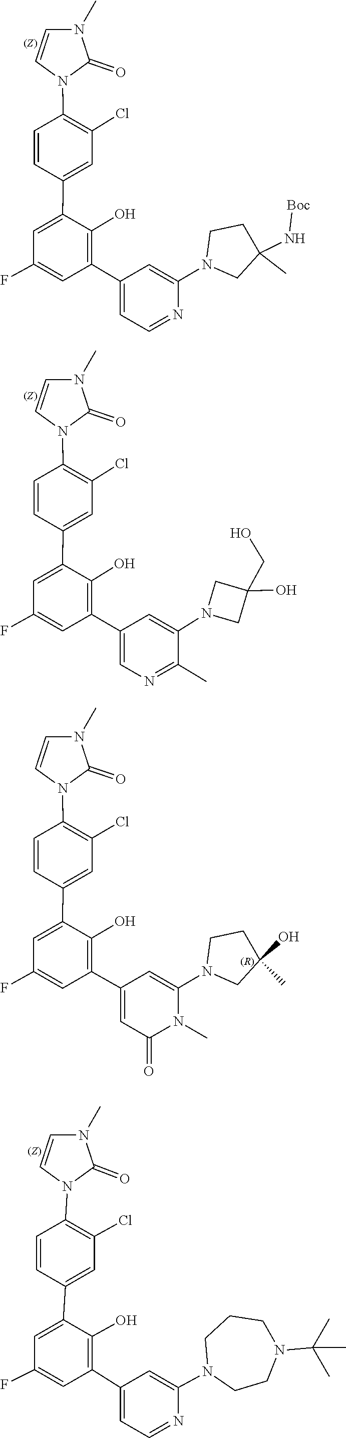

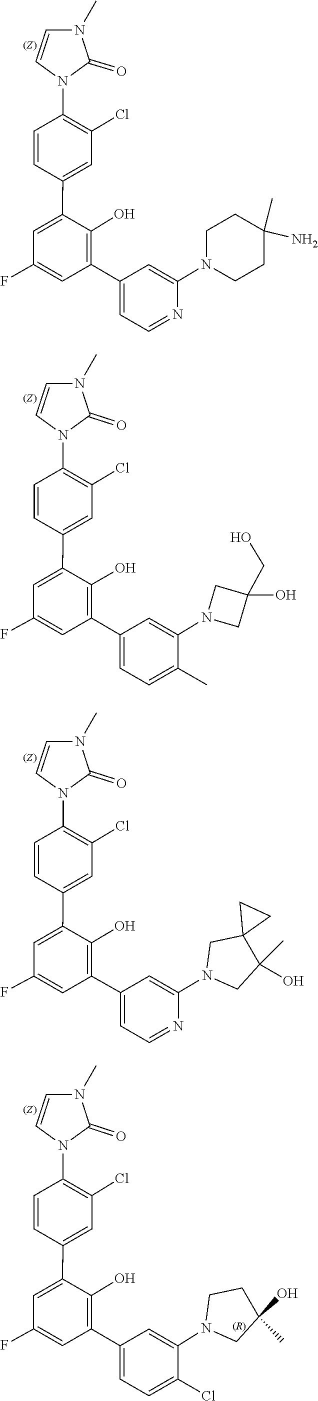

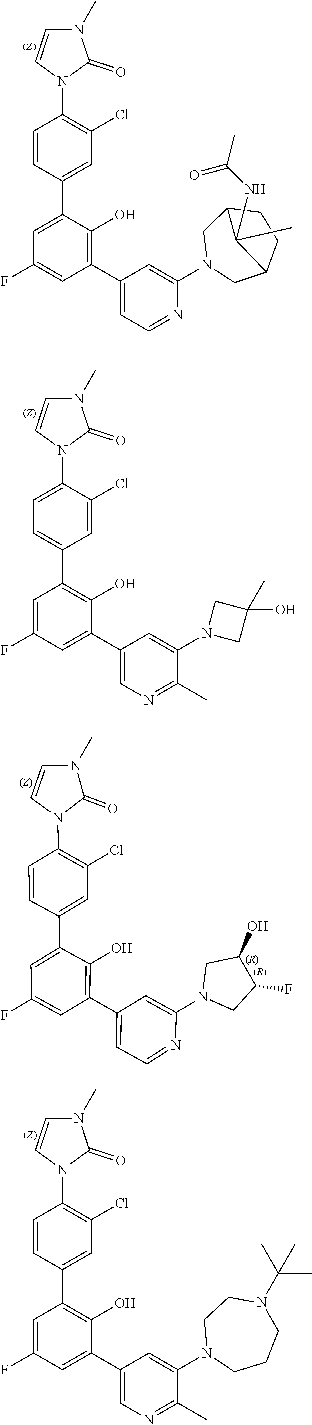

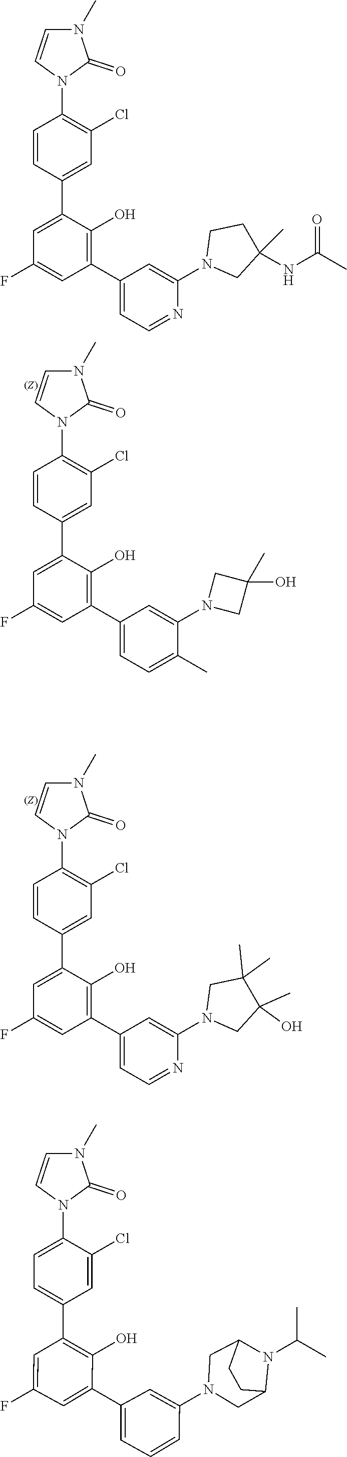

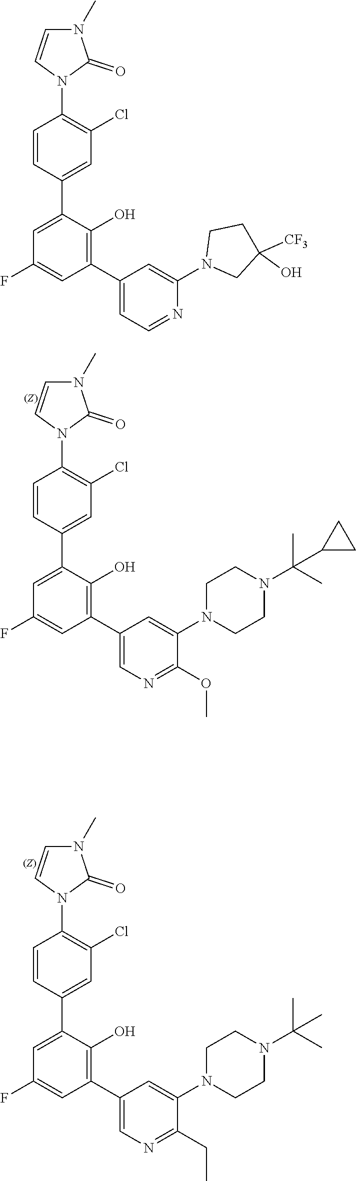

In another aspect, the present disclosure provides a method of treating an autoimmune or inflammatory disease or disorder in a subject in need thereof, comprising administering to the subject a therapeutically effective amount of a compound, wherein the compound has a structure represented by formula (II) or a pharmaceutically acceptable salt thereof:

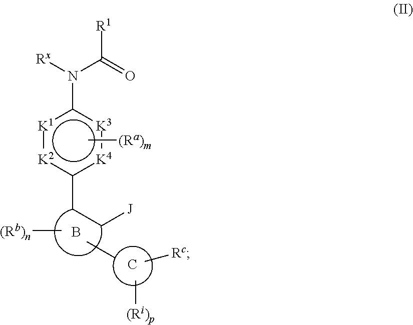

or a pharmaceutically acceptable salt thereof;
wherein:
each of K¹-K⁴ is independently CH or N;
   wherein at least one of K¹-K⁴ is CH;
Ring B represents substituted or unsubstituted phenylene or 6-membered heteroarylene;
Ring C represents substituted or unsubstituted arylene or heteroarylene;
R¹ represents alkyl, alkenyl, haloalkyl, —O(alkyl), —S(alkyl), —NH(alkyl), or —N(alkyl)₂;
R^{x} represents H, alkyl, or —C(O)alkyl;
   or R¹ and R^{x}, taken together with the intervening atoms, form an optionally substituted heterocycloalkyl ring, heterocycloalkenyl ring, or heteroaryl ring;
each occurrence of R^{a} is independently selected from the group consisting of halo, —NH₂, —NH(alkyl), —NH(cycloalkyl), —N(alkyl)₂, hydroxyl, alkyl, alkoxy, cycloalkyl, cycloalkoxy, haloalkoxy, heterocycloalkoxy, cyano, aryloxy, heteroaryloxy, and haloalkyl;
   or R¹ and an occurrence of R^{a}, taken together with the intervening atoms, form an optionally substituted heterocycloalkyl ring;
   or an occurrence of R^{a} and R^{x}, taken together with the intervening atoms, form an optionally substituted heterocycloalkyl ring;
J represents —OH, —O(alkyl), —OC(O)(alkyl), —OC(O)O(alkyl), —OC(O)NH(alkyl), —OC(O)N(alkyl)₂, —OCH₂OC(O)O(alkyl), or —NH₂;
R^{j} is alkyl, alkenyl, alkynyl, aryl, heteroaryl, arylalkyl, heteroarylalkyl, cycloalkyl, heterocycloalkyl, (cycloalkyl)alkyl, or (heterocycloalkyl)alkyl;
each occurrence of R^{b} is independently selected from the group consisting of halo, oxo, alkyl, alkoxyl, haloalkyl, cyano, cycloalkyl, aryl, aryloxy, —OH, —NH(alkyl), —C(O)H, —CO₂(alkyl) and —CO₂H;
R^{c} represents H, F, or Cl;
each occurrence of Rⁱ is independently halo, oxo, —S(alkyl), —NH₂, —NH(alkyl), —N(alkyl)₂, —OH, or cyano, or is selected from the group consisting of optionally substituted alkyl, haloalkyl, haloalkoxyl, alkoxyl, heterocycloalkyl, and cycloalkoxyl;
   or two adjacent occurrences of Rⁱ, taken together with the intervening atoms, form an optionally substituted aryl, heteroaryl, cycloalkyl, or heterocycloalkyl ring; and
m, n, and p are each independently 0, 1, or 2.

In certain embodiments, each of K¹-K⁴ is CH.

In certain embodiments, m is 1; and R^{a} is halo.

In certain embodiments, Ring B represents substituted or unsubstituted phenylene.

In certain embodiments, n is 0. In certain embodiments, J is OH.

In certain embodiments, R^{x} represents H. In certain embodiments, R¹ is alkyl.

Alternatively, R¹ and R^{x}, taken together with the intervening atoms, may form an optionally substituted heterocycloalkyl ring, heterocycloalkenyl ring, or heteroaryl ring.

In certain embodiments, the compound of formula (II) is selected from the following table:

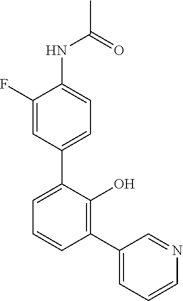 [SMILES: CC(=O)Nc1ccc(-c2cccc(-c3cccnc3)c2O)cc1F]

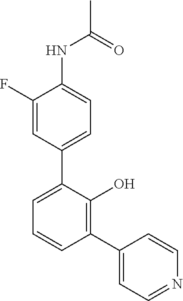 [SMILES: CC(=O)Nc1ccc(-c2cccc(-c3ccncc3)c2O)cc1F]

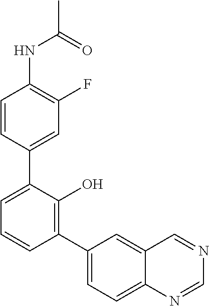 [SMILES: CC(=O)Nc1ccc(-c2cccc(-c3ccc4ncncc4c3)c2O)cc1F]

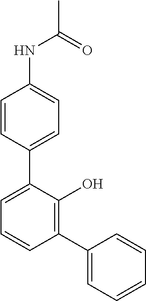 [SMILES: CC(=O)Nc1ccc(-c2cccc(-c3ccccc3)c2O)cc1]

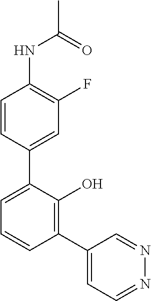 [SMILES: CC(=O)Nc1ccc(-c2cccc(-c3ccnnc3)c2O)cc1F]

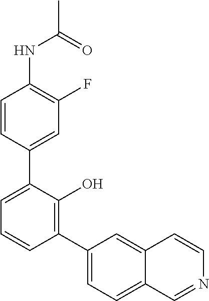 [SMILES: CC(=O)Nc1ccc(-c2cccc(-c3ccc4cnccc4c3)c2O)cc1F]

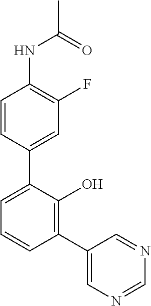 [SMILES: CC(=O)Nc1ccc(-c2cccc(-c3cncnc3)c2O)cc1F]

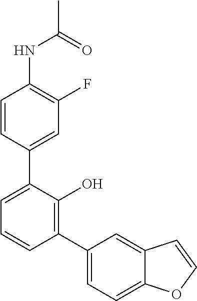 [SMILES: CC(=O)Nc1ccc(-c2cccc(-c3ccc4occc4c3)c2O)cc1F]

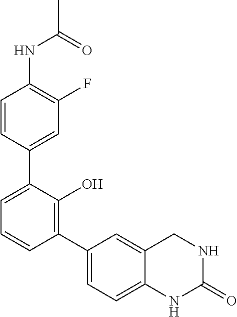 [SMILES: CC(=O)Nc1ccc(-c2cccc(-c3ccc4c(c3)CNC(=O)N4)c2O)cc1F]

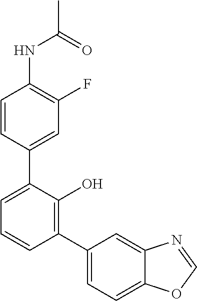 [SMILES: CC(=O)Nc1ccc(-c2cccc(-c3ccc4ocnc4c3)c2O)cc1F]

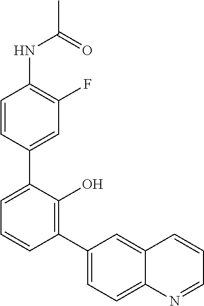 [SMILES: CC(=O)Nc1ccc(-c2cccc(-c3ccc4ncccc4c3)c2O)cc1F]

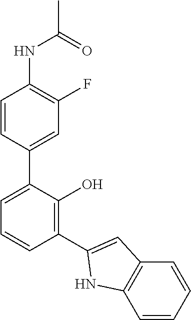 [SMILES: CC(=O)Nc1ccc(-c2cccc(-c3cc4ccccc4[nH]3)c2O)cc1F]

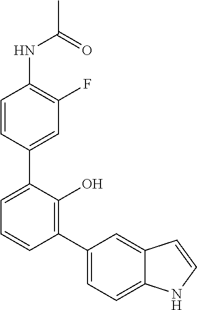 [SMILES: CC(=O)Nc1ccc(-c2cccc(-c3ccc4[nH]ccc4c3)c2O)cc1F]

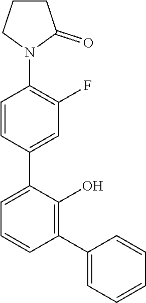 [SMILES: O=C1CCCN1c1ccc(-c2cccc(-c3ccccc3)c2O)cc1F]

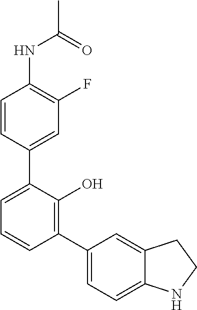 [SMILES: CC(=O)Nc1ccc(-c2cccc(-c3ccc4c(c3)CCN4)c2O)cc1F]

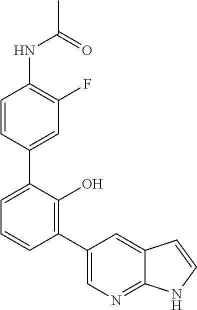 [SMILES: CC(=O)Nc1ccc(-c2cccc(-c3cnc4[nH]ccc4c3)c2O)cc1F]

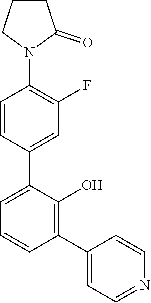 [SMILES: O=C1CCCN1c1ccc(-c2cccc(-c3ccncc3)c2O)cc1F]

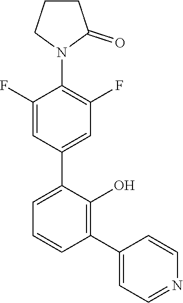 [SMILES: O=C1CCCN1c1c(F)cc(-c2cccc(-c3ccncc3)c2O)cc1F]

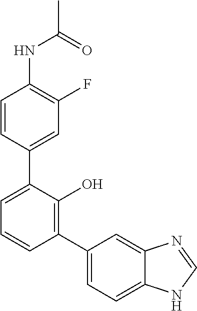 [SMILES: CC(=O)Nc1ccc(-c2cccc(-c3ccc4[nH]cnc4c3)c2O)cc1F]

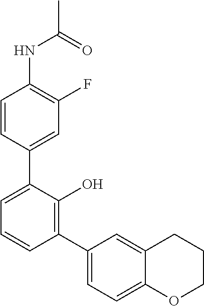 [SMILES: CC(=O)Nc1ccc(-c2cccc(-c3ccc4c(c3)CCCO4)c2O)cc1F]

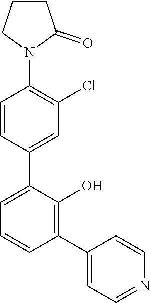 [SMILES: O=C1CCCN1c1ccc(-c2cccc(-c3ccncc3)c2O)cc1Cl]

In another aspect, the present disclosure provides a method of treating an autoimmune or inflammatory disease or disorder in a subject in need thereof, comprising administering to the subject a therapeutically effective amount of a compound, wherein the compound has a structure represented by formula (III) or a pharmaceutically acceptable salt thereof:

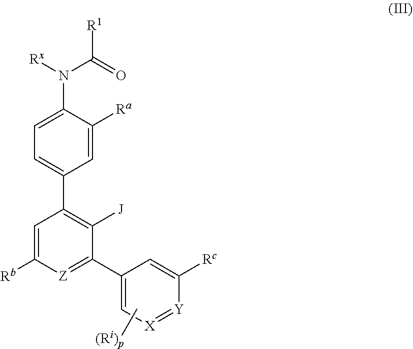

further wherein,
J is —OH, —O(alkyl), —OC(O)(alkyl), —OC(O)O(alkyl), —OC(O)NH(alkyl), —OC(O)N(alkyl)₂, —OCH₂OC(O)O(alkyl), or —NH₂;
X and Y are each independently selected from CH and N provided that at least one of X and Y is CH, or X is C(O) and Y is N(alkyl);
Z is N or CH;
R¹ is alkyl, alkenyl, haloalkyl, —O(alkyl), —S(alkyl), —NH(alkyl), or —N(alkyl)₂;
R^{x} represents H, alkyl, or —C(O)alkyl;
   or R¹ and R^{x}, taken together with the intervening atoms, form an optionally substituted heterocycloalkyl ring, heterocycloalkenyl ring, or heteroaryl ring;
R^{a} is independently selected from the group consisting of halo, —NH₂, —NH(alkyl), —NH(cycloalkyl), —N(alkyl)₂, alkyl, alkoxy, cycloalkoxy, haloalkoxy, heterocycloalkoxy, cyano, aryloxy, heteroaryloxy, and haloalkyl;
R^{b} is independently selected from the group consisting of halo, alkyl, alkoxyl, cyano, cycloalkyl, aryl, aryloxy, —CO₂(alkyl) and —CO₂H;
R^{c} is heterocycloalkyl, cycloalkyl, alkyl, aryl, heteroaryl, heterocyclyl, alkoxyl, alkynyl, aryloxy, haloalkyl, haloalkoxy, cycloalkoxyl, heterocycloalkoxyl, halo, —S(alkyl), —NH₂, —CO₂H, —CO₂(alkyl), or —NHCO(alkyl);
each Rⁱ is independently halo, oxo, —S(alkyl), cyano, alkyl, haloalkyl, haloalkoxyl, alkoxyl, heterocycloalkyl, heterocyclyl, or cycloalkoxyl;
   or R^{c} and an occurrence of Rⁱ, taken together with the intervening atoms, form an aryl, heteroaryl, cycloalkyl, or heterocycloalkyl ring;
   or two adjacent occurrences of Rⁱ, taken together with the intervening atoms, form an aryl, heteroaryl, cycloalkyl, or heterocycloalkyl ring; and
p is 0, 1, or 2.

In certain embodiments, the compound has a structure represented by formula (IIIa) or a pharmaceutically acceptable salt thereof:

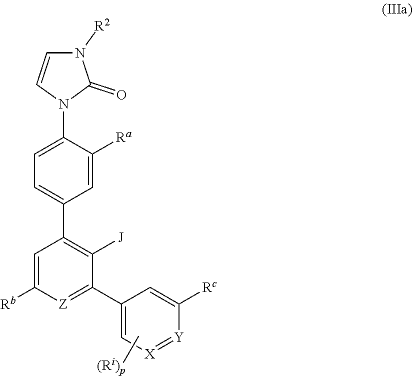

further wherein R² is H, alkyl, alkenyl, haloalkyl, or deuteroalkyl.

In certain embodiments, R² is alkyl (e.g., methyl). In certain embodiments, R² is deuteroalkyl (e.g., deuteromethyl).

In certain embodiments, R_{c} is heterocyclyl (e.g., piperazinyl).

In certain embodiments, the compound has a structure represented by formula (IIIb) or a pharmaceutically acceptable salt thereof:

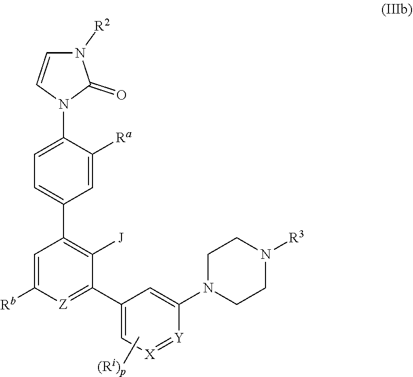

further wherein R³ is H, alkyl, alkenyl, haloalkyl, or deuteroalkyl.

In certain embodiments, R³ is alkyl (e.g., tertiary butyl).

In certain embodiments, R^{a} is halo (e.g., chloro or fluoro).

In certain embodiments, J is —OH. In certain embodiments, J is —NH₂.

In certain embodiments, R^{b} is alkyl (e.g., methyl). In certain embodiments, R^{b} is halo (e.g., chloro or fluoro).

In certain embodiments, one Rⁱ is halo (e.g., chloro or fluoro). In certain embodiments, one Rⁱ is alkyl (e.g., methyl). In certain embodiments, Rⁱ is alkoxyl (e.g., methoxy). In certain embodiments, one Rⁱ is oxo.

In certain embodiments, X is N and Y is CH. In certain embodiments, X is CH and Y is N.

In certain embodiments, X and Y are each CH. In certain embodiments, X is C(O) and Y is N(Me).

In certain embodiments, p is 0. In certain embodiments, p is 1. In certain embodiments, p is 2.

In certain embodiments, Z is N. In certain embodiments, Z is CH.

In certain embodiments, the compound has a structure represented by formula (IIIc) or a pharmaceutically acceptable salt thereof:

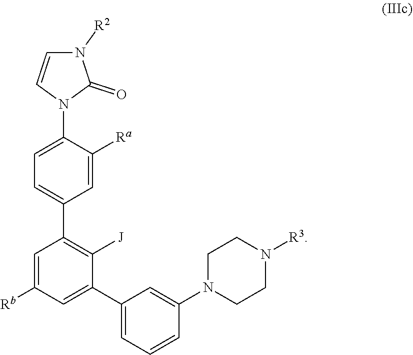

In certain embodiments, the compound has a structure represented by formula (IIId) or a pharmaceutically acceptable salt thereof:

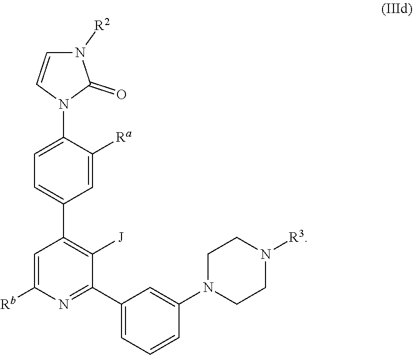

In certain embodiments, the compound has a structure represented by formula (IIIe) or a pharmaceutically acceptable salt thereof:

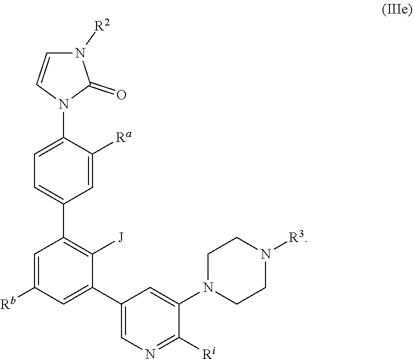

In certain embodiments, the compound is selected from the group consisting of

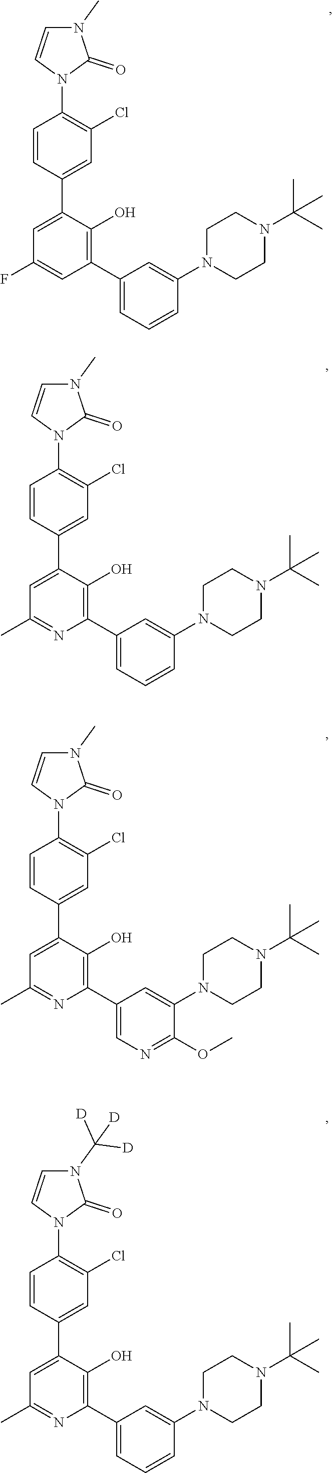

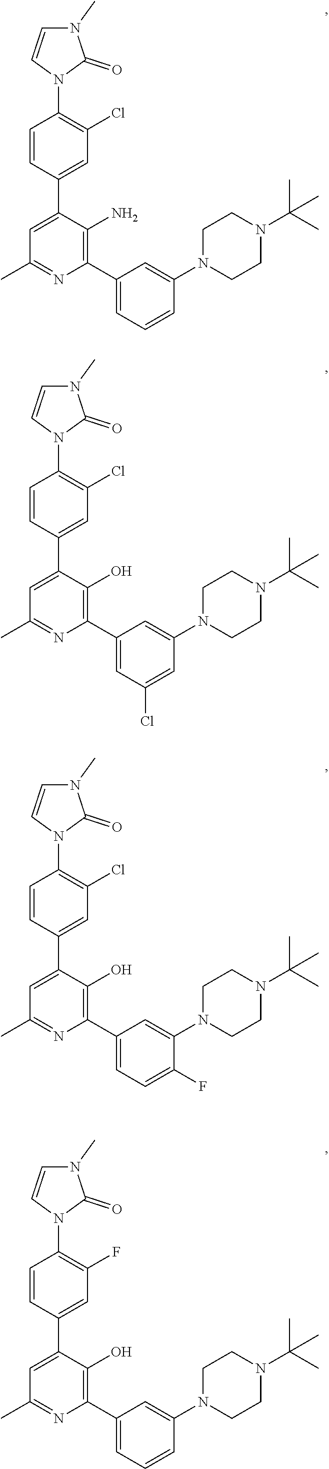

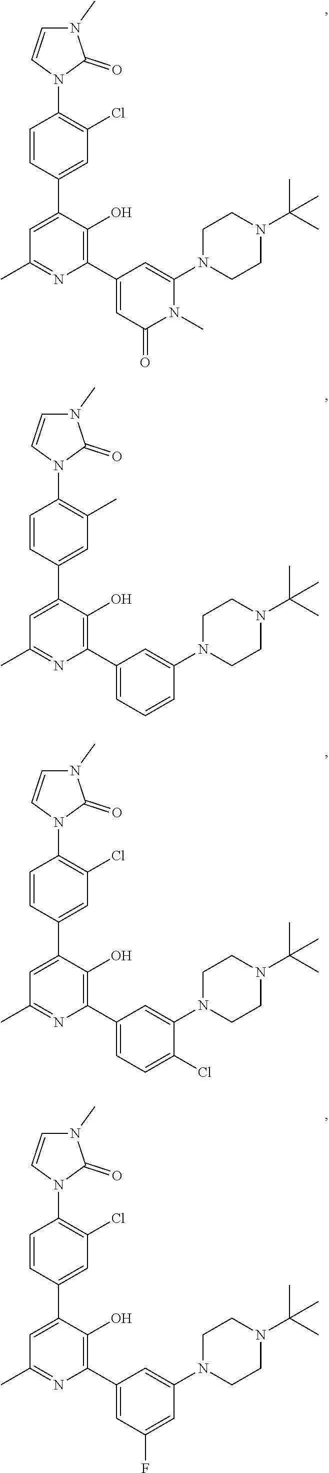

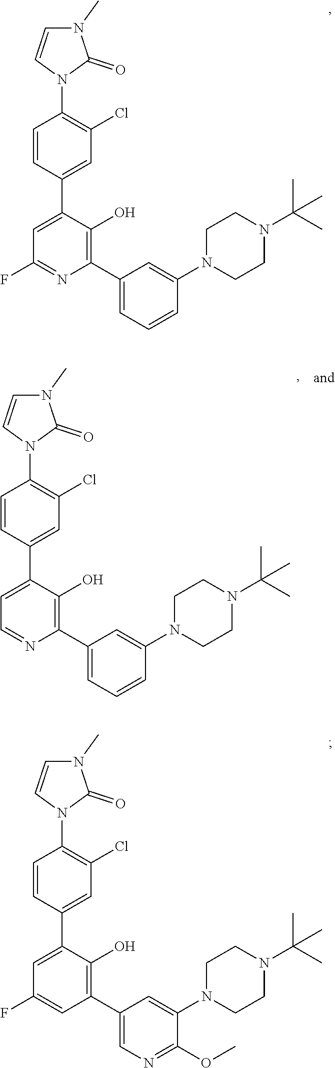

or a pharmaceutically acceptable salt thereof.

This disclosure also includes all suitable isotopic variations of a compound of the disclosure. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Such variants may also have advantageous optical properties arising, for example, from changes to vibrational modes due to the heavier isotope. Isotopic variations of a compound of the invention can generally be prepared by conventional procedures known by a person skilled in the art such as by the illustrative methods or by the preparations described in the examples hereafter using appropriate isotopic variations of suitable reagents.

Compounds disclosed herein and methods of making the compounds disclosed herein are recited in PCT/US2022/032669, the contents of which are hereby incorporated by reference in their entirety.

In certain embodiments, an autoimmune disease or disorder is treated.

In certain embodiments, an inflammatory disease or disorder is treated.

In certain embodiments, the autoimmune or inflammatory disease or disorder is cardiovascular inflammation.

In certain embodiments, the autoimmune or inflammatory disease or disorder is vascular inflammation.

In certain embodiments, the autoimmune or inflammatory disease or disorder is at least partially mediated by Th17 cells.

In certain embodiments, the autoimmune or inflammatory disease or disorder is arthritis, spondyloarthropathy, multiple sclerosis, psoriasis, lupus, vitiligo, inflammatory bowel disease (IBD), scleroderma, or systemic sclerosis.

In certain embodiments, the autoimmune or inflammatory disease or disorder is psoriasis.

In certain embodiments, the autoimmune or inflammatory disease or disorder is systemic lupus erythematosus.

In certain embodiments, the autoimmune or inflammatory disease or disorder is type I diabetes.

In certain embodiments, the autoimmune or inflammatory disease or disorder is atherosclerosis.

In certain embodiments, the autoimmune or inflammatory disease or disorder is rheumatoid arthritis (RA), Crohn's disease (CD), or asthma.

In certain embodiments, the compound is conjointly administered with an additional therapy.

In certain embodiments, the compound is conjointly administered with an additional therapy that treats the autoimmune disease or disorder.

In certain embodiments, the compound is conjointly administered with an additional therapy that treats the inflammatory disease or disorder.

In certain embodiments, the additional therapy is a JAK inhibitor. In certain embodiments, the JAK inhibitor is a JAK1 or JAK2 inhibitor. In certain embodiments, the JAK inhibitor is a JAK1 and JAK2 inhibitor.

In certain embodiments, the JAK inhibitor is ruxolitinib, fedratinib, pacritinib, momelotinib, tofacitiuib, oclacitinib, baricitinib, peficitinib, upadacitinib, delgocitinib, filgotinib, abrocitinib, or deucravacitinib.

In certain embodiments, the additional therapy is a BCL2 inhibitor. In certain embodiments, BCL2 inhibitor is navitoclax or venetoclax.

In certain embodiments, the additional therapy is a PI3K inhibitor. In certain embodiments, the PI3K inhibitor is idelalisib, copanlisib, duvelisib, alpelisib, or umbralisib.

In certain embodiments, the additional therapy is a fusion protein.

In certain embodiments, the fusion protein is abatacept, a CTLA4-Ig fusion protein.

### Pharmaceutical Compositions

The methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

An “autoimmune disease or disorder” refers to a disease or disorder wherein the body's natural immune system cannot tell the difference between its own cells and foreign cells, causing the body to mistakenly attack the body's own cells. Diseases or disorders of the adaptive immune system can cause autoimmune diseases or disorders.

An “inflammatory disease or disorder” refers to a disease or disorder wherein the body's natural immune system attacks the body's own tissue, resulting in inflammation. Diseases or disorders of the innate immune system can cause autoinflammatory diseases or disorders.

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amido”, as used herein, refers to a group

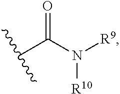

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

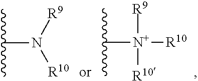

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

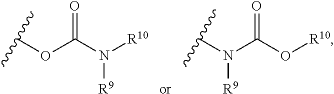

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo [2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamido” is art-recognized and refers to the group represented by the general formulae

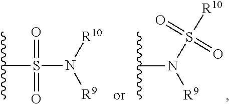

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, aphosphoryl, aphosphate, aphosphonate, aphosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

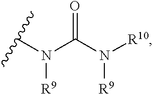

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention and are not intended to limit the invention.

### Synthesis of Exemplary Compounds of the Disclosure

### General Synthetic Methods

Exemplary compounds useful in methods of the invention will now be described by reference to the illustrative synthetic schemes for their general preparation below or the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Nuclear magnetic resonance (NMR) spectra were obtained on Bruker model AVIII 400 spectrometers. Definitions for multiplicity are as follows: s=singlet, d=doublet, t=triplet, q=quartet, dd=doublet of doublets, ddd=doublet of doublet of doublets, td=triplet of doublets, dt=doublet of triplets, spt=septet, quin=quintet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Abbreviations used in the schemes and examples, are as follows in Table 1:

TABLE 1
Table of Abbreviations

| Abbreviation | Name |
|---|---|
| ACN or MeCN | acetonitrile |
| AcOH | glacial acetic acid |
| aq. | aqueous |
| BINAP | 2,2′-bis(diphenylphosphino)-1,1′-binaphthyl |
| BTC | Triphosgene or (bis(trichloromethyl) carbonate |
| conc. | concentrated |
| Cu(OAc)₂ | copper(II) acetate |
| DCM | dichloromethane |
| DIEA | diisopropyl-ethyl amine |
| DHP | 3,4-Dihydropyran |
| DMA | dimethylaniline |
| DMAP | 4-dimethylaminopyridine |
| DMF | N,N-dimethylformamide |
| DMSO | dimethylsulfoxide |
| Dppf or DPPF | 1,1′-Bis(diphenylphosphino)ferrocene |
| EtOAc or EA | ethyl acetate |
| EtOH | ethanol |
| FCC | Flash column chromatography |
| GCMS | gas chromatography-mass spectrometry |
| h or hr(s) | hour or hours |
| HPLC | high performance liquid chromatography |
| MeOH | methanol |
| MHz | megahertz |
| min | minute or minutes |
| LCMS | Liquid chromatography mass spectrometry |
| NBS | N-Bromosuccinimide |
| NIS | N-iodosuccinimide |
| NMR | nuclear magnetic resonance |
| Pd(PPh₃)₄ | tetrakis(triphenylphosphine)palladium |
| Pd(OAc)₂ | palladium (II) acetate |
| PE | petrolum ether |
| PPh3 | triphenylphosphine |
| Pd(dppf)Cl₂ | 1,1′-bis(diphenylphosphino)ferrocene- |
| | palladium(II)dichloride |
| Pd(dppf)Cl₂•DCM | [1,1′-Bis(diphenylphosphino)ferrocene]- |
| | dichloropalladium (II) dichloromethane complex |
| Pd(TBDAP)Cl₂ | Bis(di-tert-butyl(4-dimethylaminophenyl)phosphine) |
| | dichloropalladium(II) |
| NaOtBu | Sodium tert-butoxide |
| rt or RT | room temperature |
| TBS | tert-Butyldimethylsilyl |
| TEA or Et3N | triethylamine |
| TFA | trifluoroacetic acid |
| THF | tetrahydrofuran |
| TLC | thin layer chromatography |
| UPLC | Ultra-Performance Liquid Chromatography |
| Xphos | 2-Dicyclohexylphosphino-2′,4′,6′- |
| | triisopropylbiphenyl |
| xant-phos | 4,5-Bis(diphenylphosphino)-9,9-dimethylxanthene |

Compounds of Formulas (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described herein may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate the invention and various preferred embodiments.

### Intermediate 1: 1-(2-chloro-4-(4,4,5,5-tetramethyl-1,3,2-dioxaborolan-2-yl)phenyl)-3-methyl-1,3-dihydro-2H-imidazol-2-one (int-1)

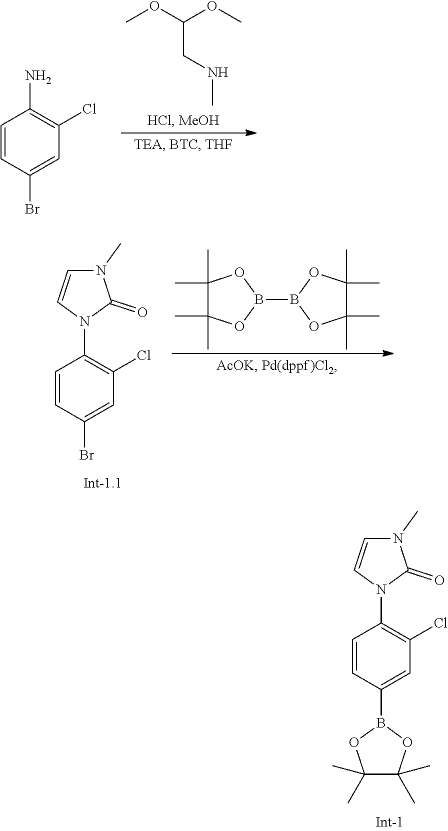

### Step a. Preparation of 1-(4-bromo-2-chlorophenyl)-3-methyl-1,3-dihydro-2H-imidazol-2-one (int-1.1)

To a solution of 4-bromo-2-chloroaniline (500 g, 2.4 mol) in THF (5.5 L) was added TEA (2 L) at −60° C. and added BTC (320 g 1.08 mol in 0.5 L THF) at −40° C. dropwise. The reaction mixture was stirred at −40° C. for 30 min. Then 2,2-dimethoxy-N-methylethan-1-amine (350 g, 2.9 mol) was added at −40° C. The mixture was stirred at −40° C. for 30 min. The reaction mixture was added to ice-H₂O (20 L) and extracted with EA (10 L). The organic layer was washed with H₂O and brine, dried over Na₂SO₄, filtered and concentrated. The residue was added to MeOH (1 L) and added HCl (12 M, 1.5 L) at 35-40° C. The mixture was stirred at rt for 30 min and then poured into ice-H₂O (8 L) and stirred for 10 min. The mixture was filtered. The filtered cake was dried to give the title product (600 g, 91% yield) as a white solid. LCMS: 286.9 (M+H)⁺.

### Step b. Preparation of 1-(2-chloro-4-(4,4,5,5-tetramethyl-1,3,2-dioxaborolan-2-yl)phenyl)-3-methyl-1,3-dihydro-2H-imidazol-2-one (int-1)

A mixture of 1-(4-bromo-2-chlorophenyl)-3-methyl-1,3-dihydro-2H-imidazol-2-one (900 g, 3.15 mol), 4,4,4′,4′,5,5,5′,5′-octamethyl-2,2′-bi(1,3,2-dioxaborolane) (900 g, 3.78 mol), Pd(dppf)Cl₂ (69 g, 0.09 mol) and KOAc (1080 g, 11.01 mol) in dioxane (12 L) was stirred at 100° C. for 1.5 h under N₂. The reaction mixture was cooled to rt and filtered. The filtrate was concentrated and purified by FCC eluting with EA. To the solution was added activated carbon (200 g) and stirred at r.t for 10 min. The mixture was filtered. The filtrate was concentrated; the residue was triturated with Et₂O (2 L) and stirred for 10 min. The solution was filtered, and the cake was dried to give the title product (560 g, 53% yield) as an off white solid. ¹H NMR (400 MHz, CDCl₃): δ 7.92 (d, J=0.8 Hz, 1H), 7.76-7.73 (m, 1H), 7.48 (d, J=8.0 Hz, 1H), 6.43 (d, J=3.2 Hz, 1H), 6.32 (d, J=2.8 Hz, 1H), 3.33 (s, 3H), 1.35 (s, 12H). LCMS: 335.2 (M+H)⁺.

### Intermediate 2: 1-(2-fluoro-4-(4,4,5,5-tetramethyl-1,3,2-dioxaborolan-2-yl)phenyl)-3-methyl-1,3-dihydro-2H-imidazol-2-one (int-2)

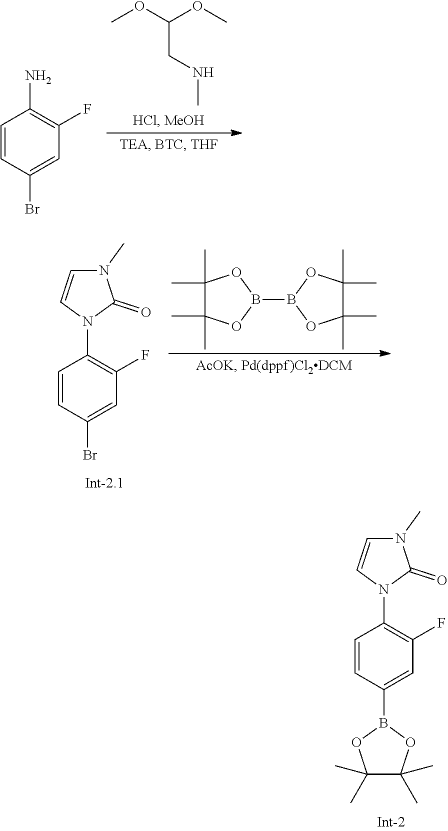

### Step a. Preparation of 1-(4-bromo-2-fluorophenyl)-3-methyl-1,3-dihydro-2H-imidazol-2-one (int-2.1)

To a solution of 4-bromo-2-fluoroaniline (1.00 g, 5.26 mmol) in THF (10 mL) at −60° C., triethylamine (0.80 mL, 5.74 mmol) was added and then triphosgene (700 mg, 2.37 mmol) was added to the reaction mixture at −50° C. The stirring was continued for −30 min at −50° C. and then 2,2-dimethoxy-N-methyl-ethanamine (1.50 g, 12.6 mmol) in THF (10 mL) was added at −50° C. The mixture was stirred at the same temperature for −30 min. The white suspension was quenched with ice cold water and extracted with ethyl acetate. The organic layer was washed with H₂O and brine, dried over Na₂SO₄, filtered, and concentrated to get the crude. The obtained crude was dissolved in methanol (6 mL) and then conc. hydrochloric acid (1.5 mL, 16.5 mmol) was added at room temperature. The resulting mixture was stirred for 30 min. Ice cold water was added to precipitated white solid, filtered, washed with cold water and then dried to get the title product (1 g, Yield 70%) as white solid. ¹H-NMR (400 MHz, DMSO-d₆): δ 7.58 (t, J=8.40 Hz, 1H), 7.40-7.36 (m, 2H), 6.50 (t, J=2.80 Hz, 1H), 6.35 (d, J=3.20 Hz, 1H), 3.35 (s, 3H). LCMS (ESI, +ve mode): Expected m/z for C₁₀H₈BrFN₂O [M+H] 270.99, 272.99 found 273.0 (M+H).

### Step b. Preparation of 1-(2-fluoro-4-(4,4,5,5-tetramethyl-1,3,2-dioxaborolan-2-yl)phenyl)-3-methyl-1,3-dihydro-2H-imidazol-2-one (int-2)

To a stirred solution of 1-(4-bromo-2-fluorophenyl)-3-methyl-1,3-dihydro-2H-imidazol-2-one (1.00 g, 3.69 mmol) in 1,4 dioxane (10 mL) at room temperature, bis(pinacolato)diboron (1.12 g, 4.41 mmol) was added followed by the addition of potassium acetate (0.724 mg, 7.38 mmol). The resulting mixture was degassed with Nitrogen for −10 mins and Pd(dppf)Cl₂·DCM (150 mg, 0.184 mmol) was added and then refluxed under nitrogen at 100° C. for 16 h. The reaction mixture was quenched with water and extracted with ethyl acetate. The organic layer was washed with brine, dried over anhyd. sodium sulphate and concentrated under reduced pressure to get crude as brown liquid. The crude obtained was purified by column chromatography (Silica gel, 100-200 mesh; elutent, 20% ethyl acetate in petroleum ether to get the title product (450 mg, Yield 28.6%) as white solid. ¹H-NMR (400 MHz, DMSO-d₆): δ 7.73 (t, J=7.60 Hz, 1H), 7.65 (dd, J=1.60, 7.80 Hz, 1H), 7.62 (dd, J=1.20, 11.40 Hz, 1H), 6.56 (t, J=3.20 Hz, 1H), 6.34 (d, J=3.20 Hz, 1H), 3.35 (s, 3H), 1.37 (s, 12H). LCMS (ESI, +ve mode): Expected m/z for C₁₆H₂₀BFN₂O₃[M+H]319.16 found 319.2 (M+H).

### Intermediate 3: 1-methyl-3-(2-methyl-4-(4,4,5,5-tetramethyl-1,3,2-dioxaborolan-2-yl)phenyl)-1,3-dihydro-2H-imidazol-2-one (int-3)

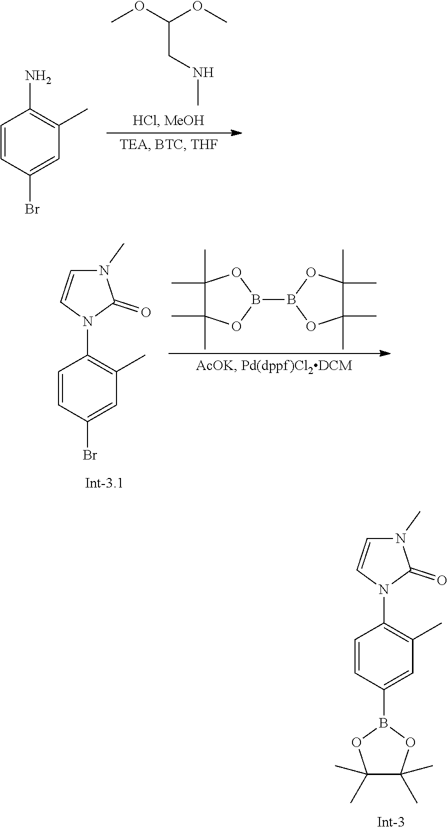

### Step a. Preparation of 1-(4-bromo-2-methylphenyl)-3-methyl-1,3-dihydro-2H-imidazol-2-one (int-3.1)

To a stirred solution of 4-bromo-2-methyl-aniline (1.00 eq, 3.00 g, 16.1 mmol) in THF (40 mL) at −78° C. were added triethylamine (5.34 eq, 12 mL, 86.1 mmol) and a solution of triphosgene (0.439 eq, 2.10 g, 7.08 mmol) in THF (15 mL). The resulting reaction mixture was stirred for 35 min at −78° C. and then 2,2-dimethoxy-N-methyl-ethanamine (1.20 eq, 2.30 g, 19.3 mmol) was added and stirred for 1 h. The progress of the reaction was monitored by TLC (50% EtOAc in pet ether). The reaction mixture was quenched with water (100 mL) and extracted with ethyl acetate (100 mL×2). The organic layer was washed with brine, dried over anhyd. sodium sulphate and concentrated under reduced pressure to get crude as brown liquid. The crude intermediate was dissolved in methanol (10 mL) and conc. HCl solution (12.2 eq, 6.0 mL, 197 mmol) was added and stirred for 30 min. The reaction was quenched with ice cold water (100 mL) and the separated solid was filtered and dried to get the title product (3.00 g, 11.1 mmol, 68.81% yield) as a brown solid. ¹H NMR (400 MHz, CDCl₃): δ 7.60 (d, J=2.40 Hz, 1H), 7.49-7.46 (m, 1H), 7.17 (d, J=11.20 Hz, 1H), 6.70 (d, J=4.00 Hz, 1H), 6.64 (d, J=4.00 Hz, 1H), 3.19 (s, 3H), 2.16 (s, 3H). LCMS (ESI): m/z calcd. For C₁₁H₁₁BrN₂O [M+H]⁺ 267.01, found 267.0 [M+H]⁺

### Step b. Preparation of 1-methyl-3-(2-methyl-4-(4,4,5,5-tetramethyl-1,3,2-dioxaborolan-2-yl)phenyl)-1,3-dihydro-2H-imidazol-2-one (int-3)

To a stirred solution of 1-(4-bromo-2-methylphenyl)-3-methyl-1,3-dihydro-2H-imidazol-2-one (1.00 eq, 3.00 g, 11.2 mmol) in 1,4-dioxane (35 mL) at room temperature were added bis(pinacolato)diboron (1.19 eq, 3.40 g, 13.4 mmol) and potassium acetate (2.98 eq, 3.29 g, 33.5 mmol). The resulting mixture was degassed with nitrogen for 10-15 min and then Pd(dppf)Cl₂·DCM (0.0997 eq, 914 mg, 1.12 mmol) was added. The reaction was heated under nitrogen atmosphere at 80° C. for 16 h. The progress of the reaction was monitored by TLC (50% EtOAc in pet ether). The reaction mixture was quenched with water (50 mL) and extracted with ethyl acetate (50 mL×3). The organic layer was washed with brine, dried over anhyd. sodium sulphate and concentrated under reduced pressure. The crude compound was purified by flash silica gel column chromatography (45% EtOAc in pet ether) to get the title product (1.50 g, 4.77 mmol, 42.51% yield) as a brown solid. ¹H-NMR (400 MHz, CDCl₃): δ 7.77 (s, 1H), 7.71 (d, J=7.60 Hz, 1H), 7.25 (d, J=7.60 Hz, 1H), 6.34 (d, J=3.20 Hz, 1H), 6.31 (d, J=2.80 Hz, 1H), 3.35 (s, 3H), 2.29 (s, 3H), 1.37 (s, 12H).

### Intermediate 4: 1-(2-chloro-4-(4,4,5,5-tetramethyl-1,3,2-dioxaborolan-2-yl)phenyl)-3-(methyl-d₃)-1,3-dihydro-2H-imidazol-2-one (int-4)

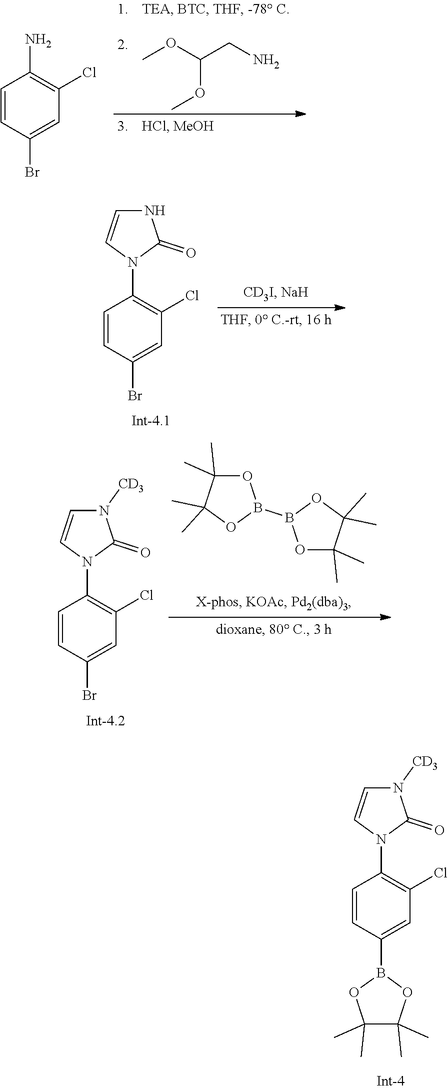

### Step a. Preparation of 1-(4-bromo-2-chlorophenyl)-1,3-dihydro-2H-imidazol-2-one (int-4.1)

To a stirred solution of 4-bromo-2-chloro-aniline (1.00 eq, 5.00 g, 24.2 mmol) in THF (150 mL) at −78° C. was added triethylamine (7.00 eq, 24 mL, 170 mmol) followed by a solution of triphosgene (0.501 eq, 3.60 g, 12.1 mmol) in THF (10 mL) dropwise. The resulting reaction mixture was stirred for 40 min at −78° C. and then 2,2-dimethoxyethanamine (1.18 eq, 3.00 g, 28.5 mmol) was added. The reaction was stirred at −60° C. for 1 h. The progress of the reaction was monitored by TLC (30% EtOAc in pet ether). The reaction mixture was quenched with water (200 mL) and extracted with ethyl acetate (200 mL×2). The organic layer was washed with brine, dried over anhyd. sodium sulfate and concentrated under reduced pressure to obtain residue as a white solid. The residue was dissolved in THF (60 mL) and conc·HCl (12M) (1.00 eq, 20 mL, 24.2 mmol) was added at room temperature. The reaction mixture was stirred for 30 min at 50° C. and then quenched with ice cold water (100 mL). The precipitate was filtered and dried to get the title product (4.65 g, 17.0 mmol, 70.20% yield) as a white solid. ¹H-NMR (400 MHz, DMSO-d6): δ 10.25 (s, 1H), 7.92 (d, J=2.00 Hz, 1H), 7.66 (dd, J=2.00, 8.40 Hz, 1H), 7.43 (d, J=8.40 Hz, 1H), 6.62-6.60 (m, 1H), 6.57-6.56 (m, 1H) ppm. LCMS (ESI): m/z calcd. For C₉H₆BrClN₂O [M+H]⁺ 272.94, found 273.0, 275.0 [M+H]⁺

### Step b. Preparation of 1-(4-bromo-2-chlorophenyl)-3-(methyl-d₃)-1,3-dihydro-2H-imidazol-2-one (int-4.2)

To a solution of 1-(4-bromo-2-chlorophenyl)-1,3-dihydro-2H-imidazol-2-one (1.00 eq, 2.00 g, 7.31 mmol) in THF (20 mL) at 0° C. was added NaH (60%, 1.47 eq, 0.43 g, 10.8 mmol) and the resulting mixture was stirred at 0° C. for 30 min. Then methyl-D3 iodide (1.20 eq, 1.27 g, 8.76 mmol) was added and the resulting mixture was stirred at room temperature for 16 h. The reaction mixture was quenched with saturated ammonium chloride solution and (20 mL) extracted with ethyl acetate (50 mL×2). The organic layer was washed with brine, dried over anhydrous sodium sulfate, and concentrated under reduced pressure to get crude as brown liquid. The crude compound was purified by flash silica gel column chromatography (60-80% EtOAc in pet ether) to get the title product (2.00 g, 6.88 mmol, 94.13% yield) as a white solid. LCMS (ESI): m/z calcd. For C₁₀H₅D₃BrClN₂O [M+H]⁺ 289.97, found 290.0 [M+H]⁺.

### Step c. Preparation of 1-(2-chloro-4-(4,4,5,5-tetramethyl-1,3,2-dioxaborolan-2-yl)phenyl)-3-(methyl-d₃)-1,3-dihydro-2H-imidazol-2-one (int-4)

To a stirred solution of 1-(4-bromo-2-chlorophenyl)-3-(methyl-d₃)-1,3-dihydro-2H-imidazol-2-one (1.00 eq, 1.00 g, 3.44 mmol) in 1,4-dioxane (10 mL) were added KOAc (2.96 eq, 1.00 g, 10.2 mmol) and bis(pinacolato)diboron (1.14 eq, 1.00 g, 3.94 mmol) at room temperature under nitrogen atmosphere. The resulting mixture was degassed with nitrogen for ˜10 min and then and PdCl₂(dppf)·DCM (0.1000 eq, 281 mg, 0.344 mmol) was added. The reaction mixture stirred at 80° C. for 3 h and the progress of the reaction was monitored by LCMS. The reaction was diluted with 1,4-dioxane and filtered through celite. The filtrate was concentrated to obtain crude compound which was purified by flash silica gel column chromatography (0-10% DCM in MeOH) to obtain mixture of the title product (850 mg, 2.52 mmol, 73.15% yield) and corresponding boronic acid as a dark liquid. LCMS (ESI): m/z calcd. For C₁₆H₁₇D₃BClN₂O₃[M+H]⁺ 338.14, found 256.1 [M+H]⁺, which matched boronic acid: m/z calcd. For C₁₀H₇D₃BClN₂O₃ [M+H]⁺ 256.1.

### Intermediate 5: 1-(3′-bromo-3-chloro-5′-fluoro-2′-hydroxy-[1,1′-biphenyl]-4-yl)-3-methyl-1,3-dihydro-2H-imidazol-2-one (int-5)

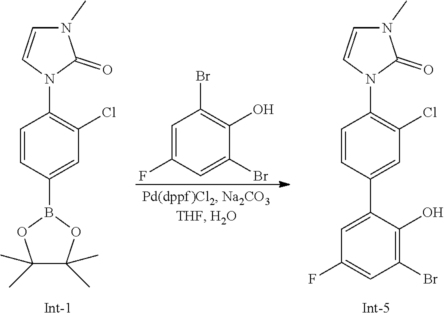 [SMILES: Cn1ccn(-c2ccc(B3OC(C)(C)C(C)(C)O3)cc2Cl)c1=O ; Oc1c(Br)cc(F)cc1Br ; Cn1ccn(-c2ccc(-c3cc(F)cc(Br)c3O)cc2Cl)c1=O]

### Preparation of 1-(3′-bromo-3-chloro-5′-fluoro-2′-hydroxy-[1,1′-biphenyl]-4-yl)-3-methyl-1,3-dihydro-2H-imidazol-2-one (int-5)

A mixture of 1-(2-chloro-4-(4,4,5,5-tetramethyl-1,3,2-dioxaborolan-2-yl)phenyl)-3-methyl-1,3-dihydro-2H-imidazol-2-one (Intermediate 1, 275 g, 0.82 mol), 2,6-dibromo-4-fluorophenol (440 g, 1.64 mol), Pd(dppf)Cl₂ (30 g, 0.04 mol) and Na₂CO₃ (174 g, 1.64 mol) in dioxane/H₂O (6 L/0.3 L) was stirred at 75° C. for 4 h under N₂. The reaction mixture was cooled to rt and filtered. To the filtrate was added EA (10 L); the resulted solution was washed with H₂O and brine, dried over Na₂SO₄, decolorization by activated carbon (50 g); after filtered and concentrated, to the residue was added DCM:MeOH 20:1 (2 L) and activated carbon (20 g). The mixture was stirred at 10 min and filtered. The filtrate was concentrated, triturated with ACN (1.5 L), after filtered and dried to give the title product (92 g, 28% yield) as a white solid. ¹H NMR (400 MHz, DMSO-d₆): δ 9.34 (s, 1H), 7.77 (d, J=2.0 Hz, 1H), 7.59-7.51 (m, 3H), 7.29-7.26 (m, 1H), 6.72-6.69 (m, 2H), 3.21 (s, 3H). LCMS: 396.9 (M+H)⁺.

### Intermediate 6: 1-(4-(2-bromo-3-hydroxy-6-methylpyridin-4-yl)-2-chlorophenyl)-3-methyl-1,3-dihydro-2H-imidazol-2-one (int-6)

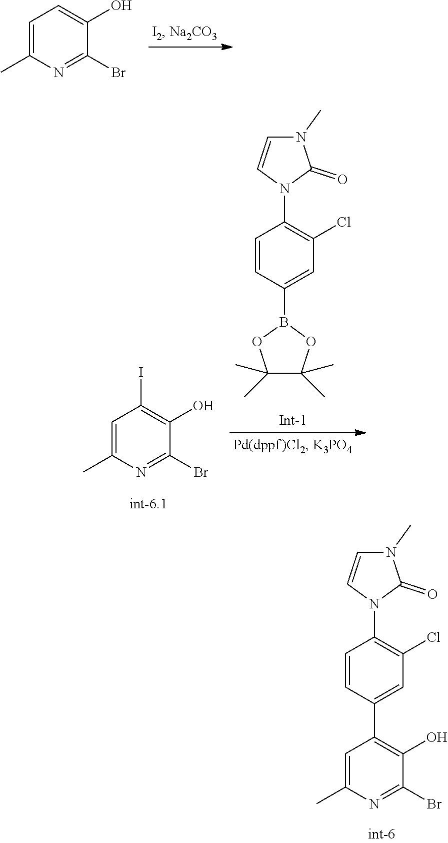

### Step a. Preparation of 2-bromo-4-iodo-6-methylpyridin-3-ol (int-6.1)

A mixture of 2-bromo-6-methylpyridin-3-ol (5.0 g, 26.6 mmol), 12 (10.0 g, 40.0 mmol) and Na₂CO₃ (8.5 g, 80.0 mmol) in water (150 mL) was stirred at 50° C. for 8 hours. After the reaction mixture was cooled to room temperature, the reaction mixture was adjusted “pH” 3-4 with 3 N HCl and extracted with EA. The combined organic layer was concentrated in vacuo. The residue was purification by FCC (EA/PE=1/3) to afford 2-bromo-4-iodo-6-methylpyridin-3-ol (5.5 g, 68% yield). LCMS: 313 (M+H)⁺.

### Step b. Preparation of 1-(4-(2-bromo-3-hydroxy-6-methylpyridin-4-yl)-2-chlorophenyl)-3-methyl-1,3-dihydro-2H-imidazol-2-one (int-6)

To a solution of 2-bromo-4-iodo-6-methylpyridin-3-ol (1.0 g, 2.5 mmol) in 1,4-dioxane/H₂O (30 mL/5 mL) was added 1-(2-chloro-4-(4,4,5,5-tetramethyl-1,3,2-dioxaborolan-2-yl)phenyl)-3-methyl-1,3-dihydro-2H-imidazol-2-one (Intermediate 1, 1.3 g, 3.1 mmol, 80% purity), Pd(dppf)Cl₂ (150 mg, 0.2 mmol) and K₃PO₄ (1.6 g, 7.5 mmol). The reaction mixture was stirred at 60° C. for 2 hours under N₂. The reaction mixture was cooled, added water, and extracted with EA. The combined organic layer was concentrated. The residue was purified by FCC (DCM/MeOH=20/1) to obtain the title product (0.4 g, 29.5% yield). LCMS: 394 (M+H)⁺.

### Intermediate 7: 1-(4-(2-bromo-3-hydroxy-6-methylpyridin-4-yl)-2-fluorophenyl)-3-methyl-1,3-dihydro-2H-imidazol-2-one (int-7)

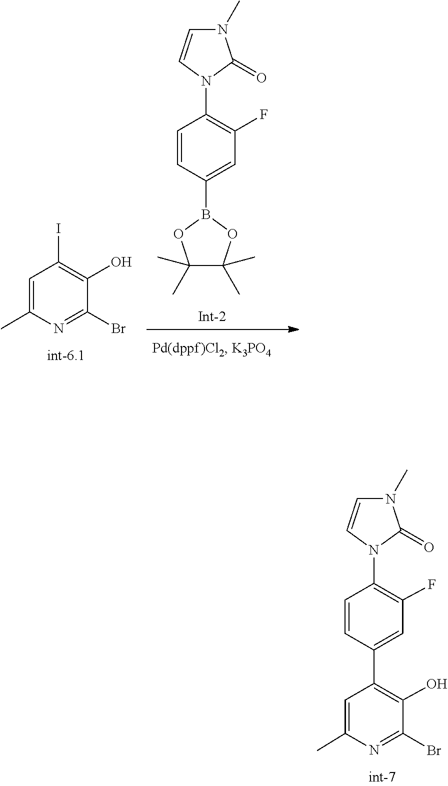

### Preparation of 1-(4-(2-bromo-3-hydroxy-6-methylpyridin-4-yl)-2-fluorophenyl)-3-methyl-1,3-dihydro-2H-imidazol-2-one (int-7)

To a solution of 2-bromo-4-iodo-6-methylpyridin-3-ol (int-6.1, 1.5 g, 4.8 mmol) in 1,4-dioxane/H₂O was added 1-(2-fluoro-4-(4,4,5,5-tetramethyl-1,3,2-dioxaborolan-2-yl)phenyl)-3-methyl-1,3-dihydro-2H-imidazol-2-one (Intermediate 2, 1.5 g, 4.8 mmol), Pd(dppf)Cl₂ (0.35 g, 0.05 mmol) and K₃PO₄ (3.0 g, 15.0 mmol). The reaction mixture was stirred at 60° C. for 3 hours under N₂. The reaction mixture was cooled to rt, added water, and extracted with EA. The combined organic layers were concentrated. The residue was purified by FCC (DCM/MeOH=20/1) to afford the title product (0.7 g, 35% yield). ¹H-NMR (400 MHz, DMSO-d6): δ 9.61 (s, 1H), 7.67-7.61 (m, 2H), 7.53-7.50 (m, 1H), 7.27 (s, 1H), 6.80-6.79 (m, 1H), 6.76-6.73 (m, 1H), 3.22 (s, 3H), 2.41 (s, 3H). LCMS (ESI): m/z calcd. For C₁₆H₁₃BrFN₃O₂ [M+H]⁺ 378.02, found 378.2 [M+H]⁺.

### Intermediate 8: 1-(4-(3-amino-2-chloro-6-methylpyridin-4-yl)-2-chlorophenyl)-3-methyl-1,3-dihydro-2H-imidazol-2-one (int-8)

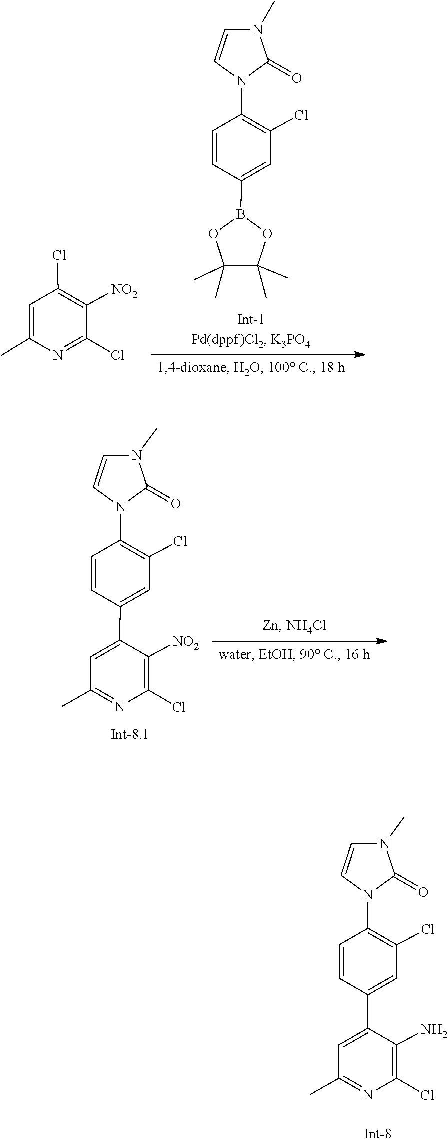 [SMILES: Cc1cc(Cl)c([N+](=O)[O-])c(Cl)n1 ; Cn1ccn(-c2ccc(B3OC(C)(C)C(C)(C)O3)cc2Cl)c1=O ; Cc1cc(-c2ccc(-n3ccn(C)c3=O)c(Cl)c2)c([N+](=O)[O-])c(Cl)n1 ; Cc1cc(-c2ccc(-n3ccn(C)c3=O)c(Cl)c2)c(N)c(Cl)n1]

### Step a. Preparation of 1-(2-chloro-4-(2-chloro-6-methyl-3-nitropyridin-4-yl)phenyl)-3-methyl-1,3-dihydro-2H-imidazol-2-one (int-8.1)

To a solution of 2,4-dichloro-6-methyl-3-nitro-pyridine (1, 1.00 eq, 3.00 g, 14.5 mmol) in 1,4-dioxane (15 mL) at room temperature were added 1-(2-chloro-4-(4,4,5,5-tetramethyl-1,3,2-dioxaborolan-2-yl)phenyl)-3-methyl-1,3-dihydro-2H-imidazol-2-one (Intermediate 1, 1.30 eq, 6.30 g, 18.8 mmol), and a solution of K₃PO₄ (2.02 eq, 6.20 g, 29.2 mmol) in water (2 mL) sequentially. This reaction mixture degassed for 10 min and Pd(dppf)Cl₂·DCM (0.0500 eq, 592 mg, 0.725 mmol) was added. The reaction mixture was heated at 100° C. for 18 h. The progress of the reaction was monitored by TLC (10% methanol in DCM) and LCMS. The reaction mixture was concentrated under reduced pressure and the residue was suspended in EtOAc (250 mL), washed with water, brine, dried over anhyd. sodium sulfate and concentrated under reduced pressure to get crude as brown solid. The crude compound was purified by flash silica gel column chromatography (10-20% MeOH in DCM) to obtain the title product (3.00 g, 7.74 mmol, 53.38% yield) as a brown solid. LCMS (ESI): m/z calcd. For C₁₆H₁₂Cl₂N₄O₃ [M+H]⁺ 379.03, found 379.0 [M+H]⁺

### Step b. Preparation of 1-(4-(3-amino-2-chloro-6-methylpyridin-4-yl)-2-chlorophenyl)-3-methyl-1,3-dihydro-2H-imidazol-2-one (int-8)

To a solution of 1-(2-chloro-4-(2-chloro-6-methyl-3-nitropyridin-4-yl)phenyl)-3-methyl-1,3-dihydro-2H-imidazol-2-one (1.00 eq, 3.00 g, 7.91 mmol) in ethanol (10 mL) at 0° C., a solution of ammonium chloride (5.20 eq, 2.20 g, 41.1 mmol) in water (10 mL) and Zn-dust (5.03 eq, 2.60 g, 39.8 mmol) were added. This reaction mixture was stirred at 90° C. for 16 h. The progress of the reaction was monitored by TLC (10% methanol in DCM) and LCMS. The reaction mixture was filtered and washed with DCM and EtOAc. The filtrate was concentrated, and the residue was dissolved in EtOAc (150 mL), washed with water, brine, dried over anhyd. sodium sulfate and concentrated under reduced pressure. The crude compound was purified by flash silica gel column chromatography (10-20% MeOH in DCM) to obtain the title product (1.50 g, 3.87 mmol, 48.86% yield) as a white solid. ¹H-NMR (400 MHz, DMSO-d₆): δ 7.71 (d, J=1.60 Hz, 1H), 7.59-7.53 (m, 2H), 7.01 (s, 1H), 6.74 (d, J=3.20 Hz, 1H), 6.67 (d, J=2.80 Hz, 1H), 5.05 (s, 2H), 3.22 (s, 3H), 2.33 (s, 3H). LCMS (ESI): m/z calcd. For C₁₆H₁₄Cl₂N₄O [M+H]⁺ 349.05, found 349.0 [M+H]⁺.

### Intermediate 9: 1-(4-(2-bromo-3-(methoxymethoxy)-6-methylpyridin-4-yl)-2-methylphenyl)-3-methyl-1,3-dihydro-2H-imidazol-2-one (int-9)

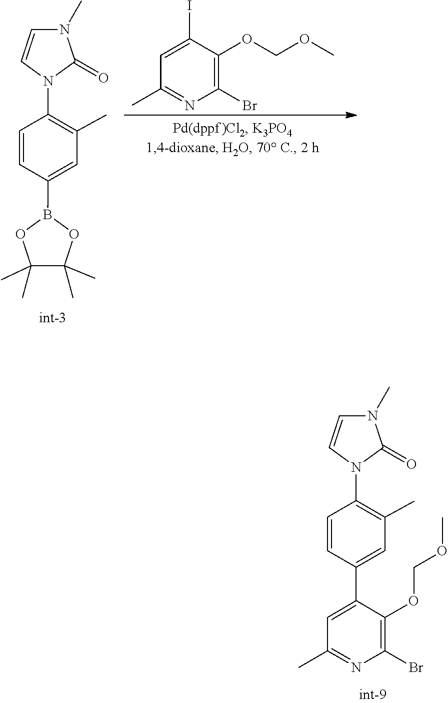

### Preparation of 1-(4-(2-bromo-3-(methoxymethoxy)-6-methylpyridin-4-yl)-2-methylphenyl)-3-methyl-1,3-dihydro-2H-imidazol-2-one (int-9)

To a stirred solution of 2-bromo-4-iodo-3-(methoxymethoxy)-6-methyl-pyridine (1.00 eq, 600 mg, 1.68 mmol) in 1,4-dioxane (10 mL) at room temperature were added 1-methyl-3-(2-methyl-4-(4,4,5,5-tetramethyl-1,3,2-dioxaborolan-2-yl)phenyl)-1,3-dihydro-2H-imidazol-2-one (Intermediate 3, 1.10 eq, 579 mg, 1.84 mmol) and a solution of potassium phosphate tribasic (3.00 eq, 1066 mg, 5.03 mmol) in water (2 mL). The resulting reaction mixture was degassed with nitrogen for 10-15 min and then Pd(dppf)Cl₂·DCM (0.0994 eq, 136 mg, 0.167 mmol) was added. The reaction was heated under nitrogen at 70° C. for 2 h. The reaction progress was monitored by TLC (50% EtOAc in pet ether) and LCMS. The reaction mixture was quenched with water (10 mL) and extracted with ethyl acetate (10 mL×3). The organic layer was washed with brine, dried over anhyd. sodium sulfate and concentrated under reduced pressure to get crude as brown liquid. The crude compound was purified by flash silica gel column chromatography (46% EtOAc in pet ether) to get the title product (400 mg, 0.871 mmol, 51.98% yield) as a brown solid. LCMS (ESI): m/z calcd. For C₁₉H₂₀BrN₃O₃ [M+H]⁺ 418.07, found 418.0, 420.0 [M+H]⁺.

### Intermediate 10: 1-(4-(2-bromo-3-(methoxymethoxy)-6-methylpyridin-4-yl)-2-chlorophenyl)-3-(methyl-d₃)-1,3-dihydro-2H-imidazol-2-one (int-10)

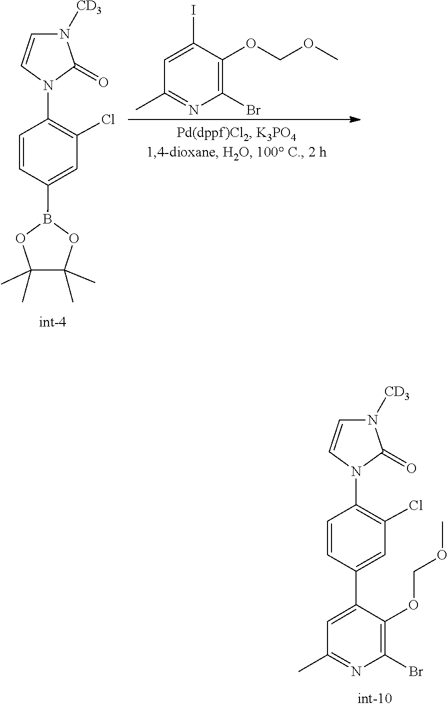

### Preparation of 1-(4-(2-bromo-3-(methoxymethoxy)-6-methylpyridin-4-yl)-2-chlorophenyl)-3-(methyl-d₃)-1,3-dihydro-2H-imidazol-2-one (int-10)

To a stirred solution of 2-bromo-4-iodo-3-(methoxymethoxy)-6-methyl-pyridine (0.899 eq, 664 mg, 1.85 mmol) in 1,4-dioxane (10 mL) and water (2 mL) were added 1-(2-chloro-4-(4,4,5,5-tetramethyl-1,3,2-dioxaborolan-2-yl)phenyl)-3-(methyl-d₃)-1,3-dihydro-2H-imidazol-2-one (Intermediate 4, 1.00 eq, 850 mg, 2.06 mmol) and a solution of K₃PO₄ (3.00 eq, 1313 mg, 6.19 mmol) in water (1 mL). The resulting mixture was degassed with nitrogen for ˜10 min and then PdCl₂(dppf)·DCM (0.0997 eq, 168 mg, 0.206 mmol) was added. The reaction mixture was heated under nitrogen at 100° C. for 2 h. The progress of the reaction was monitored by LCMS. The reaction mixture was quenched with water (25 mL) and extracted with ethyl acetate (25 mL×3). The organic layer was washed with brine, dried over anhydrous sodium sulfate, and concentrated under reduced pressure to get crude as brown gum. The crude compound was purified by flash silica gel column chromatography (60-80% EtOAc in pet ether) to get the title product (430 mg, 0.973 mmol, 47.19% yield) as a brown solid. LCMS (ESI): m/z calcd. For C₁₈H₁₄D₃BrClN₃O₃[M+H]⁺ 441.03, found 441.0 [M+H]⁺.

### Intermediate 11: 1-(4-(2-bromo-3-(methoxymethoxy)-6-methylpyridin-4-yl)-2-chlorophenyl)-3-methyl-1,3-dihydro-2H-imidazol-2-one (int-11)

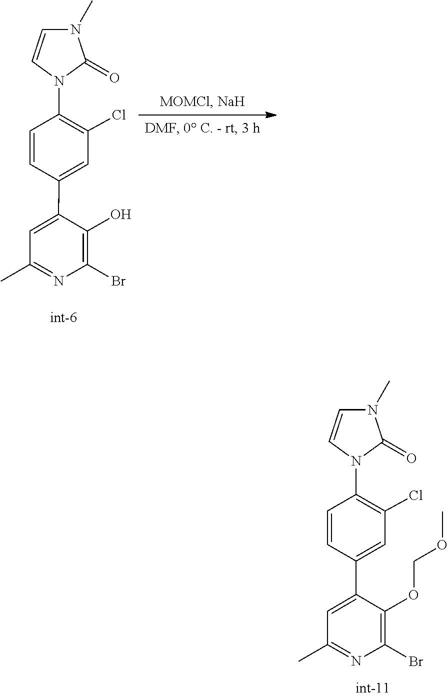

### Preparation of 1-(4-(2-bromo-3-(methoxymethoxy)-6-methylpyridin-4-yl)-2-chlorophenyl)-3-methyl-1,3-dihydro-2H-imidazol-2-one (int-11)

To a stirred solution of 1-(4-(2-bromo-3-hydroxy-6-methylpyridin-4-yl)-2-chlorophenyl)-3-methyl-1,3-dihydro-2H-imidazol-2-one (Intermediate 6, 1.00 eq, 0.45 g, 1.14 mmol) in DMF (8 mL) at 0° C., NaH (1.50 eq, 41 mg, 1.71 mmol) was added and stirred for 30 min. Then MOM chloride (1.30 eq, 0.11 mL, 1.48 mmol) was added and the resulting mixture stirred at room temperature for 3 h. The progress of the reaction was monitored by TLC (80% EtOAc in pet ether) and LCMS. The reaction mixture was quenched in ice cold water (5 mL) and the separated solid was filtered, washed with water, and dried to obtain the title product (350 mg, 82% purity) as an off while solid. LCMS (ESI): m/z calcd. For C₁₈H₁₇BrClN₃O₃[M+H]⁺ 438.01, found 438.0 [M+H]⁺

### Intermediate 12: 1-(tert-butyl)-4-(3-(4,4,5,5-tetramethyl-1,3,2-dioxaborolan-2-yl)phenyl)piperazine (int-12)

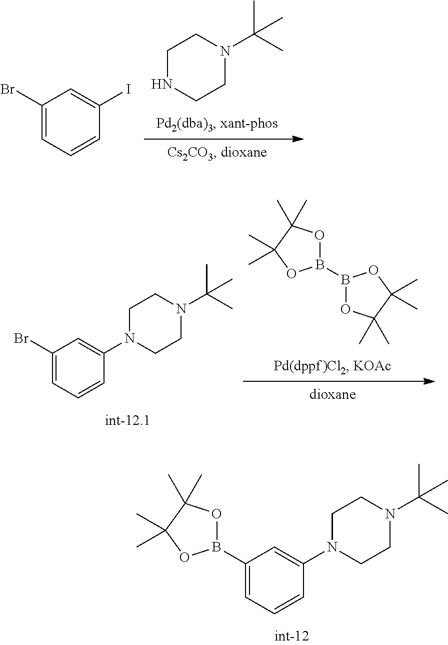

### Step a. Preparation of 1-(3-bromophenyl)-4-(tert-butyl)piperazine (int-12.1)

To a solution of 1-(tert-butyl)piperazine (20.0 g, 140.0 mmol) in 1,4-dioxane (500 mL) was added 1-bromo-3-iodobenzene (100.0 g, 350 mmol), Pd₂(dba)₃ (6.4 g, 7.0 mmol), xant-phos (8.1 g, 14.0 mmol) and Cs₂CO₃ (137.0 g, 420.0 mmol). The reaction mixture was stirred at 85° C. for 16 hours under N₂. The reaction mixture was cooled, added water (300 mL) and extracted with EA (200 mL*3). The combined organic layer was concentrated. The residue was purified by FCC (PE/EA=1/1) to obtain the title product (22.8 g, 36.3% yield). LCMS: 297 (M+H)⁺.

### Step b. Preparation of 1-(tert-butyl)-4-(3-(4,4,5,5-tetramethyl-1,3,2-dioxaborolan-2-yl)phenyl)piperazine (int-12)

To a solution of 1-(3-bromophenyl)-4-(tert-butyl)piperazine (22.8 g, 76.7 mmol) in 1,4-dioxane (400 mL) was added 4,4,4′,4′,5,5,5′,5′-octamethyl-2,2′-bi(1,3,2-dioxaborolane) (29.2 g, 115.0 mmol), Pd(dppf)Cl₂ (5.6 g, 7.7 mmol) and KOAc (22.5 g, 230.0 mmol). The reaction mixture was stirred at 90° C. for 16 hours under N₂. The reaction mixture was cooled and filtered. The filtrate was concentrated. The residue was purified by FCC (PE/EA=2/1-DCM/MeOH=20/1) to obtain the title product (13.0 g, 50% yield). LCMS: 345 (M+H)⁺.

### Intermediate 13: 1-(tert-butyl)-4-(3-fluoro-5-(4,4,5,5-tetramethyl-1,3,2-dioxaborolan-2-yl)phenyl)piperazine (int-13)

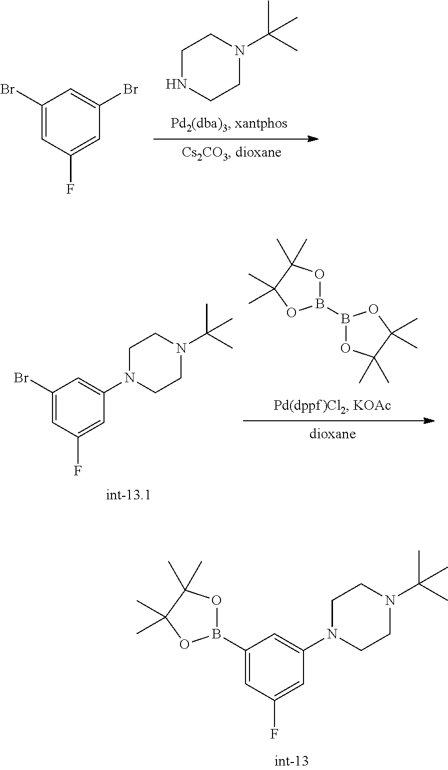

### Step a. Preparation of 1-(3-bromo-5-fluorophenyl)-4-(tert-butyl)piperazine (int-13.1)

A solution of 1,3-dibromo-5-fluorobenzene (600 mg, 2.36 mmol) and 1-(tert-butyl)piperazine (335 mg, 2.36 mmol), Pd₂(dba)₃ (173 mg, 0.19 mmol), xantphos (218 mg, 0.38 mmol) and Cs₂CO₃ (2.3 g mg, 7.084 mmol) in dioxane (30 mL) was stirred at 100° C. for 16 hours under N₂. After the reaction was complete by LCMS, the reaction mixture was cooled to room temperature, suspended in water and extracted with EA (30 mL×3). The combined organic layers were washed with water, brine, dried over Na₂SO₄, filtered and concentrated. The residue was purified by FCC to afford the title product (600 mg, 80.8% yield) as yellow oil. LCMS: 315.1 (M+H)⁺.

### Step b. Preparation of 1-(tert-butyl)-4-(3-fluoro-5-(4,4,5,5-tetramethyl-1,3,2-dioxaborolan-2-yl)phenyl)piperazine (int-13)

A solution of 1-(3-bromo-5-fluorophenyl)-4-(tert-butyl)piperazine (600 mg, 1.90 mmol) and bis(pinacolato)diboron (725 mg, 2.86 mmol), Pd(dppf)Cl₂ (140 mg, 0.19 mmol) and KOAc (560 mg, 5.70 mmol) in dioxane (20 mL) was stirred at 90° C. for 16 hours under N₂. After the reaction was complete by LCMS, the reaction mixture was cooled to room temperature, suspended in water and extracted with EA. The combined organic layers were washed with water, brine, dried over Na₂SO₄, filtered and concentrated. The residue was purified by FCC to afford the title product (430 mg, 62.5% yield) as yellow oil. LCMS: 363.3 (M+H)⁺.

### Intermediate 14: 1-(tert-butyl)-4-(3-chloro-5-(4,4,5,5-tetramethyl-1,3,2-dioxaborolan-2-yl)phenyl)piperazine (int-14)

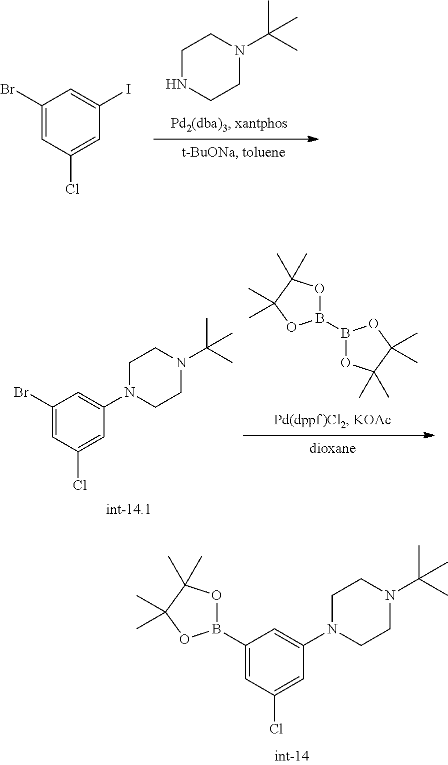

### Step a. Preparation of 1-(3-bromo-5-chlorophenyl)-4-(tert-butyl)piperazine (int-14.1)

To a solution of 1,3-dibromo-5-chlorobenzene (750 mg, 2.76 mmol) and 1-(tert-butyl)piperazine (263 mg, 1.85 mmol), in toluene (30 mL) was added Pd₂(dba)₃ (136 mg, 0.15 mmol), xantphos (171 mg, 0.30 mmol) and t-BuONa (533 mg, 5.55 mmol). The reaction mixture was stirred at 100° C. for 8 h under N₂. LCMS showed the reaction was complete. The reaction mixture was cooled to room temperature, suspended in H₂O, and extracted with EA. The combined organic layers were dried over Na₂SO₄ and concentrated. The residue was purified by silica gel column chromatography (DCM/MeOH=10/1) to afford the title compound (382 mg, 62% yield). LCMS: 331.1 (M+H)⁺.

### Step b. Preparation of 1-(tert-butyl)-4-(3-chloro-5-(4,4,5,5-tetramethyl-1,3,2-dioxaborolan-2-yl)phenyl)piperazine (int-14)

A solution of 1-(3-bromo-5-chlorophenyl)-4-(tert-butyl)piperazine (150 mg, 0.45 mmol), bis(pinacolato)diboron (138 mg, 0.54 mmol), Pd(dppf)Cl₂ (33 mg, 0.045 mmol) and KOAc (133 mg, 1.35 mmol) in 1,4-dioxane (6 mL) was stirred at 90° C. overnight under N₂. The reaction mixture was cooled to room temperature, and the solvent was removed under reduced pressure to afford the title compound (368 mg, crude). LCMS: 379.2 (M+H)⁺.

### Intermediate 15: 1-(tert-butyl)-4-(2-fluoro-5-(4,4,5,5-tetramethyl-1,3,2-dioxaborolan-2-yl)phenyl)piperazine (int-15)

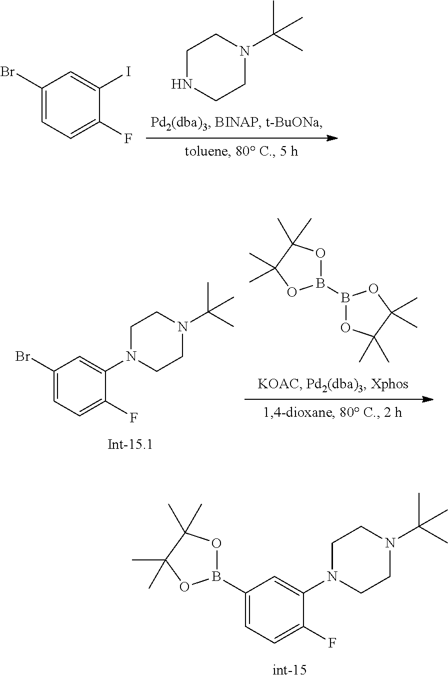

### Step a. Preparation of 1-(5-bromo-2-fluorophenyl)-4-(tert-butyl)piperazine (int-15.1)

To a stirred solution of 4-bromo-1-fluoro-2-iodo-benzene (1.00 eq, 2.00 g, 6.65 mmol) in toluene (50 mL) at room temperature, 1-tert-butylpiperazine (1.00 eq, 945 mg, 6.64 mmol) and sodium tert-butoxide (1.57 eq, 1.00 g, 10.4 mmol) were added. The resulting mixture was degassed with nitrogen for −10 min. Then BINAP (0.101 eq, 420 mg, 0.675 mmol) and Pd₂(dba)₃ (0.0509 eq, 310 mg, 0.339 mmol) were added. The resulting reaction mixture was heated 80° C. for 5 h. The progress of the reaction was monitored by TLC (50% EtOAc in pet ether). The reaction mixture was quenched with water (30 mL) and extracted with ethyl acetate (30 mL×2). The organic layer was washed with brine, dried over anhyd. sodium sulfate and concentrated under reduced pressure. The crude compound was purified by flash silica gel column chromatography (50-95% EtOAc in pet ether) to obtain the title product (800 mg, 2.54 mmol, 38.18% yield) as a pale brown gum. ¹H-NMR (400 MHz, DMSO-d6): δ 7.12-7.08 (m, 3H), 3.01 (t, J=4.80 Hz, 4H), 2.63 (t, J=4.40 Hz, 4H), 1.04 (s, 9H). LCMS (ESI): m/z calcd. For C₁₄H₂₀BrFN₂ [M+H]⁺ 315.0, found 315.1, 317.1 [M+H]⁺.

### Step b. Preparation of 1-(tert-butyl)-4-(2-fluoro-5-(4,4,5,5-tetramethyl-1,3,2-dioxaborolan-2-yl)phenyl)piperazine (int-15)

To a stirred solution of 1-(5-bromo-2-fluoro-phenyl)-4-tert-butyl-piperazine (1.00 eq, 300 mg, 0.952 mmol) in 1,4-dioxane (10 mL) at room temperature, bis(pinacolato)diboron (1.49 eq, 360 mg, 1.42 mmol) and potassium acetate (3.00 eq, 280 mg, 2.85 mmol) were added. The resulting mixture was degassed with nitrogen for −10 min and then dicyclohexyl[2′,4′,6′-tris(propan-2-yl)[1,1′-biphenyl]-2-yl]phosphane (0.110 eq, 50 mg, 0.105 mmol) and Pd₂(dba)₃ (0.103 eq, 90 mg, 0.0983 mmol) were added. The reaction mixture was heated at 80° C. for 2 h. The progress of the reaction was monitored by TLC (70% EtOAc in pet ether) and LCMS. The reaction mixture was filtered and concentrated under reduced pressure to get crude title product (340 mg) as pale brown gum. The crude product was taken for next step without any further purification. LCMS (ESI): m/z calcd. For C₂₀H₃₂BFN₂O₂[M+H]⁺ 363.25, found 363.2 [M+H]⁺.

### Intermediate 16: (3-(4-(tert-butyl)piperazin-1-yl)-4-chlorophenyl)boronic acid (int-16)

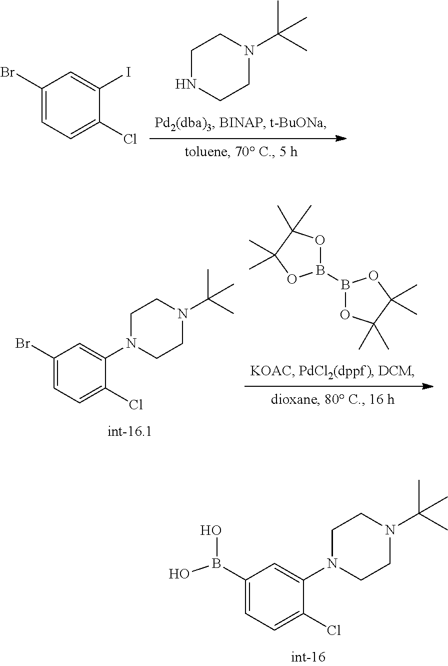

### Step a. Preparation of 1-(5-bromo-2-chlorophenyl)-4-(tert-butyl)piperazine (int-16.1)

To a stirred solution of 4-bromo-1-chloro-2-iodo-benzene (1.00 eq, 2.00 g, 6.30 mmol) in toluene (40 mL) at room temperature, 1-tert-butylpiperazine (1.00 eq, 896 mg, 6.30 mmol) and NaOtBu (1.50 eq, 908 mg, 9.45 mmol) were added. The resulting mixture was degassed with nitrogen gas for −10 min and then Pd₂(dba)₃ (0.0500 eq, 288 mg, 0.315 mmol) and BINAP (0.100 eq, 392 mg, 0.630 mmol) were added. The reaction mixture was refluxed under nitrogen at 70° C. for 5 h. The progress of the reaction was monitored by TLC (50% EtOAc in pet ether). The reaction mixture was concentrated under reduced pressure. The crude product was purified by flash silica gel column chromatography (5-70% EtOAc in pet ether) to get the title product (700 mg, 1.73 mmol, 27.38% yield) as a brown oil. LCMS (ESI): m/z calcd. For C₁₄H₂₀BrClN₂ [M+H]⁺ 331.05, found 331.1 [M+H]⁺.

### Step b. (3-(4-(tert-butyl)piperazin-1-yl)-4-chlorophenyl)boronic acid (int-16)

To a stirred solution of 1-(5-bromo-2-chloro-phenyl)-4-tert-butyl-piperazine (1.00 eq, 600 mg, 1.81 mmol) in 1,4-dioxane (12 mL) at room temperature, bis(pinacolato)diboron (1.10 eq, 505 mg, 1.99 mmol) and potassium acetate (3.00 eq, 533 mg, 5.43 mmol) were added. The resulting mixture was degassed with nitrogen for −10 min and then PdCl₂(dppf)·DCM (0.100 eq, 148 mg, 0.181 mmol) was added. The reaction mixture was stirred under nitrogen atmosphere at 80° C. for 16 h. The progress of the reaction was monitored by TLC (50% EtOAc in pet ether) and LCMS. The reaction mixture was cooled to room temperature and filtered through celite, washed with EtOAc. The filtrate was concentrated under reduced pressure to get crude title product (950 mg) as brown liquid. The product is a mixture of boronic acid and ester. LCMS shows major acid mass. LCMS (ESI): m/z calcd. For C₁₄H₂₂BClN₂O₂[M+H]⁺ 297.2, found 297.2 [M+H]⁺.

### Intermediate 17: 1-tert-butyl-4-(2-methoxy-5-(4,4,5,5-tetramethyl-1,3,2-dioxaborolan-2-yl)-3-pyridyl)piperazine (int-17)

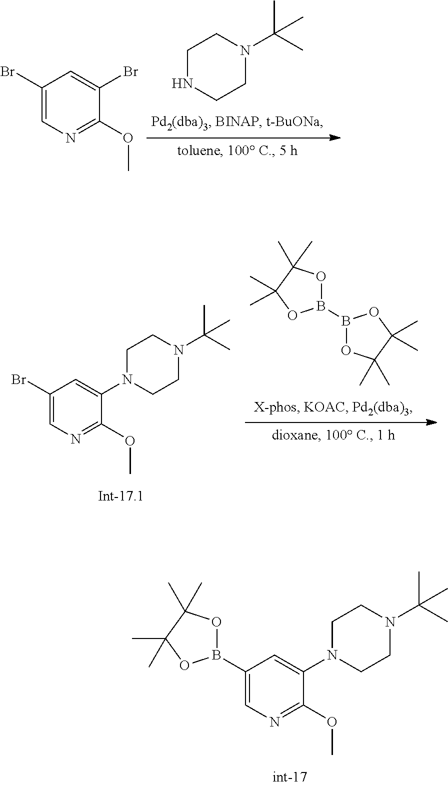

### Step a. Preparation of 1-(5-bromo-2-methoxy-3-pyridyl)-4-tert-butyl-piperazine (int-17.1)

To a solution of 3,5-dibromo-2-methoxy-pyridine (1.00 eq, 1.50 g, 5.62 mmol) in toluene (10 mL) at room temperature, 1-tert-butylpiperazine (1 eq, 800 mg, 5.62 mmol), NaOtBu (1.50 eq, 810 mg, 8.43 mmol) were added and the mixture was degassed for 10 min. Then Pd₂(dba)₃ (0.0501 eq, 258 mg, 0.282 mmol) and rac-BINAP (0.100 eq, 350 mg, 0.562 mmol) were added. Then the reaction mixture was stirred at 100° C. for 5 h. The progress of the reaction was monitored by TLC (70% EtOAc in pet ether). The reaction mixture was concentrated under reduced pressure and the residue was dissolved in EtOAc (50 mL), washed with water, brine, dried over anhyd. sodium sulfate and concentrated under reduced pressure. The crude product was purified by flash silica gel column chromatography (10-70% EtOAc in pet ether) to get the title product (1.00 g, 2.11 mmol, 37.46% yield) as a brown solid. LCMS (ESI): m/z calcd. For C₁₄H₂₂BrN₃O [M+H]⁺ 328.09, found 328.1 [M+H]⁺

### Step b. Preparation of 1-tert-butyl-4-[2-methoxy-5-(4,4,5,5-tetramethyl-1,3,2-dioxaborolan-2-yl)-3-pyridyl]piperazine (int-17)

To a solution of 1-(5-bromo-2-methoxy-3-pyridyl)-4-tert-butyl-piperazine (1.00 eq, 300 mg, 0.914 mmol) in 1,4-dioxane (15 mL)) at room temperature, bis(pinacolato)diboron (1.60 eq, 372 mg, 1.46 mmol), potassium acetate (3.01 eq, 270 mg, 2.75 mmol), Pd₂(dba)₃ (0.0801 eq, 67 mg, 0.0732 mmol) and XPhos (0.161 eq, 70 mg, 0.147 mmol) were added. This reaction mixture was degassed with nitrogen gas for 10 minutes and then stirred at 100° C. for 1 h. The progress of the reaction was monitored by TLC (70% EtOAc in pet ether) and LCMS. The reaction mixture was concentrated under reduced pressure to obtain crude title product (300 mg, 0.799 mmol, 87.46% yield). The crude product was used in the next step without purification. LCMS (ESI): m/z calcd. For C₂₀H₃₄BN₃O₃[M+H]⁺ 376.27, found 376.3 and 294.1 [M+H]⁺ boronic acid mass was also observed along with boronate ester.

### Intermediate 18: (6-(4-(tert-butyl)piperazin-1-yl)-1-methyl-2-oxo-1,2-dihydropyridin-4-yl)boronic Acid (int-18)

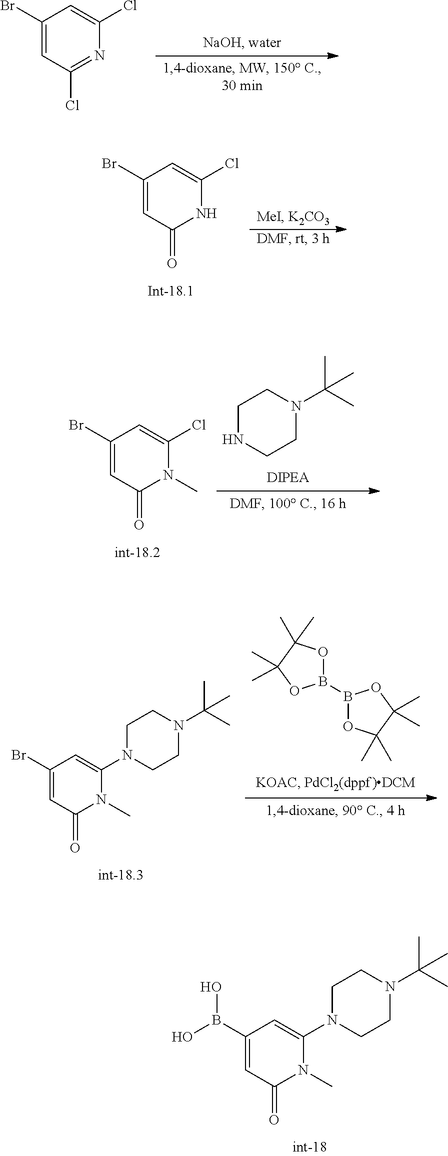

### Step a. Preparation of 4-bromo-6-chloropyridin-2(1H)-one (int-18.1)

To a stirred solution of 4-bromo-2,6-dichloro-pyridine (1.00 eq, 3.00 g, 13.2 mmol) in 1,4-dioxane (3 mL), a solution of sodium hydroxide (0.681 eq, 3.0 mL, 9.00 mmol) was added at room temperature. The reaction was subjected to microwave irradiation at 150° C. for 30 min. The progress was monitored by TLC (50% EtOAc in pet ether) and LCMS. The reaction mixture was acidified to “pH” 4-5 and the solid precipitated was filtered to get the title product (2.40 g, 11.3 mmol, 85.13% yield) as an off white solid. LCMS (ESI): m/z calcd. For C₅H₃BrClNO [M+H]⁺ 207.91, found 207.9 [M+H]⁺

### Step b. Preparation of 4-bromo-6-chloro-1-methylpyridin-2(1H)-one (int-18.2)

To a stirred solution of 4-bromo-6-chloropyridin-2(1H)-one (1.00 eq, 2.40 g, 11.5 mmol) in DMF (25 mL), potassium carbonate (2.00 eq, 3183 mg, 23.0 mmol) and methyl iodide (1.20 eq, 0.86 mL, 13.8 mmol) were added at room temperature. The resulting mixture was stirred at room temperature for 3 h. The progress of the reaction was monitored by TLC (30% EtOAc in pet ether). The reaction mixture was quenched in water (50 mL) and extracted with EtOAc (50 mL×3). The organic layer was washed with brine, dried over anhyd. sodium sulfate and concentrated under reduced pressure and purified to obtain the title product (1.00 g, 4.49 mmol, 39.04% yield) as a pale yellow solid. ¹H-NMR (400 MHz, DMSO-d6): δ 6.83 (d, J=2.80 Hz, 1H), 6.76 (d, J=2.80 Hz, 1H), 3.52 (s, 3H).

### Step c. Preparation of 4-bromo-6-(4-(tert-butyl)piperazin-1-yl)-1-methylpyridin-2(1H)-one (int-18.3)

To a stirred solution of 4-bromo-6-chloro-1-methylpyridin-2(1H)-one (1.00 eq, 1.00 g, 4.49 mmol) in DMF (10 mL), DIPEA (2.00 eq, 1.6 mL, 8.99 mmol) and 1-tert-butylpiperazine (1.00 eq, 639 mg, 4.49 mmol) were added at room temperature. The resulting mixture was stirred at 100° C. for 16 h. The progress of the reaction was monitored by TLC (50% EtOAc in pet ether) and LCMS. The reaction mixture was quenched with water (10 mL) and extracted with 10% MeOH-DCM. The organic layer was washed with brine, dried over anhyd. sodium sulfate and concentrated under reduced pressure. The crude compound was purified by flash silica gel column chromatography (0-10% MeOH in DCM) to get the title product (950 mg, 2.85 mmol, 63.36% yield) as brown solid. LCMS (ESI): m/z calcd. For C₁₄H₂₂BrN₃O [M+H]⁺ 328.09, found 328.1 and 330.2 [M+H]⁺.

### Step d. Preparation of (6-(4-(tert-butyl)piperazin-1-yl)-1-methyl-2-oxo-1,2-dihydropyridin-4-yl)boronic Acid (int-18)

To a stirred solution of 4-bromo-6-(4-(tert-butyl)piperazin-1-yl)-1-methylpyridin-2(1H)-one (1.00 eq, 300 mg, 0.914 mmol) in 1,4-dioxane (15 mL) were added bis(pinacolato)diboron (1.00 eq, 232 mg, 0.914 mmol) and potassium acetate (3.00 eq, 269 mg, 2.74 mmol) at room temperature. The resulting mixture was degassed with nitrogen for ˜10 mins and then PdCl₂(dppf)·DCM (0.1 eq, 74 mg 0.091 mmol) was added. The reaction mixture was stirred under nitrogen atmosphere at 90° C. for 4 h. The progress of the reaction was monitored by LCMS. The reaction mixture was cooled and filtered over celite and washed with EtOAc (50 mL). The filtrate was concentrated and partitioned between water and EtOAc. The aqueous layer was separated and concentrated to dryness to obtain the title product (300 mg, 71% purity), which was used as such in the next step. LCMS (ESI): m/z calcd. For C₁₄H₂₄BN₃O₃[M+H]⁺ 294.19, found 294.2 [M+H]⁺.

### Intermediate 19: 1-(4-(2-bromo-6-fluoro-3-(methoxymethoxy)pyridin-4-yl)-2-chlorophenyl)-3-methyl-1,3-dihydro-2H-imidazol-2-one (int-19)

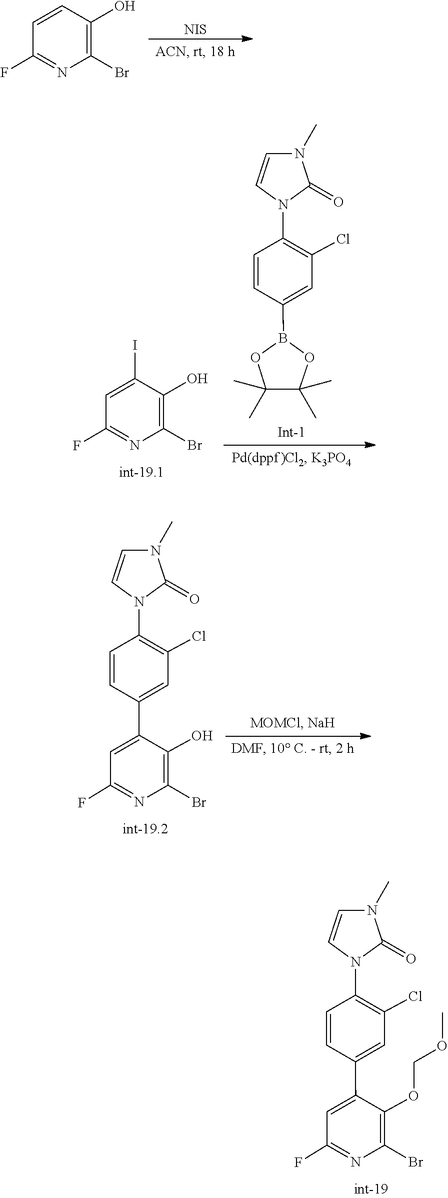

### Step a. Preparation of 2-bromo-6-fluoro-4-iodopyridin-3-ol (int-19.1)

To a solution of 2-bromo-6-fluoropyridin-3-ol (300 mg, 1.57 mmol) in ACN (15 mL) was added NIS (530 mg, 2.35 mmol). The reaction mixture was stirred at room temperature overnight. The solvent was removed in vacuo. The residue was purified by FCC (PE/EA=10/1) to obtain the title product (200 mg, 40% yield). LCMS: 318 (M+H)⁺.

### Step b. Preparation of 1-(4-(2-bromo-6-fluoro-3-hydroxypyridin-4-yl)-2-chlorophenyl)-3-methyl-1,3-dihydro-2H-imidazol-2-one (int-19.2)

To a solution of 2-bromo-6-fluoro-4-iodopyridin-3-ol (250 mg, 0.79 mmol) in 1,4-dioxane/water (5/1, 10 mL) was added 1-(2-chloro-4-(4,4,5,5-tetramethyl-1,3,2-dioxaborolan-2-yl)phenyl)-3-methyl-1,3-dihydro-2H-imidazol-2-one (Intermediate 1, 265 mg, 0.79 mmol), Pd(dppf)Cl₂ (60 mg, 0.08 mmol) and K₃PO₄ (500 mg, 2.4 mmol). The reaction mixture was stirred at 60° C. for 3 hours under N₂. The reaction mixture was cooled to room temperature, suspended in water and extracted with EA. The combined organic layers were concentrated. The residue was purified by FCC (DCM/MeOH=20/1) to afford the title product (180 mg, 57% yield). LCMS: 398 (M+H)⁺.

### Step c. Preparation of 1-(4-(2-bromo-6-fluoro-3-(methoxymethoxy)pyridin-4-yl)-2-chlorophenyl)-3-methyl-1,3-dihydro-2H-imidazol-2-one (int-19)

To a solution of 1-(4-(2-bromo-6-fluoro-3-hydroxypyridin-4-yl)-2-chlorophenyl)-3-methyl-1,3-dihydro-2H-imidazol-2-one (180 mg, 0.45 mmol) in DMF (5 mL) was added NaH (36 mg, 60%, 0.90 mmol). The reaction mixture was stirred at 10° C. for 0.5 h. Then bromo(methoxy)methane (85 mg, 0.69 mmol) was added. The reaction mixture was stirred at room temperature for 2 hours. After the reaction was complete, water was added into the reaction mixture that was extracted with EA. The combined organic layers were washed with water, brine, dried over Na₂SO₄, filtered and concentrated. The residue was purified by FCC (DCM/MeOH=20/1) to afford the title product (170 mg, 85% yield). LCMS: 442 (M+H)⁺.

### Intermediate 20: 1-(2-chloro-4-(2-chloro-3-methoxypyridin-4-yl)phenyl)-3-methyl-1,3-dihydro-2H-imidazol-2-one (int-20)

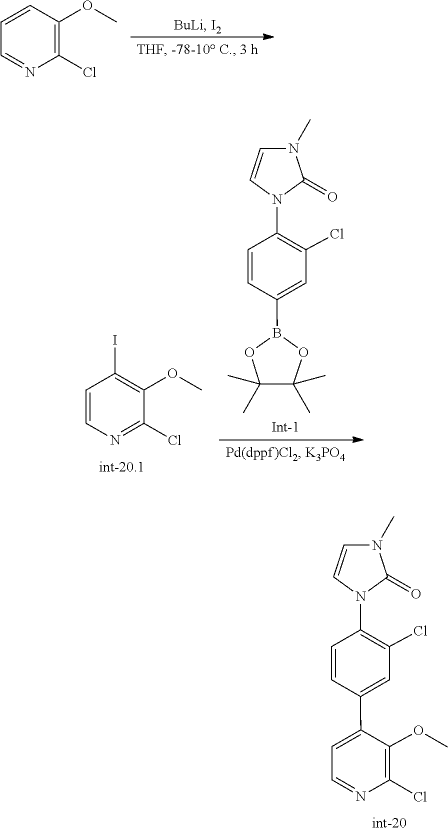

### Step a. Preparation of 2-chloro-4-iodo-3-methoxypyridine (int-20.1)

To a solution of 2-chloro-3-methoxypyridine (1.0 g, 6.99 mmol) in THF (20 mL) was added n-BuLi (7 mL, 17.48 mmol) dropwise at −78° C. and a solution of 12(3.19 g, 12.58 mmol) in THF (20 mL) was added dropwise at −78° C. under nitrogen atmosphere. The reaction mixture was stirred at 10° C. for 3 hours. After the reaction was complete by LCMS, the reaction mixture was quenched with sat NH₄Cl solution and extracted with ethyl ether. The combined organic layers were washed with water, brine and, concentrated. The residue was purified by flash column chromatography (PE:EA=15:1) to afford the title product (1.50 g, 80% yield) as a yellow solid. LCMS: 270.2 (M+H)⁺.

### Step b. Preparation of 1-(2-chloro-4-(2-chloro-3-methoxypyridin-4-yl)phenyl)-3-methyl-1,3-dihydro-2H-imidazol-2-one (int-20)

To a solution of 2-chloro-4-iodo-3-methoxypyridine (1.0 g, 3.71 mmol) in dioxane/H₂O was added 1-(2-chloro-4-(4,4,5,5-tetramethyl-1,3,2-dioxaborolan-2-yl)phenyl)-3-methyl-1,3-dihydro-2H-imidazol-2-one (Intermediate 1, 1.24 g, 3.71 mmol), Pd(dppf)Cl₂ (0.27 g, 0.4 mmol) and Na₂CO₃ (1.18 g, 11.13 mmol). The reaction mixture was stirred at 60° C. for 1 hour under nitrogen atmosphere. After the reaction was complete by LCMS, the reaction mixture was cooled and extracted with ethyl ether. The combined organic layer was washed with water, brine and concentrated. The residue was purified by flash column chromatography (DCM:MeOH=20:1) to afford the title product (1.00 g, 78% yield) as a yellow solid. LCMS: 350.2 (M+H)⁺.

### Intermediate 21: (5-(4-(tert-butyl)piperazin-1-yl)-6-methoxypyridin-3-yl)boronic Acid (int-21)

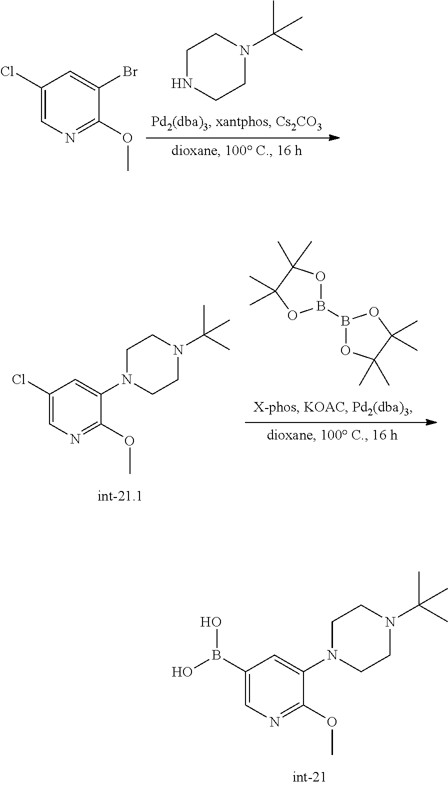

### Step a. Preparation of 1-(tert-butyl)-4-(5-chloro-2-methoxypyridin-3-yl)piperazine (int-21.1)

To a solution of 3-bromo-5-chloro-2-methoxypyridine (1.08 g, 4.84 mmol) and 1-(tert-butyl)piperazine (688 mg, 4.84 mmol) in dioxane (50 mL) was added Pd₂(dba)₃ (443 mg, 0.484 mmol), xantphos (560 mg, 0.968 mmol) and Cs₂CO₃ (4.73 g, 14.5 mmol). The reaction mixture was stirred at 100° C. for 16 hours under nitrogen atmosphere. After the reaction was complete by LCMS, the reaction mixture was cooled to room temperature and the solvent was removed under reduced pressure. The residue was purified by flash column chromatography (PE:EA=1:1) to give 1-(tert-butyl)-4-(5-chloro-2-methoxypyridin-3-yl)piperazine (700 mg, 51% yield) as yellow oil. LCMS: 284.1 (M+H)⁺.

### Step b. Preparation of (5-(4-(tert-butyl)piperazin-1-yl)-6-methoxypyridin-3-yl)boronic Acid (int-21

To a solution of 1-(tert-butyl)-4-(5-chloro-2-methoxypyridin-3-yl)piperazine (400 mg, 1.4 mmol) and bis(pinacolato)diboron (536 mg, 2.1 mmol) in dioxane (15 mL) was added Pd₂(dba)₃ (129 mg, 0.14 mmol), x-phos (134 mg, 0.28 mmol) and KOAc (415 mg, 4.23 mmol). The reaction mixture was stirred at 100° C. overnight under nitrogen atmosphere. After the reaction was complete by LCMS, the reaction mixture was cooled, filtered and concentrated to give the title product (413 mg crude, 100% yield) as brown oil which was used without further purification. LCMS: 294.2 (M+H)⁺.

### Example 1: 1-(3″-(4-(tert-butyl)piperazin-1-yl)-3-chloro-5′-fluoro-2′-hydroxy-[1,1′:3′,1″-terphenyl]-4-yl)-3-methyl-1,3-dihydro-2H-imidazol-2-one (I-1)

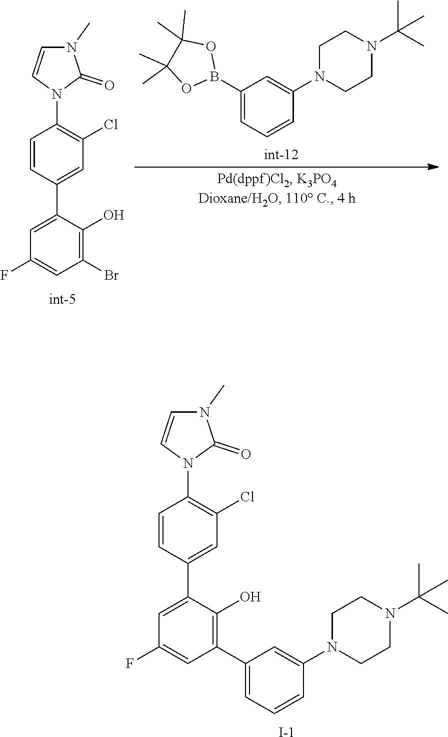

To a solution of 1-(tert-butyl)-4-(3-(4,4,5,5-tetramethyl-1,3,2-dioxaborolan-2-yl)phenyl)piperazine (Intermediate 12, 3.8 g, 11.0 mmol, 1.0 eq.) and 1-(3′-bromo-3-chloro-5′-fluoro-2′-hydroxy-[1,1′-biphenyl]-4-yl)-3-methyl-1,3-dihydro-2H-imidazol-2-one (Intermediate 5, 4.4 g, 11.0 mmol, 1.0 eq.) in Dioxane/H₂O (140 mL, v/v=8:1), was added K₂CO₃ (4.6 g, 33.0 mmol, 3.0 eq.) and Pd (dppf)Cl₂ (805 mg, 1.1 mmol, 0.1 eq.), then it was stirred at 110° C. for 4 hours under nitrogen atmosphere. After the reaction was indicated by LCMS, removed the solvent under reduced pressure, the residue was purified by flash column chromatography (EA:MeOH=4:1) to give a crude, which was further purified by reversed phase flash to give the title product (931 mg, 16% yield) as a white solid. ¹H NMR (400 MHz, DMSO-d₆) δ 8.45 (s, 1H), 7.81 (d, J=1.8 Hz, 1H), 7.62 (dd, J=8.2, 1.8 Hz, 1H), 7.51 (d, J=8.2 Hz, 1H), 7.28 (t, J=7.9 Hz, 1H), 7.20-7.05 (m, 3H), 6.94 (dd, J=14.3, 5.2 Hz, 2H), 6.70 (dd, J=10.9, 3.0 Hz, 2H), 3.21 (s, 3H), 3.17-3.14 (m, 4H), 2.66-2.64 (m, 4H), 1.05 (s, 9H). LCMS: 535.3 (M+H)⁺.

### Example 2: 1-(4-(2-(3-(4-(tert-butyl)piperazin-1-yl)phenyl)-3-hydroxy-6-methylpyridin-4-yl)-2-chlorophenyl)-3-methyl-1,3-dihydro-2H-imidazol-2-one (I-2)

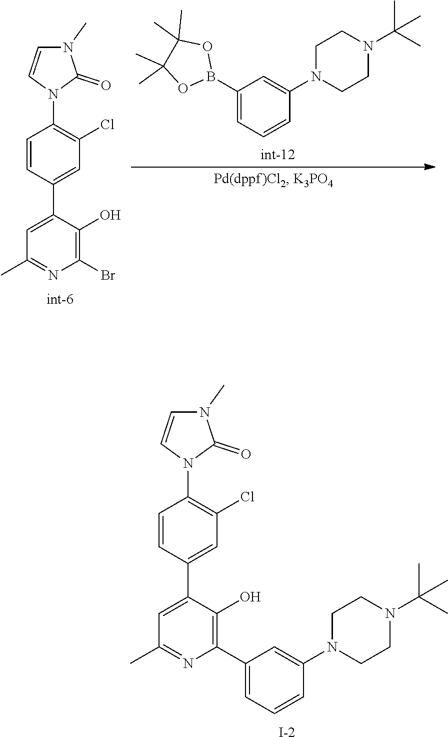

To a solution of 1-(4-(2-bromo-3-hydroxy-6-methylpyridin-4-yl)-2-chlorophenyl)-3-methyl-1,3-dihydro-2H-imidazol-2-one (Intermediate 6, 10.5 g, 26.7 mmol) in 1,4-dioxane/H₂O (300 mL/60 mL) was added 1-(tert-butyl)-4-(3-(4,4,5,5-tetramethyl-1,3,2-dioxaborolan-2-yl)phenyl)piperazine (Intermediate 12, 12.0 g, 35.0 mmol), Pd(dppf)Cl₂ (1.9 g, 2.6 mmol) and K₃PO₄ (17.0 g, 80.0 mmol). The reaction mixture was stirred at 100° C. for 6 hours under N₂. The reaction mixture was cooled, added water, and extracted with EA. The combined organic layer was concentrated. The residue was purified by FCC (DCM/MeOH=20/1) to obtain the crude product which was purified by C18 column (0.1% NH₄CO₃ H₂O/ACN) to obtain the title product (3.5 g, 25% yield) as a yellow solid. ¹H NMR (400 MHz, DMSO-d₆) δ 8.80 (s, 1H), 7.85 (d, J=2.0 Hz, 1H), 7.66 (dd, J=8.0, 2.0 Hz, 1H), 7.55 (d, J=8.0 Hz, 1H), 7.39 (s, 1H), 7.30-7.26 (m, 2H), 7.17 (s, 1H), 6.95 (d, J=6.4 Hz, 1H), 6.72 (d, J=3.2 Hz, 1H), 6.69 (d, J=3.2 Hz, 1H), 3.21 (s, 3H), 3.17-3.10 (m, 4H), 2.66-2.55 (m, 4H), 2.46 (s, 3H), 1.05 (s, 9H); LCMS: 532 (M+H)⁺.

### Example 3: 1-(4-(2-(3-(4-(tert-butyl)piperazin-1-yl)phenyl)-3-hydroxy-6-methylpyridin-4-yl)-2-chlorophenyl)-3-(methyl-d3)-1,3-dihydro-2H-imidazol-2-one (I-4)

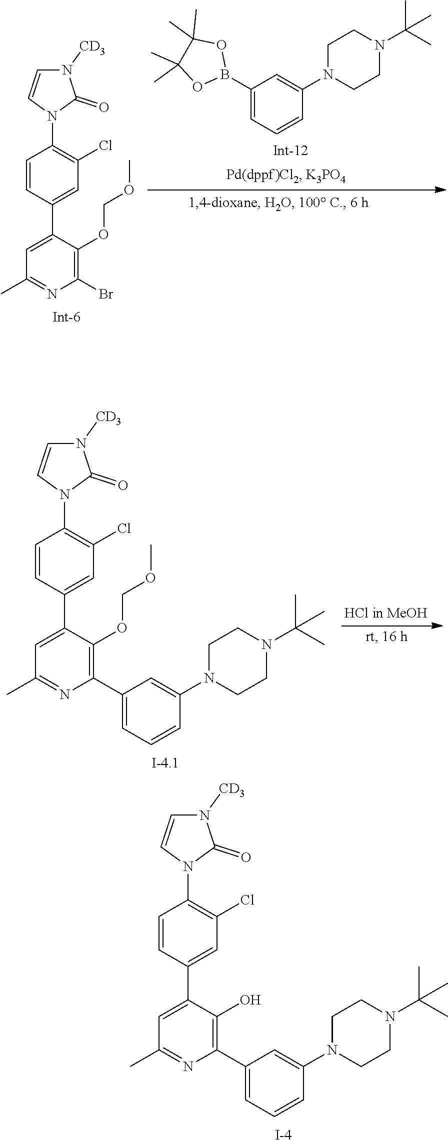

### Step a. Preparation of 1-(4-(2-(3-(4-(tert-butyl)piperazin-1-yl)phenyl)-3-(methoxymethoxy)-6-methylpyridin-4-yl)-2-chlorophenyl)-3-(methyl-d₃)-1,3-dihydro-2H-imidazol-2-one (I-4.1)

To a stirred solution of 1-(4-(2-bromo-3-(methoxymethoxy)-6-methylpyridin-4-yl)-2-chlorophenyl)-3-(methyl-d₃)-1,3-dihydro-2H-imidazol-2-one (Intermediate 10, 1.00 eq, 420 mg, 0.951 mmol) in 1,4-dioxane (6 mL) and water (1 mL) at room temperature, 1-(tert-butyl)-4-(3-(4,4,5,5-tetramethyl-1,3,2-dioxaborolan-2-yl)phenyl)piperazine (Intermediate 12, 0.999 eq, 249 mg, 0.723 mmol) and a solution of Na₂CO₃ (3.00 eq, 302 mg, 2.85 mmol) were added. The resulting mixture was degassed with nitrogen for ˜10 min and then PdCl₂(dppf)·DCM (0.0992 eq, 77 mg, 0.0943 mmol) was added. The reaction mixture was stirred under nitrogen atmosphere at 100° C. for 6 h. The progress of the reaction was monitored by UPLC analysis. The reaction mixture was quenched with water (10 mL) and extracted with ethyl acetate (10 mL×4). The organic layer was washed with brine, dried over anhydrous sodium sulfate, and concentrated under reduced pressure. The crude product was purified by flash silica gel column chromatography (0-10% MeOH in DCM) to get the title product (280 mg, 0.483 mmol, 50.85% yield) as a brown solid. LCMS (ESI): m/z calcd. For C₃₂H₃₅D₃ClN₅O₃[M+H]⁺ 579.29, found 579.2 [M+H]⁺

### Step b. Preparation of 1-(4-(2-(3-(4-(tert-butyl)piperazin-1-yl)phenyl)-3-hydroxy-6-methylpyridin-4-yl)-2-chlorophenyl)-3-(methyl-d₃)-1,3-dihydro-2H-imidazol-2-one (I-4)

To a stirred solution of 1-(4-(2-(3-(4-(tert-butyl)piperazin-1-yl)phenyl)-3-(methoxymethoxy)-6-methylpyridin-4-yl)-2-chlorophenyl)-3-(methyl-d₃)-1,3-dihydro-2H-imidazol-2-one (1.00 eq, 180 mg, 0.311 mmol) in ethyl acetate (2 mL) at 0° C. was added a solution of 4.0 M HCl in EtOAc (10.0 eq, 2.0 mL, 3.11 mmol). The resulting reaction mixture was stirred at room temperature for 16 h. The progress of the reaction was monitored by UPLC. The reaction mixture was concentrated to dryness under reduced pressure. The crude compound was purified by reverse phase prep HPLC (X-Bridge C8, 5 mM Ammonium bicarbonate in water/MeCN) to obtain the title product (45 mg, 0.0831 mmol, 26.75% yield) as a yellow solid. ¹H-NMR (400 MHz, DMSO-d6): δ 8.84 (s, 1H), 7.86 (d, J=1.60 Hz, 1H), 7.67 (dd, J=2.00, 8.20 Hz, 1H), 7.57 (d, J=8.40 Hz, 1H), 7.42 (s, 1H), 7.31 (s, 1H), 7.20 (s, 1H), 7.01 (s, 1H), 6.73 (d, J=2.80 Hz, 1H), 6.71 (d, J=3.20 Hz, 1H), 3.21-3.08 (broad m, 4H), 2.85-2.55 (broad m, 4H), 1.13 (broad s, 9H). LCMS (ESI): m/z calcd. For C₃₀H₃₁D₃ClN₅O₂[M+H]⁺ 535.26, found 535.2 [M+H]⁺.

### Example 4: 1-(4-(3-amino-2-(3-(4-(tert-butyl)piperazin-1-yl)phenyl)-6-methylpyridin-4-yl)-2-chlorophenyl)-3-methyl-1,3-dihydro-2H-imidazol-2-one (I-5)

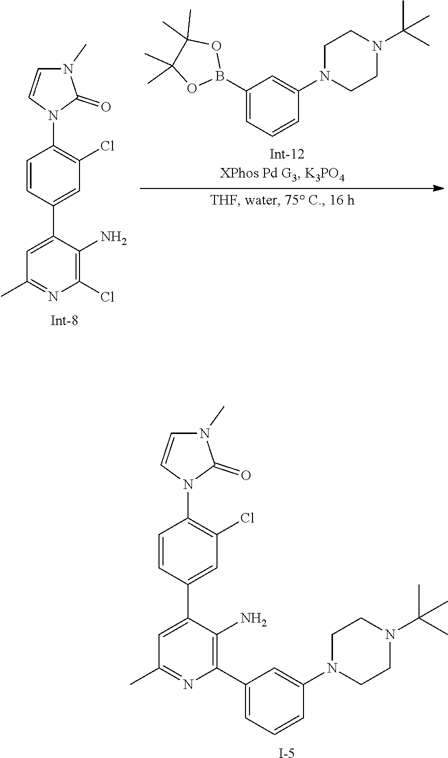

To a solution of 1-(4-(3-amino-2-chloro-6-methylpyridin-4-yl)-2-chlorophenyl)-3-methyl-1,3-dihydro-2H-imidazol-2-one (Intermediate 8, 1.00 eq, 500 mg, 1.43 mmol) in THF (5 mL) at room temperature, 1-(tert-butyl)-4-(3-(4,4,5,5-tetramethyl-1,3,2-dioxaborolan-2-yl)phenyl)piperazine (Intermediate 12, 1.10 eq, 543 mg, 1.58 mmol), a solution of K₃PO₄ (3.00 eq, 912 mg, 4.30 mmol) in water (0.5 mL) and Xphos Pd G3 (0.101 eq, 122 mg, 0.144 mmol) were added. This reaction mixture was degassed for 10 min and stirred at 75° C. for 16 h. The progress of the reaction was monitored by TLC (10% methanol in DCM) and LCMS. The reaction mixture was concentrated under reduced pressure to get crude residue was partitioned between EtOAc (100 mL) and water (100 mL). The organic layer was then washed with water, brine, dried over anhyd. sodium sulfate and concentrated under reduced pressure to get crude as brown solid. The crude compound was purified by reverse phase prep HPLC (10 mM ammonium bicarbonate in MeCN and water) to get the title product (140 mg, 0.263 mmol, 18.34% yield) as a white solid. ¹H-NMR (400 MHz, DMSO-d6): δ 7.77 (t, J=0.80 Hz, 1H), 7.59 (d, J=1.60 Hz, 2H), 7.33 (t, J=8.00 Hz, 1H), 7.11 (s, 1H), 7.02-6.96 (m, 3H), 6.73 (d, J=3.20 Hz, 1H), 6.69 (d, J=2.80 Hz, 1H), 4.38 (s, 2H), 3.22 (s, 3H), 3.16 (t, J=5.20 Hz, 4H), 2.66 (t, J=4.80 Hz, 4H), 2.39 (s, 3H), 1.06 (s, 9H). LCMS (ESI): m/z calcd. For C₃₀H₃₅ClN₆₀ [M+H]⁺ 531.26, found 531.3 [M+H]⁺.

### Example 5: 1-(4-(2-(3-(4-(tert-butyl)piperazin-1-yl)-4-fluorophenyl)-3-hydroxy-6-methylpyridin-4-yl)-2-chlorophenyl)-3-methyl-1,3-dihydro-2H-imidazol-2-one (I-7)

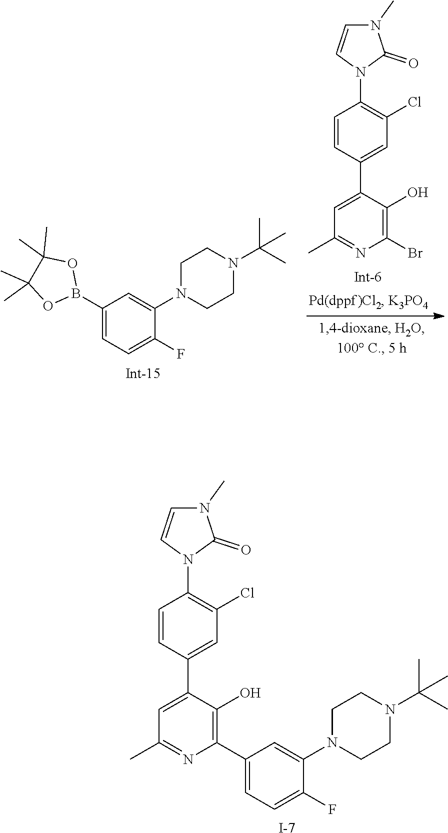

To a stirred solution of 1-(4-(2-bromo-3-hydroxy-6-methylpyridin-4-yl)-2-chlorophenyl)-3-methyl-1,3-dihydro-2H-imidazol-2-one (Intermediate 6, 1.00 eq, 300 mg, 0.760 mmol) in 1,4-dioxane (10 mL) at room temperature were added 1-(tert-butyl)-4-(2-fluoro-5-(4,4,5,5-tetramethyl-1,3,2-dioxaborolan-2-yl)phenyl)piperazine (Intermediate 15, 1.23 eq, 340 mg, 0.938 mmol) and a solution of K₃PO₄ (3.04 eq, 490 mg, 2.31 mmol) in water (3 mL). The resulting mixture was degassed with nitrogen for −10 min and then Pd(dppf)Cl₂·DCM (0.113 eq, 70 mg, 0.0857 mmol) was added. The resulting reaction mixture was heated 100° C. for 5 h. The progress of the reaction was monitored by LCMS. The reaction mixture was concentrated under reduced pressure and the residue was purified by flash silica gel column chromatography (1-25% MeOH in DCM) to enhance the purity of the product. Then the impure product was purified by reverse phase prep HPLC (Column: XBridge C8: Mobile phase: 10 mM ammonium bicarbonate in water/ACN) to get the title product (70 mg, 0.127 mmol, 16.66% yield) as a yellow solid. ¹H-NMR (400 MHz, DMSO-d6): δ 8.93 (s, 1H), 7.86 (d, J=1.60 Hz, 1H), 7.67 (dd, J=2.00, 8.40 Hz, 1H), 7.58-7.46 (m, 3H), 7.23-7.17 (m, 2H), 6.74 (d, J=2.80 Hz, 1H), 6.71 (d, J=2.80 Hz, 1H), 3.22 (s, 3H), 3.05 (broad t, 4H), 2.68 (t, J=1.60 Hz, 4H), 2.47 (s, 3H), 1.06 (s, 9H). ¹⁹F-NMR (377 MHz, DMSO-d6): δ −123.52. LCMS (ESI): m/z calcd. For C₃₀H₃₃ClFN₅O₂[M+H]⁺ 550.23, found 550.3 [M+H]⁺

### Example 6: 1-(4-(5′-(4-(tert-butyl)piperazin-1-yl)-3-hydroxy-6′-methoxy-6-methyl-[2,3′-bipyridin]-4-yl)-2-chlorophenyl)-3-methyl-1,3-dihydro-2H-imidazol-2-one (I-3)

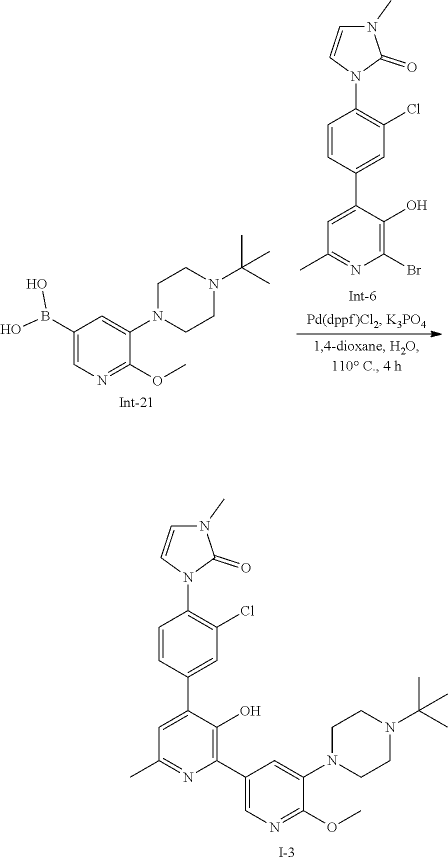

To a solution of (5-(4-(tert-butyl)piperazin-1-yl)-6-methoxypyridin-3-yl)boronic acid (Intermediate 21, 413 mg, 1.41 mmol) and 1-(4-(2-bromo-3-hydroxy-6-methylpyridin-4-yl)-2-chlorophenyl)-3-methyl-1,3-dihydro-2H-imidazol-2-one (Intermediate 6, 554 mg, 1.41 mmol) in dioxane/H₂O (v/v=8:1, 20 mL) was added Pd(dppf)Cl₂ (103 mg, 0.141 mmol) and K₃PO₄ (897 mg, 4.23 mmol). The reaction mixture was stirred at 110° C. for 4 h under nitrogen atmosphere. After the reaction was complete by LCMS, the reaction mixture was cooled to room temperature and the solvent was removed under reduced pressure. The residue was purified by flash column chromatography (DCM:MeOH=15:1) to give the crude product which was further purified by prep-HPLC to give the title product (41.5 mg, 5% yield) as a yellow solid. ¹H NMR (400 MHz, DMSO-d₆) δ 8.98 (s, 1H), 8.28 (d, J=1.8 Hz, 1H), 7.84 (d, J=1.8 Hz, 1H), 7.68-7.65 (m, 2H), 7.56 (d, J=8.2 Hz, 1H), 7.18 (s, 1H), 6.73 (d, J=3.0 Hz, 1H), 6.70 (d, J=3.0 Hz, 1H), 3.94 (s, 3H), 3.21 (s, 3H), 3.10-3.00 (m, 4H), 2.75-2.65 (m, 4H), 2.47 (s, 3H), 1.07 (s, 9H). LCMS: 563.2 (M+H)⁺.

### Example 7: 1-(4-(2-(3-(4-(tert-butyl)piperazin-1-yl)-4-chlorophenyl)-3-hydroxy-6-methylpyridin-4-yl)-2-chlorophenyl)-3-methyl-1,3-dihydro-2H-imidazol-2-one (I-11)

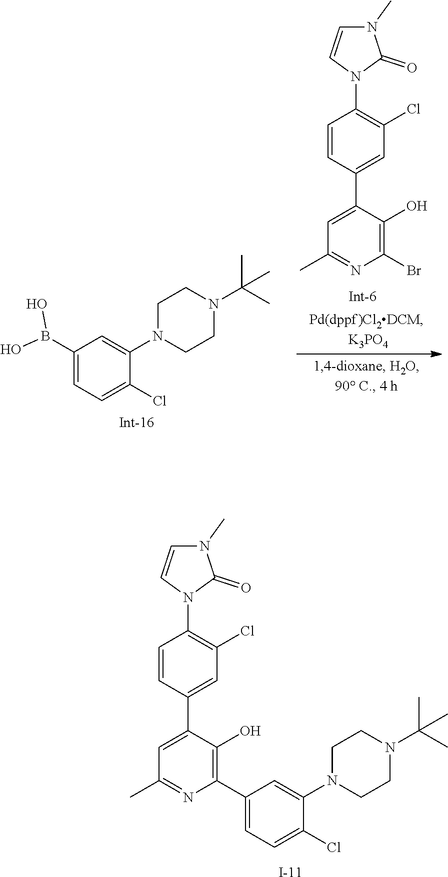

To a stirred solution of of 1-(4-(2-bromo-3-hydroxy-6-methylpyridin-4-yl)-2-chlorophenyl)-3-methyl-1,3-dihydro-2H-imidazol-2-one (Intermediate 6, 1.00 eq, 200 mg, 0.507 mmol) in 1,4-dioxane (8 mL) at room temperature, (3-(4-(tert-butyl)piperazin-1-yl)-4-chlorophenyl)boronic acid (Intermediate 16, 1.50 eq, 225 mg, 0.760 mmol) and a solution of K₃PO₄ (3.00 eq, 323 mg, 1.52 mmol) in water (2 mL) were added. The resulting mixture was degassed with nitrogen for ˜10 min and then PdCl₂(dppf)·DCM (0.100 eq, 41 mg, 0.0507 mmol) was added. The reaction mixture was refluxed under nitrogen at 90° C. for 4 h. The progress of the reaction was monitored by TLC (10% MeOH in DCM) and LCMS. The reaction mixture was concentrated under reduced pressure to get crude as brown liquid, which was passed through silica gel column (1-10% MeOH in DCM) and the product isolated was repurified by reverse phase prep HPLC(0.1% ammonium bicarbonate in MeCN/water) to obtain 1-[4-[2-[3-(4-tert-butylpiperazin-1-yl)-4-chloro-phenyl]-3-hydroxy-6-methyl-4-pyridyl]-2-chloro-phenyl]-3-methyl-imidazol-2-one (10, 30 mg) as a yellow solid. ¹H-NMR (400 MHz, DMSO-d6): δ 9.01 (s, 1H), 7.86 (d, J=1.60 Hz, 1H), 7.69-7.67 (m, 2H), 7.58-7.55 (m, 2H), 7.47 (d, J=8.40 Hz, 1H), 7.22 (s, 1H), 6.74 (d, J=2.80 Hz, 1H), 6.71 (d, J=3.20 Hz, 1H), 3.22 (s, 3H), 3.02 (broad t, 4H), 2.70 (broad t, 4H), 2.48 (s, 3H), 1.07 (s, 9H). LCMS (ESI): m/z calcd. For C₃₀H₃₃Cl₂N₅O₂ [M+H]⁺ 566.2, found 566.1 [M+H]⁺

### Example 8: 1-(4-(2-(3-(4-(tert-butyl)piperazin-1-yl)phenyl)-3-hydroxy-6-methylpyridin-4-yl)-2-methylphenyl)-3-methyl-1,3-dihydro-2H-imidazol-2-one (I-10)

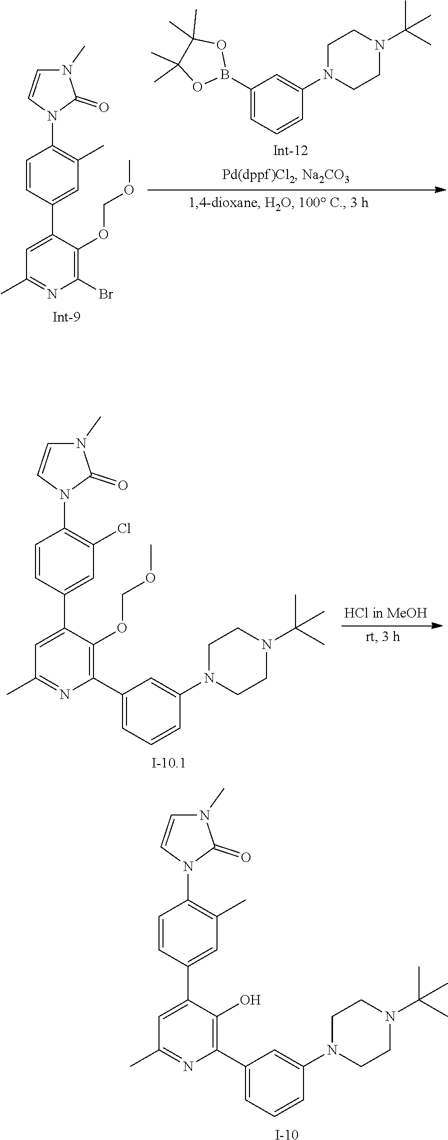

### Step a. Preparation of 1-(4-(2-(3-(4-(tert-butyl)piperazin-1-yl)phenyl)-3-(methoxymethoxy)-6-methylpyridin-4-yl)-2-methylphenyl)-3-methyl-1,3-dihydro-2H-imidazol-2-one (I-10.1)

To a stirred solution of 1-(4-(2-bromo-3-(methoxymethoxy)-6-methylpyridin-4-yl)-2-methylphenyl)-3-methyl-1,3-dihydro-2H-imidazol-2-one (Intermediate 9, 1.00 eq, 350 mg, 0.837 mmol) in 1,4-dioxane (5 mL) at room temperature were added 1-(tert-butyl)-4-(3-(4,4,5,5-tetramethyl-1,3,2-dioxaborolan-2-yl)phenyl)piperazine (Intermediate 12, 1.20 eq, 345 mg, 1.00 mmol) followed by solution of sodium carbonate (3.00 eq, 246 mg, 2.51 mmol) in water (0.5 mL). The resulting reaction mixture was degassed with nitrogen for 10 min and then Pd(dppf)Cl₂·DCM (0.0995 eq, 68 mg, 0.0833 mmol) was added. The reaction was heated under nitrogen atmosphere at 100° C. for 3 h. The reaction progress was monitored by TLC (10% MeOH in DCM). The reaction mixture was quenched with water (8 mL) and extracted with ethyl acetate (10 mL×3). The organic layer was washed with brine, dried over anhyd. sodium sulfate and concentrated under reduced pressure to get crude as brown liquid. The crude compound was purified by flash silica gel column chromatography (EtOAc neat) to get 1-[4-[2-[3-(4-tert-butylpiperazin-1-yl)phenyl]-3-(methoxymethoxy)-6-methyl-4-pyridyl]-2-methyl-phenyl]-3-methyl-imidazol-2-one (200 mg, 0.280 mmol, 33.49% yield) as a brown solid. LCMS (ESI): m/z calcd. For C₃₃H₄₁N₅O₃ [M+H]⁺ 556.32, found 556.3 [M+H]⁺.

### Step b. Preparation of 1-(4-(2-(3-(4-(tert-butyl)piperazin-1-yl)phenyl)-3-hydroxy-6-methylpyridin-4-yl)-2-methylphenyl)-3-methyl-1,3-dihydro-2H-imidazol-2-one (I-10)

To a stirred solution of 1-(4-(2-(3-(4-(tert-butyl)piperazin-1-yl)phenyl)-3-(methoxymethoxy)-6-methylpyridin-4-yl)-2-methylphenyl)-3-methyl-1,3-dihydro-2H-imidazol-2-one (1.00 eq, 150 mg, 0.270 mmol) in methanol (0.5 mL) at 0° C. was added HCl (1.0 mL). The reaction was stirred at room temperature for 3 h. The reaction progress was monitored by LCMS. The crude product was purified by reverse phase prep-HPLC (column: Xbridge C8-250, 10 mM Ammonium bicarbonate/MeCN) to get the title product (33 mg, 0.0633 mmol, 23.47% yield) as a yellow solid. ¹H-NMR (400 MHz, DMSO-d6): δ 8.62 (s, 1H), 7.57 (d, J=1.60 Hz, 1H), 7.52 (dd, J=1.60, 8.00 Hz, 1H), 7.39 (d, J=1.20 Hz, 1H), 7.31-7.26 (m, 3H), 7.12 (s, 1H), 6.97-6.94 (m, 1H), 6.72 (d, J=2.80 Hz, 1H), 6.67 (d, J=3.20 Hz, 1H), 3.22 (s, 3H), 3.16 (broad t, 4H), 2.68 (broad t, 4H), 2.46 (s, 3H), 2.23 (s, 3H), 1.07 (s, 9H). LCMS (ESI): m/z calcd. For C₃₁H₃₇N₅O₂ [M+H]⁺512.29, found 512.3 [M+H]⁺.

### Example 9: 1-(4-(2-(3-(4-(tert-butyl)piperazin-1-yl)-5-chlorophenyl)-3-hydroxy-6-methylpyridin-4-yl)-2-chlorophenyl)-3-methyl-1,3-dihydro-2H-imidazol-2-one (I-6)

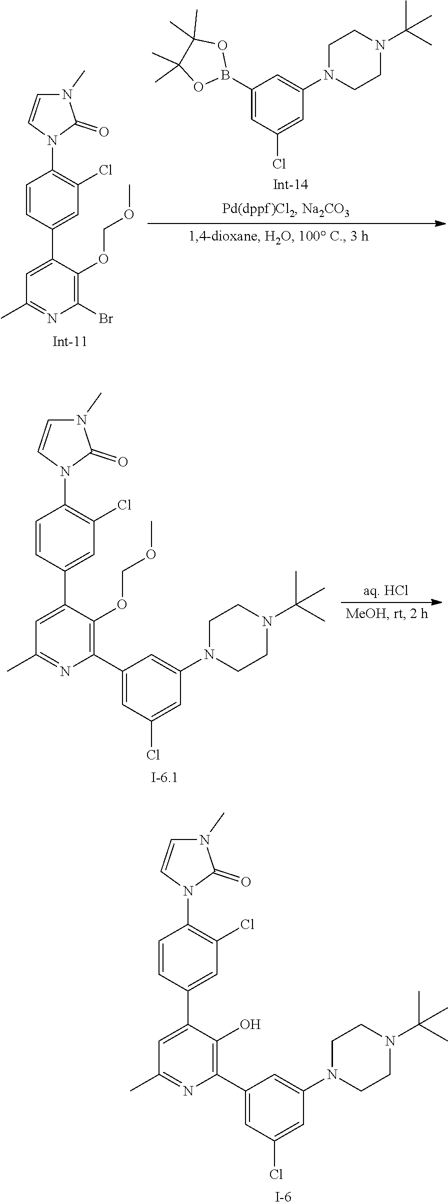

### Step a. Preparation of 1-(4-(2-(3-(4-(tert-butyl)piperazin-1-yl)-5-chlorophenyl)-3-(methoxymethoxy)-6-methylpyridin-4-yl)-2-chlorophenyl)-3-methyl-1,3-dihydro-2H-imidazol-2-one (I-6.1)

To a solution of 1-(tert-butyl)-4-(3-chloro-5-(4,4,5,5-tetramethyl-1,3,2-dioxaborolan-2-yl)phenyl)piperazine (Intermediate 14, 113 mg, 0.25 mmol) in 1,4-dioxane/water (3 mL/0.5 mL) was added 1-(4-(2-bromo-3-(methoxymethoxy)-6-methylpyridin-4-yl)-2-chlorophenyl)-3-methyl-1,3-dihydro-2H-imidazol-2-one (Intermediate 11, 100 mg, 0.23 mmol), Pd(dppf)Cl₂ (17 mg, 0.025 mmol) and Na₂CO₃ (73 mg, 0.69 mmol). The reaction mixture was stirred at 100° C. for 3 h under N₂. After the reaction was complete by LCMS, the reaction mixture was cooled to room temperature, suspended in water and extracted with EA. The combined organic layers were washed with water, brine, dried over Na₂SO₄ and concentrated. The residue was purified by FCC (DCM/MeOH=10/1) to afford the title compound (62 mg, 40% yield). LCMS: 610.2 (M+H)⁺.

### Step b. Preparation of 1-(4-(2-(3-(4-(tert-butyl)piperazin-1-yl)-5-chlorophenyl)-3-hydroxy-6-methylpyridin-4-yl)-2-chlorophenyl)-3-methyl-1,3-dihydro-2H-imidazol-2-one (I-6)

To a solution of 1-(4-(2-(3-(4-(tert-butyl)piperazin-1-yl)-5-chlorophenyl)-3-(methoxymethoxy)-6-methylpyridin-4-yl)-2-chlorophenyl)-3-methyl-1,3-dihydro-2H-imidazol-2-one (62 mg, 0.1 mmol) in MeOH (5 mL) was added HCl (12N aq.)/H₂O (2 mL/4 mL). The reaction mixture was stirred at room temperature for 2 hours under N₂. The pH of the solution was adjusted to neutral with NaHCO₃. The mixture was diluted with water and extracted with EA. The combined organic layers were washed with water, brine, dried over Na₂SO₄ and concentrated. The residue was purified by HPLC to afford the title product (8.5 mg, 15% yield). ¹H NMR (300 MHz, CD₃OD) δ 7.93 (s, 1H), 7.73 (d, J=8.2 Hz, 1H), 7.59 (d, J=8.2 Hz, 1H), 7.35 (d, J=10.4 Hz, 2H), 7.28 (s, 1H), 7.12 (s, 1H), 6.71 (dd, J=11.3, 2.8 Hz, 2H), 3.51 (s, 4H), 3.39 (s, 3H), 3.25 (s, 4H), 2.57 (s, 3H), 1.38 (s, 9H). O—H proton not observed. LCMS: 566.2 (M+H)⁺.

### Example 10: 1-(4-(2-(3-(4-(tert-butyl)piperazin-1-yl)-5-fluorophenyl)-3-hydroxy-6-methylpyridin-4-yl)-2-chlorophenyl)-3-methyl-1,3-dihydro-2H-imidazol-2-one (I-12)

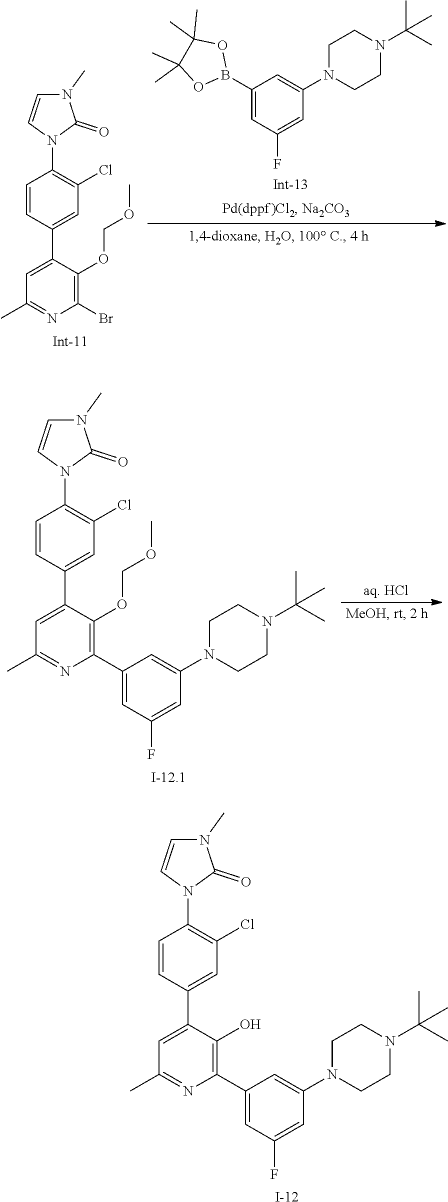

### Step a. Preparation of 1-(4-(2-(3-(4-(tert-butyl)piperazin-1-yl)-5-fluorophenyl)-3-(methoxymethoxy)-6-methylpyridin-4-yl)-2-chlorophenyl)-3-methyl-1,3-dihydro-2H-imidazol-2-one (I-12.1)

A solution of 1-(tert-butyl)-4-(3-fluoro-5-(4,4,5,5-tetramethyl-1,3,2-dioxaborolan-2-yl)phenyl)piperazine (Intermediate 13, 220 mg, 0.60 mmol), 1-(4-(2-bromo-3-(methoxymethoxy)-6-methylpyridin-4-yl)-2-chlorophenyl)-3-methyl-1,3-dihydro-2H-imidazol-2-one (Intermediate 11, 220 mg, 0.50 mmol), Pd(dppf)Cl₂ (36.6 mg, 0.05 mmol) and Na₂CO₃ (160 mg, 1.50 mmol) in dioxane/H₂O (10 mL/2 mL) was stirred at 100° C. for 4 hours under N₂. After the reaction was complete by LCMS, the reaction mixture was cooled to room temperature, suspended in water and extracted with EA. The combined organic layers were washed with water, brine, dried over Na₂SO₄, filtered and concentrated. The residue was purified by FCC to afford the title product (180 mg, 60.6% yield) as a yellow solid. LCMS: 594.3 (M+H)⁺.

### Step b. Preparation of 1-(4-(2-(3-(4-(tert-butyl)piperazin-1-yl)-5-fluorophenyl)-3-hydroxy-6-methylpyridin-4-yl)-2-chlorophenyl)-3-methyl-1,3-dihydro-2H-imidazol-2-one (I-12)

A solution of 1-(4-(2-(3-(4-(tert-butyl)piperazin-1-yl)-5-fluorophenyl)-3-(methoxymethoxy)-6-methylpyridin-4-yl)-2-chlorophenyl)-3-methyl-1,3-dihydro-2H-imidazol-2-one (180 mg, 0.30 mmol) in conc. HCl aq/MeOH (2 mL/2 mL) was stirred at rt for 2 hours. After the reaction was complete by LCMS, the reaction mixture was added aqueous NaHCO₃to adjust the pH-7 and then extracted with EA. The combined organic layers were washed with brine, dried over Na₂SO₄, filtered and concentrated. The residue was purified by HPLC to afford the title product (48.2 mg, 29.2% yield) as a yellow solid. ¹H NMR (400 MHz, DMSO-d₆) δ 8.96 (s, 1H), 7.86 (s, 1H), 7.67 (d, J=8.3 Hz, 1H), 7.56 (d, J=8.1 Hz, 1H), 7.23 (d, J=10.4 Hz, 2H), 7.01 (d, J=8.8 Hz, 1H), 6.78-6.70 (m, 3H), 3.21 (s, 3H), 3.18 (s, 4H), 2.64 (s, 4H), 2.46 (s, 3H), 1.05 (s, 9H). LCMS: 550.2 (M+H)⁺.

### Example 11: 6′-(4-(tert-butyl)piperazin-1-yl)-4-(3-chloro-4-(3-methyl-2-oxo-2,3-dihydro-1H-imidazol-1-yl)phenyl)-3-hydroxy-1′,6-dimethyl-[2,4′-bipyridin]-2′(1′H)-one (I-9)

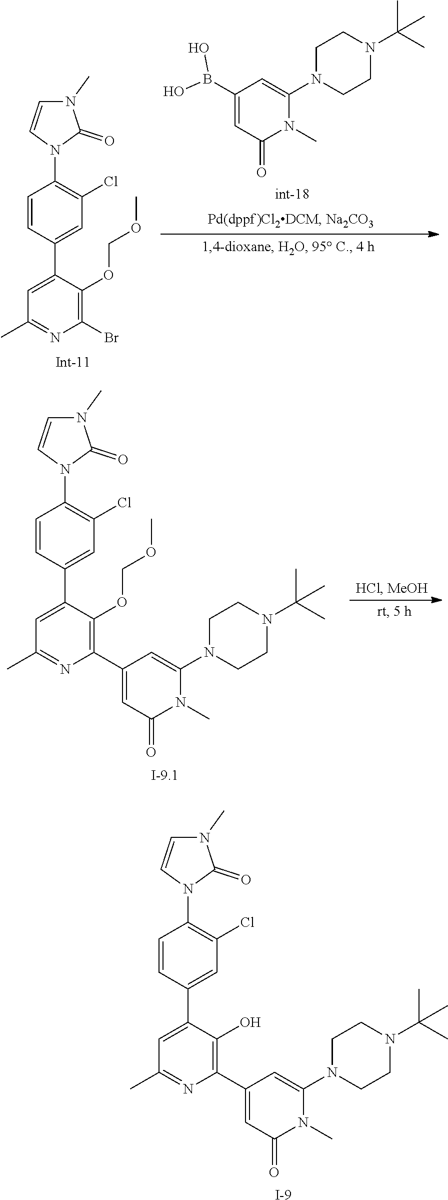

### Step a. Preparation of 6′-(4-(tert-butyl)piperazin-1-yl)-4-(3-chloro-4-(3-methyl-2-oxo-2,3-dihydro-1H-imidazol-1-yl)phenyl)-3-(methoxymethoxy)-1′,6-dimethyl-[2,4′-bipyridin]-2′(1′H)-one (I-9.1)

To a stirred solution of 1-(4-(2-bromo-3-(methoxymethoxy)-6-methylpyridin-4-yl)-2-chlorophenyl)-3-methyl-1,3-dihydro-2H-imidazol-2-one (Intermediate 11, 1.00 eq, 260 mg, 0.593 mmol) in 1,4-dioxane (12 mL) at room temperature were added (6-(4-(tert-butyl)piperazin-1-yl)-1-methyl-2-oxo-1,2-dihydropyridin-4-yl)boronic acid (Intermediate 18, 1.30 eq, 226 mg, 0.770 mmol) and a solution of sodium carbonate (3.00 eq, 174 mg, 1.78 mmol) in water (3 mL). The resulting mixture was degassed with nitrogen for ˜10 min and then PdCl₂(dppf)·DCM (0.100 eq, 48 mg, 0.0593 mmol) was added. The reaction mixture was stirred at 95° C. for 4 h. The progress of the reaction was monitored by LCMS. The reaction mixture was filtered, washed with 1,4-dioxane and concentrated under reduced pressure. The crude product was purified by flash silica gel column chromatography (0-15% MeOH in DCM) to get the title product (140 mg, 0.226 mmol, 38.13% yield) as a brown solid. LCMS (ESI): m/z calcd. For C₃₂H₃₉ClNO₄ [M+H]⁺ 607.27, found 607.3 [M+H]⁺

### Step b. Preparation of 6′-(4-(tert-butyl)piperazin-1-yl)-4-(3-chloro-4-(3-methyl-2-oxo-2,3-dihydro-1H-imidazol-1-yl)phenyl)-3-hydroxy-1′,6-dimethyl-[2,4′-bipyridin]-2′(1′H)-one (I-11)

To a stirred solution of 6′-(4-(tert-butyl)piperazin-1-yl)-4-(3-chloro-4-(3-methyl-2-oxo-2,3-dihydro-1H-imidazol-1-yl)phenyl)-3-(methoxymethoxy)-1′,6-dimethyl-[2,4′-bipyridin]-2′(1¹H)-one (1.00 eq, 135 mg, 0.222 mmol) in methanol (2 mL), 3 N HCl (3.0 mL) was added and the resulting mixture was stirred at room temperature for 5 h. The progress of the reaction was monitored by LCMS. The volatiles were removed under reduced pressure to get crude as brown solid. The crude compound was purified by reverse phase prep HPLC (Xbridge C8-250, 10 mM ammonium bicarbonate in MeCN/water) to get the title product (32 mg) as a yellow solid. ¹H-NMR (400 MHz, DMSO-d6): δ 7.88 (d, J=1.60 Hz, 1H), 7.69 (dd, J=2.00, 8.20 Hz, 1H), 7.57 (d, J=8.00 Hz, 1H), 7.28 (s, 1H), 6.85 (d, J=1.60 Hz, 1H), 6.70 (d, J=3.20 Hz, 1H), 6.67 (d, J=2.80 Hz, 1H), 6.56 (d, J=1.60 Hz, 1H), 3.65 (s, 3H), 3.37 (s, 3H), 3.17-3.09 (broad m, 4H), 2.96-2.85 (broad m, 4H), 2.55 (s, 3H), 1.20 (s, 9H). LCMS (ESI): m/z calcd. For C₃₀H₃₅ClN₆O₃ [M+H]⁺ 563.25, found 563.2 [M+H]⁺

### Example 12: 1-(4-(2-(3-(4-(tert-butyl)piperazin-1-yl)phenyl)-3-hydroxy-6-methylpyridin-4-yl)-2-fluorophenyl)-3-methyl-1,3-dihydro-2H-imidazol-2-one (I-8)

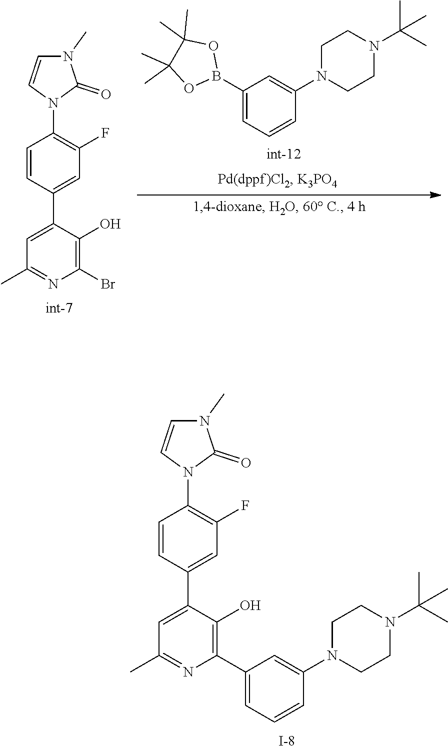

To a solution of 1-(4-(2-bromo-3-hydroxy-6-methylpyridin-4-yl)-2-fluorophenyl)-3-methyl-1,3-dihydro-2H-imidazol-2-one (Intermediate 7, 1.00 eq, 450 mg, 1.19 mmol) in 1,4-dioxane (5 mL) at room temperature under nitrogen atmosphere were added 1-(tert-butyl)-4-(3-(4,4,5,5-tetramethyl-1,3,2-dioxaborolan-2-yl)phenyl)piperazine (Intermediate 12, 1.30 eq, 533 mg, 1.55 mmol) and a solution of K₃PO₄ (3.01 eq, 760 mg, 3.58 mmol) in water (0.8 mL) sequentially. The resulting mixture was degassed with nitrogen for 5 min. Then Pd(dppf)Cl₂·DCM (0.0998 eq, 97 mg, 0.119 mmol) was added. The reaction mixture was stirred at 100° C. for 16 h. The progress of the reaction was monitored by LCMS analysis. The reaction mixture was concentrated under reduced pressure and the residue was taken in EtOAc (20 mL) and washed with water (10 mL), brine, dried over anhydrous sodium sulphate filtered and concentrated under reduced pressure to get crude as brown solid. The crude product was purified by reverse phase prep HPLC (10 mM sodium bicarbonate in water/MeCN) to get the title product (16 mg, 0.0304 mmol, 2.56% yield) as a yellow solid. ¹H-NMR (400 MHz, DMSO-d6): δ 7.95-7.85 (m, 1H), 7.78-7.66 (m, 1H), 7.66-7.53 (m, 2H), 7.51-7.46 (m, 1H), 7.21 (t, J=8.00 Hz, 1H), 7.09 (s, 1H), 6.87 (d, J=8.00 Hz, 1H), 6.80-6.72 (m, 2H), 3.22 (s, 3H), 3.13 (t, J=4.80 Hz, 4H), 2.66 (t, J=4.80 Hz, 4H), 2.40 (s, 3H), 1.06 (s, 9H). ¹⁹F-NMR (377 MHz, DMSO-d6): δ −122.94. LCMS (ESI): m/z calcd. For C₃₀H₃₄FN₅O₂[M+H]⁺ 516.27, found 516.2 [M+H]⁺

### Example 13: 1-(4-(2-(3-(4-(tert-butyl)piperazin-1-yl)phenyl)-3-hydroxy-6-methylpyridin-4-yl)-2-methylphenyl)-3-methyl-1,3-dihydro-2H-imidazol-2-one (I-13)

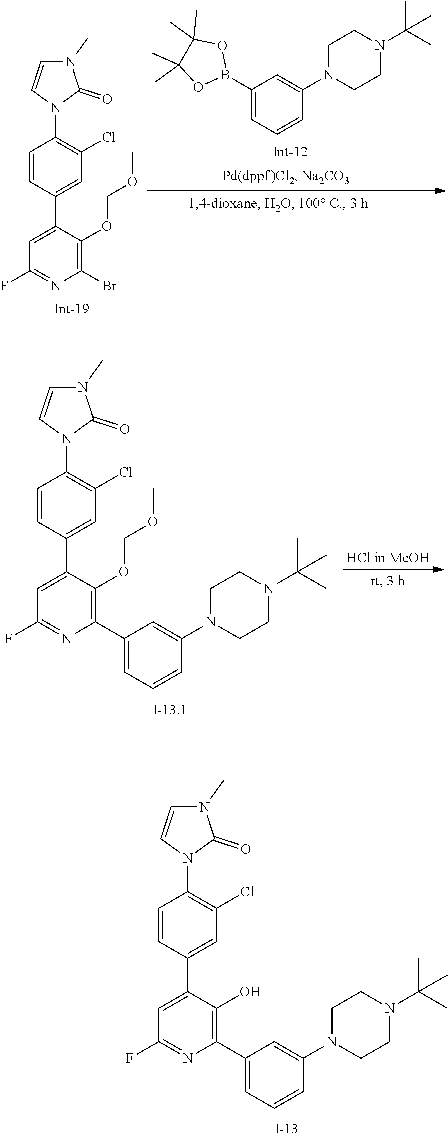

### Step a. Preparation of 1-(4-(2-(3-(4-(tert-butyl)piperazin-1-yl)phenyl)-6-fluoro-3-(methoxymethoxy)pyridin-4-yl)-2-chlorophenyl)-3-methyl-1,3-dihydro-2H-imidazol-2-one (I-13.1)

To a solution of 1-(4-(2-bromo-6-fluoro-3-(methoxymethoxy)pyridin-4-yl)-2-chlorophenyl)-3-methyl-1,3-dihydro-2H-imidazol-2-one (Intermediate-19,110 mg, 0.25 mmol) in 1,4-dioxane/water (5/1, 10 mL) was added 1-(tert-butyl)-4-(3-(4,4,5,5-tetramethyl-1,3,2-dioxaborolan-2-yl)phenyl)piperazine (Intermediate 12, 85 mg, 0.25 mmol), Pd(dppf)Cl₂ (19 mg, 0.03 mmol) and Na₂CO₃ (80 mg, 0.75 mmol). The reaction mixture was stirred at 100° C. for 3 hours under N₂. The reaction mixture was cooled to room temperature, suspended in water, and extracted with EA. The combined organic layers were washed with water, brine, dried over Na₂SO₄, filtered and concentrated. The residue was purified by FCC (EA/PE=1/1) to afford the title product (90 mg, 66% yield). LCMS: 580 (M+H)⁺.

### Step b. Preparation of 1-(4-(2-(3-(4-(tert-butyl)piperazin-1-yl)phenyl)-6-fluoro-3-hydroxypyridin-4-yl)-2-chlorophenyl)-3-methyl-1,3-dihydro-2H-imidazol-2-one (I-13)

To a solution of 1-(4-(2-(3-(4-(tert-butyl)piperazin-1-yl)phenyl)-6-fluoro-3-(methoxymethoxy)pyridin-4-yl)-2-chlorophenyl)-3-methyl-1,3-dihydro-2H-imidazol-2-one (90 mg, 0.16 mmol) in MeOH (3 mL)) was added HCl aqueous solution (2 mL, 4 N). The reaction mixture was stirred at room temperature for 3 hours. The reaction mixture was adjusted to the pH=8 with aq. NaHCO₃ and extracted with DCM. The combined organic layers were washed with water, brine, dried over Na₂SO₄, filtered and concentrated. The residue was purified by HPLC to afford the title product (46 mg, 56% yield). ¹H NMR (400 MHz, DMSO-d₆) δ 9.18 (s, 1H), 7.92 (d, J=2.0 Hz, 1H), 7.71 (dd, J=8.0, 2.0 Hz, 1H), 7.59 (d, J=8.0 Hz, 1H), 7.44 (s, 1H), 7.34-7.27 (m, 2H), 7.16 (d, J=3.2 Hz, 1H), 7.04-6.96 (m, 1H), 6.73 (d, J=3.2 Hz, 1H), 6.71 (d, J=3.2 Hz, 1H), 3.21 (s, 3H), 3.16 (s, 4H), 2.67 (s, 4H), 1.06 (s, 9H). LCMS: 536.2 (M+H)⁺.

### Example 14: 1-(4-(2-(3-(4-(tert-butyl)piperazin-1-yl)phenyl)-3-hydroxypyridin-4-yl)-2-chlorophenyl)-3-methyl-1,3-dihydro-2H-imidazol-2-one (I-14)

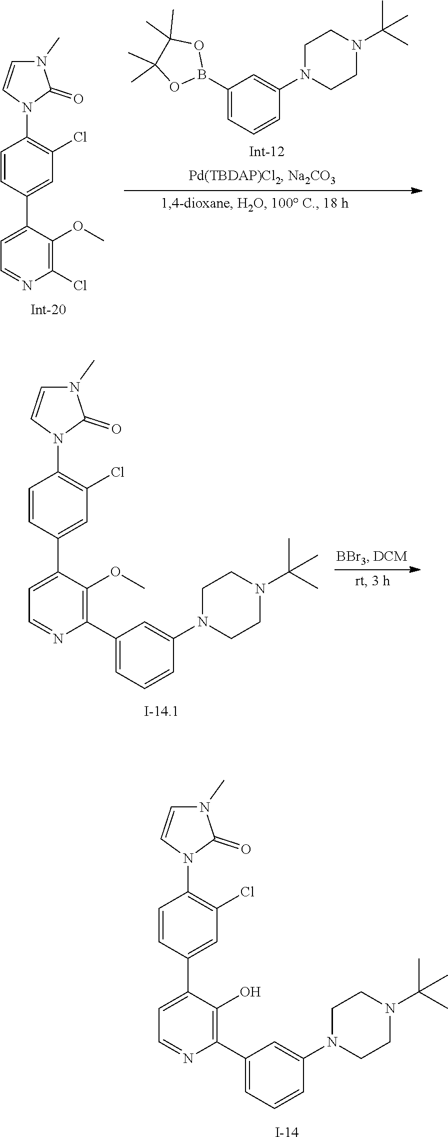

### Step a. Preparation of 1-(4-(2-(3-(4-(tert-butyl)piperazin-1-yl)phenyl)-3-methoxypyridin-4-yl)-2-chlorophenyl)-3-methyl-1,3-dihydro-2H-imidazol-2-one (I-14.1)

To a solution of 1-(2-chloro-4-(2-chloro-3-methoxypyridin-4-yl)phenyl)-3-methyl-1,3-dihydro-2H-imidazol-2-one (Intermediate-20,100 mg, 0.29 mmol) and 1-(tert-butyl)-4-(3-(4,4,5,5-tetramethyl-1,3,2-dioxaborolan-2-yl)phenyl)piperazine (Intermediate 12, 180 mg, 0.52 mmol) in dioxane/H₂O (v/v=8:1, 15 mL) was added Pd(TBDAP)Cl₂ (30 mg, 0.05 mmol) and K₂CO₃ (180, 1.28 mmol). The reaction mixture was stirred at 100° C. overnight under nitrogen atmosphere. After the reaction was complete by LCMS, the solvent was removed under reduced pressure. The residue was purified by flash column chromatography (DCM:MeOH=20:1) to afford 1-(4-(2-(3-(4-(tert-butyl)piperazin-1-yl)phenyl)-3-methoxypyridin-4-yl)-2-chlorophenyl)-3-methyl-1H-imidazol-2(3H)-one (80 mg, 51.8% yield) as yellow oil. LCMS: 532.1 (M+H)⁺.

### Step b. Preparation of 1-(4-(2-(3-(4-(tert-butyl)piperazin-1-yl)phenyl)-3-hydroxypyridin-4-yl)-2-chlorophenyl)-3-methyl-1,3-dihydro-2H-imidazol-2-one (I-14)

To a solution of 1-(2-chloro-4-(2-chloro-3-methoxypyridin-4-yl)phenyl)-3-methyl-1H-imidazol-2(3H)-one (80 mg, 0.15 mmol) in DCM (2 mL) was added BBr₃ (1 mL, 1M). The reaction mixture was stirred at room temperature overnight. After the reaction was complete by LCMS, the reaction mixture was quenched with MeOH. The solvent was removed under reduced pressure. The residue was purified by HPLC to afford the title product (27 mg, 35% yield) as yellow oil. ¹H NMR (400 MHz, CD₃OD) δ 8.33 (d, J=5.6 Hz, 1H), 7.99 (d, J=2.0 Hz, 1H), 7.82-7.70 (m, 2H), 7.63 (d, J=8.0 Hz, 1H), 7.54 (t, J=8.0 Hz, 1H), 7.47 (d, J=2.0 Hz, 1H), 7.41-7.33 (m, 1H), 7.27 (dd, J=8.0, 2.0 Hz, 1H), 6.68 (dd, J=12.0, 2.8 Hz, 2H), 4.03 (d, J=13.2 Hz, 2H), 3.74 (d, J=11.2 Hz, 2H), 3.35 (s, 3H), 3.30 (d, J=12.0 Hz, 2H), 3.16 (t, J=12.0 Hz, 2H), 1.49 (s, 9H). O—H proton not observed. LCMS: 518.1 (M+H)⁺.

### Example 15: 1-(3′-(5-(4-(tert-butyl)piperazin-1-yl)-6-methoxypyridin-3-yl)-3-chloro-5′-fluoro-2′-hydroxy-[1,1′-biphenyl]-4-yl)-3-methyl-1,3-dihydro-2H-imidazol-2-one (I-15)

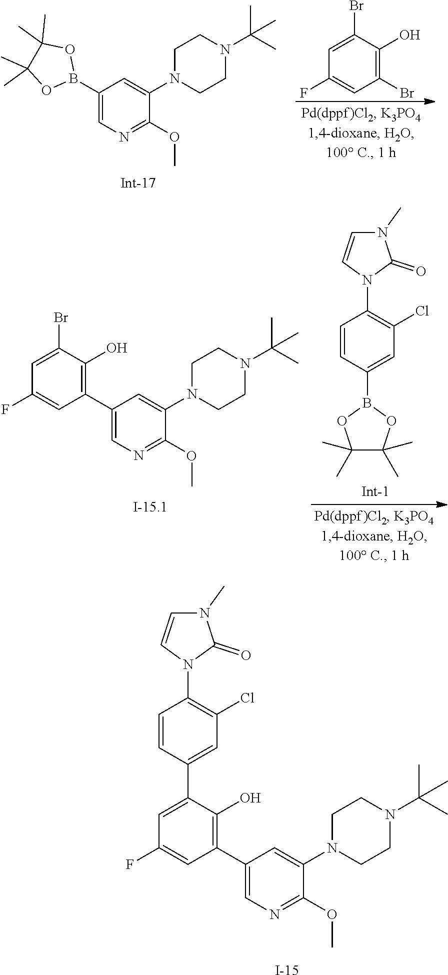

### Step a. Preparation of 2-bromo-6-(5-(4-(tert-butyl)piperazin-1-yl)-6-methoxypyridin-3-yl)-4-fluorophenol (I-15.1)

A mixture of 1-tert-butyl-4-(2-methoxy-5-(4,4,5,5-tetramethyl-1,3,2-dioxaborolan-2-yl)-3-pyridyl)piperazine (Intermediate 17, 30 g, crude), 2,6-dibromo-4-fluorophenol (62.6 g, 231.8 mmol), Pd(dppf)Cl₂ (1.7 g, 2.90 mmol), K₃PO₄ (24.6 g, 116 mmol) in dioxane/H₂O (500 mL/100 mL) was stirred at 100° C. for 1 h under N₂. The reaction mixture was cooled to rt, suspended in H₂O, and extracted with EA. The combined organic layers were washed with brine and concentrated. The residue was purified by FCC (DCM:MeOH=20:1) to afford the title compound (5 g, 17.7% yield for two steps). LCMS: 438.1 (M+H)⁺.

### Step b. Preparation of 1-(3′-(5-(4-(tert-butyl)piperazin-1-yl)-6-methoxypyridin-3-yl)-3-chloro-5′-fluoro-2′-hydroxy-[1,1′-biphenyl]-4-yl)-3-methyl-1,3-dihydro-2H-imidazol-2-one (I-15)

A mixture of 2-bromo-6-(5-(4-(tert-butyl)piperazin-1-yl)-6-methoxypyridin-3-yl)-4-fluorophenol (10 g, 22.8 mmol), 1-(2-chloro-4-(4,4,5,5-tetramethyl-1,3,2-dioxaborolan-2-yl)phenyl)-3-methyl-1,3-dihydro-2H-imidazol-2-one (Intermediate 1, 7.64 g, 22.8 mmol), K₃PO₄ (9.67 g, 45.6 mmol) and Pd(dppf)Cl₂ (834 mg, 1.14 mmol) in dioxane/H₂O (200 mL/30 mL) was stirred at 100° C. for 1 h under N₂. The reaction mixture was cooled to rt, dissolved in H₂O (30 mL) and extracted with EA. The combined organic layers were washed with brine and concentrated. The residue was purified by flash chromatography to afford the crude compound which was purified by HPLC to afford the title compound (2.05 g, 16% yield). ¹H NMR (400 MHz, DMSO-d₆) δ 8.61 (s, 1H), 7.92 (s, 1H), 7.81 (d, J=1.8 Hz, 1H), 7.62 (dd, J=8.2, 1.9 Hz, 1H), 7.52 (d, J=8.2 Hz, 1H), 7.33 (s, 1H), 7.19 (d, J=9.1 Hz, 2H), 6.70 (dd, J=12.2, 3.0 Hz, 2H), 3.93 (s, 3H), 3.21 (s, 3H), 3.05 (s, 4H), 2.65 (s, 4H), 1.05 (s, 9H). LCMS: 566.2 (M+H)⁺.

### Example 16: BET BRD and PBMC MCP-1 HTRF Assays

### Target Engagement Intracellular BET BRD Assay

3× Complete Substrate plus Inhibitor Solution in Assay Medium (Opti-MEM® I Reduced Serum Medium, no phenol red, and no serum) was prepared just before measuring BRET. This solution consisted of a 1:166 dilution of NanoBRET™ Nano-Glo® Substrate plus a 1:500 dilution of Extracellular NanoLuc® Inhibitor in Assay Medium. For a 96-well plate, 30 μl of NanoBRET™ Nano-Glo® Substrate, 10 μl of Extracellular NanoLuc® Inhibitor and 4,960 μl of Assay Medium were mixed to produce 5 ml of 3× Complete Substrate plus Inhibitor Solution, followed with gently mixing by inversion 5-10 times in a conical tube. (The final concentration of Extracellular NanoLuc® Inhibitor in the 3× solution was 60 μM, for a working concentration of 20 μM. Use 3× Complete Substrate plus Inhibitor Solution within 2 hours. Discard any remaining solution).

50 μl of 3× Complete Substrate plus Inhibitor Solution were added to each well of the 96-well plate, followed by incubation for 2-3 minutes at room temperature. Donor emission wavelength (e.g., 450 nm) and acceptor emission wavelength (e.g., 610 nm) were measured by using the GloMax® Discover System or other NanoBRET™ Assay-compatible luminometer (it is recommended measuring BRET within 10 minutes after adding NanoBRET™ Nano-Glo™ Substrate plus Extracellular NanoLuc® Inhibitor Solution. However, BRET can be measured for up to 2 hours, but there will be some loss of luminescence signal).

To generate raw BRET ratio values, the acceptor emission value (e.g., 610 nm) was divided by the donor emission value (e.g., 450 nm) for each sample [to correct for background, the BRET ratio was substracted in the absence of tracer (average of no-tracer control samples) from the BRET ratio of each sample]. Raw BRET units were converted to milliBRET units (mBU) by multiplying each raw BRET value by 1,000. NanoBRET™ ratio equation, including optional background correction is shown below:

BRET Ratio=[(Acceptorsample÷Donorsample)−(Acceptorno-tracer control÷Donorno-tracer control)]×1,000.

### PBMC MCP-1 HTRF Assay

The cryopreserved PBMC cells were thawed in a 37° C. water bath immediately after taking out from liquid nitrogen storage. A sterile pipette was used to transfer the content to sterile 10 mL centrifuge tube containing 50 mL of complete growth medium (Gibico 1640) and centrifuge at 300×g for 10 min. Supernatant was discarded, and cell pellet was resuspended in 10 mL of complete growth media in a sterile 15 mL tube. The cells were rested for 1 h at 37° C. (1×10⁶ cells/mL). The cells were spinned down at 300×g for 10 minutes after 1 h. Supernatant was discard, and cell pellet was resuspended in complete growth medium, the cells number and viability were measured by AO/PI staining. In a 384-well white plate, PBMCs at 25 k cells/well (15 μl per well) were seeded with Gibico 1640 medium. The compound stock was serial diluted into 10 mM concentrations by 3-fold dilution. 20 nL DMSO, 20 nL test compounds DMSO stock (serial diluted) were transferred in assay plate by using ECHO550, incubate for 30 min at 37° C., 5% CO2. LPS was diluted by Gibico 1640 medium.add 5 μl per well. The final concentration (200 ng/mL) was incubated for 24 hours at 37° C. with 5% CO₂.

For MCP-1 HTRF, 2.5 μl/well MCP-1 donor antibody and 2.5 μl/well MCP-1 acceptor antibody were added into 384 well plate, following by centrifuging at 190×g for 1 min and incubating at room temperature for 2 h. The plate was read on the Envision.

The inhibitory activity of exemplary compounds is shown in Table 2.

TABLE 2
Biological Activity of Exemplary Compounds of the Disclosure

| | | | BRD4 | MCP-1 |
|---|---|---|---|---|
| Example | | | BD2 TE | HTRF IC₅₀ |
| # | Structure | Compound name | IC₅₀ (nM) | (nM) |
| I-1 | 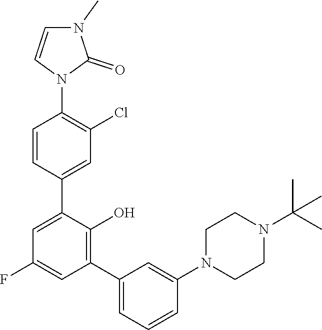 [SMILES: Cn1ccn(-c2ccc(-c3cc(F)cc(-c4cccc(N5CCN(C(C)(C)C)CC5)c4)c3O)cc2Cl)c1=O] | 1-(3″-(4-(tert- butyl)piperazin-1- yl)-3-chloro-5′- fluoro-2′-hydroxy- [1,1′:3′,1″- terphenyl]-4-yl)-3- methyl-1,3- dihydro-2H- imidazol-2-one | 0.323 | 1.508 |
| I-2 | 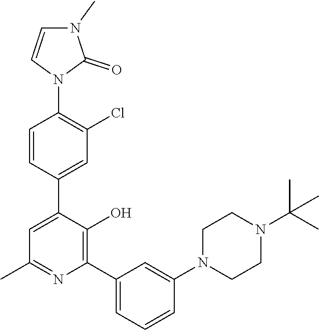 [SMILES: Cc1cc(-c2ccc(-n3ccn(C)c3=O)c(Cl)c2)c(O)c(-c2cccc(N3CCN(C(C)(C)C)CC3)c2)n1] | 1-(4-(2-(3-(4-(tert- butyl)piperazin-1- yl)phenyl)-3- hydroxy-6- methylpyridin-4- yl)-2- chlorophenyl)-3- methyl-1,3- dihydro-2H- imidazol-2-one | 0.152 | 1.223 |
| I-3 | 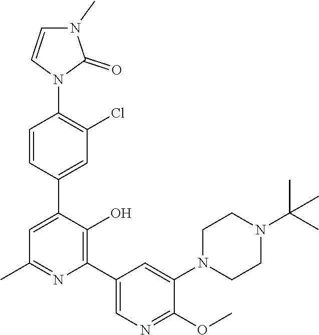 [SMILES: COc1ncc(-c2nc(C)cc(-c3ccc(-n4ccn(C)c4=O)c(Cl)c3)c2O)cc1N1CCN(C(C)(C)C)CC1] | 1-(4-(5′-(4-(tert- butyl)piperazin-1- yl)-3-hydroxy-6′- methoxy-6-methyl- [2,3′-bipyridin]-4- yl)-2- chlorophenyl)-3- methyl-1,3- dihydro-2H- imidazol-2-one | 4.496 | 4.399 |
| I-4 | 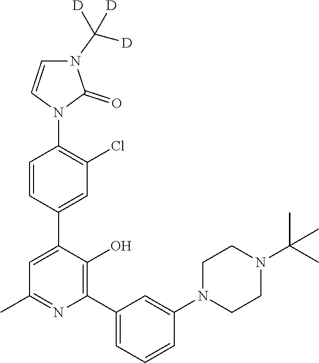 [SMILES: [2H]C([2H])([2H])n1ccn(-c2ccc(-c3cc(C)nc(-c4cccc(N5CCN(C(C)(C)C)CC5)c4)c3O)cc2Cl)c1=O] | 1-(4-(2-(3-(4-(tert- butyl)piperazin-1- yl)phenyl)-3- hydroxy-6- methylpyridin-4- yl)-2- chlorophenyl)-3- (methyl-d₃)-1,3- dihydro-2H- imidazol-2-one | 10.15 | 1.718 |
| I-5 | 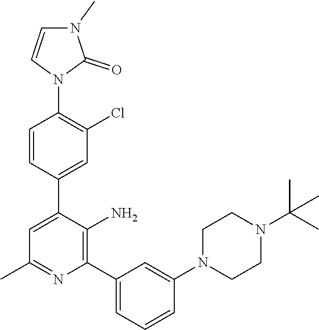 [SMILES: Cc1cc(-c2ccc(-n3ccn(C)c3=O)c(Cl)c2)c(N)c(-c2cccc(N3CCN(C(C)(C)C)CC3)c2)n1] | 1-(4-(3-amino-2-(3- (4-(tert- butyl)piperazin-1- yl)phenyl)-6- methylpyridin-4- yl)-2- chlorophenyl)-3- methyl-1,3- dihydro-2H- imidazol-2-one | 0.473 | 0.583 |
| I-6 | 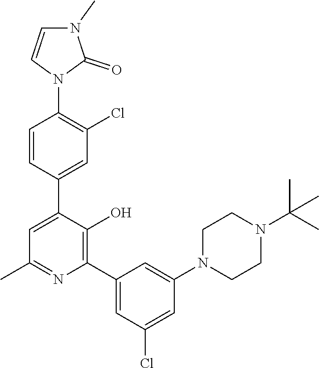 [SMILES: Cc1cc(-c2ccc(-n3ccn(C)c3=O)c(Cl)c2)c(O)c(-c2cc(Cl)cc(N3CCN(C(C)(C)C)CC3)c2)n1] | 1-(4-(2-(3-(4-(tert- butyl)piperazin-1- yl)-5- chlorophenyl)-3- hydroxy-6- methylpyridin-4- yl)-2- chlorophenyl)-3- methyl-1,3- dihydro-2H- imidazol-2-one | 9.111 | 2.250 |
| I-7 | 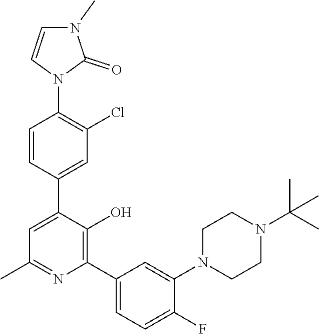 [SMILES: Cc1cc(-c2ccc(-n3ccn(C)c3=O)c(Cl)c2)c(O)c(-c2ccc(F)c(N3CCN(C(C)(C)C)CC3)c2)n1] | 1-(4-(2-(3-(4-(tert- butyl)piperazin-1- yl)-4- fluorophenyl)-3- hydroxy-6- methylpyridin-4- yl)-2- chlorophenyl)-3- methyl-1,3- dihydro-2H- imidazol-2-one | 11.0 | 1.787 |
| I-8 | 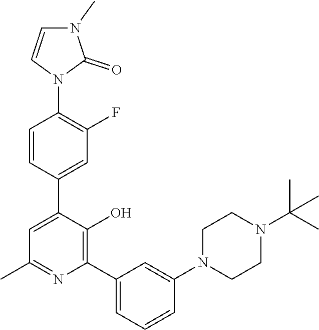 [SMILES: Cc1cc(-c2ccc(-n3ccn(C)c3=O)c(F)c2)c(O)c(-c2cccc(N3CCN(C(C)(C)C)CC3)c2)n1] | 1-(4-(2-(3-(4-(tert- butyl)piperazin-1- yl)phenyl)-3- hydroxy-6- methylpyridin-4- yl)-2- fluorophenyl)-3- methyl-1,3- dihydro-2H- imidazol-2-one | 13.0 | 5.395 |
| I-9 | 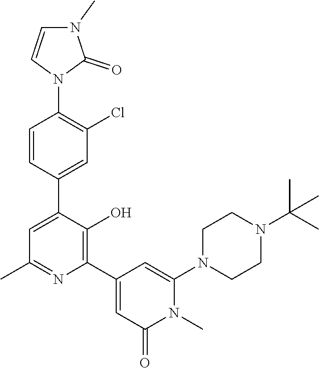 [SMILES: Cc1cc(-c2ccc(-n3ccn(C)c3=O)c(Cl)c2)c(O)c(-c2cc(N3CCN(C(C)(C)C)CC3)n(C)c(=O)c2)n1] | 6′-(4-(tert- butyl)piperazin-1- yl)-4-(3-chloro-4- (3-methyl-2-oxo- 2,3-dihydro-1H- imidazol-1- yl)phenyl)-3- hydroxy-1′,6- dimethyl-[2,4′- bipyridin]-2′(1′H)- one | 87.2 | 66.285 |
| I-10 | 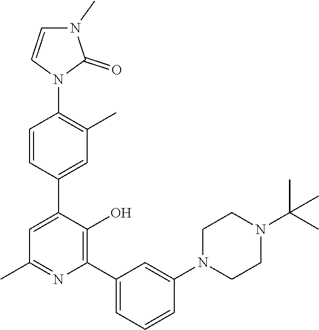 [SMILES: Cc1cc(-c2ccc(-n3ccn(C)c3=O)c(C)c2)c(O)c(-c2cccc(N3CCN(C(C)(C)C)CC3)c2)n1] | 1-(4-(2-(3-(4-(tert- butyl)piperazin-1- yl)phenyl)-3- hydroxy-6- methylpyridin-4- yl)-2- methylphenyl)-3- methyl-1,3- dihydro-2H- imidazol-2-one | 7.59 | 1.464 |
| I-11 | 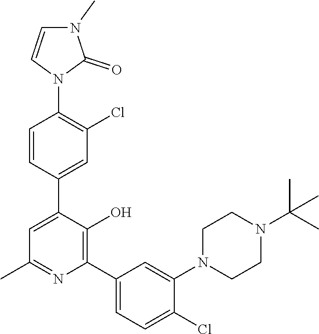 [SMILES: Cc1cc(-c2ccc(-n3ccn(C)c3=O)c(Cl)c2)c(O)c(-c2ccc(Cl)c(N3CCN(C(C)(C)C)CC3)c2)n1] | 1-(4-(2-(3-(4-(tert- butyl)piperazin-1- yl)-4- chlorophenyl)-3- hydroxy-6- methylpyridin-4- yl)-2- chlorophenyl)-3- methyl-1,3- dihydro-2H- imidazol-2-one | 4.22 | 6.337 |
| I-12 | 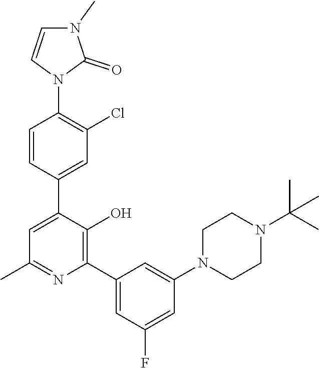 [SMILES: Cc1cc(-c2ccc(-n3ccn(C)c3=O)c(Cl)c2)c(O)c(-c2cc(F)cc(N3CCN(C(C)(C)C)CC3)c2)n1] | 1-(4-(2-(3-(4-(tert- butyl)piperazin-1- yl)-5- fluorophenyl)-3- hydroxy-6- methylpyridin-4- yl)-2- chlorophenyl)-3- methyl-1,3- dihydro-2H- imidazol-2-one | 4.31 | 4.717 |
| I-13 | 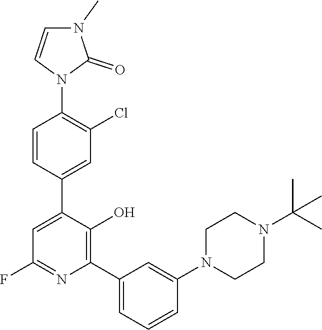 [SMILES: Cn1ccn(-c2ccc(-c3cc(F)nc(-c4cccc(N5CCN(C(C)(C)C)CC5)c4)c3O)cc2Cl)c1=O] | 1-(4-(2-(3-(4-(tert- butyl)piperazin-1- yl)phenyl)-6- fluoro-3- hydroxypyridin-4- yl)-2- chlorophenyl)-3- methyl-1,3- dihydro-2H- imidazol-2-one | 83.6 | 16.715 |
| I-14 | 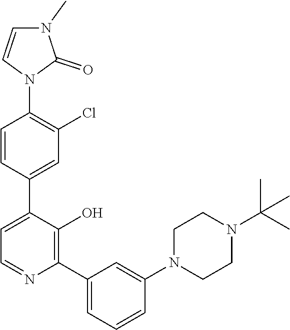 [SMILES: Cn1ccn(-c2ccc(-c3ccnc(-c4cccc(N5CCN(C(C)(C)C)CC5)c4)c3O)cc2Cl)c1=O] | 1-(4-(2-(3-(4-(tert- butyl)piperazin-1- yl)phenyl)-3- hydroxypyridin-4- yl)-2- chlorophenyl)-3- methyl-1,3- dihydro-2H- imidazol-2-one | 68.5 | 21.14 |
| I-15 | 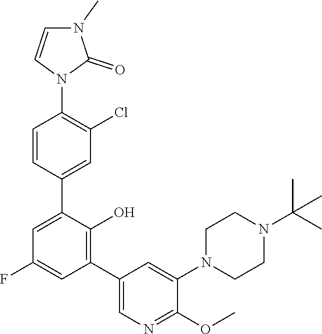 [SMILES: COc1ncc(-c2cc(F)cc(-c3ccc(-n4ccn(C)c4=O)c(Cl)c3)c2O)cc1N1CCN(C(C)(C)C)CC1] | 1-(3′-(5-(4-(tert- butyl)piperazin-1- yl)-6- methoxypyridin-3- yl)-3-chloro-5′- fluoro-2′-hydroxy- [1,1′-biphenyl]-4- yl)-3-methyl-1,3- dihydro-2H- imidazol-2-one | 0.152 | 1.09 |

### Example 17: Anti-Inflammatory Activity of BET-Inhibitors

As discussed in Gilan et al. 2020, BET-inhibitor GSK620 reduced the expression of disease relevant pro-inflammatory genes, including IL-17A, IL17F and IL-22. These pro-inflammatory cytokines are implicated in various autoimmune or inflammatory diseases or disorders.

The compounds of the present invention also reduce the expression of disease relevant pro-inflammatory genes, including IL-17A, IL17F and IL-22. Therefore, the compounds are useful in treating various autoimmune or inflammatory diseases or disorders.

### Example 18: Efficacy Study in IMQ Induced Psoriasis Mouse Model

The Th17 axis plays a major role in many autoimmune and inflammatory conditions (including but not limited to RA, psoriasis, MS, IBD, spondyloarthropathy, vitiligo, atherosclerosis, type I diabetes, systemic lupus erythematosus). Data shown here in the IMQ demonstrates inhibition of the Th17 axis.

### Establishment of Imiquimod-Induced Psoriasis Model in Mice

Except for the control group, all other groups received a daily topical dose of 60 mg of 5% commercially available IMQ cream on the shaved back for 7 consecutive days. The same amount of Vaseline was applied to the same areas of the animals in the negative control group.

### Dosing Procedure

Vehicle and DF-6129 were dosed daily (po) for 8 days, from Day −1 to Day 7. Dosing time: 1 hour prior to IMQ application.

Psoriasis Area and Severity Index (PASI) Scoring The Psoriasis Area and Severity Index (PASI) scoring system was used to evaluate the severity of skin inflammation of the mouse model. Erythema, scaling, and skin thickness was scored independently from 0-4 as (daily scoring) as below:

| Score | Erythema | Scaling | Skin thickness |
|---|---|---|---|
| 0 | None | None | Lesions parallel with the |
| | | | surface of normal skin |
| 1 | Reddish | Some lesions are covered | Slightly elevated lesion |
| | | with scales, mainly fine | than normal skin surface |
| | | scales | |
| 2 | Red | Most lesions are | Moderately elevated with |
| | | completely or partially | plaque margins being round |
| | | covered with flaky | or oblique |
| | | scaling | |
| 3 | Dark red | Almost all lesions are | Lesions with hypertrophied |
| | | covered with thick flaky | skin and prominent |
| | | scales | elevation |
| 4 | Very dark | Almost all lesions are | Lesions with thickness |
| | red | covered with very thick | of skin and very |
| | | flaky scales | prominent elevation |

### Serum for ELISA Analysis

Animals were euthanized 4 h post last dosing on day 7 (3 h after applying with imiquimod cream). Whole Blood was collected via cardiac puncture from all group animals. Serum samples were separated by centrifugation at 4° C. at 8000 g for 5 minutes, total 70 samples. All serum samples were stored at −80° C. for IL-17 analysis.

### Skin for PCR Analysis and HE Staining

Animals were euthanized 4 h post last dosing on day 7 (3 h after applying with imiquimod cream) and back skin was be collected. The skin tissues were divided into two parts. One half was flash frozen and stored at −80° C. for PCR analysis (IL-17A, IL-17F, IL-22 and IL-23), the other half was fixed in 10% formalin for HE staining and scoring.

### Data Statistical Analysis

All statistical analysis were conducted, and the level of significance was set at P<0.05. The group means and standard deviation were calculated for all measurement parameters as study designed. One-way ANOVA comparisons among the groups was performed with software SPSS 16.0.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### REFERENCES

Cochran, A. G. et al. “Bromodomains: a new target class for drug development” Nature Reviews Drug Discovery. Vol. 18, pages 609-628 (2019).
Ghimire, S. et al. “Pathophysiology of GvHD and Other HSCT-Related Major Complications” Front Immunol. 2017; 8: 79.
Gilan, O. et al. “Selective targeting of BD1 and BD2 of the BET proteins in cancer and immuno-inflammation” Science. 38, 6489, 387-394 (2020).
Sun, Y. et al. “BET bromodomain inhibition suppresses graft-versus-host disease after allogeneic bone marrow transplantation in mice” Blood. 2015 Apr. 23; 125(17): 2724-2728.

1. A method of treating an autoimmune or inflammatory disease or disorder in a subject in need thereof, comprising administering to the subject a therapeutically effective amount of a compound, wherein the compound has a structure represented by formula (III) or a pharmaceutically acceptable salt thereof: 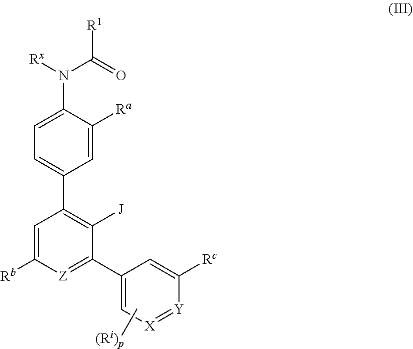
further wherein,
J is —OH, —O(alkyl), —OC(O)(alkyl), —OC(O)O(alkyl), —OC(O)NH(alkyl), —OC(O)N(alkyl)₂, —OCH₂OC(O)O(alkyl), or —NH₂;
X and Y are each independently selected from CH and N provided that at least one of X and Y is CH; or X is C(O) and Y is N(alkyl);
Z is N or CH;
R¹ is alkyl, alkenyl, haloalkyl, —O(alkyl), —S(alkyl), —NH(alkyl), or —N(alkyl)₂;
R^{x} represents H, alkyl, or —C(O)alkyl;
or R¹ and R^{x}, taken together with the intervening atoms, form an optionally substituted heterocycloalkyl ring, heterocycloalkenyl ring, or heteroaryl ring;
R^{a} is independently selected from the group consisting of halo, —NH₂, —NH(alkyl), —NH(cycloalkyl), —N(alkyl)₂, alkyl, alkoxy, cycloalkoxy, haloalkoxy, heterocycloalkoxy, cyano, aryloxy, heteroaryloxy, and haloalkyl;
R^{b} is independently selected from the group consisting of halo, alkyl, alkoxyl, cyano, cycloalkyl, aryl, aryloxy, —CO₂(alkyl) and —CO₂H;
R^{c} is heterocycloalkyl, cycloalkyl, alkyl, aryl, heteroaryl, heterocyclyl, alkoxyl, alkynyl, aryloxy, haloalkyl, haloalkoxy, cycloalkoxyl, heterocycloalkoxyl, halo, —S(alkyl), —NH₂, —CO₂H, —CO₂(alkyl), or —NHCO(alkyl);
each Rⁱ is independently halo, oxo, —S(alkyl), cyano, alkyl, haloalkyl, haloalkoxyl, alkoxyl, heterocycloalkyl, heterocyclyl, or cycloalkoxyl;
or R^{c} and an occurrence of Rⁱ, taken together with the intervening atoms, form an aryl, heteroaryl, cycloalkyl, or heterocycloalkyl ring;
or two adjacent occurrences of Rⁱ, taken together with the intervening atoms, form an aryl, heteroaryl, cycloalkyl, or heterocycloalkyl ring; and
p is 0, 1, or 2.

2. The method of claim 1, wherein the autoimmune or inflammatory disease or disorder is cardiovascular inflammation or vascular inflammation.

3. (canceled)

4. The method of claim 1, wherein the autoimmune or inflammatory disease or disorder is at least partially mediated by Th17 cells.

5. The method of claim 1, wherein the autoimmune or inflammatory disease or disorder is arthritis, spondyloarthropathy, multiple sclerosis, psoriasis, lupus, vitiligo, inflammatory bowel disease (IBD), scleroderma, or systemic sclerosis.

6. The method of claim 1, wherein the compound has a structure represented by formula (IIIa) or a pharmaceutically acceptable salt thereof: 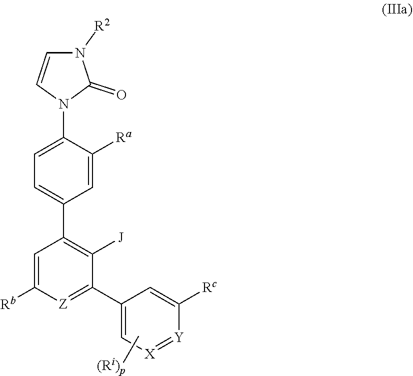
further wherein R² is H, alkyl, alkenyl, haloalkyl, or deuteroalkyl.

7. The method of claim 6, wherein R² is alkyl (e.g., methyl) or deuteroalkyl (e.g., deuteromethyl).

8. (canceled)

9. The method of claim 6, wherein R_{c} is heterocyclyl.

10. The method of claim 6, wherein the compound has a structure represented by formula (IIIb) or a pharmaceutically acceptable salt thereof: 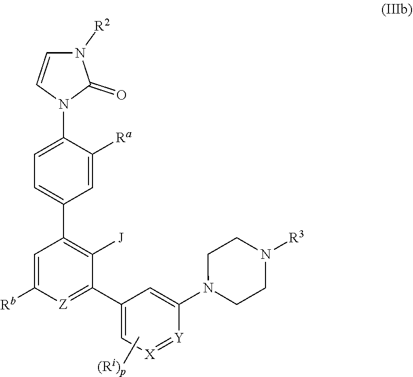
further wherein R³ is H, alkyl, alkenyl, haloalkyl, or deuteroalkyl.

11. The method of claim 10, wherein R³ is alkyl (e.g., tertiary butyl) and/or R^{a} is halo (e.g., chloro or fluoro).

12. (canceled)

13. The method of claim 1, wherein J is —OH or —NH₂.

14. (canceled)

15. The method of claim 1, wherein R^{b} is alkyl (e.g., methyl) or halo (e.g., chloro or fluoro).

16. (canceled)

17. The method of claim 1, wherein one instance of Rⁱ is halo (e.g., chloro or fluoro), one instance of Rⁱ is alkyl (e.g., methyl), one instance of Rⁱ is alkoxyl (e.g., methoxy), or one instance of Rⁱ is oxo.

18. (canceled)

19. (canceled)

20. (canceled)

21. The method of claim 1, wherein X is N and Y is CH: or X is CH and Y is N.

22. (canceled)

23. The method of claim 1, wherein X and Y are each CH: or X is C(O) and Y is N(Me).

24. (canceled)

25. The method of claim 1, wherein p is 0 or 1.

26. (canceled)

27. (canceled)

28. The method of claim 1, wherein Z is N.

29. The method of claim 1, wherein Z is CH.

30. The method of claim 1, wherein the compound has a structure represented by formula (IIIc), formula (IIId), or formula (IIIe), or a pharmaceutically acceptable salt thereof:

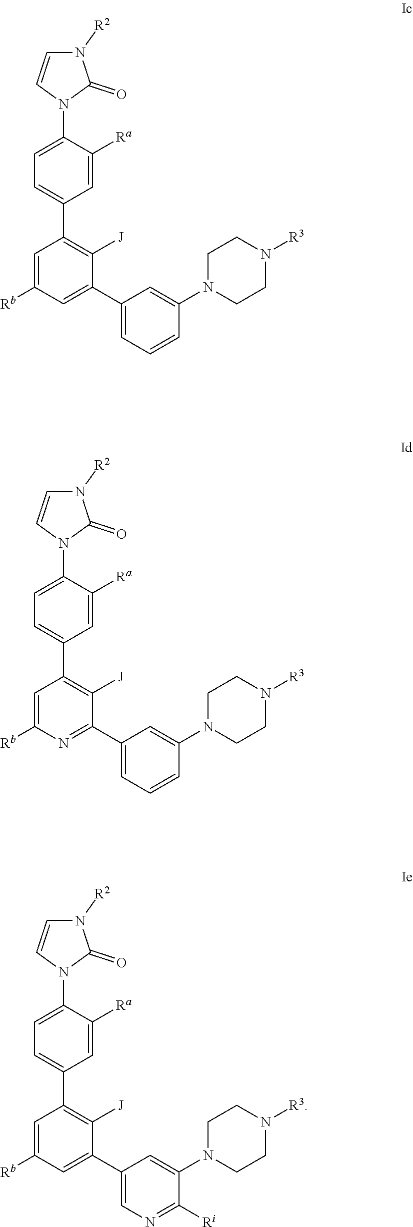

31. (canceled)

32. (canceled)

33. The method of claim 1, wherein the compound is selected from the group consisting of 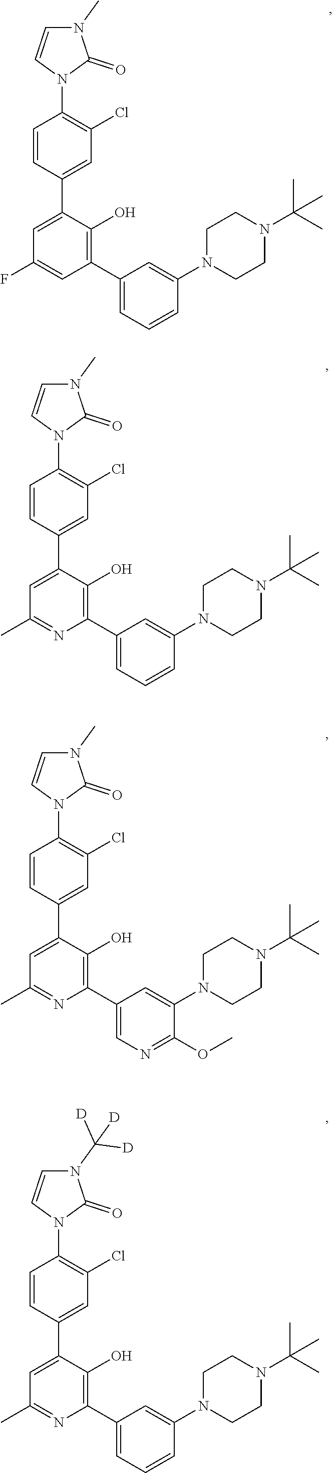 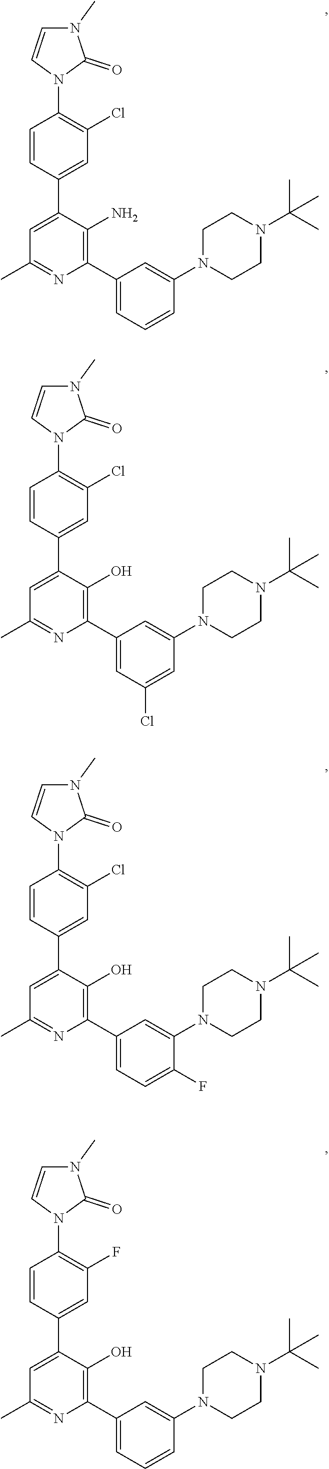 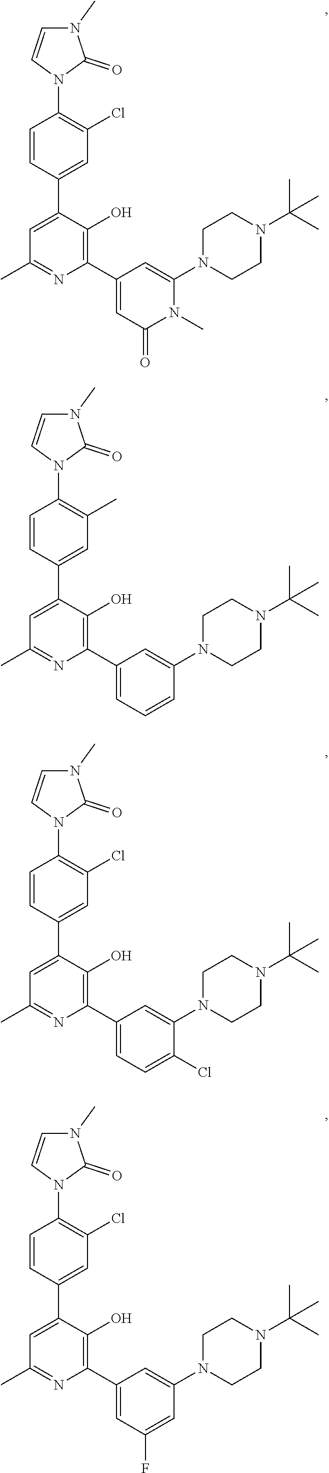 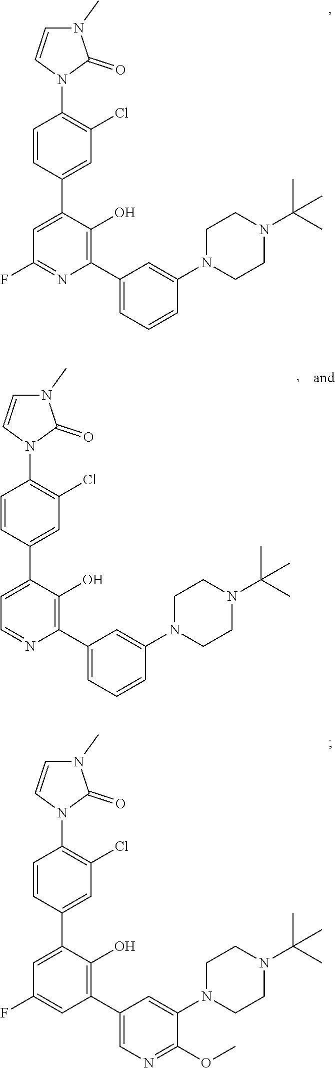
pharmaceutically acceptable salt thereof.

34. The method of claim 1, wherein the compound is administered conjointly with an additional therapy.

35. The method of claim 34, wherein the additional therapy is a JAK inhibitor (e.g., a JAK1 inhibitor or a JAK2 inhibitor).

36. The method of claim 35, wherein the JAK inhibitor is ruxolitinib, fedratinib, pacritinib, nonelotinib, tofacitinib, oclacitinib, baricitinib, peficitinib, upadacitinib, delgocitinib, filgotinib, abrocitinib, or deucravacitinib.

37. The method of claim 34, wherein the additional therapy is a BCL2 inhibitor, a PI3K inhibitor, or is a fusion protein.

38-42. (canceled)
